(19) United States
(12) Patent Application Publication
(10) Pub. No.: US 2003/0198956 A1
(43) Pub. Date: Oct. 23, 2003

(54) Staged assembly of nanostructures

(76) Inventors: Lee Makowski, Hinsdale, IL (US); Paul L. Hyman, Everett, MA (US); Mark K. Williams, Revere, MA (US)

Correspondence Address: PENNIE AND EDMONDS, 1155 AVENUE OF THE AMERICAS, NEW YORK, NY, 100362711

(21) Appl. No.: 10/080,608

(22) Filed: Feb. 21, 2002

(51) Int. Cl. C12Q 1/68; G01N 33/53; B05D 3/00

(52) U.S. Cl. 435/6; 435/7.1; 435/7.5; 427/2.11

(57) ABSTRACT

The present invention provides methods and assembly units for the construction of nanostructures. Assembly of nanostructures proceeds by sequential, non-covalent, vectorial addition of an assembly unit to an initiator or nanostructure intermediate during an assembly cycle, a process termed “staged assembly.” Attachment of each assembly unit is mediated by specific, non-covalent binding of a single pre-determined joining element of one assembly unit to a complementary joining element on a target initiator or nanostructure intermediate. Each interaction of a joining element is designed such that the joining element does not interact with any other joining element of the assembly unit. Self-association of the assembly unit is therefore obviated: only one assembly unit can be added at a time to a target initiator or nanostructure intermediate.

### 1. TECHNICAL FIELD

The present invention relates to methods for the assembly of nanostructures and assembly units for use in the construction of nanostructures.

### 2. BACKGROUND OF THE INVENTION

Nanostructures are structures with individual components having one or more characteristic dimensions in the nanometer range (from about 1-100 nm). The advantages of assembling structures in which components have physical dimensions in the nanometer range have been discussed and speculated upon by scientists for over forty years. The advantages of these structures were first pointed out by Feynman (1959, There's Plenty of Room at the Bottom, An Invitation to Enter a New Field of Physics (lecture), Dec. 29, 1959, American Physical Society, California Institute of Technology, reprinted in Engineering and Science, February 1960, California Institute of Technology, Pasadena, Calif.) and greatly expanded on by Drexler (1986, Engines of Creation, Garden City, N.Y.: Anchor Press/Doubleday). These scientists envisioned enormous utility in the creation of architectures with very small characteristic dimensions. The potential applications of nanotechnology are pervasive and the expected impact on society is huge (e.g., 2000, Nanotechnology Research Directions: IWGN Workshop Report; Vision for Nanotechnology R & D in the Next Decade; eds. M. C. Roco, R. S. Williams and P. Alivisatos, Kluwer Academic Publishers). It is predicted that there will be a vast number of potential applications for nanoscale devices and structures including electronic and photonic components; medical sensors; novel materials; biocompatible devices; nanoelectronics and nanocircuits; and computer technology.

Assembly of nanostructures presents significant problems, however, because their individual components or subunits are very small. Manipulation of individual components necessary in the fabrication of nanostructures, even when possible, is slow and tedious. Manipulation becomes particularly problematic when considering the assembly of complex nanostructures that are made up of a large number of components. Two methods of assembling components from the “bottom up” have been proposed: (i) creation of nanoscale “assemblers” that manipulate and place components individually and (ii) self-assembly of individual components that are designed to interact with one another in only one way to create, through interactions with other components, complete nanostructures.

The term “assemblers” was coined by Drexler to indicate molecular machines capable of translating molecular instructions into three-dimensional structures by analogy to the role of the ribosome in protein synthesis. Development of the initial stages leading to an assembler has proven difficult, and a practical implementation of a working assembler may be overly difficult, if not impossible, due to inherent limitations (Smalley, 2001, Of Chemistry, Love and Nanobots, Scientific American 285(3):76-77). As for self-assembling nanostructures, practical implementations have been thwarted by the need to design components that both have the desired functionalities and exhibit the necessary interactions with neighboring components needed to achieve the self-assembly process.

There is a huge gap between the popular vision of computer nanochips self-assembling by the billions out of a solution of molecular components, and the real, pragmatic problems involved in assembling complex nanodevices. This gap reflects the difference between a crystal and a true device, as diagramed in FIG. 1. Crystal assembly from solution is a result of a huge number of identical subunits interacting in identical ways. Crystal size is not readily controllable, and there is nothing to distinguish unique positions within the crystal. A periodic array of nanoparticles or nanocomponents with spacing in the nanometer range can provide only a very limited number of the potential functions envisioned for nanostructures. Non-periodic nanodevice architectures can provide a much broader range of functions needed for the advancement of nanotechnology.

A nanodevice should be of well-defined size and shape with each position in the device distinguishable from all others. Self-assembly can only be utilized for the synthesis of a nanodevice of a thousand components if the uniqueness of each component position can be encoded through the design and synthesis of a thousand distinct components. Each component is designed to interact tightly, specifically, and uniquely with its neighbors, and to be incapable of interacting with components other than its neighbors; and each harbors a functionality distinct to its position within the device. Such uniqueness of component position places significant constraints on the design of components of nanostructures, and raises problems that have not yet been solved for a real system.

The design and fabrication of many joining pairs that interact with highly specific and non-cross-reacting interactions represents a challenge at least as great as the design of the functional elements themselves. These problems led Whitesides and Love, when they analyzed the advantages and disadvantages of “bottom-up” methods of nanofabrication such as self-assembly, to state that “these methods cannot produce designed, interconnected patterns and are not well suited for building electronic devices” (Whitesides and Love, 2001, The Art of Building Small, Scientific American, 285(3): 39-47). The present invention provides for the assembly of complex nanostructures using a method that circumvents this difficult problem.

Construction of nanostructures in which components are tied together through the interactions of molecules that are biologically programmed for molecular recognition, such as the complementary base-pairing of DNA and RNA (Niemeyer, 2000, Self-assembled nanostructures based on DNA: towards the development of nanobiotechnology, Curr. Opin. Chem. Biol. 4: 609-18) provides one potential solution to these problems. A huge diversity of complementary pairs exist that do not cross-react and that can be synthesized with existing technology. Such programmed molecular building blocks have the drawback, however, that a large number of distinct components must be designed and synthesized to make true self-assembly of a nanostructure possible.

Several approaches currently exist for assembling nanostructures. All these approaches, as discussed below, have their drawback. For example, U.S. Pat. No. 5,864,013 (Goldberg, Materials for the production of nanometer structures and use thereof, issued Jan. 26, 1999), U.S. Pat. No. 5,877,279 (Goldberg, Materials for the production of nanometer structures and use thereof, issued Mar. 2, 1999), PCT WO 96/11947(A1) (Goldberg, Materials for the production of nanometer structures and use thereof, published Apr. 25, 1996), and PCT WO 00/77196(A1), Goldberg, Gene and protein sequences of phage T4 gene 35, published Dec. 21, 2000) disclose that proteins can be used as components of nanostructures that are engineered from constituents of the long tail fibers of T-even bacteriophages. Phage tail fiber proteins exhibit several characteristics that make them attractive for construction of nanocomponents: (i) they are mechanically rigid; (ii) highly resilient physically; (iii) they are very long and thin; (iv) their length can be increased or decreased using standard cloning techniques; (v) they form strong, rigid bonds to one another; (vi) these bonds are highly specific; (vii) additional functional groups or binding sites may be added at points along the rods that do not disrupt the structural rigidity of the rods, using standard directed mutagenesis and cloning techniques or other specific covalent or non-covalent modification procedures.

Nanofabrication based on T-even bacteriophage tail fiber proteins depends on their modular nature, with their terminal binding domains well-defined and separate from their intervening rigid structural elements. This arrangement suggests a general system for building by self-assembly. The ability to exchange the order of the joining members by cutting and splicing the structural elements, while maintaining rigidity of the protein provides the flexibility for rational design of assembly units and for construction based on a controlled self-assembly using a structurally relevant biomaterial. The trimeric nature of phage tail fiber proteins (Cerritelli et al., 1996, Stoichiometry and domainal organization of the long tail-fiber of bacteriophage T4: a hinged viral adhesin, J. Mol. Biol. 260(5): 767-80), however, limits the geometry to which they can be adapted in their use in a self-assembly or staged-assembly process.

U.S. Pat. No. 5,468,851 (Seeman et al., Construction of geometrical objects from polynucleotides, issued Nov. 21, 1995) discloses another approach for assembling nanostructures. It discloses the assembly of geometrical objects from polynucleotides by nucleic acid ligation. It discloses that one, two and three dimensional structures can be synthesized or modified from polynucleotides. A core structure is expanded by cleavage of a loop with a restriction endonuclease. Another polynucleotide is ligated to the sticky ends, so that the recognition site of the restriction enzyme is not reformed. This process is repeated as many times as necessary to synthesize a desired structure. U.S. Pat. No. 5,468,851 also discloses that a geometrical object assembled from a polynucleotide could provide a useful three-dimensional scaffolding upon which enzymatic or antibody binding domains could be linked to provide high density multivalent processing sites to link to and solubilize otherwise insoluble enzymes, or to entrap, protect and deliver a variety of molecular species. The limitation of this approach, however, is that the disclosed nanostructures, made of a single, double-stranded, polynucleotide lattice, lack structural rigidity and are subject to enzymatic, chemical and photo-degradation. Furthermore, the disclosed nanostructures provide only a limited range of spatial geometries.

U.S. Pat. No. 6,072,044 (Seeman et al., Nanoconstructions of geometrical objects and lattices from antiparallel nucleic acid double crossover molecules, issued Jun. 6, 2000) discloses yet another approach for assembling nanostructures. It discloses that two and three-dimensional polynucleic acid structures, such as periodic lattices, can be constructed from an ordered array of antiparallel, double-crossover molecules assembled from single-stranded oligonucleotides or polynucleotides. The construction proceeds by the creation of staggered ends by enzyme cleavage, then ligation to form a linkage. Such antiparallel double-crossover molecules have the structural rigidity necessary to serve as building block components for two- and three-dimensional structures having high translational symmetry associated with crystals. Whereas the patent discloses the assembly of nanostructures, the disclosed method does not accommodate the non-periodic placement of functional moieties within the assembly. And while a regularly repeating nanostructure is disclosed, the nanostructure cannot achieve completely defined positions of functionality within the nanostructure.

PCT publication WO 01/00876 (Mirkin et al., Nanoparticles having oligonucleotides attached thereto and uses therefore, published Jan. 4, 2001) discloses a method of synthesizing nanoparticle-oligonucleotide conjugates. The drawback of this method, however, is that although the nanoparticles are linked together with well-controlled average distances between them, the method cannot provide for controlled geometry or stoichiometry, since the DNA units that provide the specific complementary binding sites are conjugated to inorganic particles with indeterminate stoichiometry and geometry. The particles will assemble to form a nanomaterial with an indeterminate particle packing and therefore, precisely defined, three-dimensional structures cannot result from the disclosed fabrication methods.

U.S. Pat. No. 5,969,106 (Rothstein et al., Self-aligning peptides modeled on human elastin and other fibrous proteins, issued Oct. 19, 1999) discloses designs of synthetic proteins based on several naturally occurring fibrous proteins. These synthetic proteins have multiple domains, including two P-sheet joining domains and an a-helical domain to link the β-sheet domains together. The patent discloses that P-sheet domains of different subunits join together by hydrophobic interactions between interfaces of the subunits, resulting in long polymeric fibers. These fibers are then formed into biocompatible coatings for prostheses. This approach does not appear to allow for forming nanostructures, however, as no method for controlling the assembly process is described that would allow ordering of the components.

PCT publication WO 98/28320 (Heller et al., Affinity based self-assembly systems and devices for photonic and electronic applications, published Jul. 2, 1998) discloses methods for fabricating nanoscale structures using the self-assembling, hybridizing properties of nucleic acids. The publication discloses that a component that has many affinity surface identities is oriented in an electric field, and then reacted with an affinity site. According to the disclosed method, nanostructures are assembled by attaching a first affinity sequence at many locations on a support, and then cross-linked with a functionalized second affinity sequence that reacts with the first sequence and that has an unhybridized overhang sequence. The self-assembling, hybridizing properties of nucleic acids can thus be used to fabricate components for building nanostructures such as octahedron and lattice nanodevices (see FIG. 3B of WO 98/28320). The drawback of such an approach, however, is that since inorganics are used to organize these structures, it would be impossible to control the geometry or stoichiometry of the interactions to produce the disclosed nanostructures.

U.S. Pat. No. 5,712,366 (McGrath et al., Fabrication of nanoscale materials using self-assembling proteins, issued Jan. 27, 1998) discloses a method of fabricating nanoscale structural materials via spontaneous organization of self-assembling proteins. The disclosed self-assembling proteins include at least one recognition sequence, i.e., a charged residue selected from the group consisting of Glu, Lys, Arg and Asp. The disclosed method comprises admixing proteins that include species of the recognition sequence that are prone to dimerization. As disclosed in U.S. Pat. No. 5,712,366, admixed proteins are caused to spontaneously organize into nanoscale structural materials via their respective recognition sequences. U.S. Pat. No. 5,712,366 discloses that in certain embodiments, the amino acid sequences used as structural components are optimized for coiled-coil formation, and designed to mimic leucine zipper protein sequences. Specificity is introduced by controlling the identity and placement of charged residues on the faces of each helix. For example, to construct self-assembling fibers, the genes for polypeptides A2 and B2 are modified by incorporating additional recognition elements at the N- or C-termini. These added elements, which are designed to react with each other and not with polypeptides A2 or B2, impose a driving force for ordered supramolecular assembly, resulting in alignment of all of the dimers in a “head-to-tail” orientation within an assembling fibril.

Nevertheless, a major shortcoming of the method disclosed in U.S. Pat. No. 5,712,366 is that the molecular components are designed to spontaneously recognize their nearest neighbors, and these nearest-neighbor interactions can only define a repeating pattern of units. The repeated use of identical interactions among identical units does not provide, however, for the incorporation of special units possessing specific functionalities into specifically defined positions.

U.S. Pat. No. 6,107,038 (Choudhary et al., Method of binding a plurality of chemicals on a substrate by electrophoretic self-assembly, issued Aug. 22, 2000) discloses an electrophoretic technique for moving a plurality of chemicals into distinct zones for immobilization on a solid surface. The technique includes introducing a first electrolyte and a second electrolyte into a channel, and interposing between the first and second electrolytes at least one solution containing a plurality of chemicals. Under a given electric field, the first electrolyte has anions with higher effective mobility than the chemicals and the second electrolyte has anions with lower effective mobility than the chemicals. When an electrical potential is applied across the length of the channel the plurality of chemicals in the solution are moved into spatial zones. The chemicals in the zones can then be bound to the interior surface of the channel. Chemicals so bound to the wall surface can be used as the initiator or anchor to which chemical components can be added in order to build linear structures such as arrays and electrical conducting structures.

The drawback of the method disclosed in U.S. Pat. No. 6,107,038, however, is that in order to carry out the assembly process, the nanocomponents must be physically manipulated by an electric field and introduced as a plurality of components. Construction of two-dimensional arrays and higher-order architectures thus depends on the placement of anchor molecules that are separated on the range of micrometers or more. The method does not provide for precisely controlling the distance between spatial zones or the distance between anchors. To construct higher-order arrays and geometrical architectures in a stoichiometric fashion, the distances between spatial zones and anchor molecules need to be precisely controlled, both spatially and geometrically. This method for depositing chemicals onto surfaces specifically, while controlling their positions electrophoretically, cannot be used for the construction of a three-dimensional nanostructures.

PCT publication WO 00/68248 (Yeates et al., Self assembling proteins, published Jan. 16, 2001) discloses methods of constructing a fusion protein composed of at least two oligomerization domains that are rigidly linked to each other. The disclosed fusion protein is capable of self-assembling with additional fusion proteins to produce a nanostructure such as an open cage, a closed shell, a ball, a molecular sieve, a matrix, or a carrier. The disclosed methods are limited, however, to regular structures, either finite structures with elements defined by point group symmetries, or regularly repeating structures of indeterminate length in one dimension (e.g., fiber), two dimensions (e.g., thin film) or three dimensions (e.g., crystal).

The drawback of the method disclosed in WO 00/68248 is that fusion protein units are assembled into nanostructures by self-assembly and cannot spontaneously recognize where they belong within a larger framework. The units used in the method are designed only to spontaneously recognize their nearest neighbors, and these nearest-neighbor interactions can only define a repeating pattern. As discussed hereinabove, the repeated use of identical interactions among identical units does not provide for the incorporation of special units possessing specific functionalities into specifically defined positions.

U.S. Pat. No. 5,948,897 (Sen et al., Method of binding two or more DNA double helices and products formed, issued Sep. 7, 1999) discloses a nucleic acid complex having double-stranded sections with a domain of guanine nucleotides. The disclosed domain comprises a pair of substantially uninterrupted guanine sequences that bond together. This domain can interact with other similar domains such that two nucleic acid complexes comprising these domains have the ability to bind together to form DNA superstructures. The drawbacks of such a self-assembly method for building a nanostructure, however, are that it proceeds through bonding of domains (e.g., poly-G) of double-stranded DNA to form superstructures, it does not provide for the incorporation of special units possessing specific functionalities into specifically defined positions and it does not provide a diversity of spatial geometries.

PCT publication WO 01/21646 (Woolfson et al., Protein structures and protein fibres, published Mar. 29, 2001) discloses the construction of nanoscale molecular sieves, grids, and scaffolds from peptides. WO 01/21646 disclosed the formation of protein fibers through the design of specific amino acid heptads that form alpha-helical coiled-coil structures. First and second peptide monomer units are mixed and associate via self-assembly to form a protein structure. While the publication discloses construction of longitudinal fibers, the length of the fibers formed is not controllable. Moreover, incorporation of functional moieties into the monomer units, either before or after self-assembly, is not stoichiometric or specific. Oligomerization and multimerization of the monomer units occurs upon mixing of the complementary monomer unit pair (FIG. 4D, WO 01/21646). The publication discloses that upon mixing of the monomer pairs, a number of protein fibrils of various diameters were obtained (see also Pandya et al., 2000, Sticky-end assembly of a designed peptide fiber provides insight into protein fibrillogenesis, Biochemistry 39(30): 8728-34). This suggests that the self-assembly of the heterodimeric fibers does not occur, as expected, as two monomers per building block. The major drawback the method disclosed in WO 01/21646, however, is that fusion proteins are assembled into nanostructures by self-assembly, the formation of which is not readily controllable. As in other self-assembly methods, this method results in the formation of regular repeating structures that lack units at specific or selected positions in the nanostructure.

U.S. Pat. No. 6,248,529 (Connolly et al., Method of chemically assembling nanoscale devices, issued Jul. 19, 2001) discloses the construction of nanoscale devices including electronic circuits that use DNA as a support structure. U.S. Pat. No. 6,248,529 discloses fabrication of manufacturing nanocircuits, such as transistors, diodes, and inductors, utilizing DNA as the starting scaffold and support structure. The disclosed method includes masking a region of nucleic acid with a nucleic acid binding molecule. The nucleic acid binding molecule is specific for a recognition sequence on the DNA starting scaffold and hence “masks” a portion of the DNA. The unmasked portion of the DNA is then coated with a material, such as conducting or semi-conducting material, whereupon removal of the nucleic acid binding molecule reveals an uncoated portion of the DNA. Upon removal of the nucleic acid binding protein, a second coating material can be applied to the uncoated regions of the nucleic acid template to form a nanoscale device, such as a circuit element. The drawback of such an approach, however, is that it cannot be used for placing nanoparticles in arbitrary, designed positions in a three-dimensional nanodevice.

PCT publication WO 01/16155 (Erlanger et al, Antibodies specific for fullerenes, issued March 2001) discloses antibodies for a wide range of fullerenes and a method for preparing electronic or chemical nanoscale devices from single-walled fullerenes or nanotubes. According to the method, an antibody, as well as fullerene, is incorporated into the disclosed nanodevice. A disadvantage of this method, however, is that the flexibility of the incorporated antibody molecule (as opposed to an antibody fragment) would make precise location of the fullerene difficult.

Hence, despite the availability of a number of different methods for the self-assembly of nanostructures, as discussed hereinabove, there is a need in the art for a method that provides for the incorporation of structural and functional units into a nanostructure at selected, specific positions. There is a further need in the art for nanostructures containing such functional and structural elements at selected positions. The present invention provides such a method and such nanostructures.

### 3. SUMMARY OF THE INVENTION

The present invention provides compositions and methods for the staged assembly of nanostructures. According to the methods of the invention, assembly of nanostructures proceeds by sequential, non-covalent, vectorial addition of specific assembly units to an initiator unit or a nanostructure intermediate during an assembly cycle, a process that is referred to herein as “staged assembly.” Attachment of each assembly unit is, by design, mediated by the specific, non-covalent binding of one or more pre-designated joining elements of one assembly unit to a complementary joining element present on the initiator unit or assembly intermediate. To avoid self-polymerization, each assembly unit is designed so that no joining element that is a part of the assembly unit can interact with any other joining element of that same assembly unit. Self-polymerization of the assembly unit is therefore obviated: only one assembly unit can be added to a target joining element on the initiator or nanostructure intermediate during each assembly cycle, and binding of the assembly unit to the target initiator unit or nanostructure intermediate is vectorial. The process is carried out in a massively parallel fashion such that a very large number of identical assemblies are fabricated simultaneously.

One object of the staged assembly method of the invention is to fabricate nanostructures in which: a) each assembly unit occupies a specific, predetermined location in the nanostructure; b) multiple nanostructures are assembled simultaneously; and c) all the nanostructures are identical in architecture and assembly unit order. In a preferred embodiment of the staged assembly method of the invention, an initiator unit is immobilized on a substrate and additional units are added sequentially in a procedure analogous to solid phase polymer synthesis. Only a few distinct unit-unit interactions need to be used, since the size and shape of the nanodevice will be defined by the order in which units are added. The staged assembly method of the invention requires far fewer non-cross-reacting complementary pairs of joining elements than self-assembly or auto-assembly. Since the engineering or identification of complementary and non-cross-reacting pairs of joining elements constitutes a major barrier to the design of assembly units, the use of the staged assembly method of the invention represents a significant improvement over self-assembly for bottom-up assembly of nanostructures. Each position in the nanodevice can be uniquely defined through the process of staged assembly, and units of distinct functionalities can be added at any desired position. This system enables massive parallel manufacture of complex nanodevices, and different complex nanodevices can be further self-assembled into higher order architectures in a hierarchic manner.

In one embodiment, the invention provides a method for staged assembly of a nanostructure comprising:

(a) contacting a surface-bound nanostructure intermediate comprising at least one unbound joining element with a solution comprising an assembly unit comprising a plurality of different joining elements, wherein:

(i) none of the joining elements of said plurality of different joining elements can interact with itself or with another joining element of said plurality,

(ii) a single joining element of said plurality can bind non-covalently to a single unbound joining element of the surface-bound nanostructure intermediate, and

(iii) the joining elements do not consist of or comprise T-even or T-even-like bacteriophage tail fiber proteins or binding fragments thereof;

(b) removing unbound assembly units; and

(c) repeating steps (a) and (b) to form a nanostructure.

The present invention also provides assembly units for use in the staged assembly methods of the invention disclosed herein. Assembly units of the invention may further comprise structural and/or joining elements, as well as, in certain embodiments, one or more functional elements. If an assembly unit comprises a functional element, that functional element may be attached to, or incorporated within, a joining element, or, in certain embodiments, a structural element. Such an assembly unit of the invention, which may comprise a structural element and a plurality of non-identical, non-interacting, joining elements, may be, in certain embodiments, structurally rigid. The assembly unit of the invention has well-defined recognition and binding properties, i.e., joining elements that exhibit specificity, through specific non-covalent interactions, for a complementary joining element.

In another embodiment, the invention provides a nanostructure assembly unit comprising a plurality of different joining elements, wherein:

(a) none of the joining elements of said plurality can interact with itself or with another joining element of said plurality;

(b) a single joining element of said plurality can bind non-covalently to a single unbound joining element of a surface-bound nanostructure intermediate; and

(c) the joining elements do not consist of or comprise T-even or T-even-like bacteriophage tail fiber proteins or binding fragments thereof.

The invention provides structural elements comprising antibodies or binding derivatives or binding fragments thereof, including, but not limited to, structural elements comprising: monoclonal antibodies, multispecific antibodies, Fab or F(ab′)₂ antibody fragments, single-chain antibody fragments (scFvs), bispecific IgG, chimeric IgG or bispecific heterodimeric F(ab′)₂ antibodies, diabodies or multimeric scFv fragments. The invention also provides structural elements comprising bacterial pilin proteins, leucine zipper-type coiled coils, or four-helix bundles.

According to the staged-assembly method of the invention, the order in which assembly units are added is determined by the desired structure and/or activity of the nanostructure. Joining elements are chosen, by design, to permit staged assembly of the desired nanostructure. Since the choice of joining elements is generally independent of the functional elements to be incorporated into the nanostructure, assembly units are designed to comprise joining elements needed to place the assembly units in the proper place within the nanostructure and the functional elements needed to confer the desired function on the nanostructure as a whole.

The invention provides joining elements that exhibit antigen-antibody interactions, including, but not limited to, joining elements comprising: recombinantly engineered antibodies or binding derivatives or binding fragments thereof, molecules that exhibit idiotope/anti-idiotope interactions, or two non-complementary idiotopes. The invention also provides joining elements comprising peptide epitopes, bacterial pilin proteins or binding derivatives or binding fragments thereof, or peptide nucleic acids (PNAs).

In certain embodiments, the invention provides methods for staged assembly of a nanostructure wherein at least one joining element comprises a binding domain of an antibody or a pilin protein or a binding derivative or binding fragment thereof. In another embodiment, the invention provides a method for staged assembly of a nanostructure wherein at least one joining element comprises a peptide nucleic acid (PNA) or binding derivative thereof.

In yet other embodiments, the invention provides a nanostructure assembly unit wherein at least one joining element comprises a binding domain of an antibody or a pilin protein or binding derivative or binding fragment thereof. In another embodiment, the invention provides a nanostructure assembly unit wherein at least one joining element comprises a peptide nucleic acid (PNA) or binding derivative thereof.

In yet another embodiment, the invention provides a nanostructure assembly unit wherein the assembly unit comprises a first structural element that is bound to a second structural element to form a stable complex, and wherein the first structural element is covalently linked to at least one joining element.

Attachment of each assembly unit to an initiator unit or nanostructure intermediate is mediated by formation of a specific, binding-pair interaction between one joining element of the assembly unit and one or more unbound complementary joining element(s) carried by the initiator unit or nanostructure intermediate. Since according to the methods of the invention, at most only one joining element of an assembly unit will associate by specific non-covalent binding interactions to any given joining element of an initiator assembly unit or nanostructure intermediate in each assembly cycle, such addition of the assembly unit to the initiator unit or nanostructure intermediate will occur in a pre-designed, vectorial manner.

The methods of the invention make possible the fabrication of highly complex architectures with only a few distinct, non-cross-reacting joining pairs. These methods permit the precise geometric and spatial positioning of individual components in the nanometer range. The staged-assembly methods of the invention make possible the mass production of multi-dimensional, non-periodic architectures in which organic and inorganic nanocomponents are placed with precision into three-dimensional constructs.

### 3.1 Definitions

Assembly Unit: An assembly unit is an assemblage of atoms and/or molecules comprising structural elements, joining elements and/or functional elements. Preferably, an assembly unit is added to a nanostructure as a single unit through the formation of specific, non-covalent interactions.

Assembly Unit, Initiator: An initiator assembly unit is the first assembly unit incorporated into a nanostructure that is formed by the staged assembly method of the invention. It may be attached, by covalent or non-covalent interactions, to a solid substrate or other matrix as the first step in a staged assembly process. An initiator assembly unit is also known as an “initiator unit.”Bottom-up: Bottom-up assembly of a structure (e.g.,a nanostructure) is formation of the structure through the joining together of substructures using, for example, self-assembly or staged assembly.

Capping Unit: A capping unit is an assembly unit that comprises at most one joining element. Additional assembly units cannot be incorporated into the nanostructure through interactions with the capping unit once the capping unit has been incorporated into the nanostructure.

Cross-reactive: With respect to joining pairs, two joining pairs are said to be cross-reactive if a joining element from one pair can bind with specificity to a joining element from the other pair.

Functional Domain: A functional domain is a functional element comprising an amino acid sequence.

Functional Element: A functional element is a moiety exhibiting any desirable physical, chemical or biological property that may be built into, bound or placed by specific covalent or non-covalent interactions, at well-defined sites in a nanostructure.

Joining Element: A joining element is a portion of an assembly unit that confers binding properties on the unit, including, but not limited to: binding domain, hapten, antigen, peptide, PNA, DNA, RNA, aptamer, polymer or other moiety, or combination thereof, that can interact through specific, non-covalent interactions, with another joining element.

Joining Elements, Complementary: Complementary joining elements are two joining elements that interact with one another through specific, non-covalent interactions.

Joining Elements, Non-Complementary: Non-complementary joining elements are two joining elements that do not specifically interact with one another, nor demonstrate any tendency to specifically interact with one another.

Joining Pair: A joining pair is two complementary joining elements.

Nanocomponent: A nanocomponent is a substructure or portion of a nanostructure.

Nanomaterial: A nanomaterial is a material made up of a crystalline, partially crystalline or non-crystalline assemblage of nanoparticles.

Nanoparticle: A nanoparticle is an assemblage of atoms or molecules, bound together to form a structure with dimensions in the nanometer range (1-1000 nm). The particle may be homogeneous or heterogeneous. Nanoparticles that contain a single crystal domain are also called nanocrystals.

Nanostructure or Nanodevice: A nanostructure or nanodevice is an assemblage of atoms and/or molecules comprising structural, functional and/or joining elements, the elements having at least one characteristic length (dimension) in the nanometer range.

Nanostructure intermediate: A nanostructure intermediate is an intermediate substructure created during the assembly of a nanostructure to which additional assembly units can then be added.

Non-covalent Interaction, Specific: A specific non-covalent interaction is, for example, an interaction that occurs between an assembly unit and a nanostructure intermediate.

PNA: Peptide nucleic acid Self-assembly: Self-assembly is spontaneous organization of components into an ordered structure. Also known as auto-assembly.

Staged Assembly of a Nanostructure: Staged assembly of a nanostructure is a process for the assembly of a nanostructure wherein a series of assembly units are added in a pre-designated order, starting with an initiator unit that is typically immobilized on a solid matrix or substrate. Each step results in the creation of an intermediate substructure, referred to as the nanostructure intermediate, to which additional assembly units can then be added. An assembly step comprises (i) a linking step, wherein an assembly unit is linked to an initiator unit or nanostructure intermediate through the incubation of the matrix or substrate with attached initiator unit or nanostructure intermediate in a solution comprising the next assembly units to be added; and (ii) a removal step, e.g., a washing step, in which excess assembly units are removed from the proximity of the intermediate structure or completed nanostructure. Staged assembly continues by repeating steps (i) and (ii) until all of the assembly units are incorporated into the nanostructure according to the desired design of the nanostructure. Assembly units bind to the initiator unit or nanostructure intermediate through the formation of specific, non-covalent bonds. The joining elements of the assembly units are chosen so that they attach only at pre-designated sites on the nanostructure intermediate. The geometry of the assembly units, the structural elements, and the relative placement of joining elements and functional elements, and the sequence by which assembly units are added to the nanostructure are all designed so that functional units are placed at pre-designated positions relative to one another in the structure, thereby conferring a desired function on the completely assembled nanostructure.

Structural Domain : A structural domain is a structural element comprising a protein sequence.

Structural Element: A structural element is a portion of an assembly unit that provides a structural or geometric linkage between joining elements, thereby providing a geometric linkage between adjoining assembly units. Structural elements provide the structural framework for the nanostructure of which they are a part.

Subassembly: A subassembly is an assemblage of atoms or molecules consisting of multiple assembly units bound together and capable of being added as a whole to an assembly intermediate (e.g., a nanostructure intermediate). In many embodiments of the invention, structural elements also support the functional elements in the assembly unit.

Top-down: Top-down assembly of a structure (e.g.,a nanostructure) is formation of a structure through the processing of a larger initial structure using, for example, lithographic techniques.

### 4. BRIEF DESCRIPTION OF THE FIGURES

FIGS. 1(A-B). A. Diagram of a crystalline array of nanoparticles (i.e., a nanomaterial) in which there are no special positions, and the size and overall geometry of the array is not completely defined. B. Diagram of a nanodevice, an assemblage of nanoparticles or assembly units in which the positioning of each unit is completely defined according to design. The extent of the structure, and the relative positions of all of the functional units, are defined precisely according to the functional requirements of the device.

FIG. 2. Staged assembly of assembly units. In practice, each step in the staged assembly will be carried out in a massively parallel fashion. In step 1, an initiator unit is immobilized on a solid substrate. In the embodiment of the invention illustrated here, the initiator unit has a single joining element. In step 2, a second assembly unit is added. The second unit has two non-complementary joining elements, so that the units will not self-associate in solution. One of the joining elements on the second assembly unit is complementary to the joining element on the initiator unit. Unbound assembly units are washed away between each step (not shown).

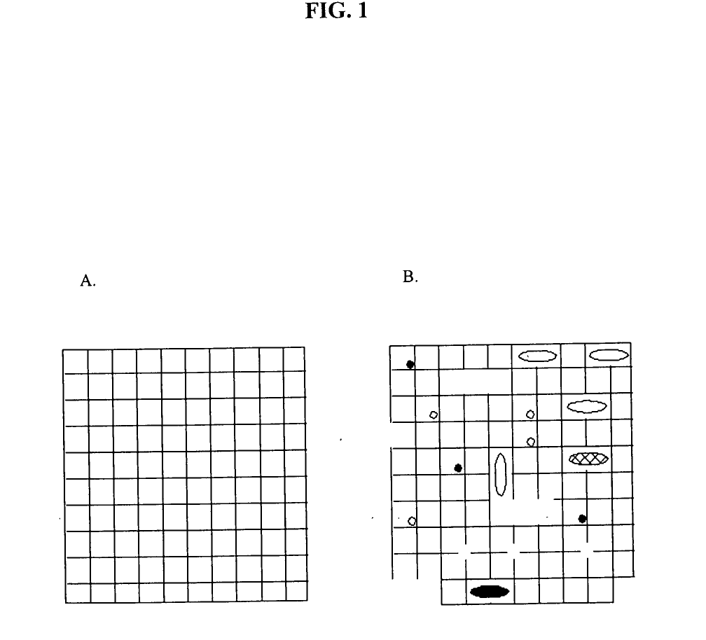

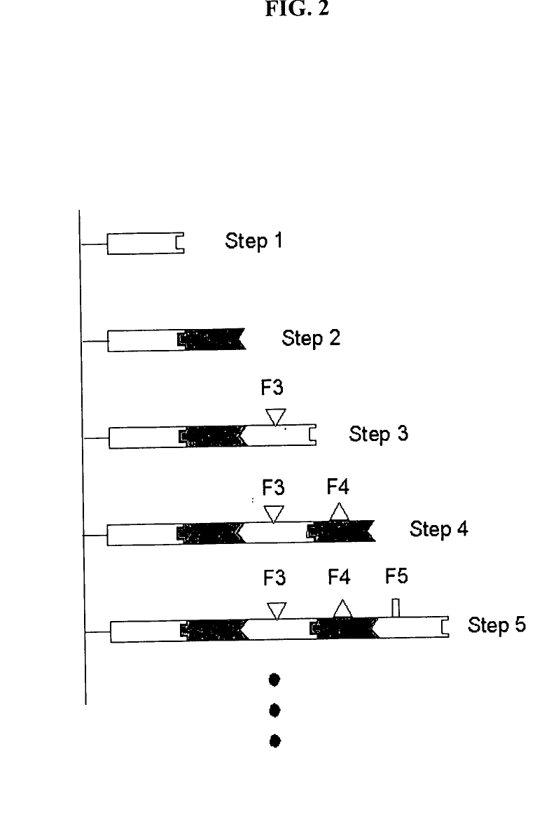

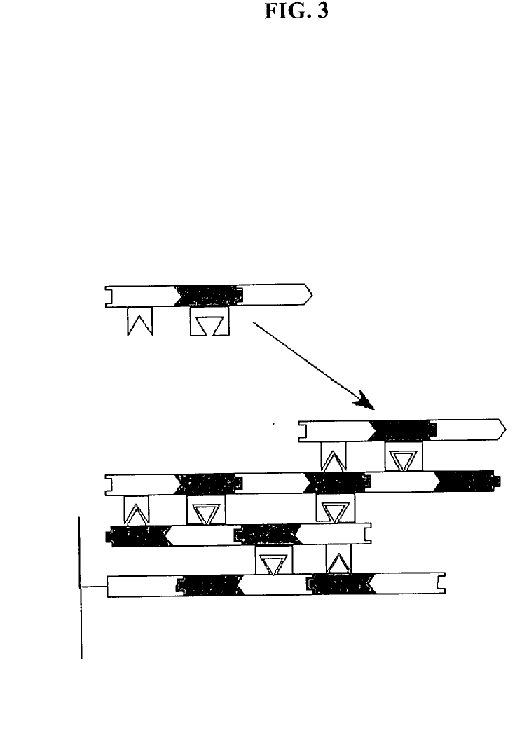

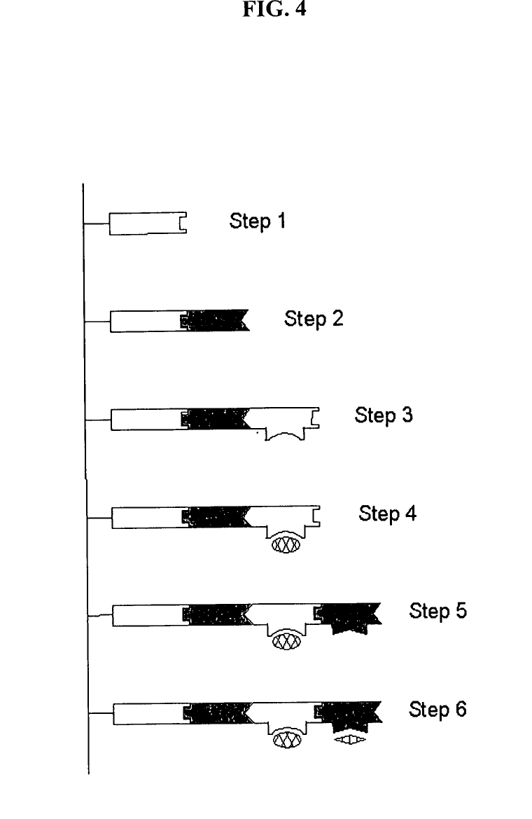

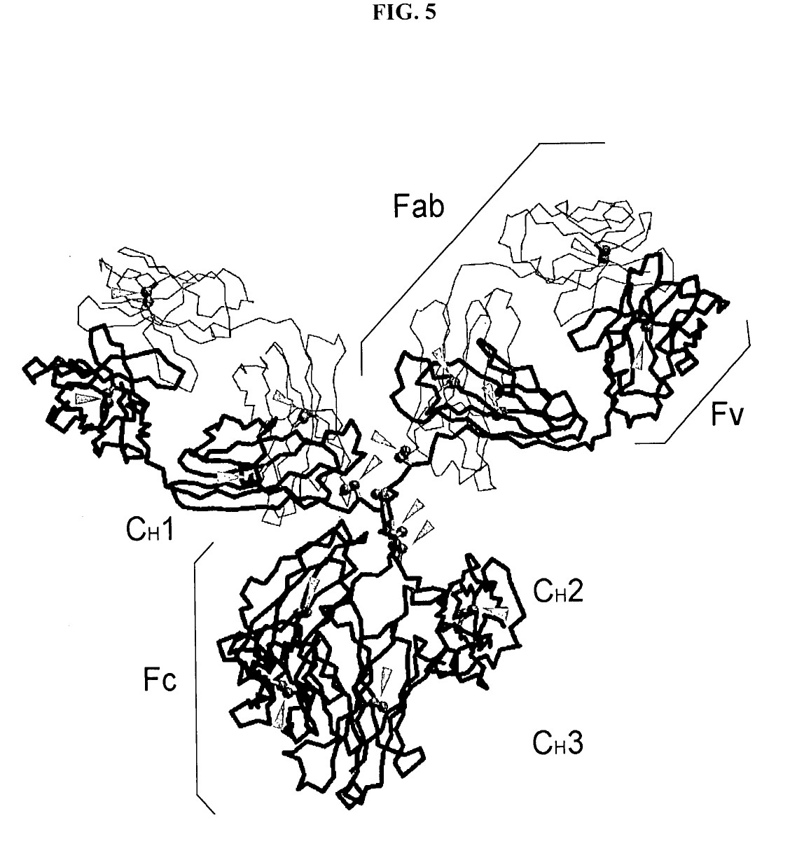

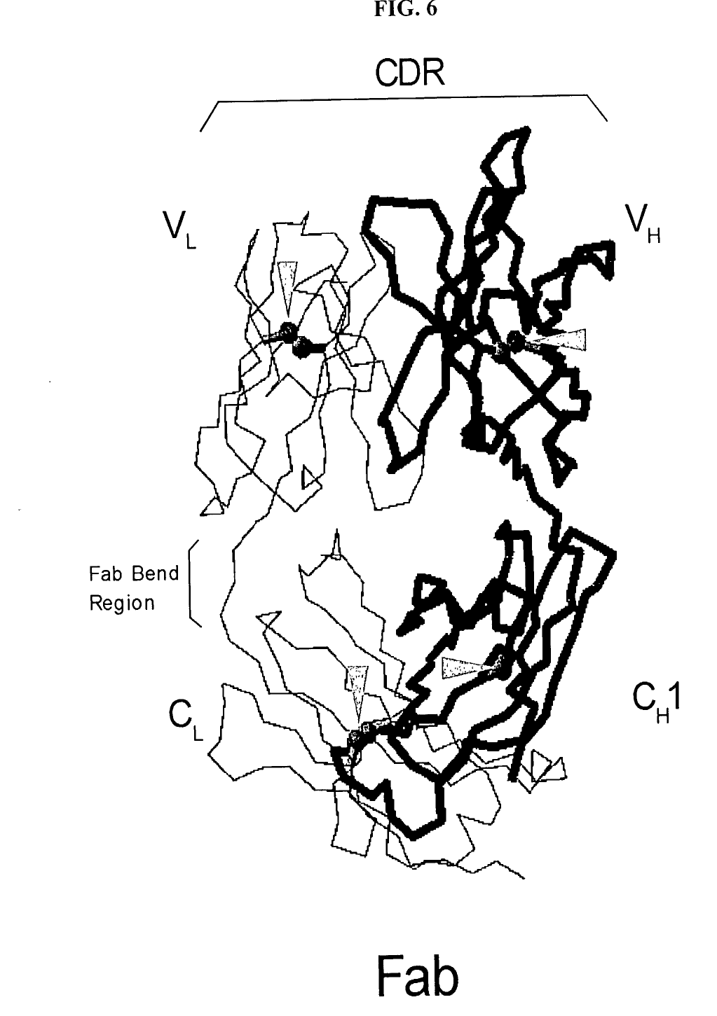

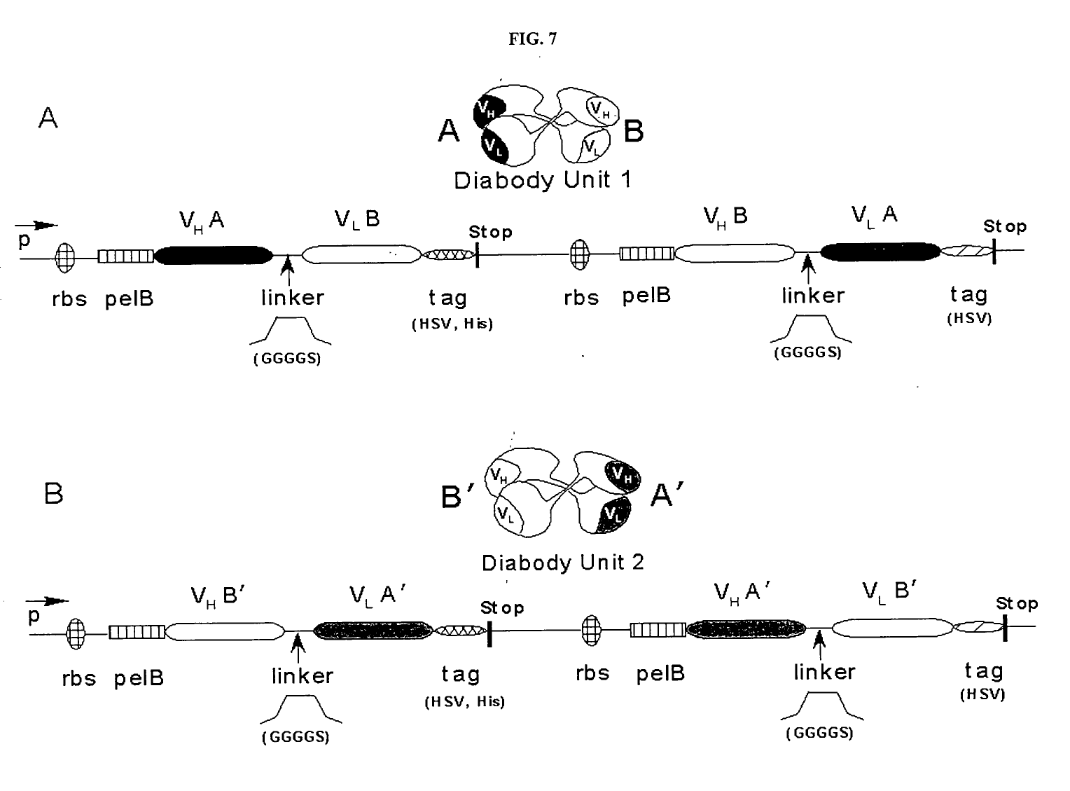

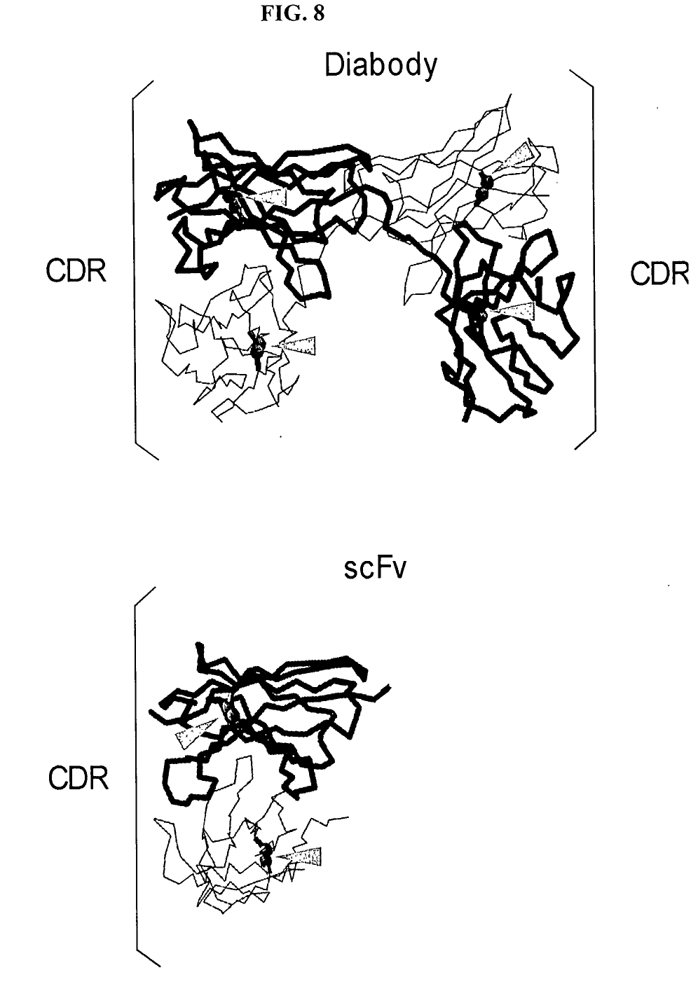

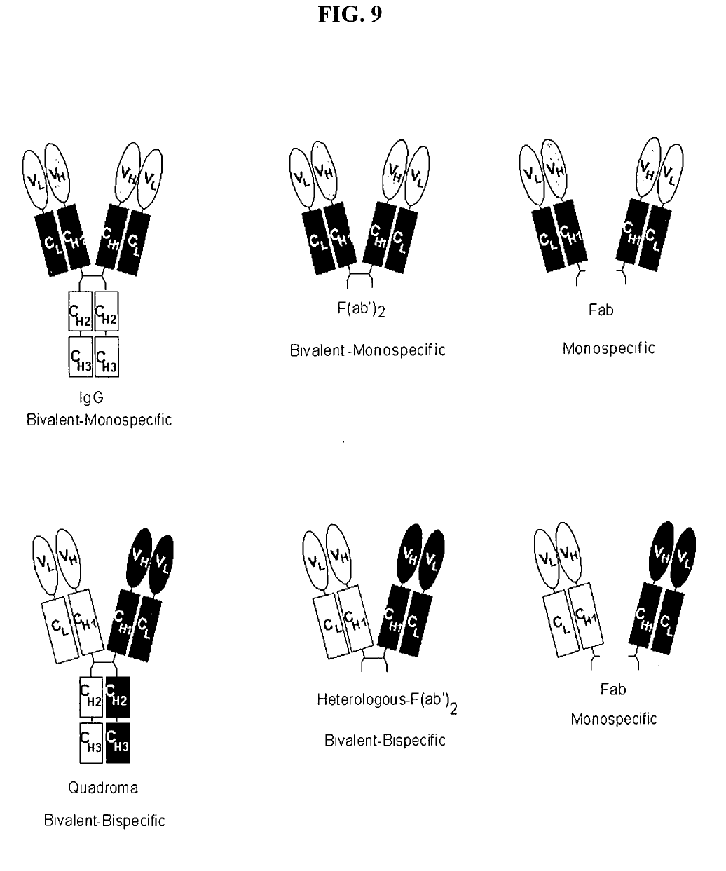

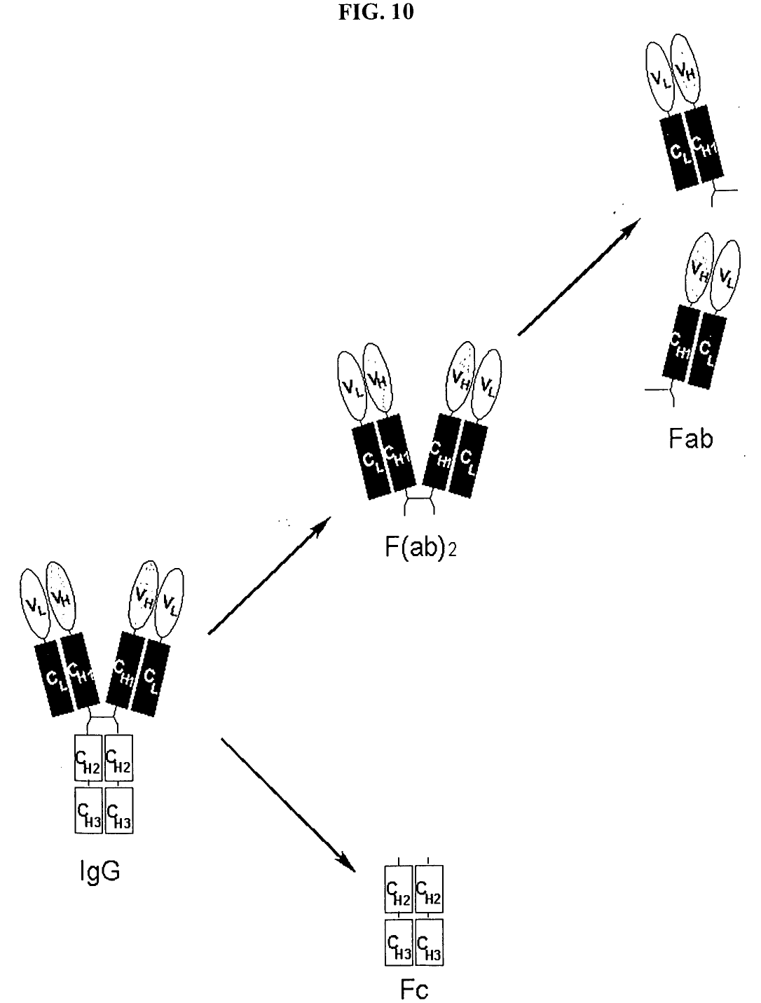

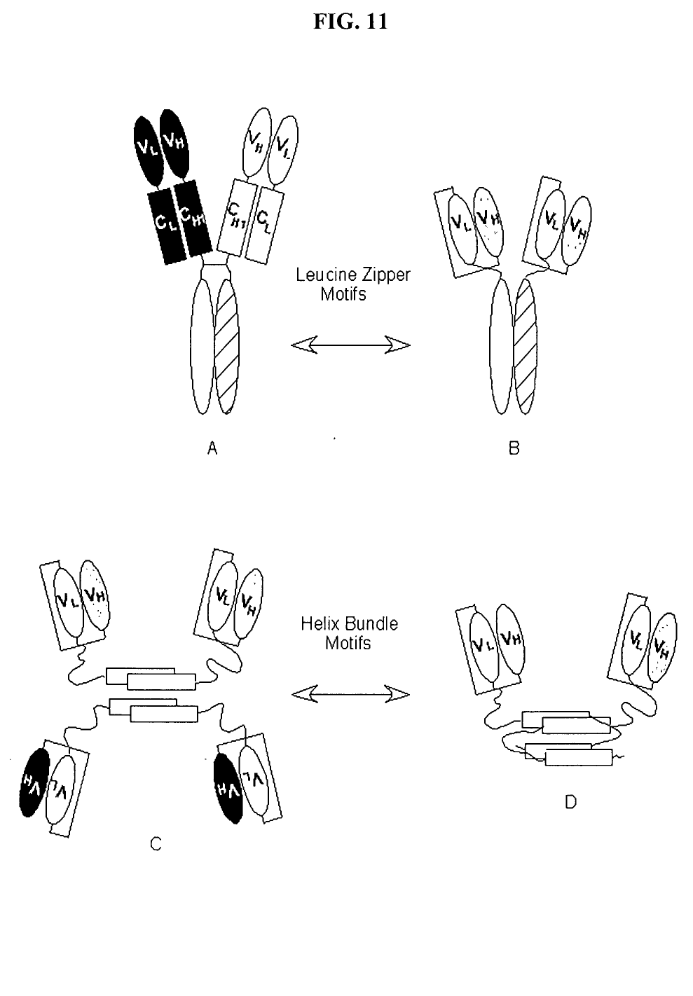

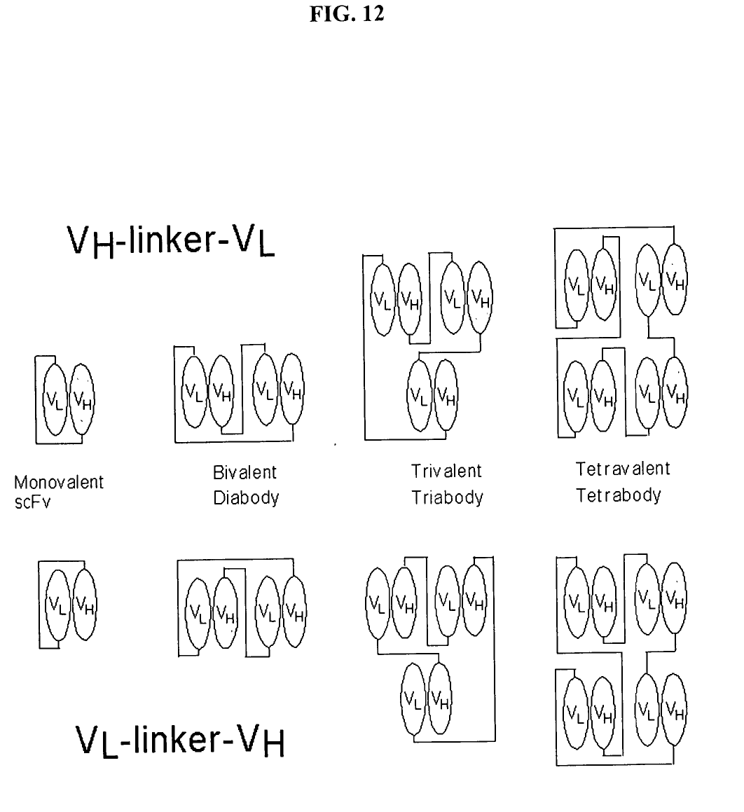

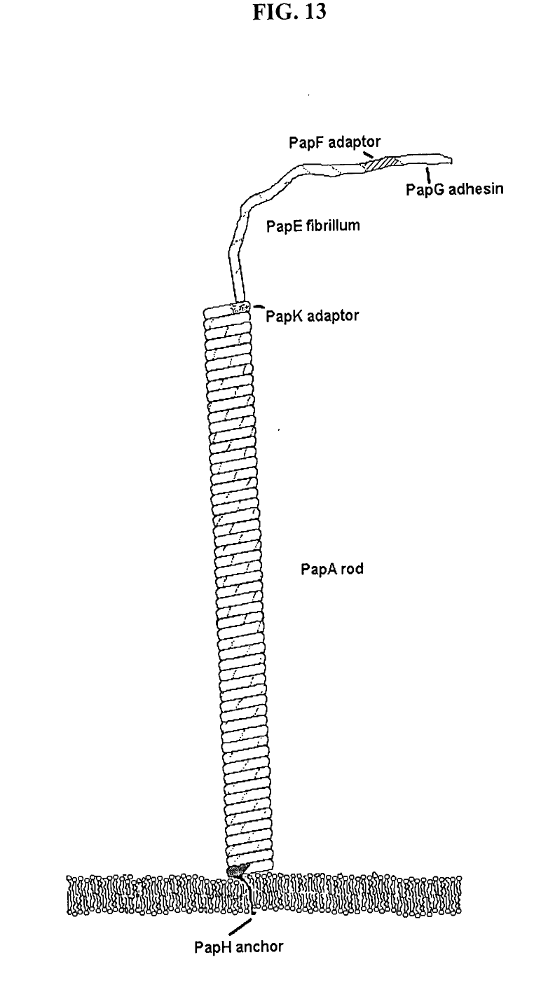

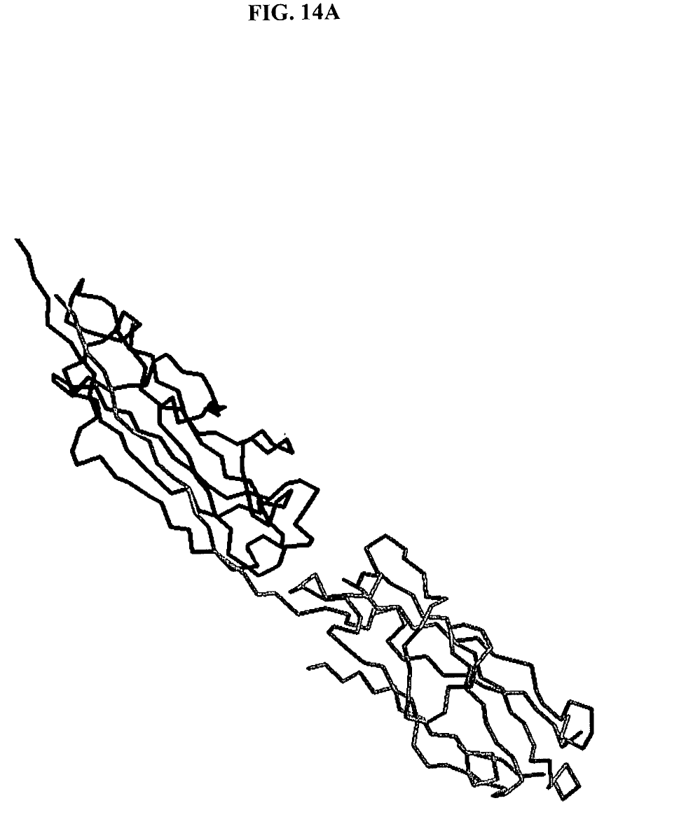

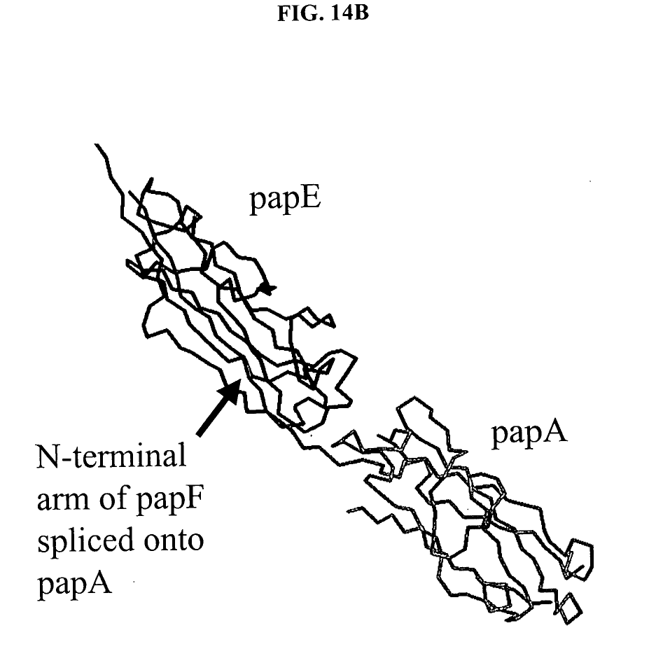

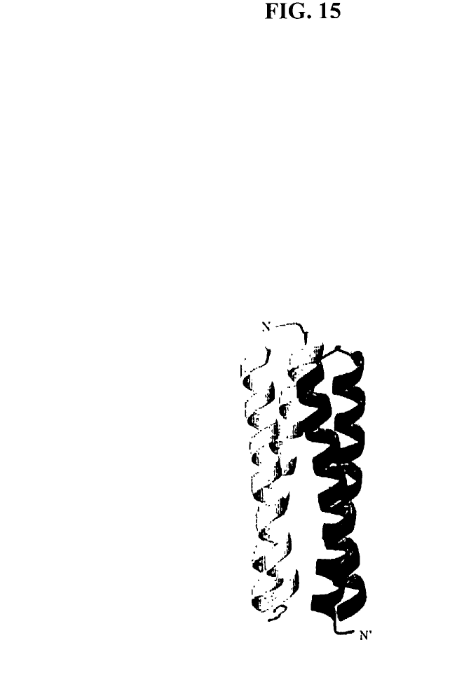

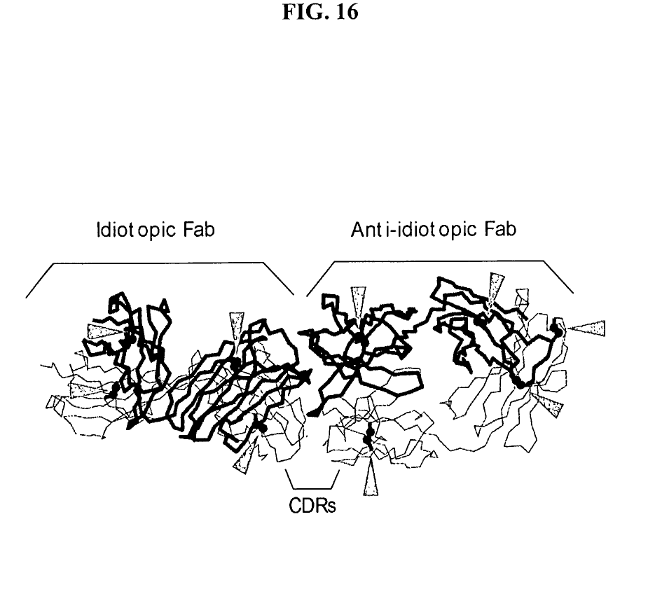

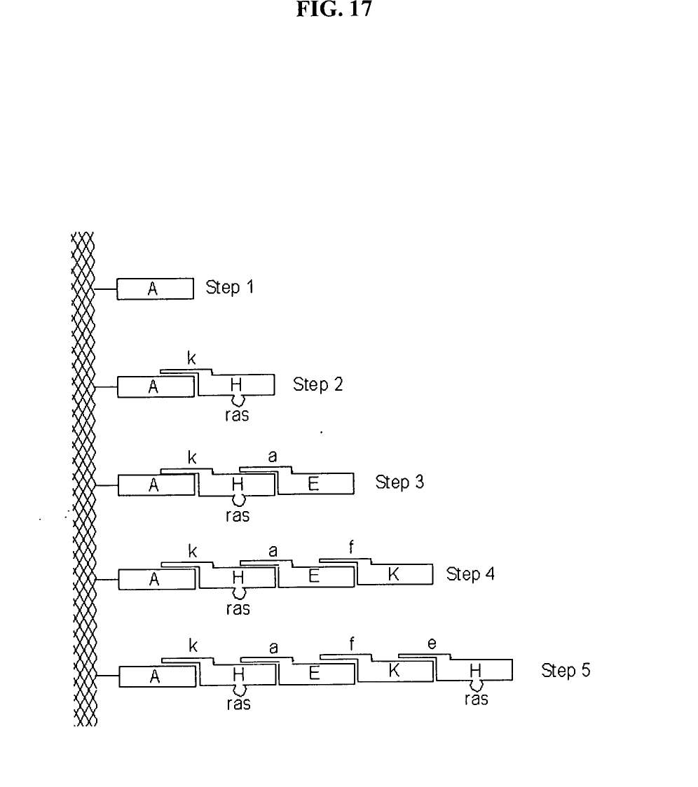

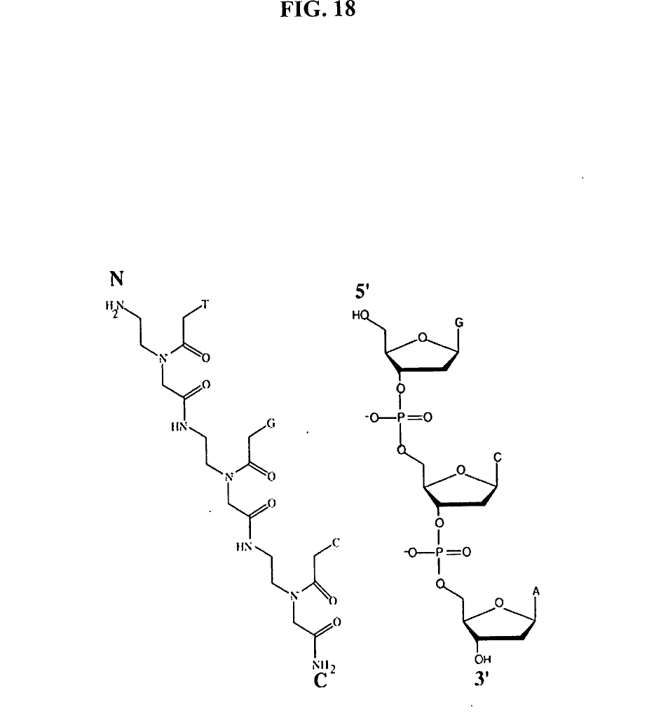

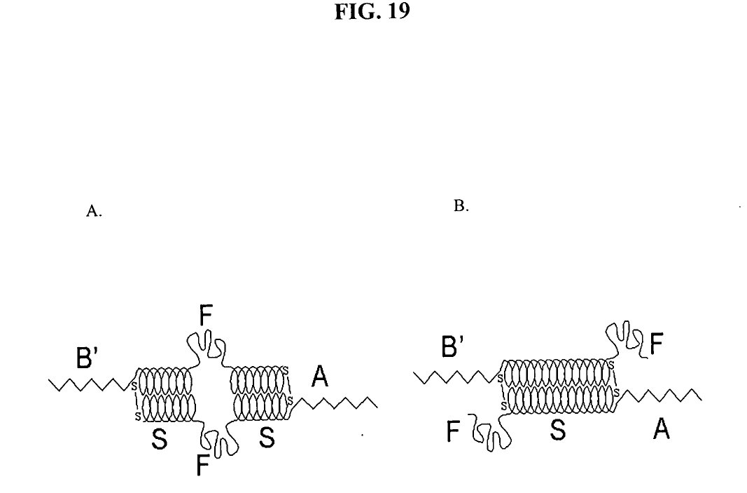

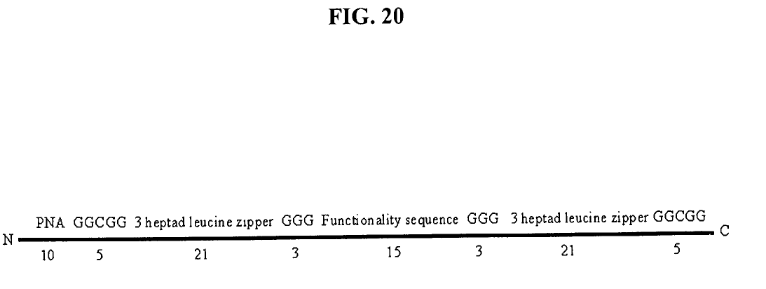

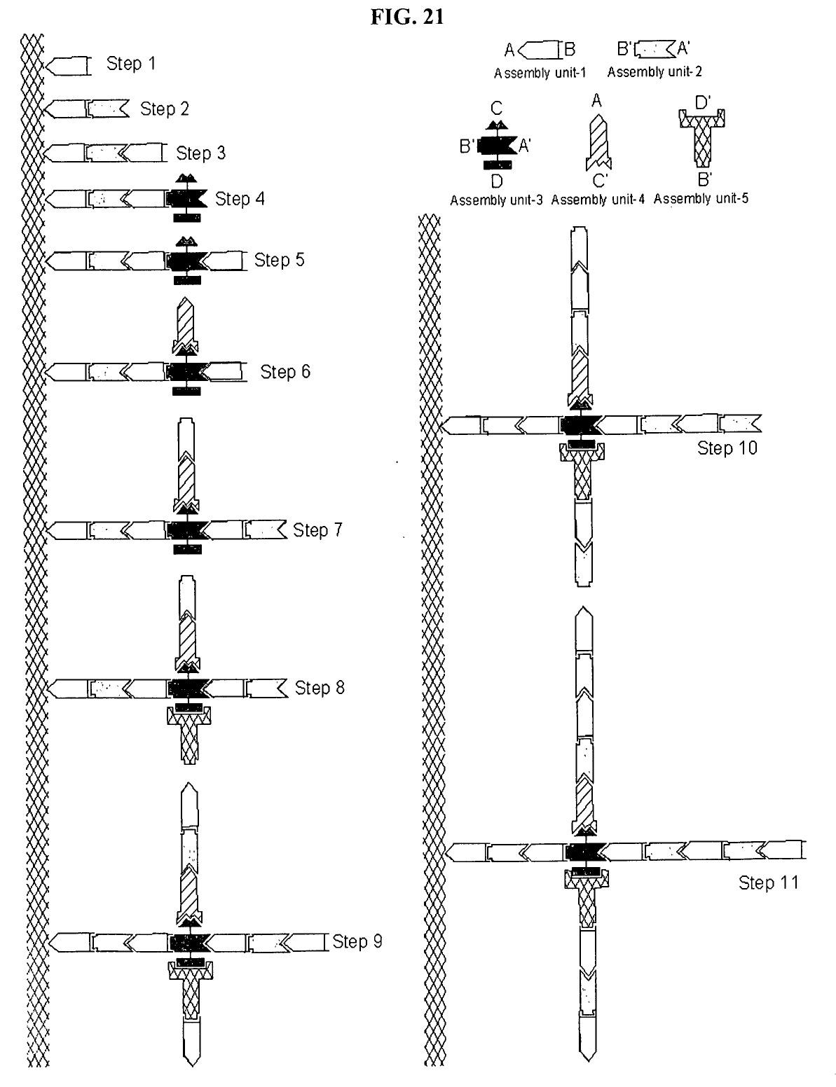

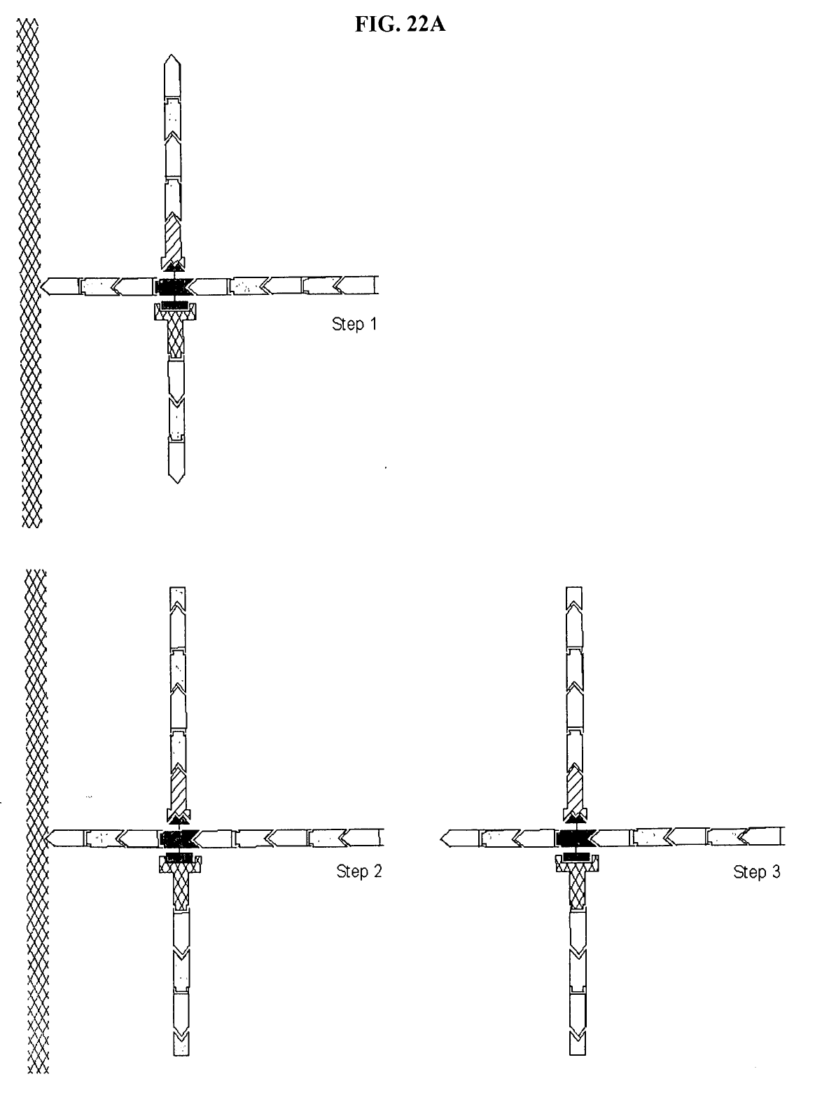

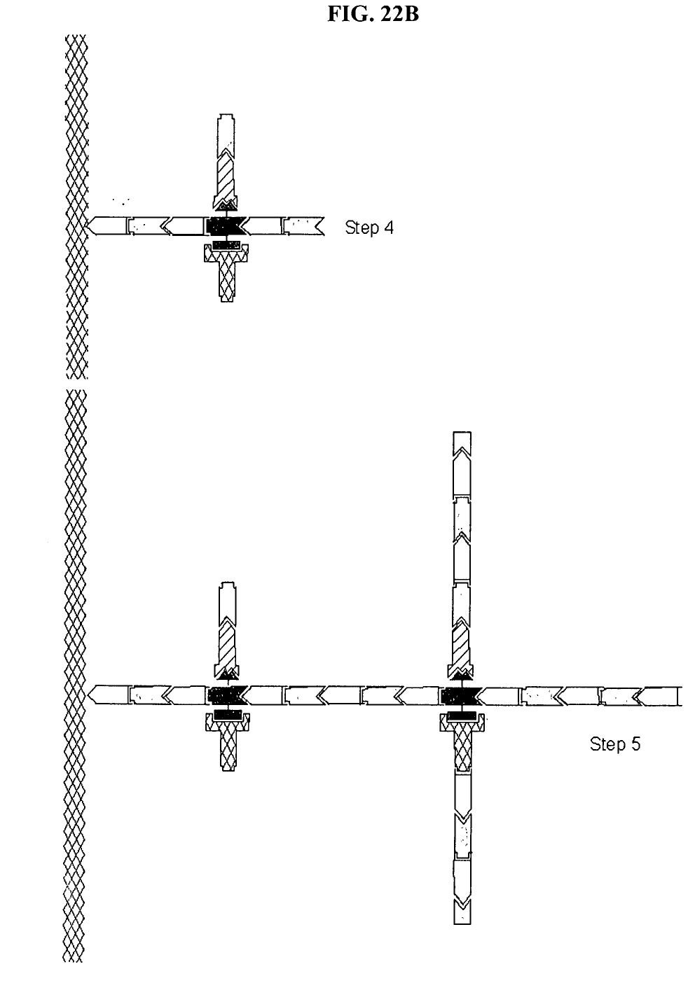

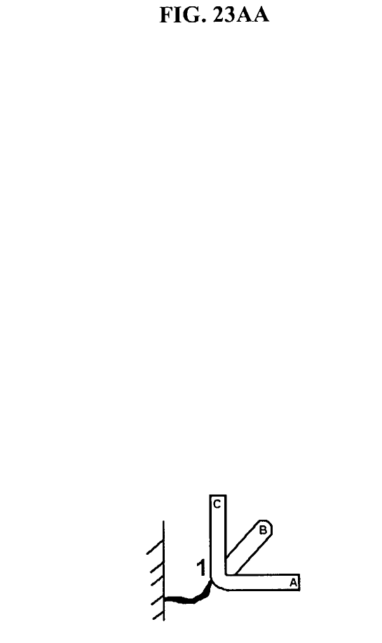

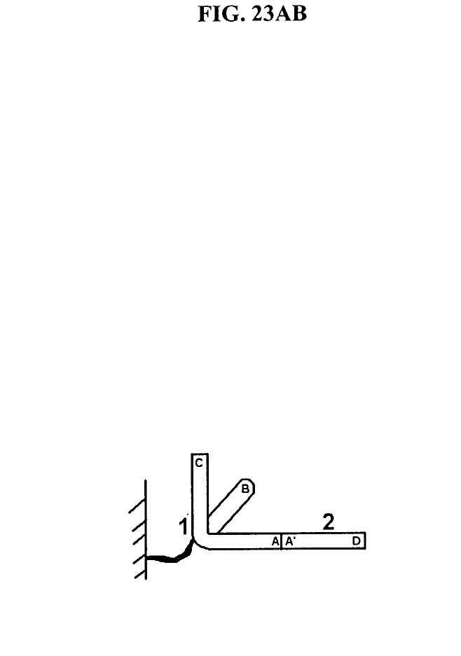

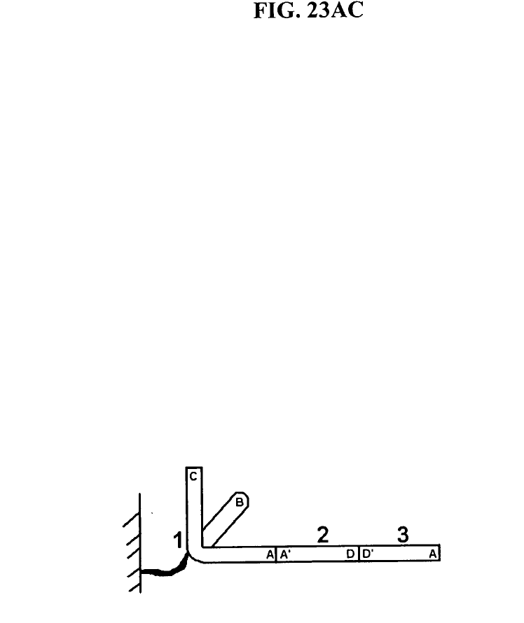

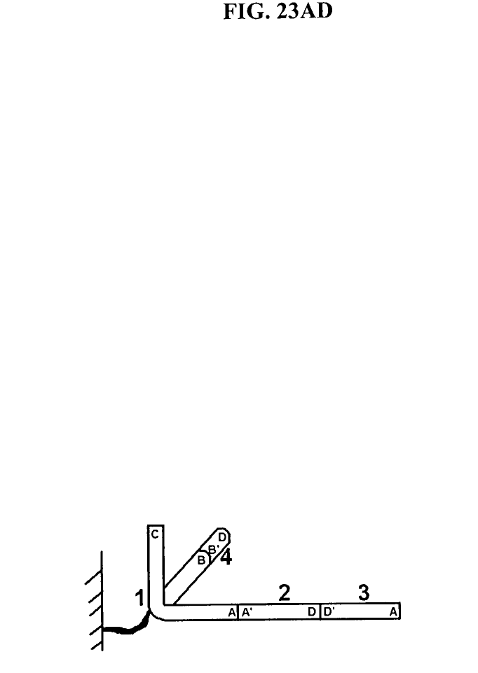

After incubation, the second assembly unit binds to the initiator unit, resulting in the formation of a nanostructure intermediate made up of two assembly units. In step 3, a third assembly unit is added. This unit has two non-complementary joining elements, one of which is complementary to the only unpaired joining element on the nanostructure intermediate. This unit also has a functional unit (“F3”).

A fourth assembly unit with functional element “F4” and a fifth assembly unit with functional element “F5” are added in steps 4 and 5, respectively, in a manner exactly analogous to steps 2 and 3. In each case, the choice of joining elements prevents more than one unit from being added at a time, and leads to a tightly controlled assembly of functional units in pre-designated positions.

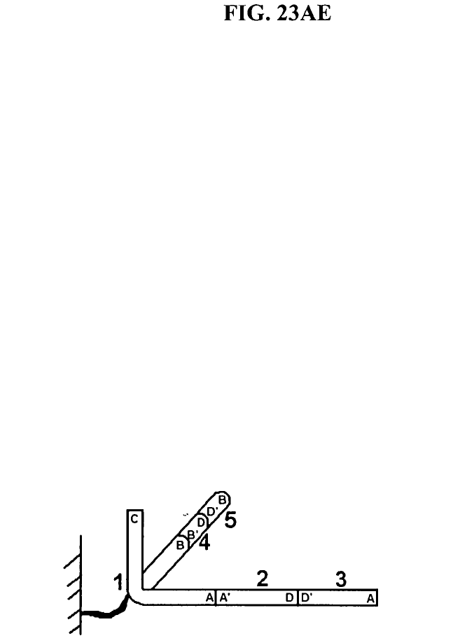

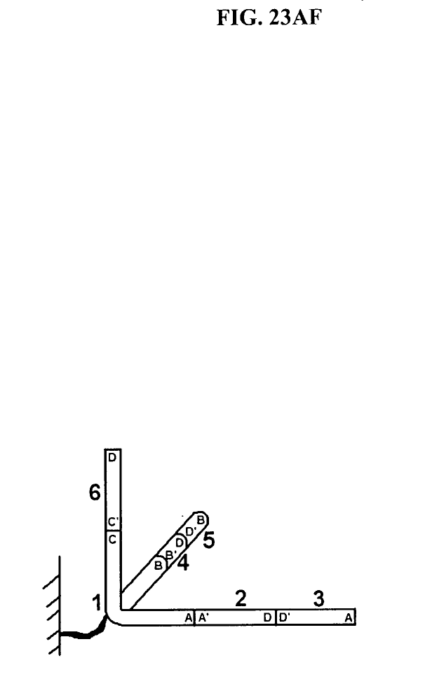

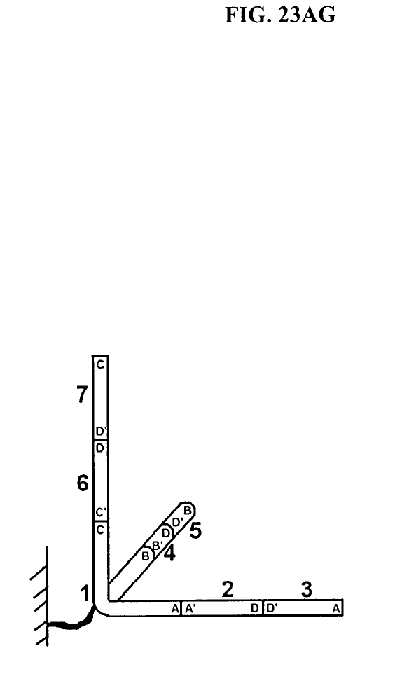

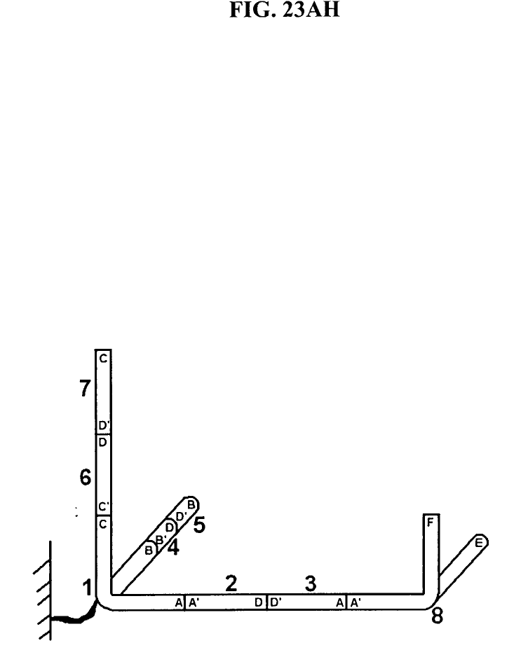

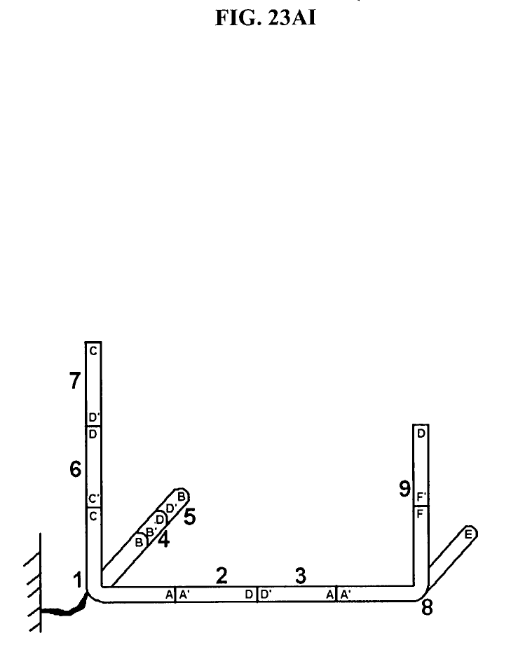

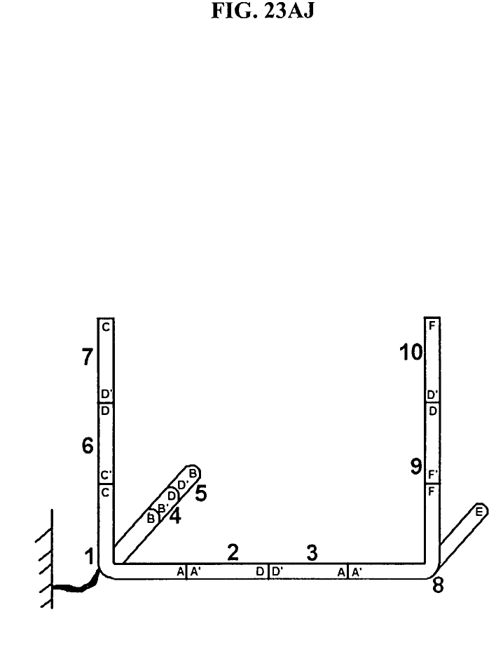

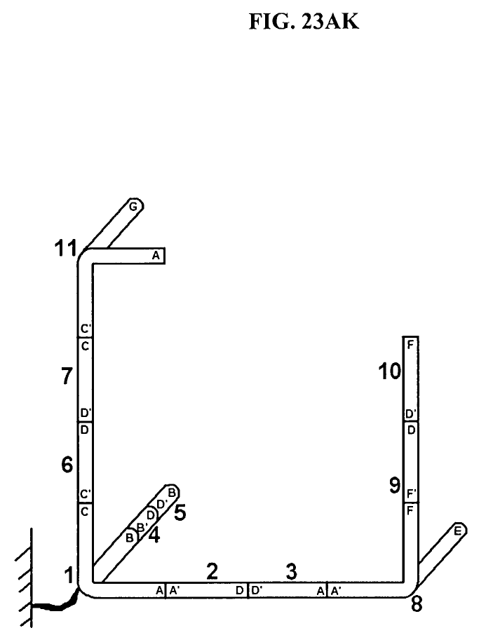

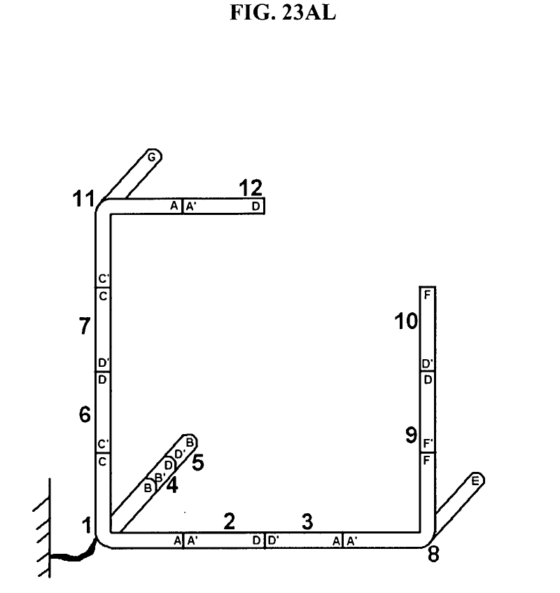

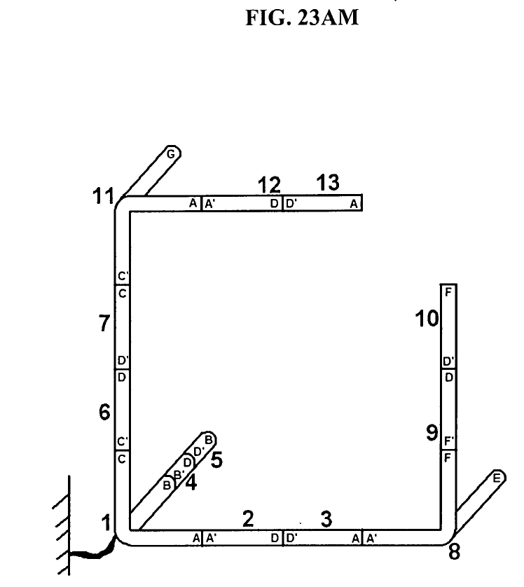

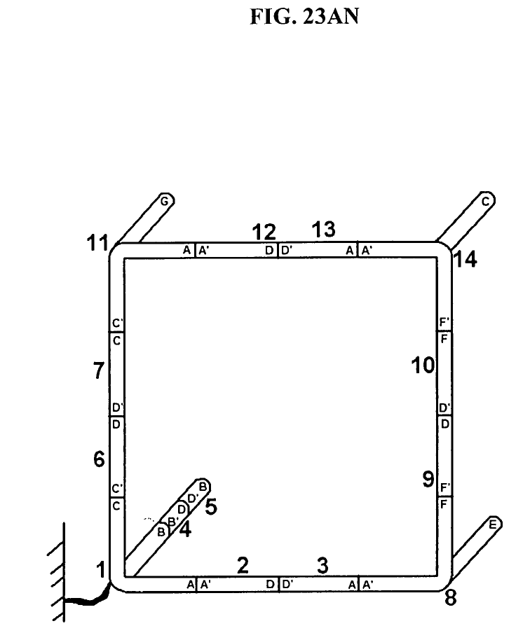

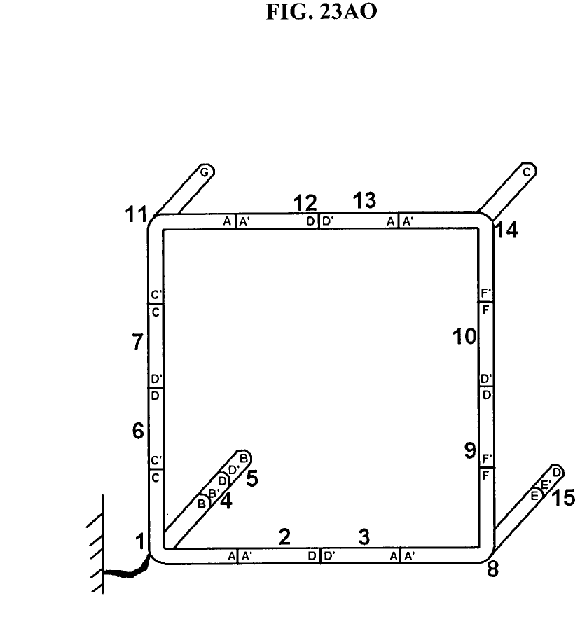

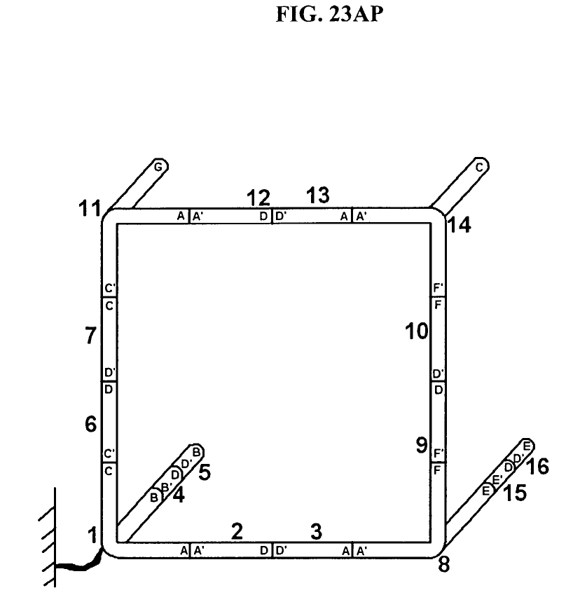

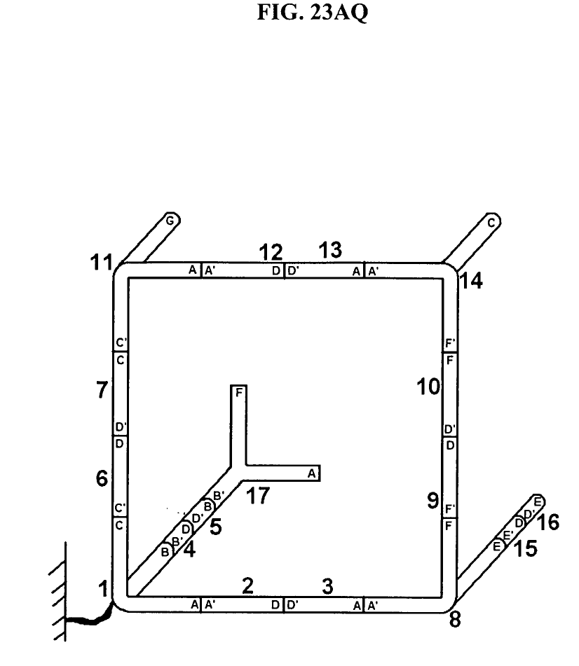

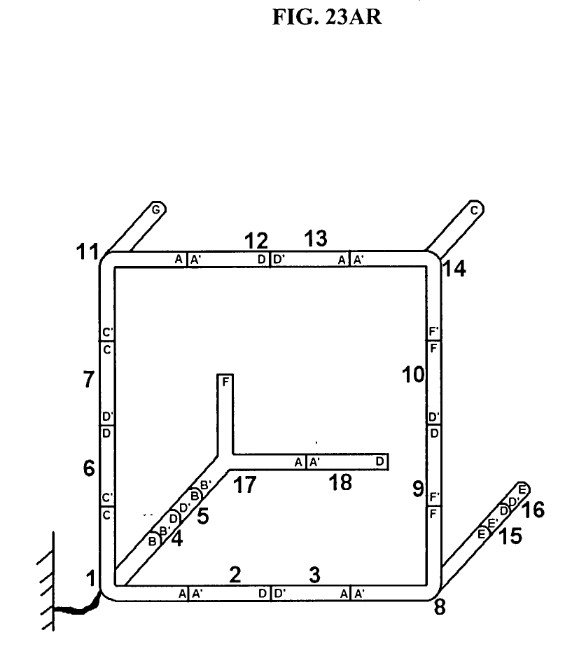

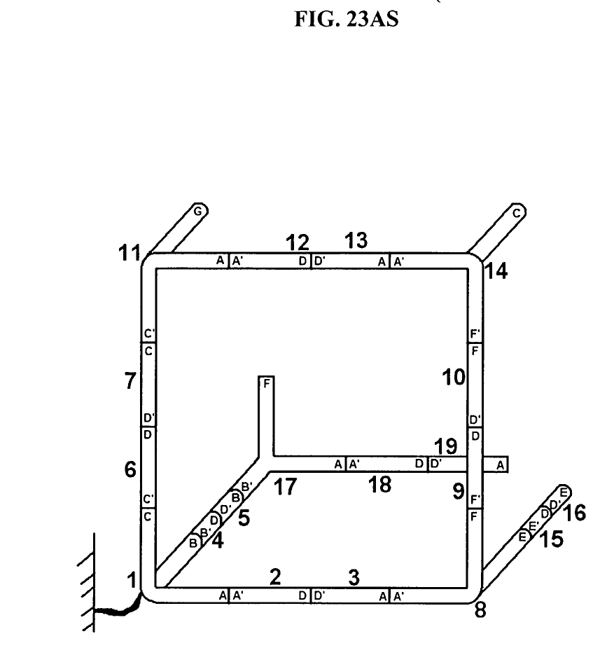

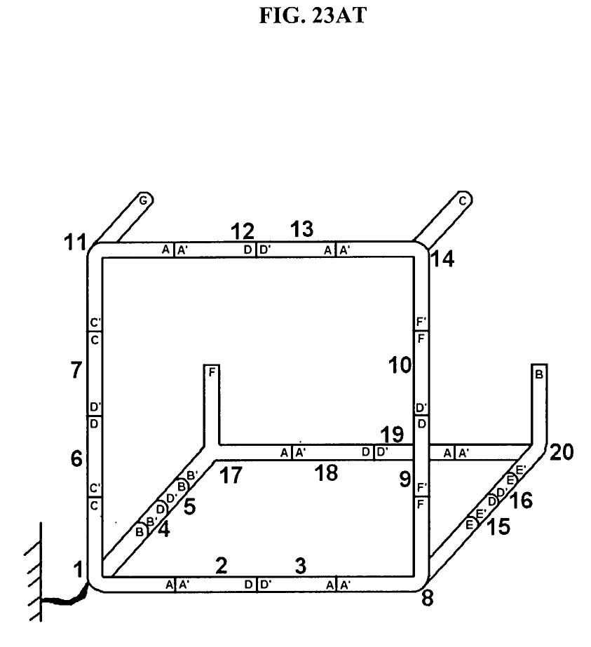

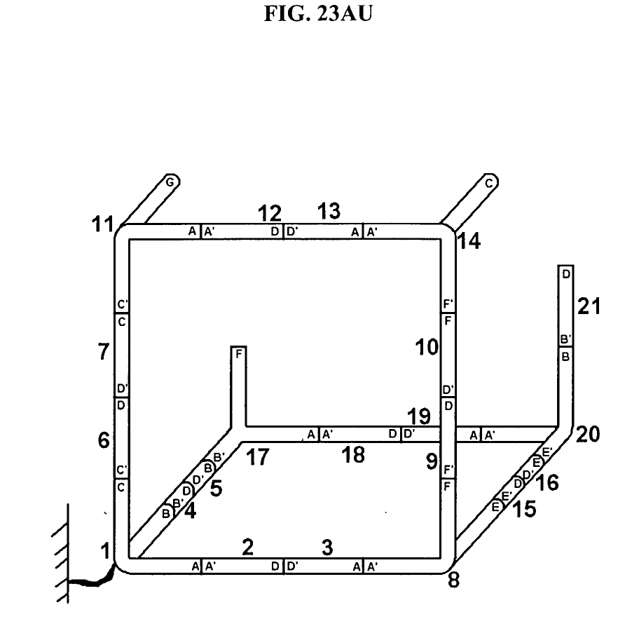

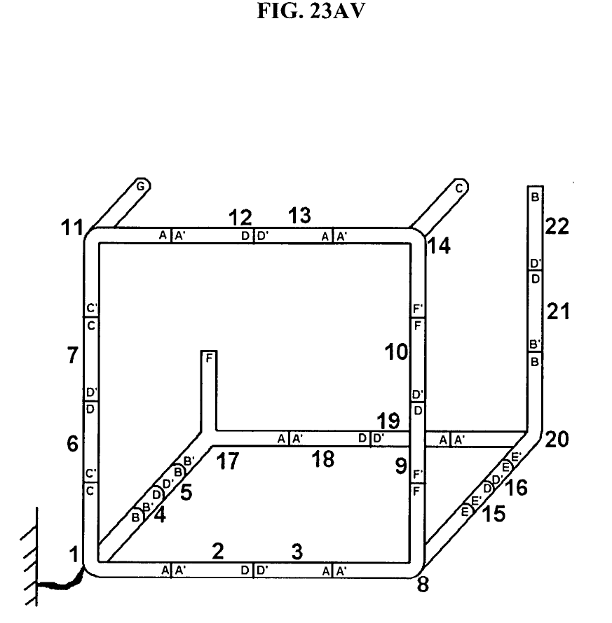

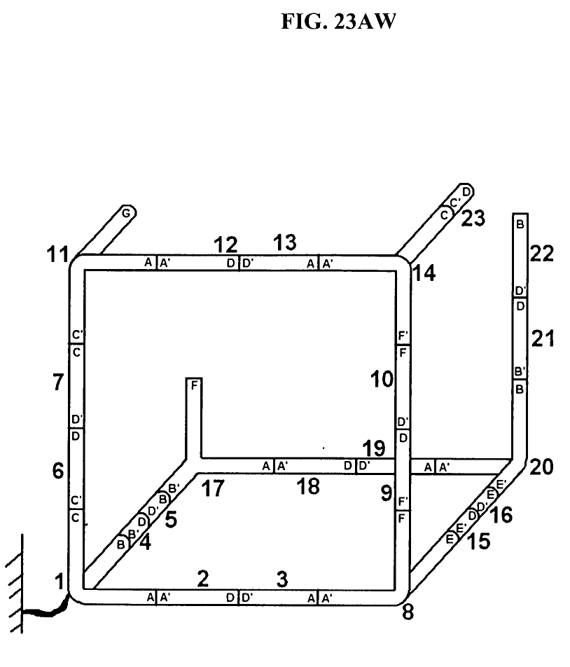

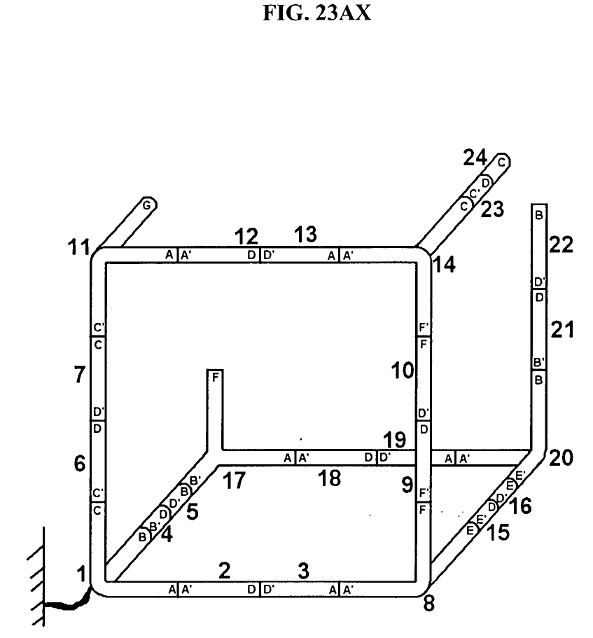

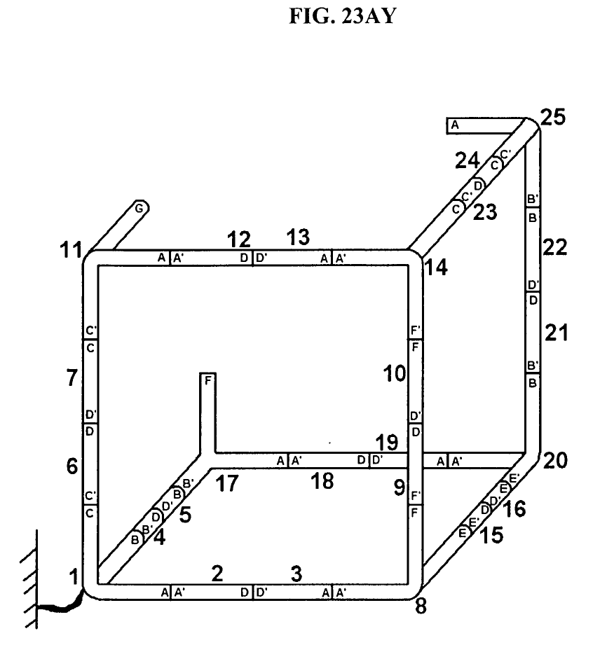

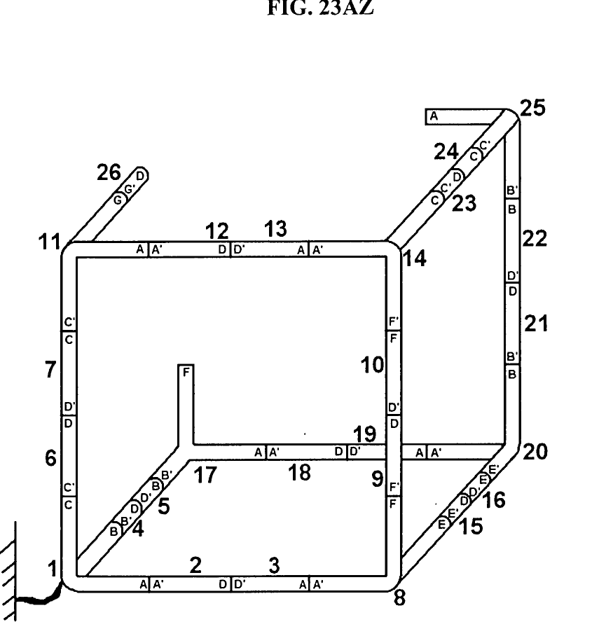

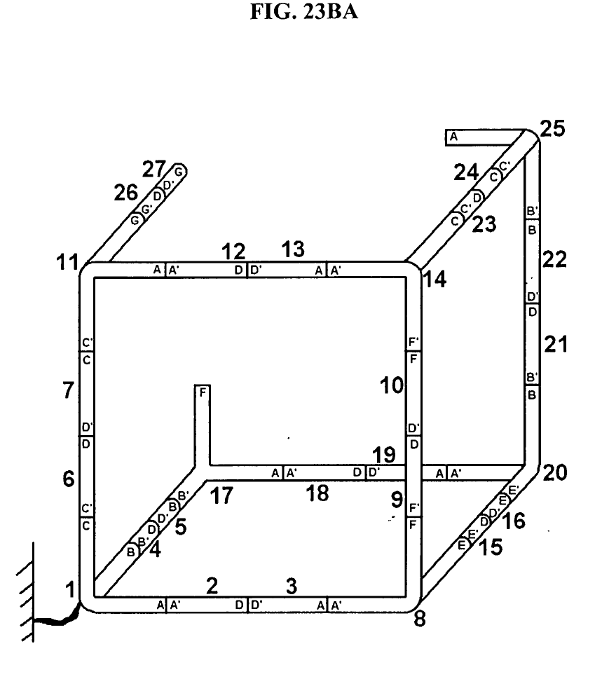

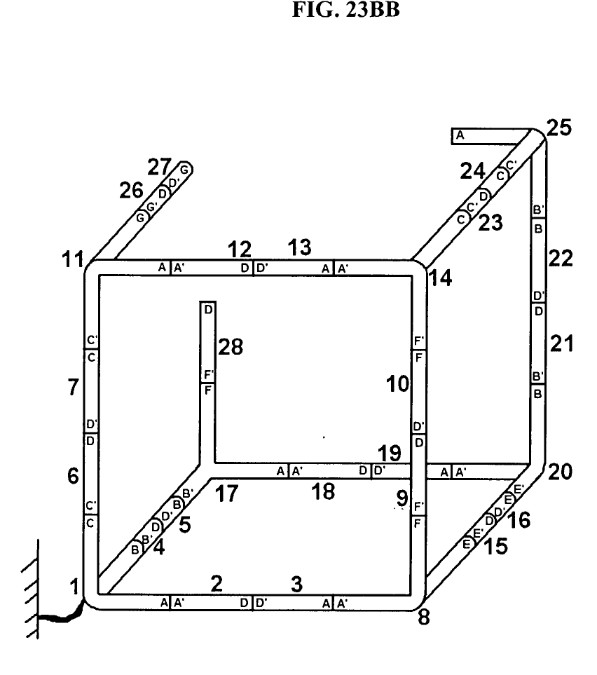

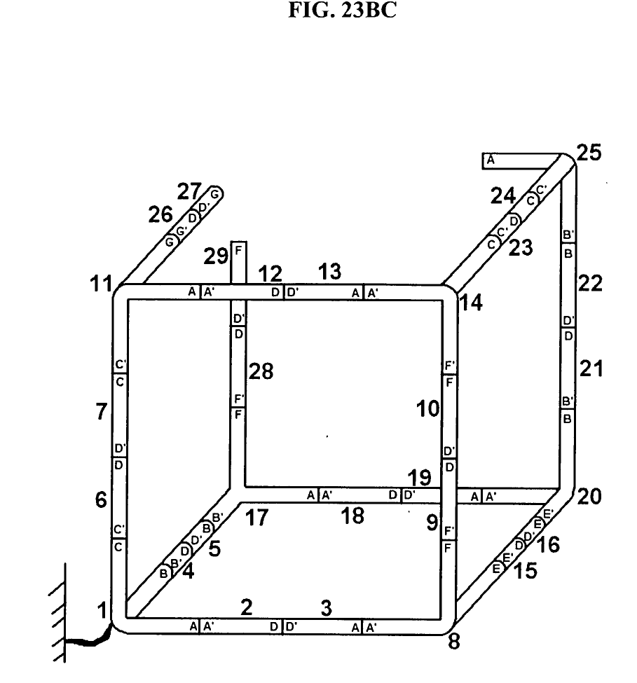

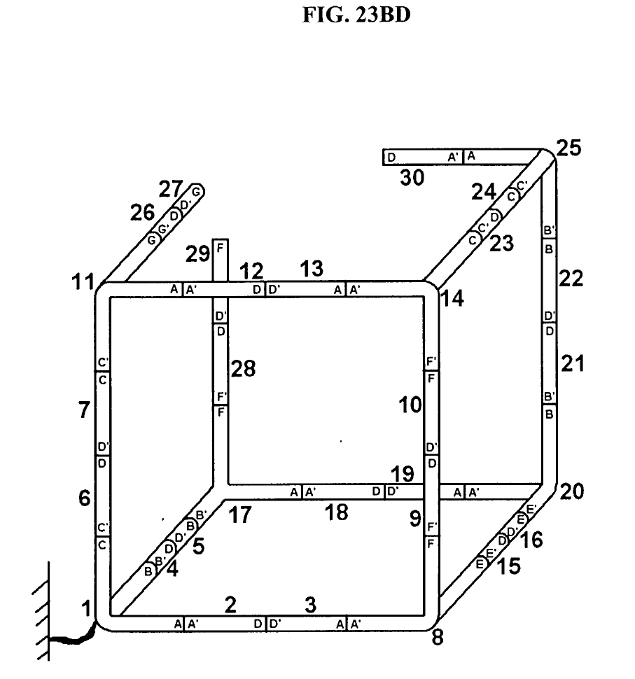

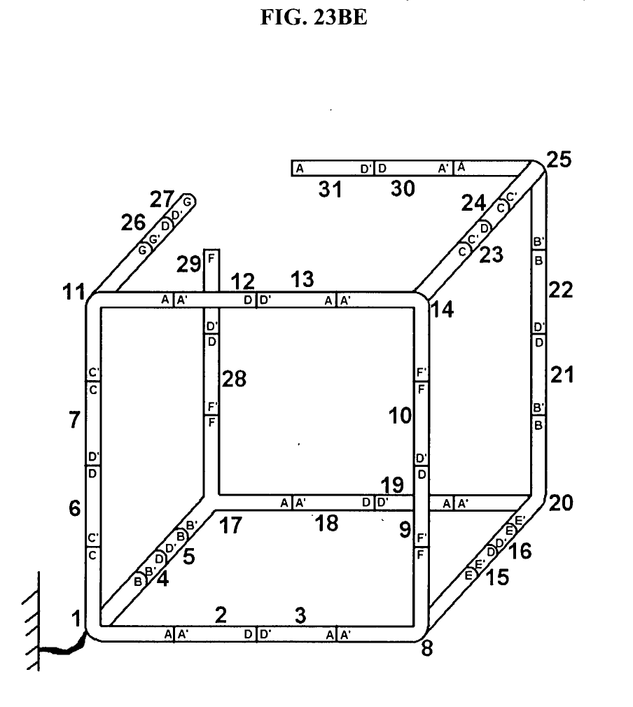

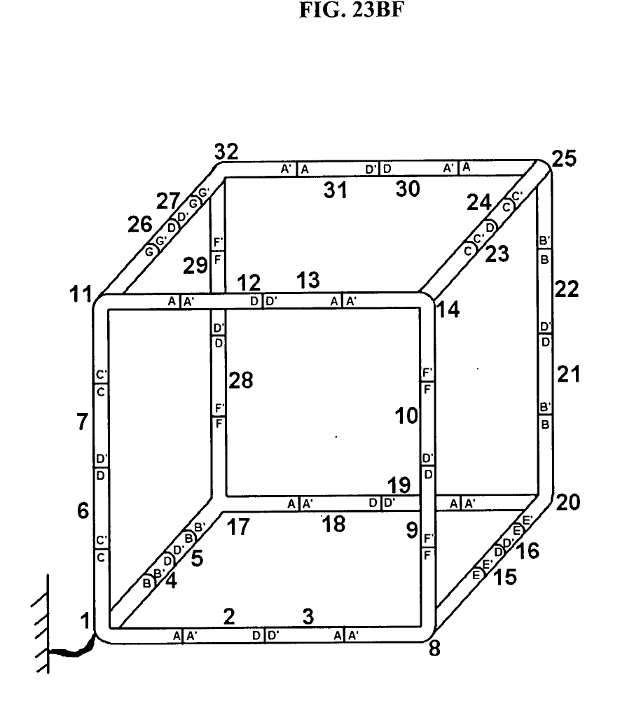

FIG. 3. Generation of a nanostructure from subassemblies. A nanostructure can be generated through the sequential addition of subassemblies, using steps analogous to those used for the addition of individual assembly units as illustrated above in FIG. 2. The arrow indicates the addition of a subassembly to a growing nanostructure.

FIG. 4. A diagram illustrating the addition of protein units and inorganic elements to a nanostructure according to the staged assembly methods of the invention. In step 1, an initiator unit is bound to a solid substrate. In step 2, an assembly unit is bound specifically to the initiator unit. In step 3, an additional assembly unit is bound to the nanostructure undergoing assembly. This assembly unit comprises an engineered binding site specific for a particular inorganic element. In step 4, the inorganic element (depicted as a cross-hatched oval) is added to the structure and bound by the engineered binding site. Step 5 adds another assembly unit with a binding site engineered for specificity to a second type of inorganic element, and that second inorganic element (depicted as a hatched diamond) is added in step 6.

FIG. 5. Line drawing representing the a-carbon trace of an intact IgGl (Protein Data Bank (pdb) entry 1IGY) (Harris et al., 1998, Crystallographic structure of an intact IgG1 monoclonal antibody, J. Mol. Biol. 275(5): 861-72). (For a description of the Protein Data Bank (pdb), see Berman et al., 2000, The Protein Data Bank, Nucl. Acids Res. 235-42; Saqi et al, 1994, PdbMotif—a tool for the automatic identification and display of motifs in protein structures, Comput. Appl. Biosci. 10(5): 545-46.) Thick lines represent the heavy chains and thin lines represent the light chains. The Fv and C_{H}1 domains of the Fab fragment and the C_{H}2 and C^{H}3 domains of the Fc fragment are labeled. Ball-and-stick modeling, indicated by gray arrowheads, represent disulfide cysteine bonds. Clusters of disulfide bridging interactions occur in the flexible hinge region located between the Fab and Fc fragments. These interactions may aid in dimerization and provide structural integrity of this otherwise highly flexible region in the immunoglobulin. Drawing was treated with the program SETOR (Evans, 1993, SETOR: Hardware lighted three-dimensional solid model representations of macromolecules, J. Mol. Graphics, 11: 134-38).

FIG. 6. Line drawing representing the α-carbon trace of a Fab fragment that can be used as the structural element for design of an assembly unit (pdb entry 1CIC ). The heavy lines represent the heavy chain and the light lines represent the light chain. The domains of the heavy chain (V_{H} and C_{H}1) and the light chain (V_{L} and C_{L}) are labeled. Also indicated is the flexible Fab “elbow” or bend region connecting the variable domains and constant domains. The Fab angle of the bend varies considerably (127-176°) even among members of the same antibody class.

FIGS. 7(A-B). Diagram of two diabody units, Unit 1 (A) and Unit 2 (B) and their associated genes. A. Unit 1 is an A×B diabody in which the V_{H} and V_{L} domains of A define a lysozyme isotopic antibody (D1.3) and in which the V_{H} and V_{L} domains of B define a virus neutralizing idiotopic antibody (730.1.4). In order to facilitate purification of the desired diabody product, the gene encoding V_{H}A and V_{L}B includes a hexahistidine tag, whereas the gene encoding V_{H}B and V_{L}A does not. B. Unit 2 is B′×A′ diabody in which the V_{H} and V_{L} domains of B′ define a virus neutralizing idiotopic antibody (409.5.3) and in which the V_{H} and V_{L} domains of A′ define a lysozyme isotopic antibody (E5.2). In order to facilitate purification of the desired diabody product, the gene encoding V_{H}B′ and V_{L}A′ includes a hexahistidine tag, whereas the gene encoding V_{H}A′ and V_{H}B′ does not.

FIG. 8. Line drawing representing the three-dimensional structures of the α-carbon trace of a diabody (pdb entry 1 LMK) (top) and a single chain Fv (scFv) antibody (pdb entry 2APA) (bottom). For the monomeric scFv structure (bottom), heavy lines represent the heavy chain and the light lines represent the light chain. For the dimeric diabody structure (top), however, the heavy lines represent both the heavy chain and light chain of one scFv, while the light lines represent both the heavy and light chain of the other scFv. scFv constructs that have the heavy-light variable domains linked together by a longer peptide linkers form stable monomers. Those with shorter linkers associate with a second scFv molecule to form a bivalent diabody as shown. Note that the immunoglobulin fold contained within both structures is very similar. scFv and diabodies, or binding derivatives or binding fragments thereof, can be used as the basic elements for the design of assembly units.

FIG. 9. Schematic representation of various IgGs including monovalent, bivalent, monospecific and bispecific antibodies. IgGs that are derived from a single cell line are homozygous for IgG. The resulting IgGs are therefore bivalent-monospecific antibodies. A hybrid hybridoma, e.g., a quadroma, arises from a fusion cell line. IgGs that are produced by hybrid hybridomas may be mixtures of heterologous bivalent-bispecific (e.g., heterologous-F(ab′)₂) and homozygous bivalent-monospecific (e.g., F(ab′)₂) IgG. Hybrid hybridoma heterodimers therefore represent a source of bivalent-bispecific F(ab′)₂. The intact IgG molecules or binding derivative or binding fragment thereof can be used as the basic elements for the design of assembly units.

FIG. 10. Schematic representation of an IgG molecule cleaved into its component fragments, F(ab′)₂ and Fc, upon limited exposure to protease. The hinge region, containing several disulfide-bond interactions, helps maintain dimerization of the Fab fragments. Subsequent exposure of the F(ab′)₂ to reducing conditions disrupts the hinge disulfide bridging interactions between the fragments to yield monomeric Fab. Separate functional fragments of the IgG can be isolated (i.e., Fab fragments) for specific uses in the design of assembly units such as creating bivalent-bispecific heterologous F(ab′)₂ by chemical cross-linking.

FIGS. 11(A-D). Dimerization motifs that have been developed to promote the multimerization of antigen-binding fragments that contain various specificities. Leucine zipper motifs (depicted as elongated ovals) such as Jun-Fos or GCN4 (Kostelny et al., 1992, Formation of a bispecific antibody by the use of leucine zippers, J. Immunol. 148(5): 1547-53; de Kruif et al., 1996, Leucine zipper, dimerized bivalent and bispecific scFv antibodies from a semi-synthetic antibody phage display library, J. Biol. Chem. 271(13): 7630-34), or four-helix bundle motifs (depicted as rectangles in (C) and (D)), such as Rop (Pack et al., 1993, Improved bivalent miniantibodies, with identical avidity as whole antibodies, produced by high cell density fermentation of Escherichia coli, Biotechnology (NY) 11(11): 1271-77; Muller et al., 1998, A dimeric bispecific miniantibody combines two specificities with avidity, FEBS Lett. 432(1-2): 45-49), may be employed to promote the stable dimerization of antigen-binding multimers. These dimerized antigen-binding multimers may be utilized as the structural and joining elements in assembly unit fabrication.

FIG. 12. Diagram of single-chain Fv fragments (scFv). The top half of the diagram shows monomeric, dimeric (diabody), trimeric (triabody) and tetrameric (tetrabody) associations among V_{H}-linker-V, scFv fragments. The bottom half of the diagram shows such associations among V_{L}-linker-V_{H} scFv fragments. These associations between scFv domains are dependent upon the length of the peptide linker joining the V_{H} and V_{L} units. Longer peptide linkers (12-15 residues) favor monomeric formation, whereas shorter linkers (0-5 residues), favor multimeric structures. The linkage order of the V_{H} and V_{L} genes also affects multimer formation, activity and stability of the resultant scFv proteins. This type of recombinant antibody represents one of the smallest functional antigen binding entities derived from an IgG and can be utilized as the structural and joining elements in assembly unit fabrication.

FIG. 13. Diagram of the structure of a P-pilus. The pilus is anchored to the outer membrane of E. coli through an N-terminal membrane anchor in paph. Most of the pilus is made up of many copies of papA. The rod is terminated by a single copy of papK that acts as an adaptor between the rod structure and a thin, distal structure called a fibrillum. The fibrillum consists of a few copies of papE, followed by a single copy of papF and a single copy of papG, which acts as the adhesin at the distal tip of the structure.

FIGS. 14 (A-B). A. Diagram of the interaction of two pilins, showing the close interaction of the N-terminal extension of one pilin (depicted in the lower right of the figure) with the groove on the surface of the other pilin (depicted in the upper left of the figure). Pilins interact through the binding of a long N-terminal extension from the pilin to the body of an adjacent pilin. This provides an extended, specific interaction with significant mechanical strength. B. Diagram of the interaction of papE with a hybrid pilin constructed from the N-terminal arm of papF spliced onto the protein body of papA. Replacing the N-terminal arm of a pilin with the N-terminal arm of a different pilin alters its binding specificity. Here, papA has had its N-terminal arm replaced by that of papF (arrow), now making it possible for the papA to interact with papE through the use of interactions normally used to stabilize the papF-papE interaction

FIG. 15. Diagram of ROP protein, a four-helix bundle.

FIG. 16. Diagram of an idiotope/anti-idiotope Fab-Fab interaction. The diagram shows the α-carbon trace of two Fab fragments interacting through idiotopic/anti-idiotopic interactions (pdb entry 1CIC). The heavy lines represent the heavy chains and the light lines represent the light chains of the Fab fragments. Most of the idiotopic/anti-idiotopic protein binding interactions occur between the loops of the heavy chains contained in the complementarity determining region (CDR). In this case, the association between Fabs results in a nearly linear association.

FIG. 17. Diagram of a staged assembly of hybrid pilin subunits. The illustrated process is described in Section 6 (Example 1). The addition of hybrid pilin subunits proceeds according to the steps indicated in the diagram. Hybrid pilins are made up of the protein body of one pilin (designated in capital letters, e.g., A, H, E, K) and the N-terminal extension of another pilin (designated in lower-case letters, e.g., k, a, f, e). The positioning of the ras epitope is indicated.

FIG. 18. Comparison of PNA (peptide nucleic acid, left) and DNA (right) structure. Note that PNA has a neutral peptide or peptide-like backbone instead of a negatively-charged sugar-phosphate backbone.

FIGS. 19(A-B). Two PNA/oligopeptide units can dimerize to form a single assembly unit. Two possible configurations for an assembly unit are shown here (FIG. 19A and FIG. 19B). The PNA portion provides joining elements A and B′, while the oligopeptide portion forms two coiled coil structural elements (S) stabilized by disulfide bonds at either end. One or more functional units (F), comprised of, e.g., protein segments, may also be incorporated into the assembly unit. In certain embodiments, the assembly unit can have a randomly coiled peptide that comprises a functional element, F, in the internal or center portion of the dimer (FIG. 19A) or at the end of the PNA molecule opposite the end comprising the joining element (FIG. 19B). In each of these diagrams, the N-terminal end of the PNA/oligopeptide unit is towards the left of the diagram and the C-terminal end is towards the right.

FIG. 20. Line diagram indicating the order of elements of the upper synthetic protein monomer forming the staged assembly subunit shown in FIG. 19A. The order of the elements in the corresponding lower unit would be identical except that the PNA element is at the C-terminus. This reflects the parallel arrangement of the leucine zippers aligning the two units. The functionality sequence encodes the region at which a functional element may be added to the assembly subunit. Glycines separate each element to reduce steric interference between elements. Numbers below the line indicate the typical length in residues of each element.

FIG. 21. Diagram of eleven steps of a staged assembly that utilizes four bispecific assembly units and one tetraspecific assembly unit to make a two-dimensional nanostructure. For details, see Section 8 (Example 3).

FIGS. 22(A-B). Diagram of a staged assembly that utilizes nanostructure intermediates as subassemblies. In Steps 1-3, a nanostructure intermediate is constructed, two joining elements are capped and the nanostructure intermediate is released from the solid substrate. In Step 5, the nanostructure intermediate from Step 3 is added to an assembly intermediate (shown in Step 4 attached to the solid substrate) as an intact subassembly. For details, see Section 9 (Example 4).

FIGS. 23(AA-BF). Diagram of the sequence of the 32 steps used in the staged assembly of an exemplary cubic nanostructure. The cubic nanostructure is assembled from assembly units comprising structural elements from engineered diabody and triabody fragments. The joining elements of the assembly units are the multispecific binding domains from diabodies or triabodies. Seven complementary joining pairs are used: A and A′, B and B′, C and C′, D and D′, E and E′, F and F′, and G and G′. The numbering (1-32) indicates the assembly unit added during each step. For details, see Section 11 (Example 6).

### 5. DETAILED DESCRIPTION OF THE INVENTION

### 5.1 Introduction

The present invention provides compositions and methods for the staged assembly of nanostructures. According to the methods of the invention, assembly of nanostructures proceeds by sequential, non-covalent, vectorial addition of specific assembly units to an initiator unit or a nanostructure intermediate during an assembly cycle, a process that is referred to herein as “staged assembly.” Attachment of each assembly unit is, by design, mediated by the specific, non-covalent binding of one or more pre-designated joining elements of one assembly unit to a complementary joining element present on the initiator unit or assembly intermediate. To avoid self-polymerization, each assembly unit is designed so that no joining element that is a part of the assembly unit can interact with any other joining element of that same assembly unit. The process is carried out in a massively parallel fashion such that a very large number of identical assemblies are fabricated simultaneously.

An “assembly unit” is herein defined as an assemblage of atoms and/or molecules comprising structural elements, joining elements and/or functional elements. In one embodiment, an assembly unit can be added to a nanostructure as a single unit through the formation of one or more specific interactions. In another embodiment, an assembly unit that comprises two or more assembly units, i.e., a subassembly, can be added to a nanostructure. An assembly unit may comprise one or more structural elements, and may further comprise one or more functional elements and one or more joining elements. If an assembly unit comprises a functional element, that functional element may be attached to or incorporated within a joining element or, in certain embodiments, a structural element. Such an assembly unit, which may comprise a structural element and one or a plurality of non-interacting joining elements, may be, in certain embodiments, structurally rigid and have well-defined recognition and binding properties. In one aspect of the invention, each joining element in the assembly unit exhibits specificity for a complementary joining element. A functional element can, in certain embodiments, be used to provide an attachment site for a moiety with a desirable physical, chemical, or biological property. Such a moiety could be, for example, a peptide, protein (e.g., enzyme), protein domain, small molecule, inorganic nanoparticle, atom, cluster of atoms, magnetic, photonic or electronic nanoparticles, or a marker such as a radioactive molecule, chromophore, fluorophore, chemiluminescent molecule, or enzymatic marker. Such functional elements can also be used for cross-linking linear, one-dimensional nanostructures to form two-dimensional and three-dimensional nanostructures.

According to the methods of the invention, the first assembly unit (i.e., the initiator unit) has one or a plurality of joining element(s) comprising the first joining element of a joining pair, which joining element is available for binding by another assembly unit comprising the second joining element of the joining pair. In preferred embodiments, the initiator unit is attached to a solid support. Attachment of each assembly unit is, by design, mediated by the specific, non-covalent binding of a single pre-determined joining element of one assembly unit to its complementary joining element. The complementary joining element is presented by an initiator or nanostructure intermediate.

Each interaction of a joining element is designed such that the joining element of an assembly unit does not interact with any other joining element of said assembly unit. Self-polymerization of the assembly unit is thereby obviated in each assembly cycle: only one assembly unit can be added to a target joining element on the initiator unit or nanostructure intermediate, and binding of the assembly unit to the target initiator or nanostructure intermediate will be vectorial.

The invention provides structural elements comprising antibodies or binding derivatives or binding fragments thereof, including, but not limited to, structural elements comprising: monoclonal antibodies, multispecific antibodies, Fab or F(ab′)₂ antibody fragments, single-chain antibody fragments (scFvs), bispecific IgG, chimeric IgG or bispecific heterodimeric F(ab′)₂ antibodies, diabodies or multimeric scFv fragments. A binding derivative of an antibody or antibody fragment is a derivative that exhibits the binding specificity of the antibody, antibody fragment, single-chain antibody fragment (scFv), etc., from which the binding derivative is derived. A binding fragment of an antibody or antibody fragment is a fragment that exhibits the binding specificity of the antibody, antibody fragment, single-chain antibody fragment (scFv), etc., from which the binding fragment is derived.

The invention also provides structural elements comprising bacterial pilin proteins, leucine zipper-type coiled coils, or four-helix bundles.

The invention provides joining elements that exhibit antigen-antibody interactions, including, but not limited to, joining elements comprising: recombinantly engineered antibodies or binding derivatives or binding fragments thereof, molecules that exhibit idiotope/anti-idiotope interactions, or two non-complementary idiotopes. The invention also provides joining elements comprising peptide epitopes, bacterial pilin proteins or binding derivatives or binding fragments thereof, or peptide nucleic acids (PNAs). A binding derivative of a molecule such as a peptide epitope, pilin protein or PNA is a derivative that exhibits the binding specificity of the peptide epitope, pilin protein or PNA from which the binding derivative is derived. A binding fragment of a molecule such as a peptide epitope or pilin protein is a fragment that exhibits the binding specificity of the peptide epitope or pilin protein from which the binding fragment is derived.

The staged-assembly methods described herein make possible the mass production of nanostructures that are multi-dimensional and have non-periodic architectures, and in which organic and inorganic nanocomponents are placed with precision in designated locations. The resulting nanostructures utilize proteins to control the assembly of structures that may, in certain embodiments, incorporate organic materials or inorganic materials such as metallic, semiconducting or magnetic nanoparticles (Bruchez et al., 1998, Semiconductor nanocrystals as fluorescent biological tags, Science 281: 2013-16; Peng et al., 2000, Shape control of CdSe nanocrystals, Nature 404(6773): 59-61; Whaley et al., 2000, Selection of peptides with semiconductor binding specificity for directed nanocrystal assembly. Nature 405: 665-68). Proteins offer many advantages over other molecules for the controlled assembly of complex architectures.

The staged-assembly methods disclosed herein do not depend on the physical manipulation of individual components and thus constitute a highly efficient and economical means for the precise geometric and spatial positioning of individual components in the nanometer range. The methods of the invention make possible the fabrication of highly complex architectures with only a few distinct, non-cross-reacting joining pairs. This greatly simplifies the problem of component design.

The staged-assembly methods disclosed herein provide a practical and sensible solution for solving the complicated and intricate problem of economic, massively parallel manufacturing of highly complex nanostructures. This is in sharp contrast to the nanoconstruction of nanostructured materials by self-assembly. With self-assembly, complete control of the material architecture is precluded. Self-assembly of nanodevices is limited, since each assembly unit in the nanostructure must have its position encoded by joining elements that form specific interactions with adjacent assembly units, but that do not interact with any other assembly unit making up the nanostructure. For example, self-assembly of a device composed of 100 assembly units would require 100 or more complementary joining pairs and furthermore, the 100 joining pairs would have to be designed so that they did not cross-react with one another. The same nanostructure could be assembled by the staged assembly process as described herein, with far fewer non-cross-reacting joining pairs. In Section 11, Example 6, an example is provided of the staged assembly of a three-dimensional, cube-shaped structure made up of 32 assembly units. Self-assembly of this structure would require the use of 32 non-cross-reacting, complementary joining pairs. As disclosed in Section 11 (Example 6), staged assembly of the same structure can be accomplished with only seven non-cross-reacting complementary joining pairs.

### 5.2 Staged Assembly of Nanostructures

The present invention provides methods for staged assembly that enable massively parallel synthesis of complex, non-periodic, multi-dimensional nanostructures in which organic and inorganic moieties are placed, accurately and precisely, into a pre-designed, three-dimensional architecture. Staged assembly requires that a series of units be added in a given pre-designed order to an initiator unit and/or nanostructure intermediate. Because a large number of identical initiators are used and because subunits are added to all initiators/intermediates simultaneously, staged assembly fabricates multiple identical nanostructures in a massively parallel manner. In preferred embodiments, the initiator units are bound to a solid substrate, support or matrix. Additional assembly units are added sequentially in a procedure akin to solid phase polymer synthesis. The intermediate stage(s) of the nanostructure while it is being assembled, and which comprises the bound assembly units formed on the initiator unit, is generally described as either a nanostructure intermediate or simply, a nanostructure. Addition of each assembly unit to the nanostructure intermediate undergoing assembly depends upon the nature of the joining element presented by the previously added assembly unit and is independent of subsequently added assembly units. Thus assembly units can bind only to the joining elements exposed on the nanostructure intermediate undergoing assembly; that is, the added assembly units do not self-interact and/or polymerize.

Since the joining elements of a single assembly unit are non-complementary and therefore do not interact with one another, unbound assembly units do not form dimers or polymers. An assembly unit to be added is preferably provided in molar excess over the initiator unit or nanostructure intermediate in order to drive its reaction with the intermediate to completion. Removal of unbound assembly units during staged assembly is facilitated by carrying out staged assembly using a solid-substrate-bound initiator so that unbound assembly units can be washed away in each cycle of the assembly process.

This scheme provides for assembly of complex nanostructures using relatively few non-cross-reacting, complementary joining pairs. Only a few joining pairs need to be used, since only a limited number of joining elements will be exposed on the surface of an assembly intermediate at any one step in the assembly process. Assembly units with complementary joining elements can be added and incubated against the nanostructure intermediate, causing the added assembly units to be attached to the nanostructure intermediate during an assembly cycle. Excess assembly units can then be washed away to prevent them from forming unwanted interactions with other assembly units during subsequent steps of the assembly process. Each position in the nanostructure can be uniquely defined through the process of staged assembly and distinct functional elements can be added at any desired position. The staged assembly method of the invention enables massive parallel manufacture of complex nanostructures, and different complex nanostructures can be further self-assembled into higher order architectures in a hierarchic manner.

FIG. 2 depicts an embodiment of the staged assembly method of the invention in one dimension. In step 1, an initiator unit is immobilized on a solid substrate. In step 2, an assembly unit is added to the initiator (i.e. the matrix bound initiator unit), resulting in a nanostructure intermediate composed of two units. Only a single assembly unit is added in this step, because the second assembly unit cannot interact (i.e. polymerize) with itself.

The initiator unit, or any of the assembly units subsequently added during staged assembly including the capping unit, may contain an added functional element and/or may comprise a structural unit of different length from previously added units. For example, in step 3 of FIG. 2, a third assembly unit is added that comprises a functional element. In steps 4 and 5, additional assembly units are added, each with a designed functional group. Thus in the embodiment of staged assembly depicted in FIG. 2, the third, fourth and fifth assembly units each carry a unique functional element (designated by geometric shapes protruding from the top of the assembly units in the figure).

The embodiment of staged assembly depicted in FIG. 2 requires only two non-cross-reacting, complementary joining pairs. Self-assembly of the structure, as it stands at the end of step 5, would require four non-cross-reacting, complementary joining pairs. This relatively modest improvement in number of required joining pairs becomes far greater as the size of the structure increases. For instance, for a linear structure of N units assembled by an extension of the five steps illustrated in FIG. 2, staged assembly would still require only two non-cross-reacting, complementary joining pairs, whereas self-assembly would require (N−1) non-cross-reacting, complementary joining pairs.

The number of nanostructures fabricated is determined by the number of initiator units bound to the matrix while the length of each one-dimensional nanostructure is a function of the number of assembly cycles performed. If assembly units with one or more different functional elements are used, then the order of assembly will define the relative spatial orientation of each functional element relative to the other functional elements.

After each step in the method of staged assembly of the invention, excess unbound assembly units are removed from the attached nanostructure intermediate by a removal step, e.g., a washing step. The substrate-bound nanostructure intermediate may be washed with an appropriate solvent (e.g., an aqueous solution or buffer). The solvent must be able to remove subunits held by non-specific interactions without disrupting the specific, interactions of complementary joining elements. Appropriate solvents may vary as to pH, salt concentration, chemical composition, etc., as required by the assembly units being used.

A buffer used for washing the nanostructure intermediate can be, for example, a buffer used in the wash steps implemented in ELISA protocols, such as those described in Current Protocols in Immunology (see Chapter 2, Antibody Detection and Preparation, Section 2.1 “Enzyme-Linked Immunosorbent Assays,” John Wiley & Sons, 2001, Editors John E. Coligan, Ada M. Kruisbeek, David H. Margulies, Ethan M. Shevach, Warren Strober, Series Editor: Richard Coico).

In certain embodiments, an assembled nanostructure is “capped” by addition of a “capping unit,” which is an assembly unit that carries only a single joining element. Furthermore, if the initiator unit has been attached to the solid substrate via a cleavable bond, the nanostructure can be removed from the solid substrate and isolated. However, in some embodiments, the completed nanodevice will be functional while attached to the solid substrate and need not be removed.

The above-described steps of adding assembly units can be repeated in an iterative manner until a complete nanostructure is assembled, after which time the complete nanostructure can be released by breaking the bond immobilizing the first assembly unit from the matrix at a designed releasing moiety (e.g,. a protease site) within the initiator unit or by using a pre-designed process for release (e.g., lowering of pH). The process of staged assembly, as illustrated in FIGS. 2 and 3 is one of the simplest embodiments contemplated for staged assembly. In other embodiments, assembly units with additional joining elements can be used to create more complex assemblies. Assembly units may be added individually or, in certain embodiments, they can be added as subassemblies (FIG. 3). The result is a completely defined nanostructure with functional elements that are distributed spatially in relationship to one another to satisfy desired design parameters. The compositions and methods disclosed herein provide means for the assembly of these complex, designed nanostructures and of more complex nanodevices formed by the staged assembly of one or a plurality of nanostructures into a larger structure. Fabrication of multidimensional nanostructures can be accomplished, e.g., by incorporating precisely-spaced assembly units containing additional joining elements into individual, one-dimensional nanostructures, where those additional joining elements can be recognized and bound by a suitable cross-linking agent to attach the individual nanostructures together. In certain preferred embodiments, such cross-linking could be, e.g., an antibody or a binding derivative or a binding fragment thereof.

In some embodiments of the staged assembly method of the invention, the initiator unit is tethered to a solid support. Such tethering is not random (i.e., is not non-specific binding of protein to plastic or random biotinylation of an assembly unit followed by binding to immobilized streptavidin) but involves the binding of a specific element of the initiator unit to the matrix or substrate. The staged assembly process is a vectorial process that requires an unobstructed joining element on the initiator unit for attachment of the next assembly unit. Random binding of initiator units to substrate would, in some cases, result in the obstruction of the joining element needed for the attachment of the next assembly unit, and thus lowering the number of initiator units on which nanostructures are assembled.

In other embodiments of the staged assembly method of the invention, the initiator unit is not immobilized to a solid substrate. In this case, a removal step, e.g., a washing step, can be carried out on a nanostructure constructed on a non-immobilized or untethered initiator unit by: (1) attaching a magnetic nanoparticle to the initiator unit and separating nanostructure intermediates from non-bound assembly units by applying a magnetic field; 2) separating the larger nanostructure intermediates from unbound assembly units by centrifugation, precipitation or filtration; or 3) in those instances in which a nanostructure intermediate or assembled nanostructure is more resistant to a destructive treatment (e.g., protease treatment or chemical degradation), unbound assembly units are selectively destroyed.

Proteins have well-defined binding properties, and the technology to manipulate the intermolecular interactions of proteins is well known in the art (Hayashi et al., 1995, A single expression system for the display, purification and conjugation of single-chain antibodies, Gene 160(1): 129-30; Hayden et al., 1997, Antibody engineering, Curr. Opin. Immunol. 9(2): 201-12; Jung et al., 1999, Selection for improved protein stability by phage display, J. Mol. Biol. 294(1): 163-80, Viti et al., 2000, Design and use of phage display libraries for the selection of antibodies and enzymes, Methods Enzymol. 326: 480-505; Winter et al., 1994, Making antibodies by phage display technology, Annu. Rev. Immunol. 12: 433-55). The contemplated staged assembly of nanostructures, however, need not be limited to components composed primarily of biological molecules, e.g., proteins and nucleic acids, that have specific recognition properties. The optical, magnetic or electrical properties of inorganic atoms or molecules will be required for some embodiments of nanostructures fabricated by staged assembly.

There will be many embodiments of this invention in which components not made up of proteins will be advantageously utilized. In other embodiments, it may be possible to utilize the molecular interaction properties of proteins or nucleic acids to construct nanostructures composed of both organic and inorganic materials.

In certain embodiments, inorganic nanoparticles are added to components that are assembled into nanostructures using the staged assembly methods of the invention. This may be done using joining elements specifically selected for binding to inorganic particles. For example, Whaley and co-workers have identified peptides that bind specifically to semiconductor binding surfaces (Whaley et al., 2000, Selection of peptides with semiconductor binding specificity for directed nanocrystal assembly, Nature 405: 665-68). In one embodiment, these peptides are inserted into protein components described herein using standard cloning techniques. Staged assembly of protein constructs as disclosed herein, provides a means of distributing these binding sites in a rigid, well-defined three-dimensional array.

Once the binding sites for a particular type of inorganic nanoparticle are all in place, the inorganic nanoparticles can be added using a cycle of staged assembly analogous to that used to add proteinaceous assembly units. To accomplish this, it may be necessary, in certain embodiments to adjust the solution conditions under which the nanostructure intermediates are incubated, in order to provide for the solubility of the inorganic nanoparticles. Once an inorganic nanoparticle is added to the nanostructure intermediate, it is not possible to add further units to the inorganic nanoparticle in a controlled fashion because of the microheterogeneities intrinsic to any population of inorganic nanoparticles. These heterogeneities would render the geometry and stoichiometry of further interactions uncontrollable.

FIG. 4 is a diagram illustrating the addition of protein units and inorganic elements to a nanostructure according to the staged assembly methods of the invention. In step 1, an initiator unit is bound to a solid substrate. In step 2, an assembly unit is bound specifically to the initiator unit. In step 3, an additional assembly unit is bound to the nanostructure undergoing assembly. This assembly unit comprises an engineered binding site specific for a particular inorganic element. In step 4, the inorganic element (depicted as a cross-hatched oval) is added to the structure and bound by the engineered binding site. Step 5 adds another assembly unit with a binding site engineered for specificity to a second type of inorganic element, and that second inorganic element (depicted as a hatched diamond) is added in step 6.

The order in which assembly units are added is determined by the desired structure and/or activity that the product nanostructure, and the need to minimize the number of cross-reacting joining element pairs used in the assembly process. Hence determining the order of assembly is an integral part of the design of a nanostructure to be fabricated by staged assembly. Joining elements are chosen, by design, to permit staged assembly of the desired nanostructure. Since the choice of joining element(s) is generally independent of the functional elements to be incorporated into the nanostructure, the joining elements are mixed and matched as needed to fabricate assembly units with the necessary functional elements and joining elements that will provide for the placement of those functional elements in the desired spatial orientation.

For example, assembly units comprising two joining elements, designed using the six joining elements that make up three joining pairs, can include any of 18 pairs of the joining elements that are non-interacting. There are 21 possible pairs of joining elements, but three of these pairs are interacting (e.g. A-A′) and their use in an assembly unit would lead to the self-association of identical assembly units with one another. In the example illustrated below, joining elements are denoted as A, A′, B, B′, C and C′, where A and A′, B and B′, and C and C′ are complementary pairs of joining elements (joining pairs), i.e. they bind to each other with specificity, but not to any of the other four joining elements depicted. Six representative assembly units, each of which comprises two joining elements, wherein each joining element comprises a non-identical, non-complementary joining element, are depicted below. In this depiction, each assembly unit further comprises a unique functional element, one of a set of six, and represented as F₁ to F₆. According to these conventions, six possible assembly units can be designated as:

A-F₁-B

B′-F₂-A′

B′-F₃-C′

C-F₄-B

B′-F₅-A′

A-F₆-C′

Staged assembly according to the methods disclosed herein can be used to assemble the following illustrative linear, one-dimensional nanostructures, in which the order and relative vectorial orientation of each assembly unit is independent of the order of the functional elements (the symbol ●- is used to represent the solid substrate to which the initiator is attached and a double colon represents the specific interaction between assembly units):

●-A-F1-B::B′-F2-A′::A-F1-B::B′-F2-A′::A-F1-B::B′-F2-A′::A-F1-B::B′-F2-A′
●-A-F1-B::B′-F2-A′::A-F6-C′::C-F4-B::B′-F2-A′::A-F1-B::B′-F5-A′::A-F6-C′
●-A-F1-B::B′-F2-A′::A-F1-B::B′-F5-A′::A-F1-B::B′-F2-A′::A-F1-B::B′-F3-C′
●-A-F1-B::B′-F3-C′::C-F4-B::B′-F3-C′::C-F4-B::B′-F3-C′::C-F4-B::B′-F2-A′

As is apparent from this illustration, a large number of unique assembly units can be constructed using a small number of complementary joining elements. Moreover, only a small number of complementary joining elements are required for the fabrication of a large number of unique and complex nanostructures, since only one type of assembly unit is added in each staged assembly cycle and, therefore, joining elements can be used repeatedly without rendering ambiguous the position of an assembly unit within the completed nanostructure.

In each of the cases illustrated above, only two or three joining pairs have been used. Self-assembly of any of these structures would require the use of seven non-cross-reacting joining pairs. If these linear structures were N units in extent, they would still only require two or three joining pairs, but for self-assembly, they would require (N−1) non-cross-reacting, complementary joining pairs.

In another aspect of the invention, by interchanging the positions of the two joining elements of an assembly unit depicted above, the spatial position and orientation of the attached functional element will be altered within the overall structure of the nanostructure fabricated. This aspect of the invention illustrates yet another aspect of the design flexibility provided by staged assembly of nanostructures as disclosed herein.

Attachment of each assembly unit to an initiator or nanostructure intermediate is mediated by formation of a specific joining-pair interaction between one joining element of he assembly unit and one or more unbound complementary joining elements carried by the initiator or nanostructure intermediate. In many embodiments, only a single unbound complementary joining element will be present on the initiator or nanostructure intermediate. However, in other embodiments, it may be advantageous to add multiple identical assembly units to multiple sites on the assembly intermediate that comprise identical joining elements. In these embodiments, the staged assembly proceeds by the parallel addition of assembly units, but only a single unit will be attached at any one site on the intermediate, and assembly at all sites that are involved will occur in a pre-designed, vectorial manner.

Structural integrity of the nanostructure is of critical importance throughout the process of staged assembly, and the assembly units are preferably connected by non-covalent interactions. A specific non-covalent interaction is, for example, an interaction that occurs between an assembly unit and a nanostructure intermediate. The specific interaction should exhibit adequate affinity to confer stability to the complex between the assembly unit and the nanostructure intermediate sufficient to maintain the interaction stably throughout the entire staged assembly process. A specific non-covalent interaction should exhibit adequate specificity such that the added assembly unit will form stable interactions only with joining elements designed to interact with it. The interactions that occur among elements during the staged assembly process disclosed herein are preferably operationally “irreversible.” A binding constant that meets this requirement cannot be defined unambiguously since “irreversible” is a kinetic concept, and a binding constant is based on equilibrium properties. Nevertheless, interactions with Kd's of the order of 10⁻⁷ or lower (i.e. higher affinity and similar to the Kd of a typical diabody-epitope complex) will typically act “irreversibly” on the time scale of interest, i.e. during staged assembly of a nanostructure.

The intermolecular interactions need not act “irreversibly,” however, on the timescale of the utilization of a nanostructure (i.e. its shelf life or working life expectancy). In certain embodiments, nanostructures fabricated according to the staged assembly methods disclosed herein are subsequently stabilized by chemical fixation (e.g., by fixation with paraformaldehyde or glutaraldehyde) or by cross-linking. The most common schemes for cross-linking two proteins involve the indirect coupling of an amine group on one assembly unit to a thiol group on a second assembly unit (see, e.g., Handbook of Fluorescent Probes and Research Products, Eighth Edition, Chapter 2, Molecular Probes, Inc., Eugene, Ore.; Loster et al., 1997, Analysis of protein aggregates by combination of cross-linking reactions and chromatographic separations, J. Chromatogr. B. Biomed. Sci. Appl. 699(1-2): 439-61; Phizicky et al., 1995, Protein-protein interactions: methods for detection and analysis, Microbiol. Rev. 59(1): 94-123).

In certain embodiments of the invention, the fabrication of a nanostructure by the staged assembly methods of the present invention involves joining relatively rigid and stable assembly units, using non-covalent interactions between and among assembly units. Nevertheless, the joining elements that are incorporated into useful assembly units can be rather disordered, that is, neither stable nor rigid, prior to interaction with a second joining element to form a stable, preferably rigid, joining pair. Therefore, in certain embodiments of the invention, individual assembly units may include unstable, flexible domains prior to assembly, which, after assembly, will be more rigid. In preferred embodiments, a nanostructure fabricated using the compositions and methods disclosed herein is a rigid structure.

According to the methods of the invention, analysis of the rigidity of a nanostructure, as well as the identification of any architectural flaws or defects, are carried out using methods well-known in the art, such as electron microscopy.

In another embodiment, structural rigidity can be tested by attaching one end of a completed nanostructure directly to a solid surface, i.e., without the use of a flexible tether. The other end of the nanostructure (or a terminal branch of the nanostructure, if it is a multi-branched structure) is then attached to an atomic force microscope (AFM) tip, which is movable. Force is applied to the tip in an attempt to move it. If the nanostructure is flexible, there will be an approximately proportional relationship between the force applied and tip movement as allowed by deflection of the nanostructure. In contrast, if the nanostructure is rigid, there will be little or no deflection of the nanostructure and tip movement as the level of applied force increases, up until the point at which the rigid nanostructure breaks. At that point, there will be a large movement of the AFM tip even though no further force is applied. As long as the attachment points of the two ends are stronger than the nanostructure, this method will provide a useful measurement of rigidity.

According to the present invention, each position in a nanostructure is distinguishable from all others, since each assembly unit can be designed to interact tightly, specifically, and uniquely with its neighbors. Each assembly unit can have an activity and/or characteristic that is distinct to its position within the nanostructure. Each position in the nanostructure is uniquely defined through the process of staged assembly, and through the properties of each assembly unit and/or functional element that is added at a desired position. In addition, the staged-assembly methods and assembly units disclosed herein are amenable to large scale, massively parallel, automated manufacturing processes for construction of complex nanostructures of well-defined size, shape, and function.

The methods and compositions of the present invention capitalize upon the precise dimensions, uniformity and diversity of spatial geometries that proteins are capable of that are used in the construction of the assembly units employed herein. Furthermore, as described hereinbelow, the methods of the invention are advantageous because genetic engineering techniques can be used to modify and tailor the properties of those biological materials used in the methods of the invention disclosed herein, as well as to synthesize large quantities of such materials in microorganisms.

### 5.3. Assembly Units

Assembly units provided by the present invention and used in the staged assembly methods disclosed herein comprise an assemblage of atoms and/or molecules comprising structural elements, joining elements and/or functional elements. In certain embodiments, assembly units can be added to a nanostructure as a single unit through the formation of specific interactions. In other embodiments, assembly units can be added as subassemblies.

In order to participate in a staged assembly, each assembly unit, other than a capping unit, should have a minimum of two joining elements or sites at which a specific intermolecular interaction can take place. Initiator units may be considered to have a minimum of two joining elements if the element conferring immobilization to the substrate or matrix is considered a joining element. Joining elements, however, are generally considered to interact via non-covalent interactions and in many embodiments, the interaction between the initiator unit and the substrate or matrix may be covalent. Capping units need to have, at most, one joining element, so that once added to a nanostructure or nanostructure intermediate, no subsequently added assembly units can extend from the capping unit assembled to the nanostructure. Therefore, in certain embodiments, an assembly unit comprising only a single joining element can be used to “cap” a completed nanostructure (or to terminate one branch of a multi-branched vectorial growth network of a nanostructure), thereby preventing further additions of assembly units to a particular position within the nanostructure.

In certain embodiments, the assembly unit comprises a joining element that exhibits antigen-antibody interactions, including, but not limited to, a joining element comprising: recombinantly engineered antibody or binding derivative or binding fragment thereof, a molecule that exhibits idiotope/anti-idiotope interactions, or two non-complementary idiotopes. In other embodiments, the assembly unit comprises a joining element comprising a peptide epitope, a bacterial pilin protein or binding derivative or binding fragment thereof, or a peptide nucleic acid (PNA).

In certain embodiments, the assembly unit also comprises a structural element comprising an antibody or binding derivative or binding fragment thereof, including, but not limited to, a structural element comprising: a monoclonal antibody, a multispecific antibody, a Fab or F(ab′)₂ antibody fragment, a single-chain antibody fragment (scFv), a bispecific IgG, a chimeric IgG or bispecific heterodimeric F(ab′)₂ antibody, a diabody or multimeric scFv fragment. The invention also provides structural elements comprising a bacterial pilin protein, a leucine zipper-type coiled coil, or a four-helix bundle.

In certain embodiments, the assembly unit comprises a multi-domain polypeptide chain in which a flexible segment, generally an oligopeptide that may comprise two to five glycine units, is disposed between different domains in order to allow independent folding of each peptide or protein domain. The number of such glycine residues is generally determined empirically, as would be apparent to those of ordinary skill in the art. Therefore, in certain embodiments, a flexible segment is disposed between a joining element and a structural element, or between a functional domain and a joining element, structural domain or a portion thereof.

The present invention provides for the staged assembly of nanostructures that utilizes assembly units comprising recombinantly-engineered antibodies and/or portions thereof. Recombinant antibodies are among the preferred sources disclosed herein of structural elements and joining elements used for fabricating nanostructures in a staged-assembly process. In certain embodiments, structural and/or joining elements comprise binding derivatives or binding fragments of any class of immunoglobulin molecules, including IgG, IgM, IgE, IgA, IgD and any subclass thereof. In specific embodiments, structural and/or joining elements comprise modified, engineered or recombinantly-derived Fab or scFv fragments of IgG molecules.

In certain embodiment, a structural and/or joining element comprises a binding derivative or binding fragment of a protein of interest, such as an antibody or pilin protein. Derivatives of a protein of interest used in the methods of the invention, e.g., an antibody or pilin protein, can be made by altering sequences by substitutions, additions or deletions that provide for functionally equivalent molecules. Due to the degeneracy of nucleotide coding sequences, other DNA sequences that encode substantially the same amino acid sequence as the gene encoding the protein of interest may be used in the practice of the present invention. These include, but are not limited to, nucleotide sequences comprising all or portions of a gene, which is altered by the substitution of different codons that encode a functionally equivalent amino acid residue within the sequence, thus producing a silent change.

Likewise, derivatives of a protein of interest include, but are not limited to, those containing, as a primary amino acid sequence, all or part of the amino acid sequence of a protein of interest including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isolcucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Derivatives or analogs of antibody or pilin proteins include but are not limited to those molecules comprising regions that are substantially homologous to the antibody or pilin protein of interest or a binding fragment thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a sequence encoding the protein of interest, under highly stringent or moderately stringent conditions. Such highly or moderately stringent conditions are commonly known in the art.

By way of example and not limitation, exemplary conditions of high stringency are as follows: Prehybridization of filters containing DNA is carried out for 8 h to overnight at 65° C. in buffer composed of 6× SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2× SSC, 0.01% PVP, 0.01% Ficoll, and 0.01% BSA. This is followed by a wash in 0.1× SSC at 50° C. for 45 min before autoradiography. Other conditions of high stringency that may be used are well known in the art.

By way of example and not limitation, exemplary conditions of moderate stringency are as follows: Filters containing DNA are pretreated for 6 h at 55° C. in a solution containing 6× SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C., and then washed twice for 30 minutes at 60° C. in a solution containing 1× SSC and 0.1% SDS. Filters are blotted dry and exposed for autoradiography. Other conditions of moderate stringency that may be used are well-known in the art.

Other conditions of high stringency that may be used are well known in the art. In general, for probes between 14 and 70 nucleotides in length the melting temperature (TM) is calculated using the formula: Tm(° C.)=81.5+16.6(log[monovalent cations (molar)])+0.41 (% G+C)−(500/N) where N is the length of the probe. If the hybridization is carried out in a solution containing formamide, the melting temperature is calculated using the equation Tm(° C.)=81.5+16.6(log[monovalent cations (molar)])+0.41 (% G+C)−(0.6 1% formamide)−(500/N) where N is the length of the probe. In general, hybridization is carried out at about 20-25 degrees below Tm (for DNA-DNA hybrids) or 10-15 degrees below Tm (for RNA-DNA hybrids).

The present invention also provides for the staged assembly of nanostructures that utilizes assembly units comprising a fragment of a protein of interest, e.g., an antibody or pilin protein. In a specific embodiment of the invention, a protein consisting of or comprising a fragment of a protein of interest consists of at least 4 contiguous amino acids of the protein of interest. In other embodiments, the fragment consists of at least 5, 6, 7, 8, 9, 10, 15, 20, 35 or 50 contiguous amino acids of the protein of interest. In specific embodiments, such fragments are not larger than 35, 100, 200, 300 or 350 amino acids.

The present invention also provides for the staged assembly of nanostructures that utilizes assembly units comprising fusion proteins. The production of fusion or chimeric protein products (comprising a desired protein (e.g., an IgG), fragment, analog, or derivative joined via a peptide bond to a heterologous protein sequence (of a different protein)). Such chimeric protein products can be made by ligating the appropriate nucleic acid sequences encoding the desired amino acid sequences to each other by methods known in the art, in the proper reading frame, and expressing the chimeric product by methods commonly known in the art. Alternatively, such a chimeric product may be made by protein synthetic techniques, e.g., by use of a peptide synthesizer.

The three-dimensional structures of IgG and its binding derivatives or binding fragments, e.g., IgG, Fab, scFv, (scFv)₂ (scFv)₃), have been solved (Braden et al., 1996, Crystal structure of an Fv-Fv idiotope-anti-idiotope complex at 1.9 A resolution, J. Mol. Biol. 264(1): 137-51; Ban et al., 1994, Crystal structure of an idiotype-anti-idiotype Fab complex, Proc. Natl. Acad. Sci. U.S.A. 91(5): 1604-08; Perisic et al., 1994, Crystal structure of a diabody, a bivalent antibody fragment, Structure 2(12): 1217-26; Harris et al., 1998, Crystallographic structure of an intact IgG1 monoclonal antibody, J. Mol. Biol. 275(5): 861-72; Pei et al., 1997, The 2.0-A resolution crystal structure of a trimeric antibody fragment with noncognate V_{H}-V_{L} domain pairs shows a rearrangement of V_{H} CDR3, Proc. Natl. Acad. Sci. USA 94(18): 9637-42). Each IgG-derived antibody fragment preferably contains at least one monovalent and monospecific complementarity determining region (CDR) or joining element. The CDR is preferably the site contained in each structure at which the highly specific intermolecular interaction can occur between the protein components.

Recombinantly engineered antibodies meet many of the basic criteria for use in the construction of assembly units for staged-assembly of nanostructures and are preferred sources of joining elements used for fabricating such nanostructures. Not only are such recombinant antibody binding domains structurally well characterized, they also have inherent binding specificities (joining elements) necessary for assembly unit addition.

For example, the known three-dimensional structure of many recombinant engineered components can serve as a guide for design of structural modifications to the antibody fragment that will enable the insertion of peptides (for example, at the site of a surface loop) that will confer novel binding, structural or functional properties to the antibody fragment. Moreover, there is a huge diversity of intermolecular specificities, such as that involving an antibody and a specific epitope, that can be either designed and constructed, or selected from a library. Advances in recombinant antibody technology have led to the creation of multivalent, multispecific and multifunctional antibodies (Chaudhary et al., 1989, A recombinant immunotoxin consisting of two antibody variable domains fused to Pseudomonas exotoxin, Nature 339(6223): 394-97; Neuberger et al. 1984, Recombinant antibodies possessing novel effector functions, Nature 312(5995): 604-08; Wallace et al., 2001, Exogenous antigen targeted to FcgammaRI on myeloid cells is presented in association with MHC class I, J. Immunol. Methods 248(1-2): 183-94) that may be used, according to the methods of the invention, as sources of structural elements and joining elements. Such multivalent, multispecific and multifunctional antibodies can be modified by the addition of functional groups for the construction of assembly units used for the fabrication of nanostructures as described herein.

### 5.4. Initiator Assembly Units and Their Immobilization to a Solid Support Matrix

An initiator assembly unit is the first assembly unit incorporated into a nanostructure that is formed by the staged assembly method of the invention. An initiator assembly unit may be attached, in certain embodiments, by covalent or non-covalent interactions, to a solid substrate or other matrix. An initiator assembly unit is also known as an “initiator unit.” Staged assembly of a nanostructure begins by the non-covalent, vectorial addition of a selected assembly unit to the initiator unit. According to the methods of the invention, an assembly unit is added to the initiator unit through (i) the incubation of an initiator unit, which in some embodiments, is immobilized to a matrix or substrate, in a solution comprising the next assembly unit to be added. This incubation step is followed by (ii) a removal step, e.g., a washing step, in which excess assembly units are removed from the proximity of the initiator unit.

Assembly units bind to the initiator unit through the formation of specific, non-covalent bonds. The joining elements of the next assembly unit are chosen so that they attach only at pre-designated sites on the initiator unit. Only one assembly unit can be added to a target joining element on the initiator unit during the first staged-assembly cycle, and binding of the assembly unit to the target initiator unit is vectorial. Staged assembly continues by repeating steps (i) and (ii) until all of the desired assembly units are incorporated into the nanostructure according to the desired design of the nanostructure.

In a preferred embodiment of the staged assembly method of the invention, an initiator unit is immobilized on a substrate and additional units are added sequentially in a procedure analogous to solid phase polymer synthesis.

An initiator unit is a category of assembly unit, and therefore can comprise any of the structural, joining, and/or functional elements described hereinbelow as being comprised in an assembly unit of the invention. An initiator unit can therefore comprise any of the following molecules, or a binding derivative or binding fragment thereof: a monoclonal antibody; a multispecific antibody, a Fab or F(ab′)₂ fragment, a single-chain antibody fragment (scFv); a bispecific, chimeric or bispecific heterodimeric F(ab′)₂; a diabody or multimeric scFv fragment; a bacterial pilin protein, a leucine zipper-type coiled coil, a four-helix bundle, a peptide epitope, or a PNA, or any other type of assembly unit disclosed herein.

In certain embodiments, the invention provides an initiator assembly unit which comprises at least one joining element. In other embodiments, the invention provides an initiator assembly unit with two or more joining elements.

Initiator units may be tethered to a matrix in a variety of ways. The choice of tethering method will be determined by several design factors including, but not limited to: the type of initiator unit, whether the finished nanostructure must be removed from the matrix, the chemistry of the finished nanostructure, etc. Potential tethering methods include, but are not limited to, antibody binding to initiator epitopes, His tagged initiators, initiator units containing matrix binding domains (e.g., chitin-binding domain, cellulose-binding domain), antibody binding proteins (e.g., protein A or protein G) for antibody or antibody-derived initiator units, streptavidin binding of biotinylated initiators, PNA tethers, and specific covalent attachment of initiators to matrix.

In certain embodiments, an initiator unit is immobilized on a solid substrate. Initiator units may be immobilized on solid substrates using methods commonly used in the art for immobilization of antibodies or antigens. There are numerous methods well known in the art for immobilization of antibodies or antigens. These methods include non-specific adsorption onto plastic ELISA plates; biotinylation of a protein, followed by immobilization by binding onto streptavidin or avidin that has been previously adsorbed to a plastic substrate (see, e.g., Sparks et al., 1996, Screening phage-displayed random peptide libraries, in Phage Display of Peptides and Proteins, A Laboratory manual, editors, B. K. Kay, J. Winter and J. McCafferty, Academic Press, San Diego, pp. 227-53). In addition to ELISA microtiter plates, protein may be immobilized onto any number of other solid supports such as Sepharose (Dedman et al., 1993, Selection of target biological modifiers from a bacteriophage library of random peptides: the identification of novel calmodulin regulatory peptides, J. Biol. Chem. 268; 23025-30) or paramagnetic beads (Sparks et al., 1996, Screening phage-displayed random peptide libraries, in Phage Display of Peptides and Proteins, A Laboratory manual, editors, B. K. Kay, J. Winter and J. McCafferty, Academic Press, San Diego, pp. 227-53). Additional methods that may be used include immobilization by reductive amination of amine-containing biological molecules onto aldehyde-containing solid supports (Hermanson, 1996, Bioconjugate Techniques, Academic Press, San Diego, p. 186), and the use of dimethyl pimelimidate (DMP), a homobifunctional cross-linking agent that has imidoester groups on either end (Hermanson, 1996, Bioconjugate Techniques, Academic Press, San Diego, pp. 205-06). This reagent has found use in the immobilization of antibody molecules to insoluble supports containing bound protein A (e.g., Schneider et al., 1982, A one-step purification of membrane proteins using a high efficiency immunomatrix, J. Biol. Chem. 257, 10766-69).

In a specific embodiment, an initiator unit is a diabody that comprises a tethering domain (T) that recognizes and binds an immobilized antigen/hapten and an opposing domain (A) to which additional assembly units are sequentially added in a staged assembly. Antibody 8F5, which is directed against the antigenic peptide VKAETRLNPDLQPTE (SEQ ID NO: 70) derived human rhinovirus (Serotype 2) viral capsid protein Vp2, is used as the T domain (Tormo et al., 1994, Crystal structure of a human rhinovirus neutralizing antibody complexed with a peptide derived from viral capsid protein VP2, EMBO J. 13(10): 2247-56). The A domain is the same lysozyme anti-idiotopic antibody (E5.2) previously described for Diabody Unit 1. The completed initiator assembly unit therefore contains 8F5×730.1.4 (T×A ) as the opposing CDRs. The initiator unit is constructed and functionally characterized using the methods described herein for characterizing joining elements and/or structural elements comprising diabodies.

In order to immobilize the initiator unit onto a solid support matrix, the rhinovirus antigenic peptide may fused to the protease recognition peptide factor Xa through a short flexible linker spliced at the N termini of the Factor Xa sequence, IEGR, (Nagai and Thogersen, 1984, Generation of beta-globin by sequence-specific proteolysis of a hybrid protein produced in Escherichia coli, Nature309(5971): 810-12) and between the Factor Xa sequence and the antigenic peptide sequence. This fusion peptide may be covalently linked to CH-Sepharose 4B (Pharmacia); a sepharose derivative that has a six-carbon long spacer arm and permits coupling via primary amines. (Alternatively, Sepharose derivatives for covalent attachment via carboxyl groups may be used.) The covalently attached fusion protein will serve as a recognition epitope for the tethering domain “8F5” in the initiator unit (T×A).

Once the initiator is immobilized, additional diabody units (diabody assembly units 1 and 2) may be sequentially added in a staged assembly, unidirectionally from binding domain A′. Upon completion of the staged assembly, the nanostructure may be either cross-linked to the support matrix or released from the matrix upon addition of the protease Factor Xa. The protease will cleave the covalently attached antigenic /Factor Xa fusion peptide, releasing the intact nanostructure from the support matrix, since, by design, there are no Factor Xa recognition sites contained within any of the designed protein assembly units.

An alternate strategy of cleaving the peptide fusion from the solid support matrix that does not require the addition of Factor Xa, can also be implemented. This method utilizes a cleavable spacer arm attached to the sepharose matrix. The antigen peptide is covalently attached through a phenyl-ester linkage to the matrix. Once the immobilized antibody binds initiator assembly unit, the initiator assembly unit remains tethered to the support matrix until chemical cleavage of the spacer arm with imidazoleglycine buffer at pH 7.4 at which point the initiator unit/antigen complex (and associated nanostructure) are released from the support matrix.

### 5.5. Structural Elements

In certain embodiments of the present invention, an assembly unit comprises a structural element. The structural element generally has a rigid structure (although in certain embodiments, described below, the structural element may be non-rigid). The structural element is preferably a defined peptide, protein or protein fragment of known size and structure that comprises at least about 50 amino acids and, generally, fewer than 2000 amino acids. Peptides, proteins and protein fragments are preferred since naturally-occurring peptides, proteins and protein fragments have well-defined structures, with structured cores that provide stable spatial relationships between and among the different faces of the protein. This property allows the structural element to maintain pre-designed geometric relationships between the joining elements and functional elements of the assembly unit, and the relative positions and stoichiometries of assembly units to which it is bound.

The use of proteins as structural elements has particular advantages over other choices such as inorganic nanoparticles. Most populations of inorganic nanoparticles are heterogeneous, making them unattractive scaffolds for the assembly of a nanostructure. In most populations, each inorganic nanoparticle is made up of a different number of atoms, with different geometric relationships between facets and crystal faces, as well as defects and impurities. A comparably sized population of proteins is, by contrast, very homogeneous, with each protein comprised of the same number of amino acids, each arranged in approximately the same way, differing in arrangement, for the most part, only through the effect of thermal fluctuations. Consequently, two proteins designed to interact with one another will always interact with the same geometry, resulting in the formation of a complex of predictable geometry and stoichiometry. This property is essential for massively parallel “bottom-up” assembly of nanostructures.

A structural element may be used to maintain the geometric relationships among the joining elements and functional elements of a nanostructure. As such, a rigid structural element is generally preferred for construction of nanostructures using the staged assembly methods described herein. This rigidity is typical of many proteins and may be conferred upon the protein through the properties of the secondary structural elements making up the protein, such as a-helices and β-sheets.

Structural elements may be based on the structure of proteins, protein fragments or peptides whose three-dimensional structure is known or may be designed ab initio. Examples of proteins or protein fragments that may be utilized as structural elements in an assembly unit include, but are not limited to, antibody domains, diabodies, single-chain antibody variable domains, and bacterial pilins.

In some embodiments, structural elements, joining elements and functional elements may be of well-defined extent, separated, for example, by glycine linkers. In other embodiments, joining elements may involve peptides or protein segments that are integral parts of a structural element, or may comprise multiple loops at one end of a structural element, such as in the case of the complementarity determining regions (CDRs) of antibody variable domains (Kabat et al., 1983, Sequences of Proteins of Immunological Interest, U.S. Department of Health and Human Services). A CDR is a joining element that is an integral part of the variable domain of an antibody. The variable domain represents a structural element and the boundary between the structural element and the CDR making up the joining element (although well-defined in the literature on the basis of the comparisons of many antibody sequences) may not always be completely unambiguous structurally. There may not always be a well-defined boundary between a structural element and a joining element, and the boundary between these domains, although well-defined on the basis of their respective utilities, may be ambiguous spatially.

Structural elements of the present invention comprise, e.g., core structural elements of naturally-occurring proteins that are then modified to incorporate joining elements, functional elements, and/or a flexible domain (e.g., a tri-, tetra- or pentaglycine), thereby providing useful assembly units. Consequently, in certain embodiments, structures of existing proteins are analyzed to identify those portions of the protein or part thereof that can be modified without substantially affecting the rigid structure of that protein or protein part.

For example, in certain embodiments, the amino acid sequence of surface loop regions of a protein or structural element are altered with little impact on the overall folding of the protein. The amino acid sequences of a surface loop of a protein are generally preferred as amino acid positions into which the additional amino acid sequence of a joining element, a functional element, and/or a flexible domain may be inserted, with the lowest probability of disrupting the protein structure. Determining the position of surface loops in a protein is carried out by examination of the three-dimensional structure of the protein or a homolog thereof, if three-dimensional atomic coordinates are available, using, for example, a public-domain protein visualization computer program such as RASMOL (Sayle et al., 1995, RasMol: Biomolecular graphics for all, Trends Biochem. Sci. (TIBS) 20(9): 374-376; Saqi et al., 1994, PdbMotif—a tool for the automatic identification and display of motifs in protein structures, Comput. Appl. Biosci. 10(5): 545-46). In this manner, amino acids included in surface loops, and the relative spatial locations of these surface loops, can be determined.

If the three-dimensional structure of the protein being engineered is not known, but that of a close homolog is known (as is the case, for example, for essentially all antibody molecules), the amino acid sequence of the molecule of interest, or a portion thereof, can be aligned with that of the molecule whose three-dimensional structure is known. This comparison (done, for example, using BLAST (Altschul et al., 1997, Gapped BLAST and PSI-BLAST: a new generation of protein database search programs, Nucleic Acids Res. 25: 3389-3402) or LALIGN (Huang and Miller, 1991, A time efficient, linear-space local similarity algorithm, Adv. Appl. Math. 12: 337-357) allows identification of all the amino acids in the protein of interest that correspond to amino acids that constitute surface loops (β-turns) in the protein of known three-dimensional structure. In regions in which there is high sequence similarity between the two proteins, this identification is carried out with a high level of certainty. Once a putative loop is identified and altered according to methods disclosed herein, the resultant construct is tested to determine if it has the expected properties. This analysis is performed even in those instances where identification of the loop is highly reliable, e.g. where that determination is based upon a known three-dimensional protein structure.

### 5.5.1. Structural Elements Comprising Antibodies or Binding Derivatives or Binding Fragments Thereof

Antibodies are multivalent molecules made up of polypeptide chains including light (L) chains of approximately 220 amino acids and heavy (H) chains of 450-575 amino acids. The average molecular weight for an intact IgG molecule is in the range 152-196 kD. Structural studies performed on antibodies have revealed that both the light and heavy chains contain a characteristic domain termed the “immunoglobulin fold.” The immunoglobulin fold is defined as a barrel-shaped sandwich consisting of two layered anti-parallel P-sheets linked together by a disulfide bond. The predominant secondary structure in an antibody is an anti-parallel β-sheet with short stretches of α-helix. (For review, see Padlan, 1994, Anatomy of the antibody molecule, Mol. Immunol. 31(3): 169-217; Padlan, 1996, X-ray crystallography of antibodies, Adv. Protein Chem. 49: 57-133; and references cited therein.)

The light chains contain two immunoglobulin domains, one at the N-terminal portion, which varies from antibody to antibody (V_{L}), and the other at the C-terminal portion, which is relatively constant (C_{L}). The heavy chains contain four or five immunoglobulin domains, depending upon the class of immunoglobulin. The N-terminal domain varies (V_{H}) and the other distal domains remain constant (C_{H}1, C_{H}2, C_{H}3, and, in certain cases C_{H}4). The units of the light and heavy chains associate through disulfide bonds as well as other non-covalent interactions to form the characteristic Y-shaped dimer composed of two light chains and two heavy chains. The antibody fragment containing the V_{L} chain and the V_{H} chain is termed the Fᵥ fragment. The portion containing the entire light chain, as well as the variable portion and first constant domain (C_{H}1) of the heavy chain, is termed the Fab fragment. Interactions of the variable domains with the constant domains in Fab are not very strong, lending a degree of flexibility and positional variability to the overall structure of the molecule. There can be a large variation (from 127-176°) in the angle between the Fab variable domain and the Fab constant domain. This angle is known as the Fab “elbow” or “bend” (Padlan, 1994, Anatomy of the antibody molecule. Mol. Immunol. 31(3): 169-217).

The N-terminal regions of the two Fab arms bind antigen (Mian et al., 1991, Structure, function and properties of antibody binding sites, J. Mol. Biol. 217(1): 133-51; Wilson et al., 1994, Structure of anti-peptide antibody complexes, Res. Immunol. 145(1): 73-8; Wilson et al., 1994, Antibody-antigen interactions: new structures and new conformational changes, Curr. Opin. Struct. Biol. 4(6): 857-67). The Fab arms, in turn, are connected by a flexible polypeptide to the third fragment, termed the Fc fragment, which is responsible for triggering effector functions that eliminate the antigen as well as dimerize the antigen binding sites.

The Fc portion of the IgG antibody molecule is made up of the two constant domains C_{H}2 and C_{H}3. The polypeptide segment connecting the Fab and Fc fragments is defined as the hinge and has variable length and flexibility depending upon the antibody class and isotype. This flexible hinge region provides a natural demarcation between the Fc and Fab fragments of the antibody. The hinge and the Fab elbow or bend contained in an intact IgG molecule allow for significant flexibility between the two antigen binding sites and thus permit numerous cross-linking geometries (FIGS. 5 and 6).

The proteins making up native and recombinant antibody fragments are candidates for the structural elements of nanostructures assembled by staged assembly. Antibodies used in the staged assembly methods of the invention include, but are not limited to, IgG monoclonal, humanized or chimeric antibodies. Binding derivatives or binding fragments of antibodies used in the staged assembly methods of the invention also include, but are not limited to, single chain antibodies (scFv) including monomeric ((scFv) fragments), dimeric ((scFv)₂ or diabodies), trimeric ((scFv)₃ or triabodies) and tetrameric ((scFv)₄ or tetrabodies) single chain antibodies; Fab fragments; F(ab′)₂ fragments; and fragments produced by a Fab expression library (Huse et al., 1989, Generation of a large combinatorial library of the immunoglobulin repertoire in phage lambda, Science, 246, 1275-81).

### 5.5.1.1. Antibody Production

General methods of antibody production and use are commonly known in the art. These methods may be used for producing structural and joining elements for use in the staged assembly methods and assembly units of the invention (see, e.g., Harlow and Lane, 1988, Antibodies: A Laboratory Manual, Cold Spring Harbor Laboratory Press, Cold Spring Harbor, New York; incorporated herein by reference in its entirety).

A molecular clone of an antibody to an antigen of interest can be prepared by techniques well-known in the art. Recombinant DNA methodology may be used to construct nucleic acid sequences that encode a monoclonal antibody molecule, or antigen binding region thereof (see, e.g., Sambrook et al., 2001, Molecular Cloning, A Laboratory Manual, Third Edition, Chapters 1, 2, 3, 5, 6, 8, 9, 10, 13, 14, 15 and 18, Cold Spring Harbor Laboratory Press, N.Y.; Ausubel et al., 1989, Current Protocols in Molecular Biology, Chapters 1, 2, 3, 5, 6, 8, 10, 11, 12, 15, 16, 19, 20 and 24, Green Publishing Associates and Wiley Interscience, N.Y.; Current Protocols in Immunology, Chapters 2, 8, 9, 10, 17 and 18, John Wiley & Sons, 2001, Editors John E. Coligan, Ada M. Kruisbeek, David H. Margulies, Ethan M. Shevach, Warren Strober, Series Editor: Richard Coico).

Antibodies can be expressed in bacteria either intracellularly or extracellularly by secretion into the bacterial periplasm (Tomlinson and Holliger, 2000, Methods for generating multivalent and bispecific antibody fragments, Methods Enzymol. 326: 461-79). Intracellular expression of recombinant antibodies, however, frequently leads to the formation of insoluble aggregates of the protein, which are referred to as inclusion bodies, presumably due to the non-reducing environment of the bacterial cytoplasm, which inhibits disulfide bond formation between antibody domains. It is possible to refold the antibodies into functional proteins through solubilization of the inclusion bodies with strong denaturants followed by exposure to renaturing conditions, by methods commonly known in the art.

In order to circumvent the need for renaturation, a coding sequence for a bacterially-derived periplasmic signal sequence can be spliced at the N-terminal portion of the gene encoding the antibody to direct the recombinant protein to the bacterial periplasm. The oxidizing environment of the periplasmic space favors proper folding of the antibody domains, including disulfide bond formation. The success of these methods in producing good yields of functional antibody can depend upon the antibody type, derivation and method of overproduction (see Ward, 1992, Antibody engineering: the use of Escherichia coli as an expression host, FASEB J. 6(7): 2422-27; Ward, 1993, Antibody engineering using Escherichia coli as host, Adv. Pharmacol. 24: 1-20; Zhu et al., 1996, High level secretion of a humanized bispecific diabody from Escherichia coli, Biotechnology (NY) 14(2): 192-96; Sheets et al., 1998, Efficient construction of a large nonimmune phage antibody library: the production of high-affinity human single-chain antibodies to protein antigens, Proc. Natl. Acad. Sci. USA 95(11): 6157-62; Tomlinson et al., 2000, Methods for generating multivalent and bispecific antibody fragments, Methods Enzymol. 326: 461-79).

Antibody molecules may be purified by techniques well-known in the art, e.g., immunoabsorption or immunoaffinity chromatography, chromatographic methods such as HPLC (high performance liquid chromatography), or a combination thereof.

### .5.1.2. Structural Elements Comprising Monoclonal Antibodies

Monoclonal antibodies (mAbs), or binding derivatives or binding fragments thereof, may be used as structural elements according to the methods of the invention. mAbs are homogeneous populations of antibodies directed against a particular antigen. A mAb to an antigen of interest can be prepared by using any technique known in the art that provides for the production of antibody molecules. These include, e.g., the hybridoma technique originally described by Kohler and Milstein (1975, Continuous cultures of fused cells secreting antibody of predefined specificity, Nature 256: 495-97; Voet and Voet, 1990, Biochemistry, John Wiley and Sons, Inc., Chapter 34), the human B cell hybridoma technique (Kozbor et al., 1983, Immunology Today 4: 72-79; Kozbor et al., U.S. Pat. No. 4,693,975, entitled “Human hybridroma [sic] fusion partner for production of human monoclonal antibodies,” issued Sep. 15, 1987), and the EBV-hybridoma technique (Cole et al., 1985, Monoclonal Antibodies and Cancer Therapy, Alan R. Liss, Inc., pp. 77-96; Roder et al., 1986, The EBV-hybridoma technique, Methods Enzymol. 121: 140-67). Such antibodies may be of any immunoglobulin class including IgG, IgM, IgE, IgA, IgD and any subclass thereof. The mAbs that may be used in the methods of the invention may be synthesized by any technique commonly known in the art. For example, human monoclonal antibodies may be made by any of numerous techniques known in the art (e.g., Teng et al., 1983, Construction and testing of mouse—human heteromyelomas for human monoclonal antibody production, Proc. Natl. Acad. Sci. USA. 80: 7308-12; Cole et al., 1984, Human monoclonal antibodies, Mol. Cell. Biochem. 62(2): 109-20; Olsson et al., 1982, Immunochemical Techniques, Meth. Enzymol. 92: 3-16).

By contrast, polyclonal antibodies cannot be used as components in the present invention. Polyclonal antibodies represent a population of antibodies in which many molecules of different precise specificity exists. Although they may all bind to a particular antigen, they will bind different parts of the antigen with different geometries, a property that is inconsistent with the precise assembly of a nanostructure.

### 5.5.1.3. Structural Elements Comprising Multispecific Antibodies

Multispecific antibodies, or binding derivatives or binding fragments thereof, may be used as structural elements for use in the staged assembly methods of the invention. “Specific” or “specificity,” as used herein, refers to the ability of an antibody to bind a defined epitope to one distinct antigen-recognition site. Bispecific antibodies, therefore, comprise two distinct antigen recognition sites, each capable of binding a different antigen. Multispecific antibodies have the ability to bind more than two different epitopes, each through the action of a distinct joining element, i.e., an antigen-recognition site.

In certain embodiments, homogeneous bispecific or multispecific mAbs can be created for use as structural elements, via immortilization of lymphocyte clones, created by fusing myeloma cells with lymphocytes raised against an antigen of interest as described above generally for the production of monoclonal antibodies. By such methods, multispecific mAbs can be produced in virtually unlimited quantities. Using methods well-known in the art, multispecific mAbs may be created that specifically target and bind a selected biological substance (see, e.g., Colcher et al., 1999, Single-chain antibodies in pancreatic cancer, Ann. NY Acad. Sci. 880: 263-80; Hudson, 1999, Recombinant antibody constructs in cancer therapy, Curr. Opin. Immunol. 11(5): 548-57; Kipriyanov et al., 1999, Bispecific tandem diabody for tumor therapy with improved antigen binding and pharmacokinetics, J. Mol. Biol. 293(1): 41-56; Segal et al., 1999, Bispecific antibodies in cancer therapy, Curr. Opin. Immunol. 11(5): 558-62; Trail et al., 1999, Monoclonal antibody drug conjugates in the treatment of cancer, Curr. Opin. Immunol. 11(5): 584-88; Hudson, 2000, Recombinant antibodies: a novel approach to cancer diagnosis and therapy, Expert Opin. Investig. Drugs 9(6): 1231-42).

In certain embodiments, a multispecific mAb for use as a structural element according to the methods of the invention may be a bispecific and/or bivalent mAb. A bispecific antibody has the ability to bind two different epitopes, each contained on a distinct antigen-recognition site. A bivalent antibody has the ability to bind to two different epitopes.

Bispecific antibodies may be created using methods well-known in the art (see, e.g., Weiner et al., 1995, Bispecific monoclonal antibody therapy of B-cell malignancy, Leuk. Lymphoma 16(3-4): 199-207; Helfrich et al., 1998, Construction and characterization of a bispecific diabody for retargeting T cells to human carcinomas, Int. J. Cancer 76(2): 232-39; Arndt et al., 1999, A bispecific diabody that mediates natural killer cell cytotoxicity against xenotransplanted human Hodgkin's tumors, Blood 94(8): 2562-8; Kipriyanov et al., 1999, Bispecific tandem diabody for tumor therapy with improved antigen binding and pharmacokinetics, J. Mol. Biol. 293(1): 41-56; Sundarapandiyan et al., 2001, Bispecific antibody-mediated destruction of Hodgkin's lymphoma cells, J. Immunol. Methods 248(1-2): 113-23).

Technologies for the production of multivalent and multispecific antibodies are well known in the art (see, e.g., Pluckthun et al., 1997, New protein engineering approaches to multivalent and bispecific antibody fragments, Immunotechnology 3(2): 83-105; Santos et al., 1998, Development of more efficacious antibodies for medical therapy and diagnosis, Prog. Nucleic Acid Res. Mol. Biol. 60: 169-94; Alt et al., 1999, Novel tetravalent and bispecific IgG-like antibody molecules combining single-chain diabodies with the immunoglobulin gammal Fc or CH3 region, FEBS Lett. 454(1-2): 90-94; Hudson et al., 1999, High avidity scFv multimers; diabodies and triabodies, J. Immunol. Methods 231(1-2): 177-89; Tomlinson et al., 2000, Methods for generating multivalent and bispecific antibody fragments, Methods Enzymol. 326: 461-79; Todorovska et al., 2001, Design and application of diabodies, triabodies and tetrabodies for cancer targeting. J. Immunol. Methods 248(1-2): 47-66). For example, genes encoding antibodies of known specificity may be rescued from hybridoma cell lines and can provide the starting material for cloning the rearranged V_{L} and V_{H} genes thorough employment of recombinant DNA technologies (Ward et al., 1989, Binding activities of a repertoire of single immunoglobulin variable domains secreted from Escherichia coli, Nature 341(6242): 544-46; Sheets et al., 1998, Efficient construction of a large nonimmune phage antibody library: the production of high-affinity human single-chain antibodies to protein antigens, Proc. Natl. Acad. Sci. USA 95(11): 6157-62). Universal DNA primers may be designed to anneal to the target V-domain genes and amplified through employment of the polymerase chain reaction. Through design of restriction sites within these primers, the resulting amplified DNA products can be cloned directly for expression in a range of different hosts including bacteria, yeast, plant and insect cells (Tomlinson et al., 2000, Methods for generating multivalent and bispecific antibody fragments, Methods Enzymol. 326: 461-79). These host cells, rather than hybridoma cell lines, can be used, for the production of recombinant engineered antibodies for use in the methods of the invention.

In certain embodiments of the invention, a structural element comprises a diabody fragment. A diabody has two CDRs, and is capable of making two highly specific, non-covalent interactions. A diabody, or a binding derivative or binding fragment thereof, may be incorporated into a nanostructure in such a way that only one of the two CDRs is used. In certain embodiments, the CDRs themselves serve as joining elements, and the body of the diabody between the two CDRs serves as a structural element.

Methods well known in the art are used for the expression, purification and characterization of diabody fragments from E. coli (Poljak, 1994, An idiotope—anti-idiotope complex and the structural basis of molecular mimicking, Proc. Natl. Acad. Sci. USA 91(5): 1599-600; Zhu et al., 1996, High level secretion of a humanized bispecific diabody from Escherichia coli, Biotechnology (NY) 14(2): 192-96; Todorovska et al., 2001, Design and application of diabodies, triabodies and tetrabodies for cancer targeting, J. Immunol. Methods 248(1-2): 47-66). Examples of a structural element comprising a diabody fragment are illustrated in FIG. 7. The diabody expression cassettes represented in FIG. 7 are designed so that the pelB signal sequence spliced to the N-terminus of the V_{H} domains genes coding the diabody fragments are targeted and secreted into the E. coli periplasmic space, where the oxidative environment allows proper folding of the diabody. After induction, the overexpressed diabodies fragments are harvested from the E coli periplasm according to established protocols well-known in the art.

In a preferred embodiment of the invention (FIG. 7), diabodies are engineered to add a hexahistidine tag (His6) at the C-terminus of the V_{L} domains to facilitate purification using an immobilized metal affinity chromatography resin (Scopes, 1994, Protein Purification, Principles and Practice, Third Edition, Springer-Verlag, London, pp. 183-85; Scopes, 1994 Protein Purification: Principles and practice (Springer Advanced texts in Chemistry), Third ed., London). Protein overexpression of diabody assembly unit-1 (FIG. 7A), for example, will contain a mixture of species including; 2 (V_{H}A×V_{L}BHis₆), 2(V_{H}B×V_{L}A), and (V_{H}B×V_{L}A, V_{H}A×V_{L}BHis₆). The number of His₆ tags determines the concentration of imidazole (20-250 mM gradient) at which each protein unit contained in the mixture will elute. Those with no hexahistidine tags will exhibit little or no affinity towards the column resin. Those with one hexahistidine tag will generally elute between 20-40 mM imidazole (bispecific diabody) and those with two hexahistidine tags will generally elute between 50 and 100 mM imidazole. Elution peaks may be detected by UV absorbance and verified with SDS-PAGE, native-PAGE or ELISA assay. Even though the purification procedure described above guards against the isolation of unwanted non-bispecific diabody byproducts, methods are employed to ensure that the isolated diabody of interest has functional bispecificity as disclosed hereinbelow.

FIG. 7A depicts an A×B diabody in which the V_{H} and V_{L} domains of A define a lysozyme isotopic antibody (D1.3) and in which the V_{H} and V_{L} domains of B define a virus neutralizing idiotopic antibody (730.1.4). In order to facilitate purification of the desired diabody product, the gene encoding V_{H}A and V_{L}B includes a hexahistidine tag, whereas the gene encoding V_{H}B and V_{L}A does not. FIG. 7B depicts a B′×A′ diabody in which the V_{H} and V_{L} domains of B′ define a virus neutralizing idiotopic antibody (409.5.3) and in which the V_{H} and V_{L} domains of A′ define a lysozyme isotopic antibody (E5.2). In order to facilitate purification of the desired diabody product, the gene encoding V_{H}B′ and V_{L} A′ includes a hexahistidine tag, whereas the gene encoding V_{H}A′ and V_{H}B′ does not.

In certain embodiments, sandwich ELISA or BlAcore protocols may be implemented to determine simultaneous and dual occupancy of both antigen-binding sites (bispecificity), as well as equilibrium constants (Abraham et al., 1996, Determination of binding constants of diabodies directed against prostate-specific antigen using electrochemiluminescence-based immunoassays, J. Mol. Recognit. 9(5-6): 456-61; McGuinness et al., 1996, Phage diabody repertoires for selection of large numbers of bispecific antibody fragments, Nat. Biotechnol. 14(9): 1149-54; McCall et al., 2001, Increasing the affinity for tumor antigen enhances bispecific antibody cytotoxicity, J. Immunol. 166(10): 6112-17). In a specific embodiment in which an idiotype/anti-idiotype binding constant is determined using the BlAcore technique, one of the antibodies is dissolved in a liquid phase and the other is coupled to the solid phase. Implementation of this technique permits the determination of the association and dissociation rates (kₒₙ and k_{off} respectively) for determination of the dissociation constant (Kd) (Goldbaum et al., 1997, Characterization of anti-anti-idiotypic antibodies that bind antigen and an anti-idiotype, Proc. Natl. Acad. Sci. USA 94(16): 8697-701). Other protocols that do not require recombinant antigens, but that can detect bispecificity may also be employed, and include the rosetting assay as described by Holliger et al. (1997, Retargeting serum immunoglobulin with bispecific diabodies, Nat. Biotechnol. 15(7): 632-36).

In a specific embodiment, a diabody may comprise one or more sites for the insertion of a joining element, a structural element or a functional element. Table 1 shows peptide regions contained in diabody units that may be used for the insertion of joining, structural or functional elements. A peptide region is a portion of a protein of interest, e.g., of an antibody or a binding derivative or binding fragment thereof. A peptide region is preferably exposed on the surface of the protein of interest, and is amenable to being re-engineered through the insertion of additional peptides or the alteration of its sequence or both. Table 1 summarizes the amino acids identified as β-turns located on the surface of a diabody with V_{H}-V_{L} variable domain linkage (pdb entry 1LMK). Residue regions are defined within the diabody fragment from analysis of the atomic coordinates and numbered according to the residue assignments deposited under entry 1LMK pdb. Chain assignments are labeled in accord with the corresponding deposited pdb coordinates.

TABLE 1
Identified Peptide Regions Contained in Diabody Structural
Elements for the Insertion of Joining, Structural or
Functional Elements

| Domain | Secondary Structure | Residue (Chain) |
|---|---|---|
| V_{H} | β-turn | Residues 13-16, 39-44, |
| | | 62-66, 73-77 |
| | | (A and C chains) |
| V_{L} | C-terminal α-C | Residue 312 |
| | | (A and C chains) |
| V_{H} | C-terminal α-C | Residue 1 |
| | | (A and C chains) |

In certain embodiments, binding sites may be added as joining elements to a diabody to make possible structural branches, forks, T-junctions, or multidimensional architectural binding sites, in addition to the two joining elements formed by the oppositely directed CDRs. Alteration of the sequence of surface loops in proteins appears to have little impact on the overall folding of a protein, and it is frequently possible to make insertion mutants at the sites of β-turns. The surface loops are the places where sequences can be added to the protein with the lowest probability of disrupting the protein structure.

Specific sites within the diabody unit have been precisely defined for insertions. For example, in certain embodiments, joining elements mays be spliced internal to, or replacing the β-turn residues as disclosed herein in Table 1. Since the general three-dimensional structure of diabodies is known, and since it is possible to homology-model the three-dimensional structure of diabodies of similar sequence (Guex and Peitsch, 1997, SWISS-MODEL and the Swiss-PdbViewer: An environment for comparative protein modelling, Electrophoresis 18: 2714-23; Guex and Peitsch, 1999, Molecular modelling of proteins, Immunology News 6: 132-34; Guex et al., 1999, Protein modelling for all, TIBS 24: 364-67, the β-turns located on the surface of a diabody of similar amino acid sequence to a diabody of known structure are readily identified by a sequence comparison (using, e.g., BLAST, Altschul et al., 1997, Gapped BLAST and PSI-BLAST: a new generation of protein database search programs, Nucleic Acids Res. 25: 3389-3402), followed by a visual investigation of the x-ray coordinates of the protein of similar sequence.

In one embodiment, a visual investigation of the three-dimensional structure of a diabody is performed with the molecular visualization package QUANTA (Accelrys Inc., San Diego, Calif.) run on a Silicon Graphics Workstation. The coordinates defining the three-dimensional positions of the atoms of a diabody molecule are included in the PDB entry 1LMK. Upon such an analysis, it is apparent that there are surface loops that include residues shown in Table 1, which represent sites with high potential for accepting the insertion of a peptide such as the HIV-1 V3 loop antigen. All amino acids included in surface loops of this diabody molecule can be determined from this information, and the relative spatial locations of these surface loops has also been determined. The information provided by the three-dimensional structure of the immunoglobulin being engineered (whether derived directly from X-ray crystallography, or from homology modeling based on a homologous structure) allows the identification of all the amino acids in the protein of interest that correspond to amino acids that constitute surface loops.

In a specific embodiment, DNA encoding a peptide epitope derived from the ras protein is inserted into a diabody assembly unit coding sequence at a site defined by visual investigation of the three-dimensional atomic coordinates as determined by x-ray crystallography. The ras epitope is flanked by four glycines on either side, to provide flexibility and accessibility for cognate antibody binding.

Once the diabody assembly unit/ras peptide protein fusion (represented as B×A) has been expressed and purified, it is characterized for retention of diabody valency and function as well as epitope recognition by the appropriate antibody by methods such as ELISA or BlAcore analysis.

Functional elements, such as enzymes, toxins, and antigenic peptides, have already been successfully spliced to the termini of scFv fragments resulting in various multifunctional antibodies (Chaudhary et al., 1989, A recombinant immunotoxin consisting of two antibody variable domains fused to Pseudomonas exotoxin, Nature 339(6223): 394-97; Suzuki et al., 1997, Construction, bacterial expression, and characterization of hapten-specific single-chain Fv and alkaline phosphatase fusion protein, J. Biochem. (Tokyo) 122(2): 322-29; Williams et al., 2001, Numerical selection of optimal tumor imaging agents with application to engineered antibodies, Cancer Biother. Radiopharm. 16(1): 25-35). Functional elements that are made up of proteins or peptides can be fused directly into the proteinaceous portion of an assembly unit using the methods of molecular biology followed by expression of the proteins in appropriate host.

### 5.5.1.4. Structural Elements Comprising Fab or F(ab′)₂ Antibody Fragments

In certain embodiments of the invention, a structural element for the staged assembly of a nanostructure comprises an antibody fragment. Such a fragment includes, but is not limited to, an Fab fragment, or an F(ab′)₂ fragment, which can be produced by pepsin digestion of an IgG antibody molecule, thereby releasing the Fc portion. Pepsin digestion can be followed by reducing the disulfide bridges between the resulting F(ab′)₂ fragments thereby generating single Fab fragments.

Fab fragments are elongated dirigible shaped molecules that contain a monovalent and monospecific CDR at the N-terminal end of the molecule. In certain embodiments, an assembly unit is engineered from a Fab fragment by inserting a peptide epitope at the C terminal portion of the Fab fragment. Consequently, a peptide fused to the C-terminus of the Fab fragment may act as a target for another engineered Fab, to provide a highly specific and tight interaction between adjacent Fabs in a nanostructure constructed by staged assembly. The size, shape and structure of the Fab fragment (FIG. 6) make it preferred for use as a structural element because it also comprises, by virtue of its structure, a naturally occurring joining element. Electron micrographic and X-ray structural studies have revealed that the proximal portion of the Fab fragment is often linearly opposed to the distal portion (Fischmann et al., 1991, Crystallographic refinement of the three-dimensional structure of the FabD1.3-lysozyme complex at 2.5 A resolution, J. Bio. Chem 266: 12915-20; Ban et al., 1994, Crystal structure of an idiotype-anti-idiotype Fab complex, Proc. Natl. Acad. Sci. U.S.A. 91(5): 1604-8; Padlan, 1996, X-ray crystallography of antibodies, Adv. Protein Chem. 49: 57-133; Harris, Skaletsky et al., 1998). The flexible elbow bend, which is located in the middle of the fragment, allows for alternative geometries (Roux et al., 1997, Flexibility of human IgG subclasses, J. Immunol. 159(7): 3372-82; Roux et al., 1998, Comparisons of the ability of human IgG3 hinge mutants, IgM, IgE, and IgA2, to form small immune complexes: a role for flexibility and geometry, J. Immunol. 161(8): 4083-90). Alternatively, Fab expression libraries may be constructed (Huse et al., 1989, Generation of a large combinatorial library of the immunoglobulin repertoire in phage lambda, Science, 246, 1275-81) to allow rapid and easy identification of monoclonal Fab fragments with the desired specificity.

### 5.5.1.5. Structural Elements comprising Single-Chain Antibody Fragments (scFvs)

According to the methods of the invention, staged assembly of nanostructures can employ, in certain embodiments, structural elements comprising single-chain scFv fragments. An scFv antibody is composed of a fusion peptide that links the carboxyl terminus of the Fv variable heavy chain (V_{H}) to the amino terminus of the Fv variable light chain (V_{L}) or vice versa (Freund et al., 1994, Structural and dynamic properties of the Fv fragment and the single-chain Fv fragment of an antibody in solution investigated by heteronuclear three-dimensional NMR spectroscopy, Biochemistry 33(11): 3296-303; Hudson et al., 1999, High avidity scFv multimers; diabodies and triabodies, J. Immunol. Methods 231(1-2): 177-89; Le Gall et al., 1999, Di-, tri- and tetrameric single chain Fv antibody fragments against human CD19: effect of valency on cell binding, FEBS Lett 453(1-2): 164-68; Worn et al., 2001, Stability engineering of antibody single-chain Fv fragments, J. Mol. Biol. 305(5): 989-1010).

Single-chain antibodies may also be used as structural elements for use in the staged assembly methods of the invention. Single-chain antibodies may be produced by the methods of, e.g., Ladner; (U.S. Pat. No. 4,946,778, entitled “Single polypeptide chain binding molecules,” issued Aug. 7, 1990); Bird (1988, Single-Chain Antigen-Binding Proteins, Science 242(4877): 423-26); Huston et al. (1988, Protein engineering of antibody binding sites: recovery of specific activity in an anti-digoxin single-chain Fv analogue produced in Escherichia coli, Proc. Natl. Acad. Sci. USA 85: 5879-83), or Ward et al., (1989, Binding activities of a repertoire of single immunoglobulin variable domains secreted from Escherichia coli, Nature 334: 544-46).

An scFv fragment is a substructure of a Fab fragment that can be visualized as a Fab fragment, cut in half at the elbow-bend, missing the terminal constant light and heavy chain domains Freund et al., 1994, Structural and dynamic properties of the Fv fragment and the single- chain Fv fragment of an antibody in solution investigated by heteronuclear three-dimensional NMR spectroscopy, Biochemistry 33(11): 3296-303; Malby et al., 1998, Three-dimensional structures of single-chain Fv-neuraminidase complexes, J. Mol. Biol. 279(4): 901-10) (FIG. 8). Rather than being elongated and dirigible shaped, as in Fab fragments, scFv are smaller and more globular shaped. While approximately half the size of a Fab fragment, a scFv fragment still contains a functional monovalent/monospecific CDR at the N-terminal portion of the molecule. The scFv represents the minimal antigen binding motif that can be expressed in E. coli.

In general, scFv fragments are monovalent, maintaining tertiary and quaternary structures similar to that found in the Fv portion of an intact antibody (FIGS. 5 and 8) (Boulot et al., 1990, Crystallization and preliminary X-ray diffraction study of the bacterially expressed Fv from the monoclonal anti-lysozyme antibody D1.3 and of its complex with the antigen, lysozyme, J. Mol. Biol. 213(4): 617-19; Braden et al., 1996, Crystal structure of an Fv-Fv idiotope-anti-idiotope complex at 1.9 A resolution, J. Mol. Biol. 264(1): 137-51; Fuchs et al., 1997, Primary structure and functional scFv antibody expression of an antibody against the human protooncogene c-myc, Hybridoma 16(3): 227-33; Hoedemaeker et al., 1997, A single chain Fv fragment of P-glycoprotein-specific monoclonal antibody C219. Design, expression, and crystal structure at 2.4 A resolution, J. Biol. Chem. 272(47): 29784-89; Malby et al., 1998, Three-dimensional structures of single-chain Fv-neuraminidase complexes, J. Mol. Biol. 279(4): 901-10). A Gly/Ser peptide linker that is, optimally, 15 amino acids in length, can be used to join the two variable fragments and help maintain favorable interactions between the V, and V_{L} domains (Perisic et al. 1994, Crystal structure of a diabody, a bivalent antibody fragment, Structure 2(12): 1217-26; Takemura et al., 2000, Construction of a diabody (small recombinant bispecific antibody) using a refolding system, Protein Eng. 13(8): 583-88; Worn et al., 2001, Stability engineering of antibody single-chain Fv fragments, J. Mol. Biol. 305(5): 989-1010). These Gly/Ser linkers can be used to provide flexibility and protease resistance. Furthermore, scFv antibody fragments have similar function, in terms of antigen recognition and binding, as that of intact antibodies.

The smaller size of the scFv fragment, as well as the relative positioning of the CDR, make it well-suited as a protein component to be incorporated into assembly units of the present invention for fabrication of nanostructures. One advantage of scFv over Fab fragments is that the technology for engineering and producing scFv's is more advanced (see, e.g., Ward, 1993, Antibody engineering using Escherichia coli as host, Adv. Pharmacol. 24: 1-20; Luo et al., 1996, Construction and expression of bi-functional proteins of single-chain Fv with effector domains, J. Biochem. (Tokyo) 120(2): 229-32; Wu et al., 2000, Designer genes: recombinant antibody fragments for biological imaging, Q. J. Nucl. Med. 44(3): 268-83; Worn et al., 2001, Stability engineering of antibody single-chain Fv fragments, J. Mol. Biol. 305(5): 989-1010). Using these art-known methods, specific CDRs may be created, and functional elements may be added to scFv's for use as protein components to be incorporated into assembly units useful in for staged assembly of nanostructures.

In another embodiment, a similar strategy is used to incorporate additional intermolecular binding sites on the scFv as was described above for Fab fragments. The C-terminal distal portion or β-turn regions can be replaced by defined peptide epitopes such as, but not limited to those provided in Table 6, below. These peptide epitopes can replace defined β-turn motifs or be directly linked to the C-terminal amino acid of the V_{H} or V_{L} heavy chain (depending upon the order of the linked heavy and light variable domains) (Table 7), by manipulation of the appropriate encoding DNA sequences using recombinant DNA procedures well known in the art. The resulting scFv fragment will contain an antigen binding recognition site on one portion of the scFv fragment and a joining element that is a peptide epitope, either replacing the defined β-turn motifs, or linked at the C-terminal portion of the scFv fragment. Thus the fused peptide epitope will serve as a highly specific joining element in the formation of a joining pair between adjacent assembly units comprising scFv in a staged assembly.

### 5.5.1.6. Structural Elements Comprising Bispecific IgG, Chimeric IgG or Bispecific Heterodimeric F(ab′)₂ Antibodies

In certain embodiments of the invention, a structural element comprises an antibody fragment such as a bispecific IgG fragment, chimeric IgG fragment or a bispecific heterodimeric F(ab′)₂ antibody fragment. Whereas naturally occurring IgG molecules are bivalent by design, but monospecific because their CDRs are identical, IgG molecules, such as those created by hybridoma technology, can be produced that are either bivalent or bispecific, using the methods of, e.g., Suresh et al. (1986, Bispecific monoclonal antibodies from hybrid hybridomas, Methods Enzymol. 121: 210-28); Holliger et al. (1993, Engineering bispecific antibodies, Curr. Opin. Biotechnol. 4(4): 446-49); Hayden et al. (1997, Antibody engineering, Curr. Opin. Immunol. 9(2): 201-12); Carter (2001, Bispecific human IgG by design, J. Immunol. Methods 248(1-2): 7-15).

Bispecific IgGs may be created by any method known in the art, e.g., by chemical coupling methodologies or through the development of hybrid hybridoma cell lines (also referred to as hybrid hybridoma technology) (Milstein et al., 1983, Hybrid hybridomas and their use in immunohistochemistry, Nature 305(5934): 537-40) (FIG. 9).

Another approach used to obtain bispecific antibodies comprises exposing IgG to limited proteolytic digestion, where the two identical Fab fragments are released from the Fc fragment upon cleavage of the hinge polypeptide (FIG. 10). These single monovalent Fab fragments can be used alone, or chemically linked together (at the hinge cysteines) with a Fab fragment of separate origin to form a bispecific heterodimeric F(ab′)₂. Chemically linked bispecific F(ab′)₂ fragments have been studied and evaluated in several small-scale clinical trials (Hudson, 1999, Recombinant antibody constructs in cancer therapy, Curr. Opin. Immunol. 11(5): 548-57; Segal et al., 1999, Bispecific antibodies in cancer therapy, Curr. Opin. Immunol. 11(5): 558-62). Several other rational design strategies have been developed in order to engineer the Fe portion of heavy chains to promote the heterodimerization of bispecific antibodies. These strategies can include, for example, steric complementarity design mutations (“knobs-into-holes” utilizing phage display technology) as well as the design of additional inter-chain disulfide bonds and/or salt-bridge interactions between the heavy chains of the Fe fragment (Carter 2001, Bispecific human IgG by design, J. Immunol. Methods 248(1-2): 7-15). The enhanced complementarity between heavy chains of a desired bispecific antibody makes bispecific antibodies a preferred source for structural elements for use in the staged assembly of nanostructures as disclosed herein.

In one embodiment, bispecific antibodies are produced by replacing the Fc dimer-forming motif with another dimerization motif. In one non-limiting example, leucine zippers that can form heterodimers, such those found in Fos and Jun proteins, are linked to two different Fab portions of an IgG molecule by gene fusion. When expressed individually in an appropriate cell line, the fusion IgG's can be isolated as Fab-(zipper)₂ homodimers. Heterodimer formation is then achieved by reduction of the disulfide bonds within the hinge region of the homodimers to release the monomeric subunits. The resulting monomers are mixed together and placed under oxidizing conditions, resulting in bispecific heterodimers containing Fos-Jun paired leucine zipper motifs as the majority of the end products. Variations of this technique can be used to produce bispecific Fab and Fv fusion proteins (Kostelny et al., 1992, Formation of a bispecific antibody by the use of leucine zippers, J. Immunol. 148(5): 1547-53; Tso et al., 1995, Preparation of a bispecific F(ab′)₂ targeted to the human IL-2 receptor, J. Hematother. 4(5): 389-94; de Kruif et al., 1996, Leucine zipper, dimerized bivalent and bispecific scFv antibodies from a semi-synthetic antibody phage display library, J. Biol. Chem. 271(13): 7630-34). Additional multimerization motifs used to promote bispecific dimer formation include, but are not limited to: transcriptional factor p53 (Rheinnecker et al., 1996, Multivalent antibody fragments with high functional affinity for a tumor-associated carbohydrate antigen, J. Immunol. 157(7): 2989-97), streptavidin (Muller et al., 1998, A dimeric bispecific miniantibody combines two specificities with avidity, FEBS Lett. 432(1-2): 45-49), or helix-bundle motifs such as Rop (Pack et al., 1993, Improved bivalent miniantibodies with identical avidity as whole antibodies produced by high cell density fermentation of Escherichia coli, Biotechnology 11: 1271-77; Dubel et al., 1995, Bifunctional and multimeric complexes of streptavidin fused to single chain antibodies (scFv), J. Immun. Methods 178: 201-09) (FIG. 11). Such antibodies are useful in the present invention as a source of a plurality of joining elements that are non-identical and that do not interact with each other.

While the above-described methodologies permit the production and isolation of bispecific antibodies, the methods also result in the creation of mixtures of IgG products, in low yields or combinations of both. Multivalent and multifunctional antibodies of high quality, quantity and purity may be created by recombinant antibody technology ((see , e.g., Morrison et al., 1989, Genetically engineered antibody molecules, Adv. Immunol. 44: 65-92; Shin et al., 1993, Hybrid antibodies, Int. Rev. Immunol. 10(2-3): 177-86; Sensel et al., 1997, Engineering novel antibody molecules, Chem. Immunol. 65: 129-58; Hudson et al., 1998, Recombinant antibody fragments, Curr. Opin. Biotechnol. 9(4): 395-402).

In other embodiments of the invention, human, humanized or chimeric (e.g., human-mouse or human-other species) monoclonal antibodies (mAbs), or binding derivatives or binding fragments thereof, may be used as structural elements for use in the staged assembly methods of the invention. Humanized antibodies are antibody molecules from non-human species having one or more complementarity determining regions (CDRs) from the non-human species and a framework region from a human immunoglobulin molecule. Humanized antibodies are also referred to as “chimeric antibodies.” Humanized or chimeric antibodies may be produced by methods well known in the art (see, e.g., Queen, U.S. Pat. No. 5,585,089, entitled “Humanized immunoglobulins,” issued Dec. 17, 1996, which is incorporated herein by reference in its entirety).

Chimeric antibodies may be used as structural elements according to the methods of the invention. A chimeric antibody is a molecule in which different portions are derived from different animal species, such as those having a variable region derived from a murine mAb and a human immunoglobulin effector or constant region. Techniques have been developed for the production of chimeric antibodies (Morrison et al., 1984, Chimeric human antibody molecules: mouse antigen-binding domains with human constant region domains, Proc. Natl. Acad. Sci. USA 81: 6851-55; Neuberger et al., 1984, Recombinant antibodies possessing novel effector functions, Nature, 312, 604-08; Takeda et al., 1985, Construction of chimaeric processed immunoglobulin genes containing mouse variable and human constant region sequences, Nature 314: 452-54) by splicing the genes from a mouse antibody molecule of appropriate antigen specificity together with genes from a human antibody molecule of appropriate biological or effector activity.

### 5.5.1.7. Structural Elements Comprising Diabodies or Multimeric scFv Fragments

In certain embodiments of the invention, structural elements comprise diabodies or multimeric scFv fragments. scFv fragments, especially those with shortened peptide linkers, e.g. 3, 4 or 5 amino acid residues in length, form dimers ((scFv₂) or diabodies) rather than monomers in solution (Dolezal et al., 2000, ScFv multimers of the anti-neuraminidase antibody NC10: shortening of the linker in single-chain Fv fragment assembled in V(L) to V(H) orientation drives the formation of dimers, trimers, tetramers and higher molecular mass multimers, Protein Eng. 13(8): 565-74). Interchain domain interactions, rather than intrachain domain interactions, occur in order to form the stable dimeric diabody fragments (Holliger et al., 1993, Diabodies: small bivalent and bispecific antibody fragments, Proc. Natl. Acad. Sci. U.S.A. 90(14): 6444-48). A shortened peptide linker may prevent intrachain domain pairing and thus allow formation of interchain interactions that result in diabody fragment formation (Perisic et al. 1994, Crystal structure of a diabody, a bivalent antibody fragment, Structure 2(12): 1217-26).

In certain embodiments, diabodies can be used as the structural elements for the staged assembly of one-, two- and three-dimensional nanostructures. As used herein, the term “diabody” refers to dimeric single-chain variable antibody fragments (scFv). An scfv fragment, as described above, is composed of a fusion peptide that links the carboxyl terminus of the Fv variable heavy chain to the amino terminus of the Fv variable light chain (V_{H}-V_{L}) or vice versa (i.e. V_{L}-V_{H}) (Pluckthun et al., 1997, New protein engineering approaches to multivalent and bispecific antibody fragments, Immunotechnology 3(2): 83-105; Hudson, 1998, Recombinant antibody fragments, Curr. Opin. Biotechnol. 9(4): 395-402; Kipriyanov et al., 1999, Generation of recombinant antibodies, Mol. Biotechnol.12(2): 173-201).

In certain embodiments, a diabody or multimeric fragment is thermostable (see, e.g., Jermutus et al., 2001, Tailoring in vitro evolution for protein affinity or stability, Proc. Natl. Acad. Sci. USA 98(1): 75-80; Worn et al., 2001, Stability engineering of antibody single-chain Fv fragments, J. Mol. Biol. 305(5): 989-1010). Thermostability is a useful characteristic for structural elements utilized in the staged assembly of one- two- and three-dimensional nanostructures.

Unlike a monovalent scFv fragment, a diabody is a bivalent molecule containing “two bodies” that include two separate antigen-binding sites in opposition to one another and related by approximately 170° about the pseudo-two-fold axis of symmetry (parallel to the interface) (Perisic et al., 1994, Crystal structure of a diabody, a bivalent antibody fragment, Structure 2(12): 1217-26; Poljak, 1994, Production and structure of diabodies Structure 2: 1121-23; Hudson et al., 1999, High avidity scFv multimers; diabodies and triabodies, J. Immunol. Methods 231(1-2): 177-89) (FIG. 8).

A monospecific diabody contains two identical antigen-binding sites, both with specificity for the same ligand/hapten. A bispecific diabody contains two antigen-binding sites, each specific for a different ligand/hapten; that is, a bispecific diabody is derived from two different non-paired scFv fragments. The first hybrid fragment contains the V_{H} coding region from a first Fv antibody and a V_{L} coding region derived from a second Fv antibody. The resulting V_{H}-V_{L} hybrid fragment is joined together by a short Gly/Ser linker. The second hybrid fragment contains the V_{L} coding region from the first Fv antibody and the V_{H} coding region derived from the second Fv antibody.

The use of bispecific links permits the creation of bispecific antibody fragments that demonstrate bispecific affinity towards each ligand (Poljak, 1994, An idiotope-anti-idiotope complex and the structural basis of molecular mimicking, Proc. Natl. Acad. Sci. U.S.A. 91(5): 1599-1600; Kipriyanov et al., 1998, Bispecific CD3×CD19 diabody for T cell-mediated lysis of malignant human B cells, Int. J. Cancer 77(5): 763-72; Arndt et al., 1999, A bispecific diabody that mediates natural killer cell cytotoxicity against xenotransplanted human Hodgkin's tumors, Blood 94(8): 2562-68; Takemura et al., 2000, Construction of a diabody (small recombinant bispecific antibody) using a refolding system, Protein Eng. 13(8): 583-88). Certain bispecific diabodies demonstrate affinities towards ligands/haptens similar to that demonstrated by whole IgG (Holliger et al., 1993, Engineering bispecific antibodies, Curr. Opin. Biotechnol. 4(4): 446-49; Yagi et al., 1994, Superantigen-like properties of an antibody bispecific for MHC class II molecules and the V beta domain of the T cell antigen receptor, J. Immunol. 152(8): 3833-41).

Diabodies exhibit several properties that make them particularly attractive for use the in staged assembly methods of the invention: (i) they are structures containing oppositely directed antigen binding sites; (ii) the geometrical opposition of the two antigen-binding sites optimizes the potential for building linear nanostructures or linear extensions of nanostructures; (iii) they have a well-defined size, shape, structure and stoichiometry; (iv) they have structural rigidity and well-defined recognition and binding properties; (v) binding motifs exhibiting specificity for a very broad range of organic and inorganic moieties can be identified and incorporated into a diabody structure (vi) their X-ray structure has been solved (FIG. 8) and can serve as a blueprint for identifying positions at which it is possible to add functional groups or binding sites; (vii) diabodies form strong intermolecular bonds to one another; (viii) the intermolecular bonds are highly specific; (ix) the immunoglobulin fold provides a structured protein core (structural element) and a stable spatial relationship among the different faces of the protein; (x) loops in which additional binding sites may be inserted are readily identified through an examination of the three-dimensional structure of a diabody (Zhu et al., 1996, High level secretion of a humanized bispecific diabody from Escherichia coli, Biotechnology (NY) 14(2): 192-96; Hudson et al., 1999, High avidity scFv multimers; diabodies and triabodies, J. Immunol. Methods 231(1-2): 177-89). Taken together, these properties are advantageous for using diabodies as structural elements for constructing complex, multidimensional nanostructures.

scFv fragments can also associate into multivalent multimers (Hudson et al., 1999, High avidity scFv multimers; diabodies and triabodies, J. Immunol. Methods 231(1-2): 177-89; Power et al., 2000, Synthesis of high avidity antibody fragments (scFv multimers) for cancer imaging, J. Immunol. Methods 242(1-2): 193-204; Todorovska et al., 2001, Design and application of diabodies, triabodies and tetrabodies for cancer targeting, J. Immunol. Methods 248(1-2): 47-66) (FIG. 12). Multimer formation is dependent upon the length of the linker used to associate the variable domains (V-domain) together, as well as the V-domain composition and orientation (V_{H}-V_{L} versus V_{L}-V_{H}). Reducing the linker length below three residues usually favors trimer or triabody formation, e.g., scFv)₃. Tetrabody formation, e.g., (scFv)₄ also has been reported in at least two instances where the linker length was 0 residues in length and the V-domain orientation was V_{L}-V_{H} (Todorovska et al., 2001, Design and application of diabodies, triabodies and tetrabodies for cancer targeting, J. Immunol. Methods 248(1-2): 47-66).

An antibody variable domain may functionally comprise both a structural element and a joining element in an assembly unit for staged assembly. Like structural elements, the extent of a joining element may not always be completely defined. For example, the β-sheet structure of an antibody variable domain maintains the geometric relationship between the CDR and the other parts of the molecule. But it is also important for maintaining the structural relationships between the loops of the CDR that provide the binding affinity and specificity of the complementary partner of the joining pair. Consequently, an antibody variable domain may functionally comprise both a structural element and a joining element in an assembly unit. Thus, although antibody molecules and binding fragments of antibodies are preferred elements of joining elements, they may also provide structural framework for many embodiments, and as described above, for an assembly unit.

### 5.5.2. Structural Elements Comprising Bacterial Pilin Proteins

In certain embodiments of the invention, structural elements comprise bacterial pilin proteins, or binding derivatives or binding fragments thereof. Pilins are the protein units making up bacterial adhesion pili. Bacterial adhesion pili (“P-pili”) are formed by the polymerization of pilins (see, e.g., Bullitt and Makowski, 1995, Structural polymorphism of bacterial adhesion pili, Nature 373: 164-67; Bullitt and Makowski, 1998, Bacterial adhesion pili are heterologous assemblies of similar units, Biophysics J. 74: 623-32). Pili units may be assembled in vitro (see, e.g., Bullitt et al., 1996, Development of pilus organelle sub-assemblies in vitro depends on chaperone uncapping of a beta zipper, Proc. Nat. Acad. Sci. USA 93: 12890-95).

P-pili expressed on the surface of E. coli are helical filaments 6.8 nm in diameter, with an ellipsoidal central cavity 2.5 nm×1.5 nm that winds about the helical axis, connecting to radial channels that extend to the surface of the pili (Hultgren and Normark, 1991, Biogenesis of the bacterial pilus, Curr. Opin. Genet. Dev. 1: 313-18; Hultgren et al., 1993, Pilus and nonpilus bacterial adhesins: assembly and function in cell recognition, Cell 73(5): 887-901; Bullitt and Makowski, 1995, Structural polymorphism of bacterial adhesion pili, Nature 373: 164-67). Each pilus comprises approximately 1000 copies of the major pilin, PapA, and one or a few copies of the minor pilins, PapH, PapK, PapE, PapF, and PapG. In the PapA-containing coiled rod region of the helix, there are 3.29 subunits per turn of the helix, with a 7.6 A rise per subunit (Bullitt and Makowski, 1995, Structural polymorphism of bacterial adhesion pili, Nature 373: 164-167). The fibrillae at the distal tip of the pilus is made up of four distinct but homologous pilins (FIG. 13). The distal end of papA will interact with the proximal end of papA or papK. The proximal end of papK will interact only with papA; its distal end only with papE; and so on as required by its remarkable architecture. These specific interactions are summarized in Table 2 below.

TABLE 2
Pilin-Pilin Protein Interactions

| Pilin | End | Interacts with Pilin |
|---|---|---|
| papA | Proximal | papA and papH |
| papA | Distal | papA and papK |
| pap H | Proximal | none |
| pap H | Distal | papA |
| papK | Proximal | papA |
| papK | Distal | papE |
| papE | Proximal | papH and papE |
| papE | Distal | papE and papF |
| papF | Proximal | papE |
| papF | Distal | papG |
| papG | Proximal | papF |
| papG | Distal | Does not interact with the |
| | | N-terminal extension of any |
| | | papA, papH, papE, papF, or |
| | | papG |

The interaction between pilin proteins is mediated by the N-terminal extension of each pilin protein that binds to the immediately adjacent pilin protein in P-pili, yielding an extended intermolecular interface that provides significant mechanical strength to the pilus (Sauer et al., 1999, Structural basis of chaperone function and pilus biogenesis, Science 285: 1058-61; Choudhury et al., 1999, X-ray structure of the FimC-FimH chaperone-adhesin complex from uropathogenic Escherichia coli, Science 285: 1061-66). The affinity and specificity of this binding are determined by the interaction between the extended N-terminal arm and the groove on the adjacent pilin protein (FIG. 14A). Consequently, replacement of the N-terminal arm of one pilin with that from another (forming a “hybrid” or “chimeric” pilin protein) provides a means of altering the specificity of binding of a pilin unit, and provides a means of designing protein units that may be used as structural and/or joining elements in staged assembly, i.e., where the N-terminus and body of one pilin do not interact to form dimers and polymers (FIG. 14B) (see also Section 5.6.6 below). A hybrid or chimeric pilin protein comprises the pilin amino terminal extension of a first pilin protein and the pilin protein body of a second pilin protein and lacks the pilin protein body of the first pilin protein and the pilin amino terminal extension of the second pilin protein, wherein the amino terminal extension of the first pilin protein does not bind to the pilin protein body of the second pilin protein.

A comparison of the sequences of all the pilins that make up a P-pilus indicate that the region that links the N-terminal extension with the body of that pilin protein is highly conserved among pilins and that the position for fusing heterologous pilin parts is well-defined based on that homology.

Functionality may be added to the pilin subunits at positions identified as being (i) on the surface of the subunits; (ii) unimportant to the interaction of the subunits with one another and (iii) unimportant for the stability of the subunits themselves. It has been shown that in many proteins, large loop insertions are tolerated and many redesigns have generated proteins that successfully fold to stable, active structures. Some redesigns have been entirely the choice of the investigators, whereas others have incorporated a randomization and selection step to identify optimal sequences (Regan, 1999, Protein redesign, Current Opinion in Structural Biology 9: 494-99). One region amenable to reengineering is a surface loop on papA comprised of gly107-ala108-gly109. This loop satisfies the criteria that must be met by a position where a heterologous peptide may be successfully inserted.

Pilin proteins may be expressed and purified by methods commonly known in the art (e.g., Bullitt and Makowski, 1995, Structural polymorphism of bacterial adhesion pili, Nature 373: 164-67; Bullitt et al., 1996, Development of pilus organelle sub-assemblies in vitro depends on chaperone uncapping of a beta zipper, Proc. Natl. Acad. Sci. USA 93: 12890-95).

### 5.5.3. Structural Elements Comprising Leucine Zipper-Type Coiled Coils

In certain embodiments, the invention encompasses structural elements comprising leucine zipper-type coiled coils for use in the construction of nanostructures using the staged assembly methods of the invention. Leucine zippers are well-known, a-helical protein structures (Oas et al., 1994, Springs and hinges: dynamic coiled coils and discontinuities, TIBS 19: 51-54; Branden et al., 1999, Introduction to Protein Structure 2and ed., Garland Publishing, Inc., New York) that are involved in the oligomerization of proteins or protein monomers into dimeric, trimeric, and tetrameric structures, depending on the exact sequence of the leucine zipper domain (Harbury et al., 1993, A switch between two-, three-, and four-stranded coiled coils in GCN4 leucine zipper mutants, Science 262: 1401-07). While only dimers are disclosed herein for simplicity, it would be apparent to one of ordinary skill in the art that trimeric and tetrameric units may also be used for the construction of assembly units for use in staged assembly of nanostructures according to the methods disclosed herein. In certain embodiments, trimeric and tetrameric units could be especially useful for incorporation of functional elements that, e.g., require two or more chemical moieties for proper activity, for example, the incorporation of two cysteine moieties for binding of gold particles. Several non-limiting examples of leucine-zipper domains are provided in Table 3 below.

Table 3 shows canonical leucine zippers and high stability dimerization sequences. The top line shows register of the repeat unit. Residues in the a and d positions are generally hydrophobic and control the oligomerization. Residues in the e and g positions are generally charged and create salt bridges to stabilize the oligomerization.

TABLE 3

Canonical Leucine Zippers and High Stability
Dimerization Sequences

| | |
|---|---|
| | abcdefgabcdefgabcdefgabcdefgabcdefg |
| GCN4 | MKQLEDKVEELLSKNYHLENEVARLKKL |
| | (SEQ ID NO: 1) |
| c-Fos | TDTLQAETDQLEDEKYALQTEIANLLKE |
| | (SEQ ID NO: 2) |
| c-Jun | AARLEEKVKTLKAQNYELASTANMLREQ |
| | (SEQ ID NO: 3) |
| C/EBPb | VLETQHKNERLTAEVEQLQKKLSTLSREFKQLRNL |
| | (SEQ ID NO: 4) |
| ATF4 | CKELTGENEALEKKADSLKERIQYLAKEIEEVKDL |
| | (SEQ ID NO: 5) |
| c-myc | CCGVQAEEQKLTSEEDLLRKRREQLKHKLEQLX |
| | (SEQ ID NO: 6) |
| Max | CGGMRRKNDTHQQDIDDLKRQNALLEQQVRALX |
| | (SEQ ID NO: 7) |
| CREB | VKSLENRVAVLENQNKTLIEELKALKDLYSHK |
| | (SEQ ID NO: 8) |
| PAP1 | VVTLKELHSSTTLENDQLRQKVRQLEEELRILK |
| | (SEQ ID NO: 9) |

Many naturally occurring leucine zippers may be used according to the methods of the invention, including those found in the yeast transcription factor GCN4 and in the mammalian Fos, Jun and Myc oncogenes. Additional proteins containing leucine zippers and other coiled coil-type oligomerization sequences can be identified by searching public protein databases such as SWISS-PROT/TrEMBL (Bairoch and Apweiler, 2000, The SWISS-PROT protein sequence database and its supplement TrEMBL in 2000, Nucl. Acids Res. 28: 45-48). Table 4 shows the results of such a search, using the keywords “coiled coil” and “dimer.”In Table 4, the common names of genes are listed, as well as their SWISS-PROT accession numbers, sequence description and sequence. The SWISS-PROT accession number is a unique identifier for a sequence record. An accession number applies to the complete record and is usually a combination of a letter(s) and numbers, such as a single letter followed by five digits (e.g., Q12345) or a combination of six letters and digits (e.g., Q1Z2F3). The coiled coil sequences are underlined.

TABLE 4

Examples of Proteins Containing Coiled Coil Dimerization Sequences
That Can Be Used for Structural Elements of Assembly Units

| | | | |
|---|---|---|---|
| Sequence | Accession | Sequence | |
| ID | number | description | Sequence |

| | | | |
|---|---|---|---|
| SWISS_PR0 | O54931 | A-kinase anchor | MEIGVSVAECKSVPGVTSTPHSKDHSSPFYSPS |
| OT: | O54932 | protein 2 | HNGLLADHHESLDNDVAREIQYLDEVLEANCCD |
| AKA2_MOU | O54933 | (Protein kinase | SSVDGTYNGISSPEPGAAILVSSLGSPAHSVTE |
| SE | | A anchoring | AEPTEKASGRQVPPHIELSRIPSDRMAEGERAN |
| | | protein | GHSTDQPQDLLGNSLQAPASPSSSTSSHCSSRD |
| | | 2) \| (PRKA2) | GEFTLTTLKKEAKFELRAFHEDKKPSKLFEEDE |
| | | (AKAP expressed | REKEQFCVRKVRPSEEMIELEKERRELIRSQAV |
| | | in kidney and | KKNPGIAAKWWNPPQEKTIEEQLDEEHLESHRR |
| | | lung) (AKAP-KL) | YKERKEKRAQQEQLQLQQQQQQQLQQQQLQQQQ |
| | | | LQQQQLQQQLQQQQLSTSQPCTAPAAHKHLDGI |
| | | | EHTKEDVVTEQIDFSAARKQFQLMENSRQTLAK |
| | | | GQSTPRLFSIKPYYKPLGSIHSDKPPTILRPAT |
| | | | VGGTLEDGGTQAAKEQKAPCVSESQSAGAGPAN |
| | | | AATQGKEGPYSEPSKRGPLSKLWAEDGEFTSAR |
| | | | AVLTVVKDEDHGILDQFSRSVNVSLTQEELDSG |
| | | | LDELSVRSQDTTVLETLSNDFSMDNISDSGASN |
| | | | ETTSALQENSLADFSLPQTPQTDNPSEGREGVS |
| | | | KSFSDHGFYSPSSTLGDSPSVDDPLEYQAGLLV |
| | | | QNAIQQAIAEQVDKAEAHTSKEGSEQQEPEATV |
| | | | EEAGSQTPGSEKPQGMFAPPQVSSPVQEKRDIL |
| | | | PKNLPAEDRALREKGPSQPPTAAQPSGPVNMEE |
| | | | TRPEGGYFSKYSEAAELRSTASLLATQESDVMV |
| | | | GPFKLRSRKQRTLSMIEEEIRAAQEREEELKRQ |
| | | | RQVRQSTPSPRAKNAPSLPSRTTCYKTAPGKIE |
| | | | KVKPPPSPTTEGPSLQPDLAPEEAAGTQRPKNL |
| | | | MQTLMEDYETHKSKRRERMDDSSYTSKLLSCKV |
| | | | TSEVLEATRVNRRKSASGLALGGRDLR |
| | | | (SEQ ID NO: 10) |
| SWISS_PR | Q99996 | A-kinase anchor | MEDEERQKKLEAGKAKIEELSLAFLVRQLAQFR |
| OT: | Q9UQQ4 | protein 9 | QRKAQSDGQSPSKKQKKKRKTSSSKHDVSAHHD |
| AKA9_HUM | Q9UQH3 | (Protein kinase | LNIDQSQCNEMYINSSQRVESTVIPESTIMRTL |
| AN | Q9YGY2 | A anchoring | HSGEITSHEQGFSVELESEISTTADDCSSEVNG |
| | O14869 | protein | CSFVMRTGKPTNLLREEEFGVDDSYSEQGAQDS |
| | O43355 | 9) \| (PRKA9) (A- | PTHLEMMESELAGKQHEIEELNRELEEMRVTYG |
| | O94895 | kinase anchor | TEGLQQLQEFEAAIKQRDGIITQLTANLQQARR |
| | Q9YGB8 | protein 450 | EKDETMREFLELTEQSQKLQIQEQQLQASETLR |
| | | kDa) (AKAP 450) | NSTHSSTAADLLQAKQQILTHQQQLEEQDHLLE |
| | | (A-kinase | DYQKKKEDFTMQTSFLQEKIKVYEMEQDKKVEN |
| | | anchor\|protein | SNKEEIQEKETTIEELNTKTIEEEKKTLELKDK |
| | | 350 kDa) (AKAP | LTTADKLLGELQEQIVQKNQEIKNMKLELTNSK |
| | | 350) (hgAKAP | QKERQSSEEIKQLMGTVEELQKRNHKDSQFETD |
| | | 350) (AKAP 120 | IVQRMEQETQRKLEQLRAELDEMYGQQIVQMKQ |
| | | like | ELTRQHMAQMEEMKTRHKGSMENALRSYSNITV |
| | | protein) \| (Hyper | NEDQIKLMNVAINSLNIKLQDTNSQKEKLKEEL |
| | | ion protein) | GLILEEKCALQRQLEDLVEELSFSREQIQRARQ |
| | | (Yotiao | TIAEQESKLNEAHKSLSTVEDLKAEIVSASESR |
| | | protein) | KELELKHEAEVTNYKIKLEMLEKEKNAVLDRMA |
| | | (Centrosome- | ESQEAELERLRTQLLESHEEELSKLKEDLEIRH |
| | | and golgi- | RINIEKLKDNLGIHYKQQIDCLQNEMSQKTETM |
| | | localized\|PKN- | QFEKDNLITKQNQLILEISKLKDLQQSLVNSKS |
| | | associated | EEMTLQINELQKETEILRQEEKEKGTLEQEVQE |
| | | protein) (CG- | LQLKTELLEKQMKEKENDLQEKFAQLEAENSIL |
| | | NAP) | KDEKKTLEDMLKIHTPVSQEERLIFLDSIKSKS |
| | | | KDSVWEKEIEILIEENEDLKQQCIQLNEEIEKQ |
| | | | RNTFSEAEKNFEVNYQELQEEYACLLKVKDDLE |
| | | | DSKNKQELEYKSKLKALNEELHLQRINPTTVKM |
| | | | KSSVFDEDKTFVAETLEMGEVVEKDTTELMEKL |
| | | | EVTKREKLELSQRLSDLSEQLKQKHGEISFLNE |
| | | | EVKSLKQEKEQVSLRCRELEIIINHNRAENVQS |
| | | | CDTQVSSLLDGVVTMTSRGAEGSVSKVNKSFGE |
| | | | ESKIMVEDKVSFENMTVGEESKQEQLILDHLPS |
| | | | VTKESSLRATQPSENDKLQKELNVLKSEQNDLR |
| | | | LQMEAQRICLSLVYSTHVDQVREYMENEKDKAL |
| | | | CSLKEELIFAQEEKIKELQKIHQLELQTMKTQE |
| | | | TGDEGKPLHLLIGKLQKAVSEECSYFLQTLCSV |
| | | | LGEYYTPALKCEVNAEDKENSGDYISENEDPEL |
| | | | QDYRYEVQDFQENMHTLLNKVTEEYNKLLVLQT |
| | | | RLSKIWGQQTDGMKLEFGEENLPKEETEFLSIH |
| | | | SQMTNLEDIDVNHKSKLSSLQDLEKTKLEEQVQ |
| | | | ELESLISSLQQQLKETEQNYEAEIHCLQKRLQA |
| | | | VSESTVPPSLPVDSVVITESDAQRTMYPGSCVK |
| | | | KNIDGTIEESGEFGVKEETNIVKLLEKQYQEQL |
| | | | EEEVAKVIVSMSIAFAQQTELSRISGCKENTAS |
| | | | SKQAHAVCQQEQHYENEMKLSQDQIGFQTFETV |
| | | | DVKFKEEEKPLSKELGEHGKEILLSNSDPHDIP |
| | | | ESKDCVLTISEEMFSKDKTFIVRQSIHDEISVS |
| | | | SMDASRQLMLNEEQLEDMRQELVRQYQEHQQAT |
| | | | QRSSIDNENLVSERERVLLEELEALKQLSLAGR |
| | | | EKLCCELRNSSTQTQNGNENQGEVEEQTFREKE |
| | | | LDRKPEDVPPEILSNERYALQKANNRLLKILLE |
| | | | VVKTTAAVEETIGRHVLGILDRSSKSQSSASLI |
| | | | WRSEAEASVKSCVHEEHTRVTDESIPSYSGSDM |
| | | | PRNDINMWSKVTEEGTELSQRLVRSGFAGTEID |
| | | | PENEELMLNISSRLQAAVEKLLEAISETSSQLE |
| | | | HAKVTQTELMRESERQKQEATESLKCQEELRER |
| | | | LHEESRAREQLAVELSKAEGVIDGYADEKTLFE |
| | | | RQIQEKTDIIDRLEQELLCASNRLQELEAEQQQ |
| | | | IQEERELLSRQKEAMKAEAGPVEQQLLQETEKL |
| | | | MKEKLEVQCQAEKVRDDLQKQVKALEIDVEEQV |
| | | | SRFIELEQEKNTELMDLRQQNQALEKQLEKMRK |
| | | | FLDEQAIDREHERDVFQQEIQKLEQQLKVVPRF |
| | | | QPISEHQTREVEQLANHLKEKTDKCSELLLSKE |
| | | | QLQRDIQERNEEIEKLEFRVRELEQALLVSADT |
| | | | FQKVEDRKHFGAVEAKPELSLEVQLQAERDAID |
| | | | RKEKEITNLEEQLEQFREELENKNEEVQQLHMQ |
| | | | LEIQKKESTTRLQELEQENKLFKDDMEKLGLAI |
| | | | KESDAMSTQDQHVLEGKFAQIIQEKEVEIDQLN |
| | | | EQVTKLQQQLKITTDNKVIEEKNELIRDLETQI |
| | | | ECLMSDQECVKRNREEEIEQLNEVIEKLQQELA |
| | | | NIGQKTSMNAHSLSEEADSLKHQLDVVIAEKLA |
| | | | LEQQVETANEEMTFMKNVLKETNFKMNQLTQEL |
| | | | FSLKRERESVEKIQSIPENSVNVAIDHLSKDKP |
| | | | ELEVVLTEDALKSLENQTYFKSFEENGKGSIIN |
| | | | LETRLLQLESTVSAKDLELTQCYKQIKDMQEQG |
| | | | QFETEMLQKKIVNLQKIVEEKVAAALVSQIQLE |
| | | | AVQEYAKFCQDNQTISSEPERTNIQNLNQLRED |
| | | | ELGSDISALTLRISELESQVVEMHTSLILEKEQ |
| | | | VEIAEKNVLEKEKKLLELQKLLEGNEKKQREKE |
| | | | KKRSPQDVEVLKTTTELFHSNEESGFFNELEAL |
| | | | RAESVATKAELASYKEKAEKLQEELLVKETNMT |
| | | | SLQKDLSQVRDHLAEAKEKLSILEKEDETEVQE |
| | | | SKKACMFEPLPIKLSKSIASQTDGTLKISSSNQ |
| | | | TPQILVKNAGIQINLQSECSSEEVTEIISQFTE |
| | | | KIEKMQELHAAEILDMESRHISETETLKREHYV |
| | | | AVQLLKEECGTLKAVIQCLRSKEVFGEYNMCES |
| | | | TLCDSGSDWGQGIYLTHSQGFDIASEGRGEESE |
| | | | SATDSFPKKIKGLLRAVHNEGMQVLSLTESPYS |
| | | | DGEDHSIQQVSEPWLEERKAYINTISSLKDLIT |
| | | | KMQLQREAEVYDSSQSHESFSDWRGELLLALQQ |
| | | | VFLEERSVLLAAFRTELTALGTTDAVGLLNCLE |
| | | | QRIQEQGVEYQAAMECLQKADRRSLLSEIQALH |
| | | | AQMNGRKITLKREQESEKPSQELLEYNIQQKQS |
| | | | QMLEMQVELSSMKDRATELQEQLSSEKMVVAEL |
| | | | KSELAQTKLELETTLKAQHKHLKELEAFRLEVK |
| | | | DRTDEVHLLNDTLASEQKKSRELQWALEKEKAK |
| | | | LGRSEERDKEELEDLKFSLESQKQRNLQLNLLL |
| | | | EQQKQLLNESQQKTESQRMLYDAQLSEEQGRNL |
| | | | ELQVLLESEKVRIREMSSTLDRERELHAQLQSS |
| | | | DGTGQSRPPLPSEDLLKELQKQLEEKHSRIVEL |
| | | | LNETEKYKLDSLQTRQQMSKDRQVHRKTLQTEQ |
| | | | EANTEGQKKMHELQSKVEDLQRQLEEKRQQVYK |
| | | | LDLEGQRLQGIMQEFQKQELEREEKRESRRILY |
| | | | QNLNEPTTWSLTSDRTRNWVLQQKIEGETKESN |
| | | | YAKLIEMNGGGTGCNHELEMIRQKLQCVASKLQ |
| | | | VLPQKASERLQFETADDEDFIWVQENIDEIILQ |
| | | | LQKLTGQQGEEPSLVSPSTSCGSLTERLLRQNA |
| | | | ELTGHISQLTEEKNDLRNMVMKLEEQIRWYRQT |
| | | | GAGRDNSSRFSLNGGANIEAIIASEKEVWNREK |
| | | | LTLQKSLKRAEAEVYKLKAELRNDSLLQTLSPD |
| | | | SEHVTLKRIYGKYLRAESFRKALIYQKKYLLLL |
| | | | LGGFQECEDATLALLARMGGQPAFTDLEVITNR |
| | | | PKGFTRFRSAVRVSIAISRMKFLVRRWHRVTGS |
| | | | VSININRDGFGLNQGAEKTDSFYHSSGGLELYG |
| | | | EPRHTTYRSRSDLDYIRSPLPFQNRYPGTPADE |
| | | | NPGSLACSQLQNYDPDRALTDYITRLEALQRRL |
| | | | GTIQSGSTTQFHAGMRR |
| | | | (SEQ ID NO: 11) |
| SWISS_PR | Q28628 | A-kinase anchor | REKLEVQCQAEKVRDDLQKQVKALEIDVEEQVC |
| OT: | | protein 9 | RFIELEQEKNAELMDLRQQNQALEKQLEKMRKM |
| AKA9_RAB | | (Protein kinase | DLRQQNQALEKQLEKMRKFLDEQAIDREHERDV |
| IT | | A anchoring | FQQEIQKLEQQLKLVPRFQPISEHOQREVEQLT |
| | | protein | NHLKEKTDKCSELLLSKEQLQRDVOQRNEEIEK |
| | | 9) \| (PRKA9) (A- | LECRVRELEQALLSVQTLSKRWRTRNSFGAVEP |
| | | kinase anchor | KAELCLEVQLQAERDAIDRKEKEITNLEEQLEQ |
| | | protein 120 | FREELENKNEEVQQLHMQLEIQKKESTTRLQEL |
| | | kDa) (AKAP 120) | EQENKLFKDEMEKLGFAIKESDAVSPQDQQVLF |
| | | (Fragment) | GKFAQIIHEKEVEIDRLNEQIIKLQQQLKITTD |
| | | | NKVIEEKNELIRDLEAQIECLMSDQERVRKNRE |
| | | | EEIEQLNEVIEKLQQELANIDQKTSVDPSSLSE |
| | | | EADSLKHQLDKVIAEKLALEHQVETTNEEMAVT |
| | | | KNVLKETNFKMNQLTQELCSLKREREKMERIQS |
| | | | VPEKSVNMSVGDLSKDKPEMDLIPTEDALAQLE |
| | | | TQTQLRSSEESSKVSLSSLETKLLQLESTVSTK |
| | | | DLELTQCYKQIQDMREQGRSETEMLQTKIVSLQ |
| | | | KVLEEKVAAALVSQVQLEAVQEYVKLCADKPAV |
| | | | SSDPARTEVPGLSQLAGNTMESDVSALTWRISE |
| | | | LESQLVEMHSSLISEKEQVEIAEKNALEKEKKL |
| | | | QELQKLVQDSETKQRERERQSRLHGDLGVLEST |
| | | | TSEESGVFGELEALRAESAAPKGELANYKELAE |
| | | | KLQEELLVKETNMASLPKELSHVRDQLTEAEDK |
| | | | LSHFSEKEDKTEVQEHGTICILEPCPGQIGESF |
| | | | ASQTEGAVQVNSHTQTPQIPVRSVGIQTHSQSD |
| | | | SSPEEVAEIISRFTEKIEQMRELHAAEILDMES |
| | | | RHISETETLKREHCIAVQLLTEECASLKSLIQG |
| | | | LRMPEGSSVPSLTHSNAYQTREVGSSDSGSDWG |
| | | | QGIYLTQSQGFDTASEARGEEGETSTDSFPKKI |
| | | | KGLLRAVHNEGMQVLSLTEGPCGDGEDYPGHQL |
| | | | SESWLEERRAYLSTISSLKDFITKMQVQREVEV |
| | | | YDSSQSHENISDWRGELLLALQQVFLRERSVLL |
| | | | AAFKTELTALGTRDAAGLLNCLEQRIPRTEY |
| | | | (SEQ ID NO: 12) |
| SWISS_PR | Q94981 | Ariadne-1 | MDSDNDNDFCDNVDSGNVSSGDDGDDDFGMEVD |
| OT: | | protein (Ani-1) | LPSSADRQMDQDDYQYKVLTTDEIVQHQREIID |
| ARI1_DRO | | | EANLLLKLPTPTTRILLNHFKWDKEKLLEKYED |
| ME | | | DNTDEEFKCAHVINPENATEAIKQKTSRSQCEE |
| | | | CEICFSQLPPDSMAGLECGHRFCMPCWHEYLST |
| | | | KIVAEGLGQTTSCAAHGCDILVDDVTVANLVTD |
| | | | ARVRVKYQQLTTNSFVECNQLLRWCPSVDCTYA |
| | | | VKVPYAEPRRVHCKCGHVECFACGENWHDPVKC |
| | | | RWLKKWIKKCDDDSETSNWIAANTKECPRCSVT |
| | | | IEKDGGCNHMVCKNQNCKNEFCWVCLGSWEPHG |
| | | | SSWYNCNRYDEDEAKTARDAQEKLRSSLARYLH |
| | | | YYNRYMNHMQSMKFENKLYASVKQKMEEMQQHN |
| | | | MSWIEVQELKKAVDILCQCRQTLMYTYVFAYYL |
| | | | KKNNQSMIFEDNQKDLESATEMLSEYLERDITS |
| | | | ENLADIKQKVQDKYRYCEKRCSVLLKHVHEGYD |
| | | | KEWWEYTE |
| | | | (SEQ ID NO: 13) |
| SWISS_PR | Q9UBS5 | Gamma- | MLLLLLLAPLPLRPPGAGGAQTPNATSSGCQII |
| OT: | Q95375 | aminobutyric | HPPWEGGIRYRGLTRDQVKAINELPVDYEIEYV |
| GBR1_HUM | Q9UQQ0 | acid type B | CRGEREVVGPKVRKCLANGSWTDMDTPSRCVRI |
| AN | Q96022 | receptor, | CSKSYLTLENGKVFLTGGDLPALDGARVDERCD |
| | Q95975 | subunit 1 | PDFHLVGSSRSICSQGQWSTPKPHCQVNRTPHS |
| | Q95468 | precursor | ERRAVYIGALEPMSGGWPGGQACQPAVEMALED |
| | | (GABA- | VNSRRDILPDYELKLIHHDSKCDPGQATKYLYE |
| | | B\|receptor 1) | LLYNDPIKIILMPGCSSVSTLVAEAARMWNLIV |
| | | (GABA-B-R1) | LSYGSSSPALSNRQRFPTFFRTHPSATLHNPTR |
| | | (Gb1) | VKLFEKWGWKKIATIQQTTEVPTSTLDDLEERV |
| | | | KEAGIEITERQSFPSDPAVPVKNLKRQDARIIV |
| | | | GLFYETEARKVFCEVYKERLFGKKYVWELIGWY |
| | | | ADNWFKIYDPSINCTVDEMTEAVEGHITTEIVM |
| | | | LNPANTRSISNMTSQEEVEKLTKRLKRHPEETG |
| | | | GEQEAPLAYDAIWALALALNKTSGGGGRSGVRL |
| | | | EDPNYNNQTITDQIYRAMNSSSPEGVSGHVVFD |
| | | | ASGSRMAWTLIEQLQGGSYKKIGYYDSTKDDLS |
| | | | WSKTDKWIGGSPPADQTLVIKTERFLSQKLEIS |
| | | | VSVLSSLGIVLAVVCLSFNIYNSHVRYIQNSQP |
| | | | NLNNLTAVGGSLALAAVEPLGLDGYHIGRNQPP |
| | | | FVCQARLWLLGLGFSLGYGSMPTKIWWVHTVFT |
| | | | KKEEKKEWRKTLEPWKLYATVGLLVGMDVLTLA |
| | | | IWQIVDPLHRTIETFAKEEPKEDIDVSILPQLE |
| | | | HCSSRKMNTWLGIFYGYKGLLLLLGIFLAYETK |
| | | | SVSTEKINDHRAVGMAIYNVAVLCLITAPVTMI |
| | | | LSSQQDAAFAFASLAIVFSSYITLVVLFVPKMR |
| | | | RLITRGEWQSEAQDTMKTGSSTNNNEEEKSRLL |
| | | | EKENRELEKIIAEKEERVSELRHQLQSRQQLRS |
| | | | RRHPPTPPEPSGGLPRGPPEPPDRLSCDGSRVR |
| | | | LLYK |
| | | | (SEQ ID NO: 14) |
| SWISS_PR | P03069 | General control | MSEYQPSLFALNPMGFSPLDGSKSTNENVSAST |
| OT: | P03068 | protein GCN4 | STAKPMVGQLIFDKFIKTEEDPIIKQDTPSNLD |
| GCN4_YEA | | (Amino acid | FDFALPQTATAPDAKTVLPIPELDDAVVESFFS |
| ST | | biosynthesis | SSTDSTPMFEYENLEDNSKEWTSLFDNDIPVTT |
| | | regulatory\|prot | DDVSLADKAIESTEEVSLVPSNLEVSTTSPLPT |
| | | ein) | PVLEDAKLTQTRKVKKPNSVVKKSHHVGKDDES |
| | | | RLDHLGVVAYNRKQRSIPLSPIVPESSDPAALK |
| | | | RARNTEAARRSRARKLQRMKQLEDKVEELLSKN |
| | | | YHLENEVARLKKLVGER |
| | | | (SEQ ID NO: 15) |
| SWISS_PR | O60282 | Kinesin heavy | MADPAECSIKVMCRFRPLNEAETLRGDKFIPKF |
| OT: | O95079 | chain isoform | KGDSTVVIGQGKPYVFDRVLPPNTTQEQVYNAC |
| KF5C_HUM | | SC (Kinesin | AKQIVKDVLEGYNGTIFAYGQTSSGKTHTMEGK |
| AN | | heavy chain | LHDPQLMGTIPRIAHDIFDHIYSMDENLEFHIK |
| | | neuron- | VSYFEIYLDKIRDLLDVSKTNLAVHEDKNRVPY |
| | | specific\|2) | VKGCTERFVSSPEEVMDVIDEGKANRHVAVTNM |
| | | | NEHSSRSHSIFLINIKQENVETEKKLSGKLYLV |
| | | | DLAGSEKVSKTGAEGAVLDEAKNINKSLSALCN |
| | | | VISALAEGTKTHVPYRDSKMTRILQDSLGGNCR |
| | | | TTIVICCSPSVENEAETKSTLMFGQRAKTIKNT |
| | | | VSVNLELTAEEWKKKYEKEKEKNKTLKNVIQHL |
| | | | EMELNRWRNGEAVPEDEQISAKDQKNLEPCDNT |
| | | | PIIDNIAPVVAGISTEEKEKYDEEISSLYRQLD |
| | | | DKDDEINQQSQLAEKLKQQMLDQDELLASTRRD |
| | | | YEKIQEELTRLQIENEAAKDEVKEVLQALEELA |
| | | | VNYDQKSQEVEDKTRANEOQTDELAQKTTTLTT |
| | | | TQRELSQLQELSNHQKKRATEILNLLLKDLGEI |
| | | | GGIIGTNDVKTLADVNCVIEEEFTMARLYISKM |
| | | | KSEVKSLVNRSKQLESAQMDSNRKMNASERELA |
| | | | ACQLLISQHEAKIKSLTDYMQNMEQKRRQLEES |
| | | | QDSLSEELAKLRAQEKMHEVSFQDKEKEHLTRL |
| | | | QDAEEMKKALEQQMESHREAHQKQLSRLRDEIE |
| | | | EKQKIIDEIRDLNQKLQLEQEKLSSDYNKLKIE |
| | | | DQEREMKLEKLLLLNDKREQAREDLKGLEETVS |
| | | | RELQTLHNLRKLFVQDLTTRVKKSVELDNDDGG |
| | | | GSAAQKQKISFLENNLEQLTKVHKQLVRDNADL |
| | | | RCELPKLEKRLRATAERVKALESALKEAKENAM |
| | | | RDRKRYQQEVDRIKEAVRAKNMARRAHSAQIAK |
| | | | PIRPGHYPASSPTAVHAIRGGGGSSSNSTHYQK |
| | | | (SEQ ID NO: 16) |
| SWISS_PR | P28738 | Kinesin heavy | MADPAECSIKVMCRFRPLNEAEILRGDKFIPKF |
| OT: | Q9Z2F8 | chain isoform | KGEETVVIGQGKPYVFDRVLPPNTTQEQVYNAC |
| KF5C_MOU | | 5C (Kinesin | AKQIVKDVLEGYNGTIFAYGQTSSGKTHTMEGK |
| SE | | heavy chain | LHDPQLMGIIPRIAHDIFDHIYSMDENLEFHIK |
| | | neuron- | VSYFEIYLDKIRDLLDVSKTNLAVHEDKNRVPY |
| | | specific\|2) | VKGCTERFVSSPEEVMDVIDEGKANRHVAVTNM |
| | | | NEHSSRSHSIFLINIKQENVETEKKLSGKLYLV |
| | | | DLAGSEKVSKTGAEGAVLDEAKNINKSLSALGN |
| | | | VISALAEGTKTHVPYRDSKMTRILQDSLGGNCR |
| | | | TTIVICCSPSVFNEAETKSTLMFGQRAKTIKNT |
| | | | VSVNLELTAEEWKKKYEKEKSKNKALKSVLQHL |
| | | | EMELNRWRNGEAVPEDEQISAKDHKSLEPCDNT |
| | | | PIIDNITPVVDGISAEKEKYDEEITSLYRQLDD |
| | | | KDDEINQQSQLAEKLKQQMLDQDELLASTRRDY |
| | | | EKIQEELTRLQIENEAAKDEVKEVLQALEELAV |
| | | | NYDQKSQSVSDKTRANSQLTDSLAQKTTTLTTT |
| | | | QRSLSQLQELSNHQKKRATEILNLLLKDLGEIG |
| | | | GIIGTNDVKTLADVNGVIEEEFTMARLYISKMK |
| | | | SEVKSLVNRSKQLESAQMDSNRKMNASERELAA |
| | | | CQLLISQHEAKIKSLTDYMQNMEQKRRQLEESQ |
| | | | DSLSEELAKLRAQEKMHEVSFQDKEKEHLTRLQ |
| | | | DAEEVKKALEQQMESHREAHQKQLSRLRDETEE |
| | | | KQRIIDEIRDLNQKLQLEQERLSSDYNKLKIED |
| | | | QEREVKLEKLLLLNDKREQAREDLKGLEETVSI |
| | | | ELQTLHNLRKLFVQDLTTRVKKSVELDSDDCCC |
| | | | SAAQKQKISFLENNLEQLTKVHKQLVRDNADLR |
| | | | CELPKLEKRLRATAERVKALESALKEAKENAMR |
| | | | DRKRYQQEVDRIKEAVRAKNMARRAHSAQIAKP |
| | | | IRPQHYPASSPTAVHAVRGGGGGSSNSTHYQK |
| | | | (SEQ ID NO: 17) |
| SWISS_PR | P34540 | Kinesin heavy | MEPRTDGAECGVQVFCRIRPLNKTEEKNADRFL |
| OT: | | chain | PKFPSEDSISLGGKVYVFDKVFKPNTTQEQVYK |
| KTNH_CAE | | | GAAYHIVQDVLSGYNGTVFAYGQTSSGKTHTME |
| EL | | | CVIGDNGLSGIIPRIVADIFNHIYSMDENLQFH |
| | | | IKVSYYEIYNEKIRDLLDPEKVNLSIHEDKNRV |
| | | | PYVKGATERFVGGPDEVLQAIEDGKSNRMVAVT |
| | | | NMNEHSSRSHSVFLITVKQEHQTTKKQLTGKLY |
| | | | LVDLAGSEKVSKTGAQGTVLEEAKNINKSLTAL |
| | | | GIVISALAEGTKSHVPYRDSKLTRILQESLGGN |
| | | | SRTTVIICASPSHFNEAETKSTLLFGARAKTIK |
| | | | NVVQINEELTAEEWKRRYEKEKEKNTRLAALLQ |
| | | | AAALELSRWRAGESVSEVEWVNLSDSAQMAVSE |
| | | | VSGGSTPLMERSIAPAPPMLTSTTGPITDEEKK |
| | | | KYEEERVKLYQQLDEKDDEIQKVSQELEKLRQQ |
| | | | VLLQEEALGTMRENEELIREENNRFQKEAEDKQ |
| | | | QEGKEMMTALEEIAVNLDVRQAECEKLKRELEV |
| | | | VQEDNQSLEDRMNQATSLLNAHLDECGPKIRHF |
| | | | KEGIYNVIREFNIADIASQNDQLPDHDLLNHVR |
| | | | IGVSKLFSEYSAAKESSTAAEHDAEAKLAADVA |
| | | | RVESGQDAGRMKQLLVKDQAAKEIKPLTDRVNM |
| | | | ELTTLKNLKKEFMRVLVARCQANQDTEGEDSLS |
| | | | GPAQKQRIQPLENNLDKLTKVHKQLVRDNADLR |
| | | | VELPKMEARLRGREDRIKILETALRDSKQRSQA |
| | | | ERKKYOQEVERTKEAVRORNMRRMNAPQIVKPI |
| | | | RPGQVYTSPSAGMSQGAPNGSNA |
| | | | (SEQ ID NO: 18) |
| SWISS_PR | P17210 | Kinesin heavy | MSAEREIPAEDSIKVVCRFRPLNDSEEKAGSKF |
| OT: | Q9V7L9 | chain | VVKFPNNVEENCISIAGKVYLFDKVFKPNASQE |
| KINH_DRO | | | KVYNEAAKSIVTDVLAGYNGTIFAYGQTSSGKT |
| ME | | | HTMEGVIGDSVKQGIIPRIVNDIFNHIYAMEVN |
| | | | LEFHIKVSYYEIYMDKIRDLLDVSKVNLSVHED |
| | | | KNRVPYVKGATERFVSSPEDVFEVIEEGKSNRH |
| | | | IAVTNMNEHSSRSHSVFLINVKQENLENQKKLS |
| | | | GKLYLVDLAGSEKVSKTGAEGTVLDEAKNINKS |
| | | | LSALGNVISALADGNKTHIPYRDSKLTRILQES |
| | | | LGGNARTTIVICCSPASFNESETKSTLDFGRRA |
| | | | KTVKNVVCVNEELTAEEWKRRYEKEKEKNARLK |
| | | | GKVEKLEIELARWRAGETVKAEEQINMEDLMEA |
| | | | STPNLEVEAAQTAAAEAALAAQRTALANMSASV |
| | | | AVNEQARLATECERLYQQLDDKDEEINQQSQYA |
| | | | EQLKEQVMEQEELIANARREYETLQSEMARIQQ |
| | | | ENESAKEEVKEVLQALEELAVNYDQKSQEIDNK |
| | | | NKDIDALNEELQQKQSVFNAASTELQQLKDMSS |
| | | | HQKKRITEMLTNLLRDLGEVGQAIAPGESSIDL |
| | | | KMSALAGTDASKVEEDFTMARLFISKMKTEAKN |
| | | | IAQRCSNMETQQADSNKKISEYEKDLGEYRLLI |
| | | | SQHEARMKSLQESMREAENKKRTLEEQIDSLRE |
| | | | ECAKLKAAEHVSAVNAEEKQRAEELRSMEDSQM |
| | | | DELREAHTRQVSELRDEIAAKQHEMDEMKDVHQ |
| | | | KLLLAHQQMTADYEKVRQEDAEKSSELQNIILT |
| | | | NERREQARKDLKGLEDTVAKELQTLHNLRKLFV |
| | | | QDLQQRIRKNVVNEESEEDGGSLAQKQKISFLE |
| | | | NNLDQLTKVHKQLVRDNADLRCELPKLEKRLRC |
| | | | TMERVKALETALKEAKEGAMRDRKRYQYEVDRI |
| | | | KEAVRQKHLGRRGPQAQIAKPIRSGQGAIAIRG |
| | | | GGAVGGPSPLAQVNPVNS |
| | | | (SEQ ID NO: 19) |
| SWISS_PR | P33176 | Kinesin heavy | MADLAECNIKVMCRFRPLNESEVNRGDKYIAKF |
| OT: | | chain | QGEDTVVTASKPYAPDRVFQSSTSQEQVYNDCA |
| KINH_HUM | | (Ubiquitous | KKIVKDVLEGYNGTIFAYGQTSSGKTHTMEGKL |
| AN | | kinesin heavy | HDPEGMGIIPRIVQDIFNYIYSMDENLEEHIKV |
| | | chain) (UKHC) | SYFETYLDKIRDLLDVSKTNLSVHEDKNRVPYV |
| | | | KGCTERFVCSPDEVMDTIDEGKSNRHVAVTNMN |
| | | | EHSSRSHSIFLINVKQENTQTEQKLSGKLYLVD |
| | | | LAGSEKVSKTGAEGAVLDEAKNINKSLSALGNV |
| | | | ISALAEGSTYVPYRDSKMTRILQDSLGGNCRTT |
| | | | IVICCSPSSYNESETKSTLLFGQRAKTIKNTVC |
| | | | VNVELTAEQWKKKYEKEKEKNKILRNTIQWLEN |
| | | | ELNRWRNGETVPIDEQFDKEKANLEAFTVDKDI |
| | | | TLTNDKPATAIGVIGNFTDAERRKCEEEIAKLY |
| | | | KQLDDKDEEINQQSQLVEKLKTQMLDQEELLAS |
| | | | TRRDQDNMQAELNRLQAENDASKEEVKEVLQAL |
| | | | EELAVNYDQKSQEVEDKTKEYELLSDELNQKSA |
| | | | TLASIDAELQKLKEMTNHQKKRAAEMMASLLKD |
| | | | LAEIGIAVGNNDVKQPEGTGMIDEEFTVARLYI |
| | | | SKMKSEVKTMVKRCKQLESTQTESNKKMEENEK |
| | | | ELAACQLRISQHEAKIKSLTEYLQNVEQKKRQL |
| | | | EESVDALSEELVQLRAQEKVHEMEKEHLNKVQT |
| | | | ANEVKQAVEQQIQSHRETHQKQISSLRDEVEAK |
| | | | AKLITDLQDQNQKMMLEQERLRVEHEKLKATDQ |
| | | | EKSRKLHELTVMQDRREQARQDLKGLEETVAKE |
| | | | LQTLHNLRKLFVQDLATRVKKSAEIDSDDTGGS |
| | | | AAQKQKISFLENNLEQLTKVHKQLVRDNADLRC |
| | | | ELPKLEKRLRATAERVKALESALKEAKENASRD |
| | | | RKRYQQEVDRIKEAVRSKNMARRGHSAQIAKPI |
| | | | RPGQHPAASPTHPSAIRGGGAFVQNSQPVAVRG |
| | | | GGGKQV |
| | | | (SEQ ID NO: 20) |
| SWISS_PR | P21613 | Kinesin heavy | MDVASECNIKVICRVRPLNEAEERAGSKFILKF |
| OT: | | chain | PTDDSISIAGKVFVFDKVLKPNVSQEYVYNVGA |
| KINH_LOL | | | KPIVADVLSGCNGTIFAYGQTSSGKTHTMEGVL |
| PE | | | DKPSMHGIIPRIVQDIFNYTYGMDENLEPHIKI |
| | | | SYYEIYLDKIRDLLDVTKTNLAVHEDKNRVPFV |
| | | | KGATERPVSSPEEVMEVIDEGKNNRHVAVTNMN |
| | | | EHSSRSHSVFLTNVKQENVETQKKLSGKLYLVD |
| | | | LAGSEKVSKTGAEGAVLDEAKNINKSLSALGNV |
| | | | ISALADGNKSHVPYRDSKLTRILQESLGGNART |
| | | | TMVICCSPASYNESETKSTLLPGQRAKTIKNVV |
| | | | SVNEELTADEWKRRYEKEKERVTKLKATMAKLE |
| | | | AELQRWRTGQAVSVEEQVDLKEDVPAESPATST |
| | | | TSLAGGLIASMNEGDRTQLEEERLKLYQQLDDK |
| | | | DDEINNQSQLIEKLKEQMMEQEDLTAQSRRDYE |
| | | | NLQQDMSRIQADNESAKDEVKEVLQALEELAMN |
| | | | YDQKSQEVEDKNKENENLSEELNQKLSTLNSLQ |
| | | | NELDQLKDSSMHHRKRVTDMMINLLKDLGDTCT |
| | | | IVGGNAAETKPTAGSGEKIEEEFTVARLYISKM |
| | | | KSEVKTLVSRNNQLENTQQDNFKKIETHEKDLS |
| | | | NCKLLTQQHEAKMASLQEAIKDSENKKRMLEDN |
| | | | VDSLNEEYAKLKAQEQMHLAALSEREKETSQAS |
| | | | ETREVLEKQMEMHREQHQKQLQSLRDEISEKQA |
| | | | TVDNLKDDNQRLSLALEKLQADYDKLKQEEVEK |
| | | | AAKLADLSLQTDRREQAKQDLKGLEETVAKELQ |
| | | | TLHNLRKLFVQDLQNKVKKSCSKTEEEDEDTGG |
| | | | NAAQKQKISFLENNLEQLTKVHKQLVRDNADLR |
| | | | CELPKLEKRLRATMERVKSLESALKDAKEGAMR |
| | | | DRKRYQHEVDRIKEAVRQKNLARRGHAAQIAKP |
| | | | IRPGQHQSVSPAQAAAIRGGGGLSQNGPMTTST |
| | | | PTRMAPESKA |
| | | | (SEQ ID NO: 21) |
| SWISS_PR | Q61768 | Kinesin heavy | MADPAECNIKVMCRPRPLNESEVNRGDKYVAKF |
| OT: | Q08711 | chain | QGEDTVVIASKPYAPDRVFQSSTSQEQVYNDCA |
| KTNH_MOU | Q61580 | (Ubiquitous | KKIVKDVLEGYNGTIFAYGQTSSGKTHTMEGKL |
| SE | | kinesin heavy | HDPEGMGIIPRIVQDIFNYTYSMDENLEFHIKV |
| | | chain) (UKHC)Z | SYFEIYLDKTRDLLDVSKTNLSVHEDKNRVPYV |
| | | | KGCTERFVCSPDEVMDTIDEGKSNRHVAVTNMN |
| | | | EHSSRSHSIFLINVKQENTQTEQKLSGKLYLVD |
| | | | LAGSEKVSKTGAEGAVLDEAKNINKSLSALGNV |
| | | | ISALAEGSTYVPYRDSKMTRILQDSLGGNCRTT |
| | | | IVICCSPSSYNESETKSTLLFGQRAKTIKNTVC |
| | | | VNVELTAEOWKKKYEKEKEKNKTLRNTIQWLEN |
| | | | ELNRWRNGETVPIDEQFDKEKANLEAFTADKDI |
| | | | AITSDKGAAAVGMAGSFTDAERRKCEEELAKLY |
| | | | KQLDDKDEEINQQSQLVEKLKTQMLDQEELLAS |
| | | | TRRDQDNMQAELNRLQAENDASKEEVKEVLQAL |
| | | | EELAVNYDQKSQEVEDKTKEYELLTDEFNQKSA |
| | | | TLASIDAELQKLKEMTNHQKKRAAEMMASLLKD |
| | | | LAEIGIAVGNNDVKQPEGTGMIDEEFTVARLYI |
| | | | SKMKSEVKTMVKRCKQLESTQTESNKKMEENEK |
| | | | ELAACQLRISQHEAKIKSLTEYLQNDEQKKRQL |
| | | | EESLDSLGEELVQLRAQEKVHEMEKEHLNKVQT |
| | | | ANEVKQAVEQQIQSHRETHQKQISSLRDEVEAK |
| | | | EKLITDLQDQNQKMVLETERLRVEHERLKATDQ |
| | | | EKSRKLHELTVMQDRREQARQDLKGLEETVAKE |
| | | | LQTLHNLRKLFVQDLATRVKKSAEVDSDDTGGS |
| | | | AAQKQKISFLENNLEQLTKVHKQLVRDNADLRC |
| | | | ELPKLEFRLRATAERVKALESALKEAKENASRD |
| | | | RKRYQQEVDRIKEAVRSKNMARRGHSAQIAKPI |
| | | | RPGQHPAASPTHPGTVRGGGSFVQNNQPVGLRG |
| | | | GGGKQS |
| | | | (SEQ ID NO: 22) |
| SWISS_PR | P48467 | Kinesin heavy | MSSSANSIKVVARERPQNRVEIESGGQPIVTEQ |
| OT: | | chain | GPDTCTVDSKEAQGSFTFDRVEDMSCKQSDIFD |
| KINH_NEU | | | FSIKPTVDDILNGYNGTVFAYGQTGAGKSYTMM |
| CR | | | GTSIDDPDGRGVIPRIVEQIFTSILSSAANIEY |
| | | | TVRVSYMEIYMERIRDLLAPQNDNLPVHEEKNR |
| | | | GVYVKGLLEIYVSSVQEVYEVMRRGGNARAVAA |
| | | | TNMNQESSRSHSIFVTTTTQKNVETGSAKSGQL |
| | | | FLVDLAGSEKVGKTGASGQTLEEAKKINKSLSA |
| | | | LGMVINALTDGKSSHVPYRDSKLTRILQESLGG |
| | | | NSRTTLIINCSPSSYNDAETLSTLFEGMRAKSI |
| | | | KNKAKVNAELSPAELKWMLAKAKTQITSFENYI |
| | | | VNLESEVQVWRGGETVPKEKWVPPLELAITPSK |
| | | | SASTTARPSTPSRLLPESRAETPAISDRAGTPS |
| | | | LPLDKDEREEFLRRENELQDQIAEKESIAAAAE |
| | | | RQLRETKEELIALKDHDSKLGKENERLISESNE |
| | | | FKMQLERLAEENKEAQITIDGLKDANSELTAEL |
| | | | DEVKQQMLDMKMSAKETSAVLDEKEKKKAEKMA |
| | | | KMMAGFDLSGDVFSDNERAVADAIAQLDALEEI |
| | | | SSAGDAIPPEDIKALREKLVETQCFVRQAFLSS |
| | | | FSAASSDAEARKRAELEARLEALQQEHEELLSR |
| | | | NLTEADKEEVKALLAKSLSDKSAVQVELVEQLK |
| | | | ADIALKNSETEHLKALVDDLQRRVKAGGAGVAM |
| | | | ANGKTVQQQLAEFDVMKKSLMRDLQNRCERVVE |
| | | | LEISLDETREQYNNVLRSSNNRAQQKKMAFLER |
| | | | NLEQLTQVQRQLVEQNSALKKEVAIAERKLMAR |
| | | | NERIQSLESLLQESQEKMAQANHKFEVQLAAVK |
| | | | DRLEAAKAGSTRGLGTDAGLGGFSIGSRIAKPL |
| | | | RGGGDAVAGATATNPTIATLQQNPPENKRSSWF |
| | | | FQKS |
| | | | (SEQ ID NO: 23) |
| SWISS_PR | P35978 | Kinesin heavy | MADPAECNIKVVCRVRPMNATEQNTSHICTKFI |
| OT: | | chain | SEEQVQIGGKLNMFDRIFKPNTTQEEVYNKAAR |
| KINH_STR | | | QIVKDVLDGYNGTIFAYGQTSSGKTFTMEGVMG |
| PU | | | NPQYMGIIPRIVQDIFNHIYQMDESLEFHIKVS |
| | | | YFEIYMDRIRDLLDVSKTNLSVHEDKNRVPFVK |
| | | | GATERFASSPEEVMDVIEEGKSNRHIAVTNMNE |
| | | | HSSRSHSIFLIQVKQENMETKKKLSGKLYLVDL |
| | | | AGSEKVSKTGAEGTVLDEAKNINKSLSALGNVI |
| | | | SALADGKKSHIPYRDSKMTRILQESLGGNARTT |
| | | | IVICCSPSSFNESESKSTLMFGQRAKTIKNTVT |
| | | | VNMELTAEEWRNRYEKEKEKNGRLKAQLLILEN |
| | | | ELQRWRAGESVPVKEQGNKNDEILKEMMKPKQM |
| | | | TVHVSEEEKNKWEEEKVKLYEQLDEKDSEIDNQ |
| | | | SRLTEKLKQQMLEQEELLSSMQRDYELLQSQMG |
| | | | RLEAENAAAKEEAKEVLQALEEMAVNYDEKSKE |
| | | | VEDKNRMNETLSEEVNEKMTALHTTSTELQKLQ |
| | | | ELEQHQRRRITEMMASLLKDLGEIGTALCGNAA |
| | | | DMKPNVENIEKVDEEFTMARLFVSKMKTEVKTM |
| | | | SQRCKILEASNAENETKIRTSEDELDSCRMTIQ |
| | | | QHEAKMKSLSENIRETEGKKRHLEDSLDMLNEE |
| | | | IVKLRAAEEIRLTDQEDKKREEEDKMQSATEMQ |
| | | | ASMSEQMESHRDAHQKQLANLRTEINEKEHQME |
| | | | ELKDVNQRMTLQHEKLQLDYEKLKIEEAEKAAK |
| | | | LRELSQQFDRREQAKQDLKGLEETVAKELQTLH |
| | | | NLRKLFVSDLQNRVKKALEGGDRDDDSGGSQAC |
| | | | KQKISFLENNLEQLTKVHKQLVRDNADLRCELP |
| | | | KLERRLRATSERVKALEMSLKETKEGAMRDRKR |
| | | | YQQEVDRIREAVRQRNFAKRGSSAQIAKAIRAG |
| | | | HPPPSPGGSTGIRGGGYSGIRGGGSPVIRPPSH |
| | | | GSPEPISHNNSFEKSLNPNDAENMEKKANKRLP |
| | | | KLPPGGNKLTESDIAAMKARSKARNNTPGKAPL |
| | | | TTSGEQGS |
| | | | (SEQ ID NO: 24) |
| SWISS_PR | 043093 | Kinesin heavy | MSGNNIKVVCRFRPQNSLEIREGGTPIIDIDPE |
| OT:KINH_{—} | | chain (Synkin) | GTQLELKGKEFKGNFNFDKVFGMNTAQKDVFDY |
| SYNRA | | | SIKTIVDDVTAGYNGTVFAYGQTGSGKTFTMMG |
| | | | ADIDDEKTKGIIPRIVEQIFDSIMASPSNLEFT |
| | | | VKVSYMEIYMEKVRDLLNPSSENLPIHEDKTKG |
| | | | VYVKGLLEVYVGSTDEVYEVMRRGSNNRVVAYT |
| | | | NMNAESSRSHSIVMFTITQKNVDTGAAKSGKLY |
| | | | LVDLAGSEKVGKTGASGQTLEEAKKINKSLTAL |
| | | | GMVINALTDGKSSHVPYRDSKLTRILQESLGGN |
| | | | SRTTLIINCSPSSYNEAETLSTLRFGARAKSIK |
| | | | NKAKVNADLSPAELKALLKKVKSEAVTYQTYIA |
| | | | ALEGEVNVWRTGGTVPEGKWVTMDKVSKGDFAG |
| | | | LPPAPGFKSPVSDEGSRPATPVPTLEKDEREEF |
| | | | IKRENELMDQISEKETELTNREKLLESLREEMG |
| | | | YYKEQEQSVTKENQQMTSELSELRLQLQKVSYE |
| | | | SKENAITVDSLKEANQDLMAELEELKKNLSEMR |
| | | | QAHKDATDSDKEKRKAEKMAQMMSGFDPSGILN |
| | | | DKERQIRNALSKLDGEQQQTLTVEDLVSLRREL |
| | | | AESKMLVEQHTKTISDLSADKDAMEAKKIELEG |
| | | | RLGALEKEYEELLDKTIAEEEANMQNADVDNLS |
| | | | ALKTKLEAQYAEKKEVQQKEIDDLKRELDRKQS |
| | | | GHEKLSAAMTDLRAANDQLQAALSEQPFQAPQD |
| | | | NSDMTEKEKDIERTRKSMAQQLADFEVMKKALM |
| | | | RDLQNRCEKVVELEMSLDETREQYNNVLRASNN |
| | | | KAQQKKMAFLERNLEQLTNVQKQLVEQNASLKK |
| | | | EVALAERKLIARNERIQSLETLLHNAQDKLLNQ |
| | | | NKKFEQQLATVRERLEQARSQKSQNSLAALNES |
| | | | RIAKPLRGNGAAIDNGSDDGSLPTSPTDKRDKR |
| | | | SSWMPGFMNSR |
| | | | (SEQ ID NO: 25) |
| SWISS_PR | Q12840 | Neuronal | MAETNNECSIKVLCRFRPLNQAEILRGDKFIPI |
| OT: | | kinesin heavy | FQGDDSVVIGGKPYVFDRVFPPNTTQEQVYHAC |
| KINN_HUM | | chain (NKHC) | AMQIVKDVLAGYNGTIFAYGQTSSGKTHTMEGK |
| AN | | (Kinesin heavy | LHDPQLMGIIPRIARDIFNHIYSMDENLEPHIK |
| | | chain isoform | VSYFEIYLDKIRDLLDVTKTNLSVHEDKNRVPF |
| | | 5A) \| (Kinesin | VKGCTERFVSSPEEILDVIDEGKSNRHVAVTNM |
| | | heavy chain | NEHSSRSHSIFLINIKQENMETEQKLSGKLYLV |
| | | neuron-specific | DLAGSEKVSKTGAEGAVLDEAKNINKSLSALGN |
| | | 1) | VISALAEGTKSYVPYRDSKMTRILQDSLGGNCR |
| | | | TTMFICCSPSSYNDAETKSTLMEGQRAKTIKNT |
| | | | ASVNLELTAEQWKKKYEKEKEKTKAQKETIAKL |
| | | | EAELSRWRNGENVPETERLAGEEAALGAELCEE |
| | | | TPVNDNSSIVVRIAPEERQKYEEEIRRLYKQLD |
| | | | DKDDEINQQSQLIEKLKQQMLDQEELLVSTRGD |
| | | | NEKVQRELSHLQSENDAAKDEVKEVLQALEELA |
| | | | VNYDQKSQEVEEKSQQNQLLVDELSQKVATMLS |
| | | | LESELQRLQEVSGHQRKRIAEVLNGLMKDLSEF |
| | | | SVIVGNGEIKLPVEISGAIEEEFTVARLYTSKI |
| | | | KSEVKSVVKRCRQLENLQVECHRKMEVTGRELS |
| | | | SCQLLISQHEAKIRSLTEYMQSVELKKRHLEES |
| | | | YDSLSDELAKLQAQETVHEVALKDKEPDTQDAD |
| | | | EVKKALELQMESHREAHHRQLARLRDEINEKQK |
| | | | TIDELKDLNQKLQLELEKLQADYEKLKSEEHEK |
| | | | STKLQELTFLYERHEQSKQDLKGLEETVARELQ |
| | | | TLHNLRKLFVQDVTTRVKKSAEMEPEDSGGIHS |
| | | | QKQKISPLENNLEQLTKVHKQLVRDNADLRCEL |
| | | | PKLEKRLRATAERVKALEGALKEAKEGAMKDKR |
| | | | RYQQEVDRIKEAVRYKSSGKRAHSAQIAKPVRP |
| | | | GHYPASSPTNPYGTRSPECISYTNSLFQNYQNL |
| | | | YLQATPSSTSDMYFANSCTSSGATSSGCPLASY |
| | | | QKANMDNCNATDINDNRSDLPCGYEAEDQAKLF |
| | | | PLHQETAAS |
| | | | (SEQ ID NO: 26) |
| SWISS_PR | P33175 | Neuronal | MAETNNECSIKVLCRFRPLNQAEILRGDKFIPI |
| OT:KINN_{—} | Q9Z2F9 | kinesin heavy | FQGDDSVIIGGKPYVFDRVFPPNTTQEQVYHAC |
| MOUSE | | chain (NKHC) | AMQIVKDVLAGYNGTIFAYGQTSSGKTHTMEGK |
| | | (Kinesin heavy | LHDPQLMGIIPRIARDIENHIYSMDENLEPHIK |
| | | chain isoform | VSYFEIYLDKIRDLLDVTKTNLSVHEDKNRVPF |
| | | 5A) | VKGCTERFVSSPEEILDVIDEGKSNRHVAVTNM |
| | | (Kinesin heavy | NEHSSRSHSIPLINIKQENVETEQKLSGKLYLV |
| | | chain | DLAGSEKVSKTGAEGAVLDEAKNINKSLSALGN |
| | | neuron-specific | VISALAEGTKSYVPYRDTKMTRILQDSLGGNCR |
| | | 1) | TTMFICCSPSSYNDAETKSTLMFGQRAKTIKNT |
| | | | ASVNLELTAEQWKKKYEKEKEKTKAQKETIANV |
| | | | EAELSRWRNGENVPETERLAGEDSALGAELCEE |
| | | | TPVNDNSSIVVRIAPEERQKYEEEIRRLYKQLD |
| | | | DKDDEINQQSQLIEKLKQQMLDQEELLVSTRGD |
| | | | NEKVQRELSHLQSENDAAKDEVKEVLQALEELA |
| | | | VNYDQKSQEVEEKSQQNQLLVDELSQKVATMLS |
| | | | LESELQRLQEVSGHQRKRIAEVLNGLMRDLSEF |
| | | | SVIVGNGEIKLPVEISGAIEEEFTVARLYISKI |
| | | | KSEVKSVVKRCRQLENLQVECHRKMEVTGRELS |
| | | | SCQLLISQHEAKIRSLTEYMQTVELKKRHLEES |
| | | | YDSLSDELARLQAHETVHEVALKDKEPDTQDAE |
| | | | EVKKALELQMENHREAHHRQLARLRDEINEKQK |
| | | | TIDELKDLNQKLQLELEKLQADYERLKNEENEK |
| | | | SAKLQELTFLYERHEQSKQDLKGLEETVARELQ |
| | | | TLHNLRKLFVQDVTTRVKKSAEMEPEDSGGIHS |
| | | | QKQKISFLENNLEQLTKVHKQLVRDNADLRCEL |
| | | | PKLEKRLRATAERVKALEFALKEAKEGAMKDKR |
| | | | RYQQEVDRIKEAVRYKSSGKRGHSAQIAKPVRP |
| | | | GHYPASSPTNPYGTRSPECISYTNNLFQNYQNL |
| | | | HLQAAPSSTSDMYFASSGRTSVAPLASYQKANM |
| | | | DNGNATDINDNRSDLPCGYEAEDQAKLEPLHQE |
| | | | TAAS |
| | | | (SEQ ID NO: 27) |
| SWISS_PR | P28742 | Kinesin-like | MARSSLPNRRTAQFEANKRRTIAHAPSPSLSNG |
| OT: | | protein KIP1 | MHTLTPPTCNNGAATSDSNIHVYVRCRSRNKRE |
| KIP1_YEA | | | TEEKSSVVISTLGPQGKEIILSNGSHQSYSSSK |
| ST | | | KTYQFDQVFGAESDQETVFNATAKNYIKEMLHG |
| | | | YNCTIFAYGQTGTGKTYTMSGDTNILGDVQSTD |
| | | | NLLLGEHAGIIPRVLVDLFKELSSLNKEYSVKT |
| | | | SFLELYNENLKDLLSDSEDDDPAVNDPKRQIRT |
| | | | FDNNNNNSSIMVKGMQEIFINSAHEGLNLLMQG |
| | | | SLKRKVAATKCNDLSSRSHTVFTITTNIVEQDS |
| | | | KDHGQNKNFVKIGKLNLVDLAGSENINRSGAEN |
| | | | KRAQEAGLINKSLLTLGRVINALVDHSNHIPYR |
| | | | ESKLTRLLQDSLGGMTKTCIIATISPAKISMEE |
| | | | TASTLEYATRAKSIKNTPQVNQSLSKDTCLKDY |
| | | | IQEIEKLRNDLKNSRNKQGIFITQDQLDLYESN |
| | | | SILIDEQNLKIHNLREQIKKFKENYLNQLDINN |
| | | | LLQSEKEKLIAIIQNFNVDFSNFYSEIQKIHHT |
| | | | NLELMNEVIQQRDFSLENSQKQYNTNQNMQLKI |
| | | | SQQVLQTLNTLQGSLNNYNSKCSEVIKGVTEEL |
| | | | TRNVNTHKAKHDSTLKSLLNITTNLLMNQMNEL |
| | | | VRSISTSLEIFQSDSTSHYRKDLNEIYQSHQQF |
| | | | LKNLQNDTKSCLDSIGSSILTSINEISQNCTTN |
| | | | LNSMNVLIENQQSGSSKLIKEQDLEIKKLKNDL |
| | | | INERRISNQFNQQLAEMKRYFQDHVSRTRSEFH |
| | | | DELNKCIDNLKDKQSKLDQDIWQKTASIFNETD |
| | | | IVVNKIHSDSIASLAHNAENTLKTVSQNNESFT |
| | | | NDLISLSRGMNMDISSKLRSLPINEFLNKISQT |
| | | | ICETCGDDNTIASNPVLTSIKKFQNIICSDIAL |
| | | | TNEKIMSLIDETQSQIETISNENNINLIAINEN |
| | | | FNSLCNFILTDYDENIMQISKTQDEVLSEHCEK |
| | | | LQSLKILGMDIFTAHSIEKPLHEHTRPEASVIK |
| | | | ALPLLDYPKQFQIYRDAENKSKDDTSNSRTCIP |
| | | | NLSTNENFPLSQFSPKTPVPVPDQPLPKVLIPK |
| | | | SINSAKSNRSKTLPNTEGTGRESQNNLKRRFTT |
| | | | EPILKGEETENNDTLQNKKLHQ |
| | | | (SEQ ID NO: 28) |
| SWISS_PR | P28743 | Kinesin-like | MIQKMSPSLRRPSTRSSSGSSNIPQSPSVRSTS |
| OT: | | protein KIP2 | SFSNLTRNSIRSTSNSGSQSISASSTRSNSPLR |
| KIP2_YEA | | | SVSAKSDPFLHPGRIRIRRSDSINNNSRKNDTY |
| ST | | | TGSITVTIRPKPRSVGTSRDHVGLKSPRYSQPR |
| | | | SNSHHGSNTFVRDPWFITNDKTIVHEEIGEFKF |
| | | | DHVEASHCTNLEVYERTSKPMIDKLLMGFNATI |
| | | | FAYGMTGSGKTFTMSGNEQELGLIPLSVSYLET |
| | | | NIMEQSMNGDKKEDVIISYLEIYNERIYDLLES |
| | | | GLEESGSRISTPSRLYMSKSNSNGLGVELKIRD |
| | | | DSQYGVKVIGLTERRCESSEELLRWIAVGDKSR |
| | | | KIGETDYNARSSRSHAIVLIRLTSTNVKNGTSR |
| | | | SSTLSLCDLAGSERATGQQERRKEGSFINKSLL |
| | | | ALGTVISKLSADKMNSVGSNIPSPSASGSSSSS |
| | | | GNATNNGTSPSNHIPYRDSKLTRLLQPALSGDS |
| | | | IVTTICTVDTRNDAAAETMNTLRFASRAKNVAL |
| | | | HVSKKSIISNGNNDGDKDRTIELLRRQLEEQRR |
| | | | MISELKNRSNIGEPLTKSSNESTYKDIKATGND |
| | | | GDPNLALMRAENRVLKYKLENCEKLLDKDVVDL |
| | | | QDSEIMEIVEMLPFEVGTLLETKFQGLESQIRQ |
| | | | YRKYTQKLEDKIMALEKSGHTAMSLTGCDGTEV |
| | | | IELQKMLERKDKMIEALQSAKRLRDRALKPLIN |
| | | | TQQSPHPVVDNDK |
| | | | (SEQ ID NO: 29) |
| SWISS_PR | P46822 | Kinesin light | MSNMSQDDVTTGLRTVQQGLEALREEHSTISNT |
| OT: | Q18088 | chain (KLC) | LETSVKGVKEDEAPLPKQKLSQINDNLDKLVCG |
| KLC_CAEE | | | VDETSLMLMVFQLTQGMDAQHQKYQAQRRRLCQ |
| L | | | ENAWLRDELSSTQIKLQQSEQMVAQLEEENKHL |
| | | | KYMASIKQLDDGTQSDTKTSVDVGPQPVTNETL |
| | | | QELGFGPEDEEDMNASQFNQPTPANQMAASANV |
| | | | GYEIPARLRTLHNLVIQYASQGRYEVAVPLCKQ |
| | | | ALEDLEKTSGHDHPDVATMLNILALVYRDQNKY |
| | | | KEAANLLNEALSIREKCLGESHPAVAATLNNLA |
| | | | VLFGKRGKFKDAEPLCKRALEIREKVLGDDHPD |
| | | | VAKQLNNLALLCQNQGKYEEVEKYYKRALEIYE |
| | | | SKLGPDDPNVAKTKNNLSSAYLKQGKYKEAEEL |
| | | | YKQILTRAHEREFGQISGENKPIWQIAEEREEN |
| | | | KHKGEGATANEQAGWAKAAKVDSPTVTTTLKNL |
| | | | GALYRRQGKYEAAETLEDVALRAKKQHEPLRSG |
| | | | AMGGIDEMSQSMMASTIGGSRNSMTTSTSQTGL |
| | | | KNKLMNALGFNS |
| | | | (SEQ ID NO: 30) |
| SWISS_PR | P46824 | Kinesin light | MTQMSQDEIITNTKTVLQGLEALRVEHVSIMNG |
| OT: | Q9VU05 | chain (KLC) | IAEVQKDNEKSDMLRKNIENIELGLSEAQVMMA |
| KLC_DROM | | | LTSHLQNIEAEKHKLKTQVRRLHQENAWLRDEL |
| E | | | ANTQQKFQASEQLVAQLEEEKKHLEFMASVKKY |
| | | | DENQEQDDACDKSRTDPVVELFPDEENEDRHNM |
| | | | SPTPPSQFANQTSGYEIPARLRTLHNLVIQYAS |
| | | | QGRYEVAVPLCKQALEDLERTSGHDRPDVATML |
| | | | NILALVYRDQNKYKEAANLLNDALSIRGKTLGE |
| | | | NHPAVAATLNNLAVLYGKRGKYKDAEPLCKRAL |
| | | | EIREKVLGKDHPDVAKQLNNLALLCQNQGKYDE |
| | | | VEKYYQRALDIYESKLGPDDPNVAKTKNNLAGC |
| | | | YLKQGRYTEAEILYKQVLTRAHEREFGAIDSKN |
| | | | KPIWQVAEEREEHKFDNRENTPYGEYCGWHKAA |
| | | | KVDSPTVTTTLKNLGALYRRQCMFEAAETLEDC |
| | | | AMRSKKEAYDLAKQTKLSQLLTSNEKRRSKAIK |
| | | | EDLDFSEEKNAKP |
| | | | (SEQ ID NO: 31) |
| SWISS_PR | P46825 | Kinesin light | MEVTQTVKSYRIKKIEEIGKMTALSQEEIISNT |
| OT: | | chain (KLC) | KTVIQGLDTLKNEHNQILNSLLTSMKTIRKENG |
| KLC_LOLP | | | DTNLVEEKANILKKSVDSIELGLGEAQVMMALA |
| E | | | NHLQHTEAEKQKLRAQVRRLCQENAWLRDELAN |
| | | | TQQKLQMSEQKVATIEEEKKHLEFMNEMKKYDT |
| | | | NEAQVNEEKESEQSSLDLGFPDDDDDGGQPEVL |
| | | | SPTQPSAMAQAASGGCEIPARLRTLHNLVIQYA |
| | | | SQGRYEVAVPLCKQALEDLEKTSGHDHPDVATM |
| | | | LNILALVYRDQGKYKEAANLLNDALGIREKTLG |
| | | | PDHPAVAATLNNLAVLYGKRGKYKDAEPLCKRA |
| | | | LVIREKVLGKDHPDVAKQLNNLALLCQNQGKYE |
| | | | EVERYYQRALEIYQKELGPDDPNVAKTKNNLAS |
| | | | AYLKQGKYKQAEILYKEVLTRAHEKEFGKVDDD |
| | | | NKPIWMQAEEREENKAKYKDGAPQPDYGSWLKA |
| | | | VKVDSPTVTTTLKNLGALYRRQGKYEAAETLEE |
| | | | CALRSRKSALEVVRQTKISDVLGSDFSKGQSPK |
| | | | DRKRSNSRDRNRRDSMDSVSYEKSGDGDEHEKS |
| | | | KLHVGTSHKQ |
| | | | (SEQ ID NO: 32) |
| SWISS_PR | Q05090 | Kinesin light | MSGSKLSTPNNSGGGQGNLSQEQIITGTREVIK |
| OT: | Q05089 | chain (KLC) | GLEQLKNEHNDILNSLYQSLKMLKKDTPGDSNL |
| KLC_STRP | Q05088 | | VEEKTDIIEKSLESLELGLGEAKVMMALGHHLN |
| U | Q04801 | | MVEAEKQKLRAQVRRLVQENTWLRDELAATQQK |
| | | | LQTSEQNLADLEVKYKHLEYMNSIKKYDEDRTP |
| | | | DEEASSSDPLDLGFPEDDDGGQADESYPQPQTG |
| | | | SGSVSAAAGGYEIPARLRTLHNLVIQYASQSRY |
| | | | EVAVPLCKQALEDLEKTSGHDHPDVATMLNILA |
| | | | LVYRDQNKYKEAGNLLHDALAIREKTLGPDHPA |
| | | | VAATLNNLAVLYGKRGKYKEAEPLCKRALEIRE |
| | | | KVLGKDHPDVAKQLNNLALLCQNQGKYEEVEWY |
| | | | YQRALEIYEKKLGPDDPNVAKTKNNLAAAYLKQ |
| | | | GKYKAAETLYKQVLTRAHEREFGLSADDKDNKP |
| | | | IWMQAEEREEKGKFKDNAPYGDYGGWHKAAKVD |
| | | | SRSRSSPTVTTTLKNLGALYRRQGKYDAAEILE |
| | | | ECAMKSRRNALDMVRETKVRELLGQDLSTDVPR |
| | | | SEAMAKERHHRRSSGTPRHGSTESVSYEKTDGS |
| | | | EEVSIGVAWKAKRKAKDRSRSIPAGYVEIPRSP |
| | | | PHVLVENGDGKLRRSGSLSKLRASVRRSSTKLL |
| | | | NKLKGRESDDDGGMKRASSMSVLPSRGNDESTP |
| | | | APIQLSQRGRVGSHDNLSSRRQSGNF |
| | | | (SEQ ID NO: 33) |
| SWISS_PR | Q42401 | Matrilin-3 | MRRALGTLGCCLALLLPLLPAARGVPHRHRRQP |
| OT: | | precursor | LGSGLGRHGAADTACKNRPLDLVFIIDSSRSVR |
| MTN3_CHI | | | PEEFEKVKIELSKMIDTLDVGERTTRVAVMNYA |
| CK | | | STVKVEFPLRTYFDKASMKEAVSRIQPLSAGTM |
| | | | TGLAIQAAMDEVFTEEMGTRPANFNIPKVVIIV |
| | | | TDGRPQDQVENVAANARTAGIEIYAVGVGRADM |
| | | | QSLRIMASEPLDEHVFYVETYGVIEKLTSKFRE |
| | | | TFCAANTCALGTHDCEQVCVSNDGSYLCDCYEG |
| | | | YTLNPDKRTCSAVDVCAPGRHECDQICVSNNGS |
| | | | YVCECFEGYTLNPDKKTCSAMDVCAPGRHDCAQ |
| | | | VCRRNGGSYSCDCFEGFTLNPDKKTCSAVDVCA |
| | | | PGRHDCEQVCVRDDLFYTCDCYQGYVLNPDKKT |
| | | | CSRATTSSLVTDEEACKCEAIAALQDSVTSRLE |
| | | | ALSTKLDEVSQKLQAYQDRQQVV |
| | | | (SEQ ID NO: 34) |
| SWISS_PR | O15232 | Matrilin-3 | MPRPAPARRLPGLLLLLWPLLLLPSAAPDPVAR |
| OT: | | precursor | PGFRRLETRGPGGSPGRRPSPAAPDGAPASGTS |
| MTN3_HUM | | | EPGRARGAGVCKSRPLDLVFIIDSSRSVRPLEF |
| AN | | | TKVKTFVSRIIDTLDIGPADTRVAVVNYASTVK |
| | | | IEFQLQAYTDKQSLKQAVGRITPLSTGTMSGLA |
| | | | IQTAMDEAFTVEAGAREPSSNIPKVAIIVTDGR |
| | | | PQDQVNEVAARAQASGIELYAVGVDRADMASLK |
| | | | MMASEPLEEHVFYVETYGVIEKLSSRPQETFCA |
| | | | LDPCVLGTHQCQHVCISDGEGKHHCECSQGYTL |
| | | | NADKKTCSALDRCALNTHGCEHICVNDRSGSYH |
| | | | CECYEGYTLNEDRKTCSAQDKCALGTHGCQHIC |
| | | | VNDRTGSHHCECYEGYTLNADKKTCSVRDKCAL |
| | | | GSHGCQHICVSDGAASYHCDCYPGYTLNEDKKT |
| | | | CSATEEARRLVSTEDACGCEATLAFQDKVSSYL |
| | | | QRLNTKLDDILEKLKINEYGQIHR |
| | | | (SEQ ID NO: 35) |
| SWISS_PR | O35701 | Matrilin-3 | MLLSAPLRHLPGLLLLLWPLLLLPSLAAPGRLA |
| OT: | Q9JHM0 | precursor | RASVRRLGTRVPGGSPGHLSALATSTRAPYSGG |
| MTN3_MOU | | | RGAGVCKSRPLDLVFIIDSSRSVRPLEFTKVKT |
| SE | | | FVSRIIDTLDIGATDTRVAVVNYASTVKIEPQL |
| | | | NTYSDKQALKQAVARITPLSTGTMSGLAIQTAM |
| | | | EEAFTVEAGARGPMSNIPKVAIIVTDGRPQDQV |
| | | | NEVAARARASGIELYAVGVDRADMESLKMMASK |
| | | | PLEEHVFYVETYGVIEKLSARFQETFCALDQCM |
| | | | LGTHQCQHVCVSDGDGKHHCECSQGYTLNADGK |
| | | | TCSAIDKCALSTHGCEQICINDRNGSYHCECYG |
| | | | GYALNADRRTCAALDKCASGTHGCQHICVNDGA |
| | | | GSHHCECFEGYTLNADKKTCSVRNKCALGTHGC |
| | | | QHICVSDGAVAYHCDCFPGYTLNDDKKTCSDIE |
| | | | EARSLISIEDACGCGATLAFQEKVSSHLQKLNT |
| | | | KLDNILKKLKVTEYGQVHR |
| | | | (SEQ ID NO: 36) |
| SWISS_PR | Q24167 | Similar protein | MVSLTDTIEAAAEKQKQSQAVVTNTSASSSSCS |
| OT: | Q9VAA5 | | SSESSSPPSSSVGSPSPGAPKTNLTASGKPKEK |
| SIMA_DRO | | | RRNNEKRKEKSRDAARCRRSKETEIFMELSAAL |
| ME | | | PLKTDDVNQLDKASVMRTTIAELKIREMLQFVP |
| | | | SLRDCNDDTKQDIETAEDQQEVKPKLEVGTEDW |
| | | | LNGAEARELLKQTMDGELLVLSHEGDITYVSEN |
| | | | VVEYLGITKIDTLGQQIWEYSHQCDHAEIKEAL |
| | | | SLKRELAQKVKDEPQQNSGVSTHHRDLFVRLKC |
| | | | TLTSRGRSINIKSASYKVIHITGHLVVNAKGER |
| | | | LLMAIGRPIPHPSNIEIPLGTSTELTKHSLDMR |
| | | | FTYVDDKMHDLLGYSPKDLLDTSLFSCQHGADS |
| | | | ERLMATFKSVLSKGQGETSRYRFLGKYGGYCWI |
| | | | LSQATIVYDKLKPQSVVCVNYVISNLENKHEIY |
| | | | SLAQQTAASEQKEQHHQAAETEKEPEKAADPEI |
| | | | IAQETKETVNTPIHTSELQAKPLQLESEKAEKT |
| | | | IEETKTIATIPPVTATSTADQIKQLPESNPYKQ |
| | | | ILQAELLIKRENHSPGPRTITAQLLSGSSSGLR |
| | | | PEEKRPKSVTASVLRPSPAPPLTPPPTAVLCKK |
| | | | TPLGVEPNLPPTTTATAAIISSSNQQLQIAQQT |
| | | | QLQNPQQPAQDMSKGFCSLFADDGRGLTMLKEE |
| | | | PDDLSHHLASTNCIQLDEMTPESDMLVGLMGTC |
| | | | LLPEDINSLDSTTCSTTASGQHYQSPSSSSTSA |
| | | | PSNTSSSNNSYANSPLSPLTPNSTATASNPSHQ |
| | | | QQQQHHNQQQQQQQQQQHHPQHHDNSNSSSNID |
| | | | PLFNYREESNDTSCSQHLHSPSITSKSPEDSSL |
| | | | PSLCSPNSLTQEDDFSEEAFAMRAPYIPIDDDM |
| | | | PLLTETDLMWCPPEDLQTMVPKEIDAIQQQLQQ |
| | | | LQQQHHQQYAGNTGYQQQQQQPQLQQQHFSNSL |
| | | | CSSPASTVSSLSPSPVQQHHQQQQAAVFTSDSS |
| | | | ELAALLCGSGNGTLSILAGSGVTVAEECNERLQ |
| | | | QHQQQQQQTSGNEERTFQQLQQELQLQEEQQQR |
| | | | QQQQQQQQQQQQQQQLLSLNIECKKEKYDVQMG |
| | | | GSLCHPMEDAEENDYSKDSANLDCWDLIQMQVV |
| | | | DTEPVSPNAASPTPCKVSAIQLLQQQQQLQQQQ |
| | | | QQQQNIILNAVPLITIQNNKELMQQQQQQQQQQ |
| | | | QQEQLQQPAIKLLNGASIAPVNTKATIRLVESK |
| | | | PPTTTQSRMAKVNLVPQQQQHGNKRHLNSATGA |
| | | | GNPVESKRLKSGTLCLDVQSPQLLQQLIGKDPA |
| | | | QQQTQAAKRAGSERWQLSAESKQQKQQQQQSNS |
| | | | VLKNLLVSGRDDDDSEAMITDEDNSLVQPIPLG |
| | | | KYGLPLHCHTSTSSVLRDYHNNPLISGTNFQLS |
| | | | PVPGGSDSSGGDGETGSVVSLDDSVPPGLTACD |
| | | | TDASSDSGIDENSLMDGASGSPRKRLSSTSNST |
| | | | NQAESAPPALDVETPVTQKSVEEEFEGGGSGSN |
| | | | APSRKTSISFLDSSNPLLHTPAMMDLVNDDYIM |
| | | | GEGGFEFSDNQLEQVLGWPEIA |
| | | | (SEQ ID NO: 37) |
| SWISS_PR | P23497 | Nuclear | MAGGGGDLSTRRLNECISPVANEMNHLPAHSHD |
| OT: | Q13343 | autoantigen Sp- | LQRMPTEDQGVDDRLLYDIVFKHEKRNKVEISN |
| SP10_HUM | O75450 | 100 (Speckled | AIKKTFPFLEGLRDRDLITNKMFEDSQDSCRNL |
| AN | Q9UE32 | 100 kDa) | VPVQRVVYNVLSELEKTFNLPVLEALFSDVNMQ |
| | | (Nuclear dot- | EYPDLIHIYKGFENVIHDKLPLQESEEEEREER |
| | | associated\|Sp10 | SGLQLSLEQGTGENSFRSLTWPPSGSPSHAGTT |
| | | 0 protein) | PPENGLSEHPCETEQINAKRKDTTSDKDDSLGS |
| | | (Lysp100b) | QQTNEQCAQKAEPTESCEQIAVQVNNGDAGREM |
| | | | PCPLPCDEESPEAELHNHGIQINSCSVRLVDIK |
| | | | KEKPFSNSKVECQAQARTHHNQASDIIVISSED |
| | | | SEGSTDVDEPLEVFISAPRSEPVINNDNPLESN |
| | | | DEKEGQEATCSRPQIVPEPMDFRKLSTFRESFK |
| | | | KRVIGQDHDFSESSEEEAPAEASSGALRSKHGE |
| | | | KAPMTSRSTSTWRIPSRKRRFSSSDFSDLSNGE |
| | | | ELQETCSSSLRRGSGSQPQEPENKKCSCVMCFP |
| | | | KGVPRSQEARTESSQASDMMDTMDVENNSTLEK |
| | | | HSGKRRKKRRHRSKVNGLQRGRKKDRPRKHLTL |
| | | | NNKVQKKRWQQRGRKANTRPLKRRRKRGPRIPK |
| | | | DENINEKQSELPVTCGEVKGTLYKERFKQGTSK |
| | | | KCIQSEDKKWFTPREFEIEGDRGASKNWKLSIR |
| | | | CGGYTLKVLMENKPLPEPPSTRKKRILESHNNT |
| | | | LVDPCEEHKKKNPDASVKFSEPLKKCSETWKTI |
| | | | FAKEKGKFEDMAKADKAHYEREMKTYIPPKGEK |
| | | | KKKFKDPNAPKRPPLAFFLPCSEYRPKIKGEHP |
| | | | GLSIDDVVKKLAGMWNNTAAADKQFYEKKAAKL |
| | | | KEKYKKDIAAYRAKGKPNSAKKRVVKAEKSKKK |
| | | | KEEEEDEEDEQEEENEEDDDK |
| | | | (SEQ ID NO: 38) |
| SWISS_PR | P04267 | Tropomyosin 1, | MEAIKKKMQMLKLDKENAIDRAEQAEADKKQAE |
| OT: | | smooth muscle | DRCKQLEEEQQGLQKKLKGTEDEVEKYSESVKE |
| TPM1_CR1 | | Gizzard beta- | AQEKLEQAEKKATDAEAEVASLNRRIQLVEEEL |
| CR | | tropomyosin) | DRAQERLATALQKLEEAEKAADESERGMKVIEN |
| | | (Smooth- | RAMKDEEKMELQEMQLKEAKHTAEEADRKYEEV |
| | | muscle\|alpha- | ARKLVVLEGELERSEERAEVAESRVRQLEEELR |
| | | tropomyosin) | TMDQSLKSLIASEEEYSTKEDKYEEEIKLLGEK |
| | | (Tropomyosin | LKEAETRAEPAERSVAKLEKTIDDLEESLASAK |
| | | beta chain, | EENVGIHQVLDQTLLELNNL |
| | | smooth muscle) | (SEQ ID NO: 39) |
| SWISS_PR | P09493 | Tropomyosin | MDAIKKKMQMLKLDKENALDRAEQAEADKKAAE |
| OT: | | alpha chain, | DRSKQLEDELVSLQKKLKGTEDELDKYSEALKD |
| TPM1_HUM | | skeletal muscle | AQERLELAEKKATDAEADVASLNRRIQLVEEEL |
| AN | | (Tropomyosin 1, | DRAQERLATALQKLEEAEKAADESERGMKVTES |
| | | skeletal\| | RAQKDEEKMEIQEIQLKEAKHIAEDADRKYEEV |
| | | muscle) | ARKLVIIESDLERAEERAELSEGKCAELEEELK |
| | | | TVTNNLKSLEAQAEKYSQKEDRYEEEIKVLSDK |
| | | | LKEAETRAEFAERSVTKLEKSIDDLEDELYAQK |
| | | | LKYRAISEELDHALNDMTSI |
| | | | (SEQ ID NO: 40) |
| SWISS_PR | P04268 | Tropomyosin 2, | MDAIKKRMQMLKLDKENALDRAEQAEADKKAAE |
| OT: | | smooth muscle | ERSKQLEDDIVQLEKQLRVTEDSRDQVLEELHK |
| TPM2_CRI | | (Gizzard gamma- | SEDSLLSAEENAAKAESEVASLNRRTQLVEEEL |
| CK | | tropomyosin) | DRAQERLATALQKLEEAEKAADESERCMKVIEN |
| | | (Smooth-\|muscle | RAQKDEEKMEIQEIQLKEAKHIAEEADRKYEEV |
| | | beta- | ARKLVILEGDLERAEERAELSESKCAELEEELK |
| | | tropomyosin | LVTNEAKSLEAQAEKYSQKEDKYEEEIKVLTDK |
| | | | LKEAETRAEFAERSVTKLEKSIQDLEERVAHAK |
| | | | EENLNMHQMLDQTLLELNNM |
| | | | (SEQ ID NO: 41) |
| SWISS_PR | P19353 | Tropomyosin | MAGISSIDAVKKKIQSLQQVADEAEERAEHLQR |
| QT: | | beta 3, | EADAERQARERAEAEVASLNRRIQLVEEELDRA |
| TPM3C_RI | | fibroblast | QERLATALQKLEEAEKAADESERGMKVIENRAM |
| CK | | | KDEEKMELQEMQLKEARHTAEEADRKYEEVARK |
| | | | LVVLEGELERSEERAEVAESRVRQLEEELRTMD |
| | | | QSLKSLIASEEEYSTKEDKYEEEIKLLGEKLKE |
| | | | AETRAEFAERSVAKLEKTIDDLEESLASAKEEN |
| | | | VGIHQVLDQTLLELNNL |
| | | | (SEQ ID NO: 42) |
| SWISS_PR | P06753 | Tropomyosin | MEAIKKKMQMLKLDKENALDRAEQAEAEQKQAE |
| OT: | | alpha chain, | ERSKQLEDELAAMQKKLKGTEDELDKYSEALKD |
| TPM3_HUM | | skeletal muscle | AQEKLELAEKKAADAEAEVASLNRRIQLVEEEL |
| AN | | type | DRAQERLATALQKLEEAEKAADESERGMKVIEN |
| | | (Tropomyosin 3, | RALKDEEKMELQEIQLKEAKHIAEEADRKYEEV |
| | | skeletal\| | ARKLVIIEGDLERTEERAELAESKCSELEEELK |
| | | muscle) | NVTNNLKSLEAQAEKYSQKEDKYEEEIKILTDK |
| | | | LKEAETRAEFAERSVAKLEKTIDDLEDELYAQK |
| | | | LKYKAISEELDHALNDMTSI |
| | | | (SEQ ID NO: 43) |
| SWISS_PR | P49438 | Tropomyosin | MAALSSLEAVRKKIRSLQEQADAAEERAGKLQR |
| OT: | | alpha chain, | EVDQERALREEAESEVASLNRRIQLVEEELDRA |
| TPM5_CHI | | major brain | QERLATALQKLEEAEKAADESERGMKVIENRAQ |
| CK | | isoform | KDEEKMEIQEIQLKEAKHIAEEADRKYEEVARK |
| | | | LVIIEGDLERAEERAELSESKCAELEEELKTVT |
| | | | NNLKSLEAQAEKYSQKEDKYEEEIKVLTDKLKE |
| | | | AETRAEFAERSVTKLEKSIDDLEDQLYQQLEQN |
| | | | SRLTNELKLALNED |
| | | | (SEQ ID NO: 44) |
| SWISS_PR | P49439 | Tropomyosin | MDAIKKKMQMLKLDKENALDRAEQAEADKKAAE |
| OT: | | alpha chain, | ERSKQLEDELVALQKKLKGTEDELDKYSESLKD |
| TPM6_CHI | | minor brain | AQEILELADKKATDAESEVASLNRRIQLVEEEL |
| CK | | isoform | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEFAERSVTKLEKSIDDLEDQLYQQL |
| | | | EQNSRLTNELKLALNED |
| | | | (SEQ ID NO: 45) |
| SWISS_PR | P13104 | Tropomyosin | MDAIKKKMQMLKLDKENALDRAEQAETDKKAAE |
| OT: | | alpha chain, | ERSKQLEDDLVALQKKLKATEDELDKYSEALKD |
| TPMA_BRA | | skeletal muscle | AQEKLELAEKKATDAEGDVASLNRRIQLVEEEL |
| RE | | | DRAQERLATALQKLEEAEKAADESERGMKVIEN |
| | | | RALKDEEKMELQEIQLKEAKHIAEEADRKYEEV |
| | | | ARKLVIVEGELERTEERAELNECKCSELEEELK |
| | | | TVTNNMKSLEAQAEKYSAKEDKYEEEIKVLTDK |
| | | | LKEAETRAEFAERSVAKLEKTIDDLEDELYAQK |
| | | | LKYKAISEELDHALNDMTSI |
| | | | (SEQ ID NO: 46) |
| SWISS_PR | P02559 | Tropomyosin | MDAIKKKMQMLKLDKENALDRAEQAEADKKAAE |
| OT: | P18442 | alpha chain, | ERSKQLEDELVALQKKLKGTEDELDKYSESLKD |
| TPMA_COT | | skeletal muscle | AQEKLELADKKATDAESEVASLNRRIQLVEEEL |
| JA | | | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEFAERSVTKLEKSTDDLEDELYAQK |
| | | | LKYKAISEELDHALNDMTSI |
| | | | (SEQ ID NO: 47) |
| SWISS_PR | P02558 | Tropomyosin | MDAIKKKMQMLKLDKENALDRAEQAEADKKAAE |
| OT: | P46902 | alpha chain, | DRSKQLEDELVSLQKKLKGTEDELDKYSEALKD |
| TPMA_MOU | P99034 | skeletal and | AQEKLELAEKKATDAEADVASLNRRIQLVEEEL |
| SE | | cardiac muscle | DRAQERLATALQKLEEAEKAADESERGMKVIES |
| | | | RAQKDEEKMEIQEIQLKEAKHIAEDADRKYEEV |
| | | | ARKLVIIESDLERAEERAELSEGKCAELEEELK |
| | | | TVTNNLKSLEAQAEKYSQKEDKYEEEIKVLSDK |
| | | | LKEAETRAEFAERSVTKLEKSIDDLEDELYAQK |
| | | | LKYKAISEELDHALNDMTSI |
| | | | (SEQ ID NO: 48) |
| SWISS_PR | P13105 | Tropomyosin | MDAIKKKMQMLKLDKENALDRAEQAEADKKGAE |
| OT: | | alpha chain, | DKSKQLEDELVAMQKKMKGTEDELDKYSEALKD |
| TPMA_RAN | | skeletal muscle | AQEKLELAEKKATDAEADVASLNRRIQLVEEEL |
| TE | | | DRAQERLATALQKLEEAEKAADESERGMKVIEN |
| | | | RALKDEEKIELQEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEFAERTVAKLEKSIDDLEDELYAQK |
| | | | LKYKAISEELDHALNDMTSI |
| | | | (SEQ ID NO: 49) |
| SWISS_PR | P04692 | Tropomyosin | MDAIKKKMQMLKLDKENALDRAEQAEADKKAAE |
| OT: | | alpha chain, | DRSKQLEDELVSLQKKLKGTEDELDKYSEALKD |
| TPMA_RAT | | skeletal muscle | AQEKLELAEKKATDAEADVASLNRRIQLVEEEL |
| | | | DRAQERLATALQKLEEAEKAADESERGMKVIES |
| | | | RAQKDEEKMEIQEIQLKEAKHIAEDADRKYEEV |
| | | | ARKLVIISSDLERAEERAELSEGKCAELEEELK |
| | | | TVTNNLKSLEAQAEKYSQKEDKYEEEIKVLSDK |
| | | | LKEAETRAEFAERSVTKLEKSIDDLEDELYAQK |
| | | | LKYKAISEELDHALKDMTSI |
| | | | (SEQ ID NO: 50) |
| SWISS_PR | Q01173 | Tropomyasin | MDAIKKKMQMLKLDKENALDRAEQAEADKKGAE |
| OT: | | alpha chain, | DKSKQLEDELVALQKKLKGTEDELDKYSEALKD |
| TPMA_XEN | | skeletal muscle | AQEKLELSDKKATDAEGDVASLNRRIQLVEEEL |
| LA | | | DRAQERLSTALQKLEEAEKAADESERGMKVIEN |
| | | | RALEDEERMELCEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEPAERTVAKLEKSIDDLEDELYAQK |
| | | | LKYKAISEELDHALNDMTSI |
| | | | (SEQ ID NO: 51) |
| SWISS_PR | P19352 | Tropomyosin | MEAIKKKMQMLKLDKENAIDRAEQAEADKKQAE |
| OT: | | beta chain, | DRCKQLEEEQQGLQKKLKGTEDEVEKYSESVKE |
| TPMB_CHI | | skeletal muscle | AQEKLEQAEKKATDAEAEVASLNRRIQLVEEEL |
| CK | | | DRAQERLATALQKLEEAEKAADESERGMKVIEN |
| | | | RAMKDEEKMELQEMQLKEAKHIAEEADRKYEEV |
| | | | ARKLVVLEGELERSEERAEVAESKCGDLEEELK |
| | | | IVTNNLKSLEAQADKYSTKEDKYEEEIKLLGEK |
| | | | LKEAETRAEFAERSVAKLEKTIDDLEDEVYAQK |
| | | | MKYKAISEELDNALNDITSL |
| | | | (SEQ ID NO: 52) |
| SWISS_PR | P07951 | Tropomyosin | MDAIKKKMQMLKLDKENAIDRAEQAEADKKQAE |
| OT: | | beta chain, | DRQKQLEEEQQALQKKLKGTEDEVEKYSESVKE |
| TPMB_HUM | | skeletal muscle | AQEKLEQAEKKATDAEADVASLNRRIQLVEEEL |
| AN | | (Tropomyosin 2, | DRAQERLATALQKLEEAEKAADESERGMKVIEN |
| | | skeletal\| | RAMKDEEKMELQEMQLKEAKHIAEDSDRKYEEV |
| | | muscle) | ARKLVILEGELERSEEPAEVAESKCGDLEEELK |
| | | | IVTNNLKSLEAQADKYSTKEDKYEEEIKLLEEK |
| | | | LKEAETRAEFAERSVAKLEKTIDDLEDEVYAQK |
| | | | MKYKAISEELDNALNDITSL |
| | | | (SEQ ID NO: 53) |
| SWISS_PR | P02560 | Tropomyosin | MDAIKKKMQMLKLDKENAIDRAEQAEADKKQAE |
| OT: | | beta chain, | DRCKQLEEEQQALQKKLKGTEDEVEKYSESVKD |
| TPMB_MOU | | skeletal muscle | AQEKLEQAEKKATDAEADVASLNRRIQLVEEEL |
| SE | | | DRAQERLATALQKLEEAEKAADESERGMKVIEN |
| | | | RAMKDEEKMELQEMQLKEAKHIAEDSDRKYEEV |
| | | | ARKLVILEGELERSEERAEVAESKCGDLEEELK |
| | | | IVTNNLKSLEAQADKYSTKEDKYEEEIKLLEEK |
| | | | LKEAETRAEPAERSVAKLEKTIDDLEDEVYAQK |
| | | | MKYKAISEELDNALNDITSL |
| | | | (SEQ ID NO: 54) |
| SWISS_PR | P42639 | Tropomyosin | MDAIKKKMQMLKLDKENALDRADEAEADKKAAE |
| CT: | | alpha chain, | DRSKQLEDELVSLQKKLKATEDELDKYSEALKD |
| TPMC_PIG | | cardiac muscle | AQEKLELAEKKATDAEADVASLNRRIQLFEEEL |
| | | | DRAQERLATALQKLEEAEKAADESERGMKVIES |
| | | | RAQKDEEKMEIQEIQLKEAKHIAEDADRKYEEV |
| | | | ARKLVIIESDLERAEERAELSEGKCAELEEELK |
| | | | TVTNNLKSLEAQAEKYSQKEDKYEEEIKVLSDK |
| | | | LKEAETRAEFAERSVTKLEKSIDDLEDELYAQK |
| | | | LKYKAISEELDHALNDMTSI |
| | | | (SEQ ID NO: 55) |
| SWISS_PR | P18441 | Tropomyosin | MDAIKKKMQMLKLDKENALDRAEQAEADKKAAE |
| CT: | | alpha chain, | ERSKQLEDELVALQKKLKGTEDELDKYSESLKD |
| TPMF_CHI | | fibroblast | AQEKLELADKKATDAESEVASLNRRIQLVEEEL |
| CK | | isoform Fl | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESQVRQLEEQLR |
| | | | IMDQTLKALMAAEDKYSQKEDKYEEEIKVLTDK |
| | | | LKEAETRAEFAERSVTKLEKSIDDLEEKVAHAK |
| | | | EENLNMHQMLDQTLLELNNM |
| | | | (SEQ ID NO: 56) |
| SWISS_PR | P08942 | Tropomyosin | MDAIKKKMQMLKLDKENALDRAEQAEADKKAAE |
| CT: | | alpha chain, | ERSKQLEDELVALQKKLKGTEDELDKYSESLKD |
| TPMG_COT | | fibroblast | AQEKLELADKKATDAESEVASLNRRIQLVEEEL |
| JA | | isoform F2 | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESKQAELEEELK |
| | | | TVTNNLKSLEAQAEKYSQKEDKYEEEIKVLTDK |
| | | | LKEAETRAEFAERSVTKLEKSIDDLEEKVAHAK |
| | | | EENLNMHQMLDQTLLELNNM |
| | | | (SEQ ID NO: 57) |
| SWISS_PR | Q01174 | Tropomyosin | MAGITSLEAVKRKIKCLQDQADEAEERAEKLQR |
| OT: | | alpha chain, | ERDMERKLREAAEGDVASLNRRIQLVEEELDRA |
| TPMN_XEN | | non-muscle | QERLSTALQKLEEAEKAADESERGMKVIENRAL |
| LA | | | KDEEKMELQEIQLKEAKHIAEEADRKYEEVARK |
| | | | LVIIEGDLERAEERAELSESHYRQLEDQQRIMD |
| | | | QTLKTLIASEEKYSQKEDKYEEEIKVLTDKLKE |
| | | | AETRAEFAERTVAKLEKSIDDLEEKVAHAKEEN |
| | | | LNMHQMLDQTLLELNNM |
| | | | (SEQ ID NO 58) |
| SWISS_PR | P49436 | Tropomyosin | MDAIKKKMQMLKLDKENALDRAEQAEADKKAAE |
| OT: | | alpha chain, | ERSKQLEDDIVQLEKQLRVTEDSRDQVLEELHK |
| TPMS_CHI | | smooth muscle | SEDSLLFAEENAAKAESEVASLNRRIQLVEEEL |
| CK | | | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEFAERSVTKLEKSIDDLEEKVAHAK |
| | | | EENLNMHQMLDQTLLELNNM |
| | | | (SEQ ID NO: 59) |
| SWISS_PR | P49437 | Tropomyosin | MDAIKKKMQMLKLDKENALDRAEQAEADKKAAE |
| OT: | | alpha chain, | ERSKQLEDDIVQLEKQLRVTEDSRDQVLEELHK |
| TPMS_COT | | smooth muscle | SEDSLLSAEEIAAKAESEVASLNRRIQLVEEEL |
| JA | | | DRAQERLATALQKLEEAEKAADESERGMKVIEN |
| | | | RAQKDEEKMEIQEIQLKEAKHIAEEADRKYEEV |
| | | | ARKLVIIEGDLERAEERAELSESKCAELEEELK |
| | | | TVTNNLKSLEAQAEKYSQKEDKYEEEIKVLTDK |
| | | | LKEAETRAEFAERSVTKLEKSIDDLEEKVAHAK |
| | | | EENLNMHQMLDQTLLELNNM |
| | | | (SEQ ID NO: 60) |
| SWISS_PR | P10469 | Tropomyosin | CRLRIFLRTASSEHLHERKLRETAEADVASLNR |
| OT: | | alpha chain, | RIQLVEEELDRAQERLATVLQKLEEAEKAADES |
| TPMS_HUM | | smooth muscle | ERGMKVIESRAQKDEEKMEIQEIQLKEAKHIAE |
| AN | | (Tropomyosin 1, | DADRKYEEVARKLVIIESDLERAEERAELSEGQ |
| | | smooth muscle) \| | VRQLEEQLRIMDSDLESINAAEDKYSQKEDRYE |
| | | (Fragment) | EEIKVLSDKLKEAETRAEFAERSVTKLEKSIDD |
| | | | LEEKVAHAKEENLSMHQMLDQTLLELNNM |
| | | | (SEQ ID NO: 61) |
| SWISS_PR | P06469 | Tropomyosin | MDAIKKKMQMLKLDKENALDRAEQAEADKKAAE |
| OT: | | alpha chain, | DRSKQLEEDISAKEKLLRASEDERDRVLEELHK |
| TPMS_RAT | | smooth muscle | AEDSLLAADETAAKAEADVASLNRRIQLVEEEL |
| | | | DRAQERLATALQKLEEAEKAADESERGMKVIES |
| | | | RAQKDEEKMEIQEIQLKEAKHIAEDADRKYEEV |
| | | | ARKLVIIESDLERAEERAELSEGKCAELEEELK |
| | | | TVTNNLKSLEAQAEKYSQKEDKYEEEIKVLSDK |
| | | | LKEAETRAEFAERSVTKLEKSIDDLEEKVAHAK |
| | | | EENLSMHQMLHQTLLELNNM |
| | | | (SEQ ID NO: 62) |
| SWISS_PR | P18342 | Tropomyosin | MDAIKKKMQMLKLDKENALDRAEQAEADKKAAE |
| OT: | | alpha chain, | DRSKQLEDELVSLQKKLKATEDELDKYSEALKD |
| TPMX_RAT | | brain-1 | AQEKLELAEKKATDAEADVASLNRRIQLVEEEL |
| | | (TMBR-1) | DRAQERLATALQKLEEAEKAADESERGMKVIES |
| | | | RAQKDEEKMEIQETQLKEAKHIAEDADRKYEEV |
| | | | ARKLVIIESDLERAEERAELSEGKCAELEEELK |
| | | | TVTNNLKSLEAQAEKYSQKEDKYEEEIKVLSDK |
| | | | LKEAETRAEFAERSVTKLEKSIDDLEDQLYHQL |
| | | | EQNRRLTNELKLALNED |
| | | | (SEQ ID NO: 63) |
| SWISS_PR | P18343 | Tropomyosin | MAGSSSLEAVRRKIRSLQEQADAAEERAGSLQR |
| OT: | | alpha chain, | ELDQERKLRETAEADVASLNRRIQLVEEELDRA |
| TPMY_RAT | | brain-2 | QERLATALQKLEEAEKAADESERGMKVIESRAQ |
| | | (TMBR-2) | KDEEKMEIQEIQLKEAKHIAEDADRKYEEVARK |
| | | | LVIIESDLERAEERAELSEGKCAELEEELKTVT |
| | | | NNLKSLEAQAEKYSQKEDKYEEEIKVLSDKLKE |
| | | | AETRAEFAERSVTKLEKSIDDLEDKFLCFSPPK |
| | | | TPSSSRMSHLSELCICLLSS |
| | | | (SEQ ID NO: 64) |
| SWISS_PR | P18344 | Tropomyosin | MAGSSSLEAVRRKIRSLQEQADAAEERAGSLQR |
| OT: | | alpha chain, | ELDQERKLRETAEADVASLNRRIQLVEEELDRA |
| TPMZ_RAT | | brain-3 (TMBR- | QERLATALQKLEEAEKAADESERGMKVIESRAQ |
| | | 3) | KDEEKMEIQEIQLKEAKHIAEDADRKYEEVARK |
| | | | LVIIESDLERAEERAELSEGKCAELEEELKTVT |
| | | | NNLKSLEAQAEKYSQKEDKYEEEIKVLSDKLKE |
| | | | AETRAEFAERSVTKLEKSIDDLEDQLYHQLEQN |
| | | | RRLTNELKLALNED |
| | | | (SEQ ID NO: 65) |
| SWISS_PR | P41541 | General | MNFLRGVMGGQSAGPQHTEAETIQKLCDRVASS |
| OT: | | vesicular | TLLDDRRNAVRALKSLSKKYRLEVGIQAMEHLI |
| VDP_BOVI | | transport | HVLQTDRSDSEIIGYALDTLYNIISNDEEEEVE |
| N | | factor p115 | ENSTRQSEDLGSQFTEIFIKQQENVTLLLSLLE |
| | | (Transcytosis | EFDFHVRWPGVKLLTSLLKQLGPQVQQIILVSP |
| | | associated\| | MGVSRLMDLLADSREVTRNDGVLLLQALTRSNG |
| | | protein)(TAP) | AIQKIVAFENAEERLLDIITEEGNSDGGIVVED |
| | | (Vesicle | CLILLQNLLKNNNSNQNFFKEGSYIQRMKPWEE |
| | | docking | VGDENSGWSAQKVTNLHLMLQLVRVLVSPNNPP |
| | | protein) | GATSSCQKAMFQCGLLQQLCTTLMATGVPADIL |
| | | | TETINTVSEVIRGCQVNQDYEASVNAPSNPPRP |
| | | | AIVVLLMSMVNERQPFVLRCAVLYCFQCFLYKN |
| | | | QKGQGEIVSTLLPSTIDATGNTVSAGQLLCGGL |
| | | | FSTDSLSNWCAAVALAHALQENATQKEQLLRVQ |
| | | | LATSIGNPPVSLLQQCTNILSQGSKIQTRVGLL |
| | | | MLLCTWLSNCPIAVTHFLHNSANVPFLTGQIAE |
| | | | NLGEEEQLVQGLCALLLGISIYFNDNSLETYMK |
| | | | EKLKQLIEKRIGKENFIEKLGFISKHELYSRAS |
| | | | QKPQPNFPSPEYMIFDHEFTKLVKELEGVITKA |
| | | | IYKSSEEDKKEEEVKKTLEQHDSIVTHYKNMIR |
| | | | EQDLQLEELKQQISTLKCQNEQLQTAVTQQVSQ |
| | | | IQQHKDQYNLLKVQLGKDSQHQGPYTDGAQMNG |
| | | | VQPEBISRLREEIEELKSNRELLQSQLAEKDSL |
| | | | IENLKSSQLSPGTNEQSSATAGDSEQIAELKQE |
| | | | LATLKSQLNSQSVEITKLQTEKQELLQKTEAPA |
| | | | KSAPVPGESETVIATKTTDVEGRLSALLQETKE |
| | | | LKNEIKALSEERTAIKEQLDSSNSTIAILQNEK |
| | | | NKLEVDITDSKKEQDDLLVLLADQDQKIFSLKN |
| | | | KLKELGHPVEEEDELESGDQDDEDDEDEDDGKE |
| | | | QGHI |
| | | | (SEQ ID NO: 66) |
| SWISS_PR | P41542 | General | MNFLRGVMGGQSAGPQHTEAETIQKLCDRVASS |
| OT: | | vesicular | TLLDDRRNAVRALKSLSKKYRLEVGIQAMEHLI |
| VDP_RAT | | transport | HVLQTDRSDSEIIAYALDTLYNIISNDEEEEVE |
| | | factor p115 | ENSTRQSEDLGSQFTEIFIKQPENVTLLLSLLE |
| | | (Transcytosis | EFDFHVRWPGVRLLTSLLKQLGPPVQQIILVSP |
| | | associated\|pro- | MGVSKLMDLLADSREIIRNDGVLLLQALTRSNG |
| | | tein) (TAP) | AIQKIVAFENAFERLLDIITEEGNSDGGIVVED |
| | | (Vesicle | CLILLQNLLKNNNSNQNFFKEGSYIQRMKAWFE |
| | | docking | VGDENPGWSAQKVTNLHLMLQLVRVLVSPTNPP |
| | | protein) | GATSSCQKAMFQCGLLQQLCTILMATGIPADIL |
| | | | TETINTVSEVIRGCQVNQDYFASVNAPSNPPRP |
| | | | AIVVLLMSMVNERQPFVLRCAVLYCFQCFLYKN |
| | | | EKGQGEIVATLLPSTIDATGNSVSAGQLLCGGL |
| | | | ESTDSLSNWCAAVALAHALQGNATQKEQLLRVQ |
| | | | LATSIGNPPVSLLQQCTNILSQGSKIQTRVGLL |
| | | | MLLCTWLSNCPIAVTHFLHNSANVPPLTGQIAE |
| | | | NLGEEEQLVQGLCALLLGISIYFNDNSLENYTK |
| | | | EKLKQLIEKRIGKENYIEKLGFISKHELYSRAS |
| | | | QKPQPNFPSPEYMIFDHEFTKLVKELEGVITKA |
| | | | IYKSSEEDKKEEEVKKTLEQFIDNIVTHYKNMIR |
| | | | EQDLQLEELKQQVSTLKCQNEQLQTAVTQQASQ |
| | | | IQQHKDQYNLLKVQLGKDNHHQGSHSDGAQVNG |
| | | | IQPEEISRLREEIEELRSHQVLLQSQLAEKDTV |
| | | | IENLRSSQVSGMSEQALATCSPRDAEQVAELKQ |
| | | | ELSALKSQLCSQSLEITRLQTENSELQQRAETL |
| | | | AKSVPVEGESELVTAAKTTDVEGRLSALLQETK |
| | | | ELKNEIKALSEERTAIQKQLDSSNSTIAILQTE |
| | | | KDKLYLEVTDSKKEQDDLLVLLADQDQKILSLK |
| | | | SKLKDLGHPVEEEDESGDQEDDDDELDDGDRDQ |
| | | | DI |
| | | | (SEQ ID NO: 67) |
| TREMBL: | Q21049 | PUTATIVE | LIPRINMSYSNGNINCDIMPTISEDGVDNGGPIDEPSDR |
| Q21049 | | ALPHA (LAR- | DNIEQLMMNMLEDRDKLQEQLENYKVQLENAGL |
| | | INTERACTING | RTKEVEKERDMMKRQEEVHTQNLPQELQTMTRE |
| | | PROTEIN ALPHA) | LCLLKEQLLEKDEEIVELKAERNNTRLLLEHLE |
| | | | CLVSRHERSLRMTVMKRQAQNHAGVSSEVEVLK |
| | | | ALKSLFEHHKALDEKVRERLRVAMERVATLEEE |
| | | | LSTKGDENSSLKARIATYAAEAEEAMASNAPIN |
| | | | GSISSESANRLIEMQEALERMKTELANSLKQST |
| | | | EITTRNAELEDQLTEDAREKHAAQESIVRLKNQ |
| | | | ICELDAQRTDQETRITTFESRFLTAQRESTCIR |
| | | | DLNDKLEHQLANKDAAVRLNEEKVHSLQERLEL |
| | | | AEKQLAQSLKKAESLPSVEAELQQRMEALTAAE |
| | | | QKSVSAEERIQRLDRNIQELSAELERAVQRERM |
| | | | NEEHSQRLSSTVDKLLSESNDRLQLHLKERMQA |
| | | | LDDKNRLTQQLDGTKKIYDQAERIKDRLQRDNE |
| | | | SLRQEIEALRQQLYNARTAQFQSRMHAIPFTHA |
| | | | QNIVQQQPQASIAQQSAYQMYKQQPAQQYQTVG |
| | | | MRRPNKGRISALQDDPNKVQTLNEQEWDRLQQA |
| | | | HVLANVQQAFSSSPSLADVGQSTLPRPNTAVQH |
| | | | QQDDMMNSGMGMPSGMQGGMQGGMGGGQDAQML |
| | | | ASMLQDRLDAINTEIRLIQQEKHHAERVAEQLE |
| | | | RSSREFYDDQGISTRSSPRASPQLDNMRQHKYN |
| | | | TLPANVSGDRRYDIYGNPQFVDDRMVRDLDYEP |
| | | | RRGYNQFDEMQYERDRMSPASSVASSTDGVLGG |
| | | | KKKRSNSSSGLKTLGRFFNKKKNSSSDLFKRNG |
| | | | DYSDGEQSGTEGNQKADYDRRKKKKHELLEEAM |
| | | | KARTPFALWNGPTVVAWLELWVGMPAWYVAACR |
| | | | ANVKSGAIMSALSDQEIQKEIGISNPLHRLKLR |
| | | | LAIQEMVSLTSPSAPRTARLTLAFGDMNHEYIG |
| | | | NDWLPCLGLAQYRSAFMECLLDARMLEHLSKRD |
| | | | LRTHLRMVDTFHRTSLQYGIMCLKKVNYDKKVL |
| | | | ADRRKACDNINTDLLVWSNERVQRWVEEIGLGV |
| | | | FSRNLVDSGIHGALIALDETFDASAFAYALQIG |
| | | | SQDVPNRQLLEKKFIGLVNDHRQQSDPHPRSGS |
| | | | SRKNDSIAKSYEFHLYT |
| | | | (SEQ ID NO: 68) |
| TREMBL: | Q94071 | PUTATIVE LIPRIN | MYSRHSISDAYGAVCILPEDTLTVSSSQNSHID |
| Q94071 | | BETA (LAR- | AFAALVDRERDSSRSSGSGNIFKDNGSIKRRQA |
| | | INTERACTING | LPYVTHYSDSGFGSAPSAGSSCSYLPPPPPYRM |
| | | PROTEIN BETA) | RGSGGLSSKPQHKIHRSLSDSKYTASLMTTGVP |
| | | | TLPLLSMTPFNQLQSRDARGASWISLVRAPNFH |
| | | | LYCFFVFFFSFNIDETFRNSNISSPSPSMSTVS |
| | | | CPEYPELQDKLHRLAMARDSLQLQVSVLSEQVG |
| | | | AQKEKIKDLETVIALKRNNLTSTEELLQDKYHR |
| | | | IDECQELESKKMDLLAEVSSLKLRYATLEREKN |
| | | | ETEKKLRLSQNEMDRVNQSMHCMVVQQCLARHT |
| | | | NGHSSGGYMSPLREHRSEKNDDEMSQLRTAVQR |
| | | | LMADNEHKSLQINTLRNALDEQMRSRSQQEDFY |
| | | | ASQRNYTDNFDVNAQIRRILMDEPSDSMSHSTS |
| | | | FPVSLSSTTSNGKGPRSTVQSSSSYNSSLSAVS |
| | | | PQHNWSSAGAGTPRQLHPIGGNQRVNNITSAQY |
| | | | CSPSPPAARQLAAELDELRRNGNEGANHNYSSA |
| | | | SLPRGVGKASSTLTLPSKKLSVASGTSVVESDD |
| | | | EIARGRNLNNATSQSNLKNFSRERTRSSLRNIF |
| | | | SKLTRSTSQDQSNSFRRGSAARSTSTARLGSTN |
| | | | HLGTVSKRPPLSQFVDWRSEQLADWIAEIGYPQ |
| | | | YMNEVSRHVRSGRHFLNMSMNEYEGVLNIKNPV |
| | | | HRKRVAILLRRIEEDIMEPANKWDVHQTLRWLD |
| | | | DIGLPQYKDVFAENVVDGPLLLSLTANDAVEMK |
| | | | VVNAHHYATLARSIQFLKKADFRFNAMEKLIDQ |
| | | | NIVEKYPCPDVVVRWTHSATCEWLRKIDLAEFT |
| | | | QNLLFAGVPGALMIYEPSFTAESLAEILQMPPH |
| | | | KTLLRRHLTSHFNQLLGPKIIADKRDFLAAGNY |
| | | | PQISPTGRVKVVKKGFSLTRKKAKNEICLEPEE |
| | | | LLCPQVLVHKYPTGAGDNSSFESSNV |
| | | | (SEQ ID NO: 69) |

High stability leucine zippers may be derived using procedures known to those of ordinary skill in the art (see, e.g., Newman et al., 2000, A computationally directed screen identifying interacting coiled coils from Saccharomyces cerevisiae, Proc. Natl. Acad. Sci. USA 97, 13203-08). Computer programs such as PAIRCOIL (Berger et al., 1995, Predicting Coiled Coils by Use of Pairwise Residue Correlations, Proc. Natl. Acad. Sci. USA, 92: 8259-63) and MULTICOIL (Wolf et al., 1997, MultiCoil: A program for predicting two- and three-stranded coiled coils, Protein Science 6: 1179-89), may be used to predict how coiled coils will interact to form dimers and/or trimers, etc.

Leucine zippers can be described as seven residue repeat units. Of the seven amino acids in each heptad derived from a leucine zipper, the residues in the a and d positions are generally hydrophobic amino acids (alanine, valine, phenylalanine, methionine, isoleucine and leucine) while the amino acids in the e and g positions are usually charged amino acids (aspartic acid, glutamic acid, lysine and arginine). The specific sequence of hydrophobic a and d residues determines whether two members of a pair interact. Accordingly, many coiled coils are already known and computer software analyses may be used to identify, design, and test potential novel coiled coils (Newman et al., 2000, A computationally directed screen identifying interacting coiled coils from Saccharomyces cerevisiae, Proc. Natl. Acad. Sci. USA 97: 13203-08).

The residues in the e and g position of the heptad determine how strongly coiled coils bind to each other by forming salt bridges that stabilize the binding between the coils (see, e.g., Krylov et al., 1998, Interhelical interactions in the leucine zipper coiled coil dimer: pH and salt dependence of coupling energy between charged amino acids, J. Mol. Biol. 279: 959-72). Predictive formulae for interhelical binding strength of leucine zippers based on the zipper sequence, particularly the e and g positions, have been derived, and are known to those of skill in the art. These can be used to determine the length of the leucine zipper needed for construction of a particular assembly unit. The number of heptads in a leucine zipper affects the binding strength between molecules comprising those heptads; generally, about four heptads are sufficient at normal temperatures. In certain embodiments, nanostructures that will be subjected to higher temperatures (>40° C.) are constructed using assembly units comprising longer coiled coils or coiled coils stabilized in another manner such as, but not limited to, the introduction of one or more intermolecular disulfide bonds.

Isolated leucine zippers generally do not form stable dimers outside of a protein milieu (Branden and Tooze 1999, Introduction to Protein Structure, 2nd ed., Garland publishing, Inc. New York, p. 37). Therefore, in order to stabilize assembly units of the invention that are formed with leucine zippers, flanking cysteines are inserted, in preferred embodiments, to form disulfide bridges. Once these bonds have formed, the designed assembly units should be stable unless exposed to reducing agents. Therefore, in certain embodiments, cysteines are added to the end of the leucine zipper or between the α-helix of a leucine zipper and a PNA joining element, for the formation of stabilizing disulfide bonds.

The precise position of the cysteines in an assembly unit can be determined by modeling the assembly unit or assembly subunits using molecular modeling software such as SIBYL (Tripos Inc., St. Louis, Mo.), RasMol (Sayle et al., 1995, RasMol: Biomolecular graphics for all, Trends Biochem. Sci. (TIBS) 20(9): 374-76), or PdbMotif (Saqi et al., 1994, PdbMotif—a tool for the automatic identification and display of motifs in protein structures, Comput. Appl. Biosci. 10(5): 545-46), and then tested empirically. Conversion of two cysteines into a disulfide bridge is well-known to those skilled in the art and is controlled by altering the redox potential of the solution. Under oxidizing conditions (e.g. in the presence of oxygen) the sulfur atoms will bond. Under reducing conditions (e.g. with the addition of a reducing agent such as dithiothreitol (DTT)) the two sulfur atoms will not bond together.

Generally, two disulfide bonds are sufficient to hold the coiled-coils of an assembly unit together. In preferred embodiments, the cysteine residues are disposed at the ends of the leucine zippers and are used to bind together the assembly unit. However, in other embodiments cysteine residues are placed at the border of any domain within the assembly unit. In certain embodiments, such added cysteine residues are flanked or bracketed by one or more, preferably two to five, glycine residues.

Dimer formation by leucine zippers is a cooperative process, and, therefore, the length of the leucine zipper affects the stability of the binding between two helices (Su et al., 1994, Effect of chain length on the formation and stability of synthetic a-helical coiled coils, Biochemistry 33: 15501-10). There is a significant increase in temperature stability between three and four heptads but a lesser increase for longer helices. In certain embodiments of the invention, four heptads can be used for a single uninterrupted unit dimerization region, while two three-heptad regions will be required when the functional sequence interrupts the heptad (see below).

### 5.5.4. Structural Elements Comprising Four-Helix Bundles

The design and construction of leucine zippers represent one type of a coiled coil oligomerization peptide useful in the construction of a structural element of an assembly unit. Another type is a four-helix bundle, a non-limiting example of which is shown in FIG. 15. Because there are one or more loop segments (i.e. non-helical segments) joining the helices to form an assembly unit, this structure is also called a “helix-loop-helix” structure. The loop sections contribute to the stability of the overall structure by keeping the helices near each other and, therefore, at a functionally high concentration. Examples of helix-loop-helix proteins include, but are not limited to: the bacterial Rop protein (a homodimer containing two helix-loop-helix molecules) (Lassalle et al., 1998, Dimer-to-tetramer transformation: loop excision dramatically alters structure and stability of the ROP four alphα-helix bundle protein, J. Mol. Biol. 279(4): 987-1000); the eukaryotic cytochrome b562 (a monomeric protein made up of a single helix-loop-helix-loop-helix-loop-helix structure) (Lederer et al., 1981, Improvement of the 2.5 A resolution model of cytochrome b562 by redetermining the primary structure and using molecular graphics, J. Mol. Biol. 148(4): 427-48); Max (Lavigne et al., 1998, Insights into the mechanism of heterodimerization from the 1 H-NMR solution structure of the c-Myc-Max heterodimeric leucine zipper, J. Mol. Biol. 281(1): 165-81); MyoD DNA-binding domain (Ma et al., 1994, Crystal structure of MyoD bHLH domain-DNA complex: perspectives on DNA recognition and implications for transcriptional activation, Cell 77(3): 451-59); USF1 and USF2 DNA-binding domains (Ferre-D'Amare et al., 1994, Structure and function of the b/HLH/Z domain of USF, EMBO J. 13(1): 180-9; Kurschner et al., 1997, USF2/FIP associates with the b-Zip transcription factor, c-Maf, via its bHLH domain and inhibits c-Maf DNA binding activity, Biochem. Biophys. Res. Commun.231(2): 333-39); and Mit-f transcription factor DNA-binding domains (Rehli et al., 1999, Cloning and characterization of the murine genes for bHLH-ZIP transcription factors TFEC and TFEB reveal a common gene organization for all MiT subfamily members, Genomics 56(1): 111-20).

Both helical regions and loop regions of the Rop protein exhibit properties that indicate that the Rop protein, or fragments thereof, may be used as structural elements in the construction of assembly units in the staged assembly methods of the invention. In one embodiment, the methods of Munson et al. (1996, What makes a protein a protein? Hydrophobic core designs that specify stability and structural properties, Protein Science 5: 1584-93) are used to mutagenize the a and d residues in the helical regions of the Rop protein to produce variant polypeptides having both increased and decreased thermal stability.

In another embodiment, the methods of Betz et al. (1997, De novo design of native roteins: Characterization of proteins intended to fold into antiparallel, Rop-like, four-helix bundles, Biochemistry 36: 2450-58) are used to design synthetic 55-residue proteins that are based on the Rop protein and that form dimers in the predicted anti-parallel arrangement.

Assembly units for staged assembly based on a Rop protein-like four-helix bundle are constructed with synthetic proteins and oligopeptides including, but not limited to, those of Betz et al. (1997, De novo design of native proteins: Characterization of proteins intended to fold into antiparallel, Rop-like, four-helix bundles, Biochemistry 36: 2450-58). As disclosed in Betz and DeGrado (1996, Controlling topology and native-like behavior of de novo-designed peptides: design and characterization of antiparallel four-stranded coiled coils, Biochemistry 35: 6955-62) and Betz et al. (1997, De novo design of native proteins: Characterization of proteins intended to fold into antiparallel, Rop-like, four-helix bundles, Biochemistry 36: 2450-58), synthetic four-helix bundles can be made from two peptides that have the general form of:

Ncap-(AₐZ_{b}Z_{c}L_{d}YₑZ_{f}Y_{g})₃-Turn-(XₐZ_{b}Z_{c}L_{d}YₑZ_{f}Y_{g})₃-Ccap-CONH₂

where the a-g subscripts refer to heptad position, X is either alanine or valine, Y is glutamic acid, arginine, tyrosine or lysine, Z is any amino acid, Ncap and Ccap are alphα-helix ending residues as defined by Richardson and Richardson (1988, Amino acid preferences for specific locations at the ends of alpha helices, Science 240: 1648-52) and turns are 3-5 glycines.

In certain embodiments, PNA sequences are added to the amino terminus of one assembly unit and the carboxy terminus of the other assembly unit. This leaves the other two ends of the molecules, as well as the loop regions, available for the insertion of one or more functional elements. Proper folding of such four-helix bundles can be monitored by CD spectroscopy, ELISA analysis of the constructed assembly unit, and by electron microscopic analysis of the assembly unit and/or nanostructure fabricated from such assembly units.

### 5.6. Joining Elements

According to the present invention, a joining element is defined as a portion of an assembly unit that confers binding properties on the assembly unit including, but not limited to: binding domain, hapten, antigen, peptide, PNA, DNA, RNA, aptamer, polymer or other moiety, or combination thereof, that can interact through specific non-covalent interactions, with another joining element.

Complementary joining elements are two joining elements that interact with one another through specific non-covalent interactions. The pair of joining elements involved in a specific interaction are sometimes referred to as a joining pair. Conversely, a pair of joining elements that do not specifically interact with one another, nor demonstrate any tendency to specifically interact with one another, are sometimes referred to as non-complementary joining elements. Two joining pairs are said to be cross-reactive if a joining element from one pair can bind with specificity to a joining element from the other pair.

Examples of complementary joining elements include, but are not limited to, antibody-antigen binding pairs, antibody-hapten binding pairs, antibody-peptide epitope binding pairs, antibody-functional element binding pairs, antibody-structural element binding pairs, idiotope-anti-idiotope binding pairs, protein-protein interaction binding pairs, domain-domain interaction binding pairs, PNA-PNA interaction binding pairs, protein-inorganic moiety interaction binding pairs, inorganic moiety-inorganic moiety binding pairs, pilin-pilin interaction binding pairs, antibody-pilin interaction binding pairs, pilin-protein interaction binding pairs and the like.

According to the methods of the invention, the number of joining pairs required for the staged assembly of a linear nanostructure needs to be no higher than two. The number of non-cross-reacting joining pairs required for self-assembly of the same structure is equal to the number of assembly units minus one.

In certain embodiments, an assembly unit having more than two joining elements is used to build a nanostructure. The additional joining elements can be used, for example: (i) as an attachment point for addition or insertion of a functional element or functional moiety (see Table I above); (ii) as the attachment point of the initiator to a solid substrate; or (iii) as attachment points for subassemblies.

### 5.6.1. Joining Elements Exhibiting Antigen-Antibody Interactions

In certain embodiments of the invention, joining elements are derived from antibodies, or binding derivatives or binding fragments thereof, and exhibit antigen-antibody interactions. Structural information is readily available for a variety of antibody-antigen complexes. Such structural information may be used to design joining elements for the fabrication of nanostructures according to the methods of the invention. The variable domains of antibodies are designed to interact with specificity to an antigenic target. Their structure and stability are well-characterized in the art, and antibodies and antibody binding fragments may be engineered using methods well known in the art. Consequently, the variable domains of antibodies represent a class of molecules with great potential as joining elements for use as nanostructure assembly units. Such elements provide the basis for specific binding interactions between assembly units and initiators or nanostructure intermediates and are described herein.

It is well known in the art that binding of antibody to antigen is highly specific (Davies et al., 1990, Antibody-antigen complexes, Ann. Rev. Biochem. 59: 439-73; Mian et al., 1991, Structure, function and properties of antibody binding sites, J. Mol. Biol. 217(1): 133-51; Wilson et al., 1994, Antibody-antigen interactions: new structures and new conformational changes, Curr. Opin. Struct. Biol. 4(6): 857-67; Davies et al., 1996, Interactions of protein antigens with antibodies, Proc. Natl. Acad. Sci. USA 93(1): 7-12). This high specificity has been shown to correlate with the high complementarity between the antibody combining site and the antigenic determinant, i.e., the epitope or hapten. This complementarity is defined by the antibody determinant face, defined as the complementarity determining region (CDR) and the antigenic determinant surface, which are in contact, so that the depressions in one are filled by the protrusions from the other. Complementarity also exists by physical and chemical properties such as opposed, oppositely charged side-chain interactions that form ionic bonds. The specificity occurring between the CDR and the antigenic determinant surface can define one type or pair of non-complementary joining element interactions.

Many aromatic side-chain residues, forming hydrophobic interactions, are present in these antibody-antigen interactions. Complementarity between some antigen-antibody complexes is so precise that even water molecules are excluded access from the interface. This particular feature, along with the structural and chemical diversity of the residues within in the CDR loop, including the insertions and deletions, permit specificity and diversity of ligand binding by different antibodies (Winter et al., 1991, Man-made antibodies, Nature 349(6307): 293-99; Davies et al., 1996, Interactions of protein antigens with antibodies, Proc. Natl. Acad. Sci. USA 93(1): 7-12); Wedemayer et al., 1997, Structural insights into the evolution of an antibody combining site, Science 276(5319): 1665-69). Such known specificity and diversity of ligand binding by different antibodies can be used in designing joining elements for use in constructing nanostructures according to the methods of the invention.

Antibodies or portions thereof used in the methods of the invention can be multispecific (i.e., demonstrate binding affinity towards more than one ligand) or monospecific (i.e., demonstrate binding affinity towards only one ligand). In general, antibodies demonstrate binding affinity in the 10⁻¹ to 10⁻⁴ nM range or better (Padlan, 1994, Anatomy of the antibody molecule, Mol. Immunol. 31(3): 169-217).

An immunoglobulin light or heavy chain variable region consists of a “framework” region interrupted by three hypervariable regions, the CDRs. The Fv fragment contains six variable loop regions, three from the V_{L} chain and three from the V_{H} chain. Each of the variable polypeptide loop regions contained in the variable chains display variability in residue sequence and length. Residues within this region are assigned either to hypervariable, complementarity-determining-regions (CDRs) or to non-hypervariable or framework regions (Wu et al., 1970, An analysis of the sequences of the variable regions of Bence Jones proteins and myeloma light chains and their implications for antibody complementarity, J. Exp. Med 132(2): 211-50; Wu et al., 1975, Similarities among hypervariable segments of immunoglobulin chains, Proc. Natl. Acad. Sci. USA 72(12): 5107-10; Wu et al., 1993, Length distribution of CDRH3 in antibodies, Proteins 16(1): 1-7). The extent of the framework region and CDRs has been precisely defined (see Kabat et al., 1983, Sequences of Proteins of Immunological Interest, U.S. Department of Health and Human Services).

Together, these variable loop regions define, almost entirely, the antigen-recognition site of the antibody. Both CDR3s (CDR3-L and CDR3-H) are the most prominent in antibody-antigen recognition interactions and are the most variable in sequence and conformation. The contributions from the CDR loops from both the V_{L} and the V_{H} chains on binding to antigen are relatively consistent. Structural analyses of antibodies complexed with antigen have determined that approximately 41-44% of the interacting surface area is contributed by the light chain with the heavy chain contributing 56-59% (Davies et al., 1990, Antibody-antigen complexes, Annu. Rev. Biochem. 59: 439-73). The overall number of residues that interact with the antigen is rather small. Structural analysis of antibody-antigen complexes have revealed that, on average, only 15 antibody residues interact with antigen. Other residues within the CDR loops, however, may offer additional antibody-antigen interactions, as well as provide a structural role in order to maintain the antibody combining site structure ((Davies et al., 1990, Antibody-antigen complexes, Annu. Rev. Biochem. 59: 439-73; Wilson and Stanfield, 1994, Antibody-antigen interactions: new structures and new conformational changes, Curr. Opin. Struct. Biol. 4(6): 857-67; Davies and Cohen, 1996, Interactions of protein antigens with antibodies, Proc. Natl. Acad. Sci. USA 93(1): 7-12).

### 5.6.2. Joining Elements Comprising a Recombinantly Engineered Antibody or Binding Derivative or Binding Fragment Thereof

In certain embodiments of the invention, a joining element comprises a recombinantly engineered antibody or binding derivative or binding fragment thereof. There are many examples of recombinantly engineered antibodies known in the art that are multivalent, multispecific and/or multifunctional, and that are suitable as joining elements for use in the design of assembly units for staged assembly of nanostructures. Such assembly units may either be unmodified or be modified as described herein, for use in the methods of the invention for fabrication of a desired nanostructure.

Some examples of recombinantly engineered antibodies, or binding derivatives or binding fragments thereof, for use as joining elements include, but are not limited to:

(i) immunoglobulins from any class including IgG, IgM, IgE, IgA, IgD or any subclass thereof, including immunoglobulins derived from a hybrid hybridoma or from a quadroma (which is a cell line that produces a particular bispecific antibody, i.e. an antibody molecule with two different Fab binding segments);

(ii) monovalent and monospecific antibodies such as Fv, scFv and Fab (Ban, et al., 1994, Crystal structure of an idiotype-anti-idiotype Fab complex, Proc. Natl. Acad. Sci. USA 91(5): 1604-08, Freund et al., 1994, Structural and dynamic properties of the Fv fragment and the single-chain Fv fragment of an antibody in solution investigated by heteronuclear three-dimensional NMR spectroscopy, Biochemistry 33(11): 3296-303; Boulot et al., 1990, Crystallization and preliminary X-ray diffraction study of the bacterially expressed Fv from the monoclonal anti-lysozyme antibody D1.3 and of its complex with the antigen, lysozyme, J. Mol. Biol. 213(4): 617-19; Padlan, 1994, Anatomy of the antibody molecule, Mol. Immunol. 31(3): 169-217);

(iii) bivalent, trivalent, mono-, bi-, or tri-specific antibodies with or without added functionalities, such as IgGs derived from hybrid hybridomas, F(ab′)₂, diabodies, triabodies, tetrabodies, heterologous-F(ab′)₂, Fab-scFv fusions or F(ab′)₂-scFv fusions (Milstein and Cuello, 1983, Hybrid hybridomas and their use in immunohistochemistry, Nature 305(5934): 537-40; Neuberger et al., 1984, Recombinant antibodies possessing novel effector functions, Nature 312(5995): 604-08; Weiner, 1992, Bispecific IgG and IL-2 therapy of a syngeneic B-cell lymphoma in immunocompetent mice, Int. J. Cancer Suppl. 7: 63-66, Holliger and Winter, 1993, Engineering bispecific antibodies, Curr. Opin. Biotechnol. 4(4): 446-49; Dolezal et al., 1995, Escherichia coli expression of a bifunctional Fab-peptide epitope reagent for the rapid diagnosis of HIV-1 and HIV-2, Immunotechnology 1(3-4): 197-209; Tso et al., 1995, Preparation of a bispecific F(ab′)₂ targeted to the human IL-2 receptor, J. Hematother. 4(5): 389-94; Atwell et al., 1996, Design and expression of a stable bispecific scFv dimer with affinity for both glycophorin and N9 neuraminidase, Mol. Immunol. 33(17-18): 1301-12; de Kruif et al., 1996, Leucine zipper dimerized bivalent and trispecific scFv antibodies from a semi-synthetic antibody phage display library, J. Biol. Chem. 271(13): 7630-34; Kipriyanov et al., 1998, Bispecific CD3×CD19 diabody for T cell-mediated lysis of malignant human B cells, Int. J. Cancer 77(5): 763-72; Muller et al., 1998, A dimeric bispecific miniantibody combines two specificities with avidity, FEBS Lett. 432(1-2): 45-49; Carter 2001, Bispecific human IgG by design, J. Immunol. Methods 248(1-2): 7-15; (Fell et al., 1991, Genetic construction and characterization of a fusion protein consisting of a chimeric F(ab′) with specificity for carcinomas and human IL-2, J. Immunol. 146(7): 2446-52; Iliades eet al., 1997, Triabodies: single chain Fv fragments without a linker form trivalent trimers, FEBS Lett. 409(3): 437-41; Hudson and Kortt, 1999, High avidity scFv multimers; diabodies and triabodies, J. Immunol. Methods 231(1-2): 177-89; Schoonjans et al., 2000, Efficient heterodimerization of recombinant bi- and trispecific antibodies, Bioseparation 9(3): 179-83; Schoonjans et al., 2000, Fab chains as an efficient heterodimerization scaffold for the production of recombinant bispecific and trispecific antibody derivatives, J. Immunol. 165(12): 7050-57);

(iv) tetravalent antibodies that are either, mono-, bi-, tri- or tetraspecific antibodies, with or without added functionalities, such as tetrabodies, Ig-G binding derivative-scFv fusions or IgG-scFv fusions (Pack et al., 1995, Tetravalent miniantibodies with high avidity assembling in Escherichia coli, J. Mol. Biol. 246(1): 28-34, Coloma and Morrison, 1997, Design and production of novel tetravalent bispecific antibodies, Nat. Biotechnol. 15(2): 159-63; Alt et al., 1999, Novel tetravalent and bispecific IgG-like antibody molecules combining single-chain diabodies with the immunoglobulin gammal Fc or CH3 region, FEBS Lett. 454(1-2): 90-4; Le Gall et al., 1999, Di-, tri- and tetrameric single chain Fv antibody fragments against human CD19: effect of valency on cell binding, FEBS Lett. 453(1-2): 164-68; Santos et al., 1999, Generation and characterization of a single gene-encoded single-chain-tetravalent antitumor antibody, Clin. Cancer Res. 5(10 Suppl): 3118s-3123s; Goel et al., 2000, Genetically engineered tetravalent single-chain Fv of the pancarcinoma monoclonal antibody CC49: improved biodistribution and potential for therapeutic application, Cancer Res. 60(24): 6964-71; Todorovska et al., 2001, Design and application of diabodies, triabodies and tetrabodies for cancer targeting, J. Immunol. Methods 248(1-2): 47-66); and

(v) fusions of an scFv and a binding derivative of an IgG (see, e.g., Huston et al., 1991, Protein engineering of single-chain Fv analogs and fusion proteins, Methods Enzymol. 203: 46-88); fusions of a cytokine and a binding derivative of an IgG (wherein the cytokine is, e.g., a BCDF (B-cell differentiation factor), a BCGF (B-cell growth factor), a motogenic cytokine, a chemotactic cytokine or chemokine, a CSF (colony stimulating factor), an angiogenesis factor, a TRF (T-cell replacing factor), an ADF (adult T-cell leukemia-derived factor), a PD-ECGF (platelet-derived endothelial cell growth factor), a neuroleukin, an interleukin, a lymphokine, a monokine, an interferon, etc.)(see, e.g., Penichet and Morrison, 2001, Antibody-cytokine fusion proteins for the therapy of cancer, J. Immunol. Methods 248(1-2): 91-101; Penichet et al., 1998, An IgG3-IL-2 fusion protein recognizing a murine B cell lymphoma exhibits effective tumor imaging and antitumor activity, J. Interferon Cytokine Res. 18(8): 597-607; Fell et al., 1991, Genetic construction and characterization of a fusion protein consisting of a chimeric F(ab′) with specificity for carcinomas and human IL-2, J. Immunol. 146(7): 2446-52); fusions of a scFv and a leucine zipper (de Kruif and Logtenberg, 1996, Leucine zipper dimerized bivalent and bispecific scFv antibodies from a semi-synthetic antibody phage display library, J. Biol. Chem. 271(13): 7630-34; see also Section 5.5.3); and fusions of a scFv and a Rop protein (see, e.g., Huston et al., 1991, Protein engineering of single-chain Fv analogs and fusion proteins, Methods Enzymol. 203: 46-88; see also Section 5.5.4).

### 5.6.3. Joining Elements Exhibiting Idiotope/Anti-Idiotope Interactions

In certain embodiments of the invention, idiotope/anti-idiotope interactions are used to design joining elements for the construction of nanostructures according to the methods of the invention. Since antibodies can recognize virtually any antigen, they have the ability to recognize other antigenic determinants contained on other antibodies. The immune responses that arise from the potential antigenic determinants on antibodies are called “idiotopic” (Jerne, 1974, Towards a network theory of the immune system, Ann. Immunol. (Paris) 125C(1-2): 373-89; Davie et al., 1986, Structural correlates of idiotopes, Annu. Rev. Immunol. 4: 147-65). Idiotopes are the antigenic determinants unique to a particular antibody or group of antibodies. Antibodies bearing idiotopes can react with antibodies that recognize the idiotope as antigen and are therefore termed “anti-idiotopic” antibodies. In most cases, the idiotope has been shown by immunological and structural techniques to associate partially or entirely with the CDR of a specific mAb (FIG. 8). Idiotopic antibodies are known to have as great or greater affinity toward their specific anti-idiotopic antibody as toward their specific antigen (Braden et al., 1996, Crystal structure of an Fv-Fv idiotope-anti-idiotope complex at 1.9 A resolution, J. Mol. Biol. 264(1): 137-51).

In some cases, the CDR anti-idiotope adopts a structural conformation of an “internal-image” of the external antigen (Bentley et al., 1990, Three-dimensional structure of an idiotope-anti-idiotope complex, Nature 348(6298): 254-57; Ban et al., 1994, Crystal structure of an idiotope-anti-idiotope Fab complex, Proc. Natl. Acad. Sci. USA 91(5): 1604-08; Poljak, 1994, An idiotope—anti-idiotope complex and the structural basis of molecular mimicking, Proc. Natl. Acad. Sci. USA 91(5): 1599-1600; Braden et al., 1996, Crystal structure of an Fv-Fv idiotope-anti-idiotope complex at 1.9 Å resolution, J. Mol. Biol. 264(1): 137-51; Iliades et al., 1998, Single-chain Fv of anti-idiotype 11-1G10 antibody interacts with antibody NC41 single-chain Fv with a higher affinity than the affinity for the interaction of the parent Fab fragments, J. Protein Chem. 17(3): 245-54). In certain embodiments, idiotopic antibodies are used that have equal or greater affinity towards antigen as anti-idiotopic antibody (Braden et al., 1996, Crystal structure of an Fv-Fv idiotope-anti-idiotope complex at 1.9 Å resolution, J. Mol. Biol. 264(1): 137-51, and references cited therein).

For example, antibodies that bind to a peptide of interest and competitively inhibit the binding of the peptide to its receptor can be used to generate anti-idiotope antibodies that “mimic” the peptide receptor and, therefore, bind the peptide. Anti-idiotope antibodies may be generated using techniques well known to those skilled in the art (see, e.g., Greenspan and Bona, 1993, Idiotypes: structure and immunogenicity, FASEB J. 7(5): 437-44; and Nissinoff, 1991, Idiotypes: concepts and applications, J. Immunol. 147(8): 2429-38).

Illustrative, non-limiting examples of idiotope/anti-idiotope binding pairs useful in the compositions of joining elements and methods of the present invention are provided below in Table 5.

TABLE 5
Idiotope/Anti-Idiotope Interactions

| Idiotope/Anti-Idiotope Complex | Reference |
|---|---|
| Idiotope-Anti-Idiotope Fab-Fab Complex; | Bentley et al., 1990, Three-dimensional |
| D1.3-E225 (Mus musculus) | structure of an idiotope-anti-idiotope |
| | complex, Nature 348(6298): 254-57 |
| Idiotopic Antibody D1.3 Fv | Braden et al., 1996, Crystal structure of an |
| Fragment-Anti-idiotopic Antibody E5.2 Fv | Fv-Fv idiotope-anti-idiotope complex at |
| Fragment Complex (Mus musculus) | 1.9 Å resolution, J. Mol Biol. 264(1): |
| | 137-51 |
| Fab of YsT9.1 (Ab1) and the Fab of its | Evans et al. 1994, Exploring the mimicry |
| anti-idiotopic monoclonal antibody | of polysaccharide antigens by anti-idiotypic |
| T91AJ5 (Ab2) | antibodies. The crystallization, molecular |
| | replacement, and refinement to 2.8 Å |
| | resolution of an idiotope-anti-idiotope Fab |
| | complex and of the unliganded |
| | anti-idiotope Fab, J. Mol. Biol. 241(5): |
| | 691-705 |
| Idiotope-Anti-idiotope complex of | Poljak, 1994, An idiotope—anti-idiotope |
| antibody fragments | complex and the structural basis of |
| | molecular mimicking, Proc. Natl. Acad. |
| | Sci. USA 91(5): 1599-600 |
| Fab fragment of the mouse | Ban et al., 1996, Crystal structure of an |
| anti-anti-idiotypic monoclonal antibody | anti-anti-idiotype shows it to be |
| (mAb) GH1002 | self-complementary, J. Mol. Biol. 255(4): |
| | 617-27 |
| Anti-idiotopic Fab 409.5.3, made against | Ban et al., 1995, Structure of an |
| an E2 specific feline infectious peritonitis | anti-idiotypic Fab against feline peritonitis |
| virus-neutralizing antibody 730.1.4 | virus-neutralizing antibody and a |
| | comparison with the complexed Fab, |
| | FASEB J. 9(1): 107-14 |

In certain embodiments, specific idiotope/anti-idiotope intermolecular interactions are used as the joining elements to link assembly units together in the staged assembly of a nanostructure (FIG. 16). Each derived assembly unit is designed to contain two specific idiotope/anti-idiotope binding surfaces that are non-cross-reacting. This provides a means of creating a system for the staged assembly of assembly units to form complex nanostructures comprising various and diverse functional elements. Multiple joining pairs can be created by standard methods of phage display (Winter et al., 1994, Making antibodies by phage display technology, Ann. Rev. Immunol. 12: 433-55;Viti et al., 2000, Design and use of phage display libraries for the selection of antibodies and enzymes, Methods Enzymol. 326: 480-505). Furthermore, the three-dimensional structure of antibodies and antibody derivatives are well-characterized (see, e.g., Braden et al. 1996, Crystal structure of an Fv-Fv idiotope-anti-idiotope complex at 1.9 Å resolution, J. Mol. Biol. 264(1): 137-51; Ban et al., 1994, Crystal structure of an idiotype-anti-idiotype Fab complex, Proc. Natl. Acad. Sci. USA 91(5): 1604-08; Perisic et al. 1994, Crystal structure of a diabody, a bivalent antibody fragment, Structure 2(12): 1217-26; Harris et al., 1998, Crystallographic structure of an intact IgG1 monoclonal antibody, J. Mol. Biol. 275(5): 861-72; Pei et al., 1997, The 2.0-A resolution crystal structure of a trimeric antibody fragment with noncognate V_{H}-V_{L} domain pairs shows a rearrangement of V_{H} CDR3, Proc. Natl. Acad. Sci. USA 94(18): 9637-42) and positions for engineering additional functional elements may be identified by visual investigation of the available X-ray coordinates.

In certain embodiments, one of the CDR domains (i.e., one of the joining elements) of an antibody-derived assembly unit can be engineered as an idiotope. The other CDR can be engineered as a non-complementary anti-idiotope joining element. Since the joining elements are non-identical and non-interactive with each other, this design prevents self-polymerization of the protein component. Such joining elements can be fabricated using combinations of molecular biology and phage display technologies (Winter et al., 1994, Making antibodies by phage display technology, Ann. Rev. Immunol. 12: 433-55; Viti et al., 2000, Design and use of phage display libraries for the selection of antibodies and enzymes, Methods Enzymol. 326: 480-505). The resulting antibody-derived assembly unit will contain both an idiotopic CDR or joining element and a non-complementary anti-idiotopic CDR joining element.

In certain embodiments of the invention, the assembly unit to be coupled in the next addition cycle can be designed in an analogous fashion, with a joining element that is an idiotope and a joining element that is a non-complementary anti-idiotope. One CDR of this assembly unit, however, can be engineered to associate with one of the previous CDR components that functions as joining elements. Therefore, in certain embodiments, the CDRs of two adjacent assembly units can be designed to have joining elements that have complementary idiotope/anti-idiotope interactions. Using assembly units of this design allows for a defined directionality or orientation of the linked assembly unit and of the staged assembly as a whole, i.e., vectorial addition of each assembly unit. Since the CDRs of diabodies are geometrically opposed, the assembly units can be added to an initiator or nanostructure intermediate in known orientation and direction.

### 5.6.4. Joining Elements Comprising Two Non-Complementary Idiotopes

In certain embodiments, an assembly unit is fabricated that comprises a diabody unit, wherein the non-complementary joining elements are comprised of two non-complementary idiotopes. A diabody, or a binding derivative or binding fragment thereof, may be incorporated into a nanostructure in such a way that only one of the two CDRs is used. In certain embodiments, the CDRs themselves serve as joining elements, and the body of the diabody between the two CDRs serves as a structural element.

Bispecific diabodies are derived from two non-paired scFv fragments. The first portion of the hybrid fragment contains the V_{H} coding region from one Fv antibody and the second portion contains the V_{L} coding region derived from another Fv antibody. The resulting V_{H}-V_{L} hybrid fragment is joined together by a short Gly₄Ser linker. The second hybrid fragment will contain linkage of the analogous but opposite coding region pair also joined together by a short Gly₄Ser linker (FIGS. 8 and 12). The set of hybrid scFv fragments pair by intermolecular interactions between the V_{H} and V_{L} domains.

In a specific embodiment illustrated in FIG. 7, the genes used to create a first assembly unit (“Diabody Unit 1”) are derived from the lysozyme idiotopic antibody D1.3 (represented as V_{H}A and V_{L}A in FIG. 7A) (Braden et al., 1996, Crystal structure of an Fv-Fv idiotope-anti-idiotope complex at 1.9 Å resolution, J. Mol. Biol. 264(1): 137-51) and the feline infectious peritonitis virus-neutralizing idiotopic antibody 730.1.4 (represented as V_{H}B and V_{L}B in FIG. 7A) (Ban et al., 1994, Crystal structure of an idiotype-anti-idiotype Fab complex, Proc. Natl. Acad. Sci. USA 91(5): 1604-08). The linker sequences joining the hybrid V_{H}A and V_{L}B units and the hybrid V_{H}B and V_{L}A units are designed based on those published by Huston et al. (1988, Protein engineering of antibody binding sites: recovery of specific activity in an anti-digoxin single-chain Fv analogue produced in Escherichia coli, Proc. Natl. Acad. Sci. USA 85(16): 5879-83). The construct of Diabody Unit 1 is represented as A×B in FIG. 7A. The locations of the promoter (p), ribosome binding site (rbs), pelB leader (pelB), HSV and histidine (his) tags and stop codons (Stop) are also indicated in FIG. 7. The vector system used to engineer the diabody is pET25b (Novagen), which contains a T7 promoter, ribosome binding site, pelB leader sequence, HSV and His tag sequences.

FIG. 7B illustrates a second assembly unit (Diabody Unit 2) comprises a diabody, wherein the non-complementary joining elements are designed to contain two non-complementary anti-idiotopes. The genes used to create this second assembly unit are derived from the lysozyme anti-idiotopic antibody E5.2 (represented as V_{H}A′ and V_{L}A′ in FIG. 7B) (Braden et al., 1996, Crystal structure of an Fv-Fv idiotope-anti-idiotope complex at 1.9 Å resolution, J. Mol. Biol. 264(1): 137-51) and the feline infectious peritonitis virus-neutralizing anti-idiotopic antibody 409.5.3 (represented as V_{H}B′ and V_{L}B′ in FIG. 7B) (Ban et al., 1994, Crystal structure of an idiotype-anti-idiotype Fab complex, Proc. Natl. Acad. Sci. USA 91(5): 1604-08). The construct of Diabody Unit 2 is represented as A′×B′ . These two exemplary assembly units can be used in conjunction with an initiator unit to fabricate a nanostructure by the methods of staged assembly described herein.

### 5.6.5. Joining elements Comprising a Peptide Epitope

In certain embodiments of the invention, joining elements comprise peptide epitopes. Peptide epitopes may be engineered into assembly units to act as joining elements that form a complementary pair with an antibody or antibody binding fragment, the CDR of which binds to the peptide epitope with specificity. Peptide epitopes can be spliced into multiple defined regions contained within the assembly units described above. Peptides epitopes are particularly preferred as joining elements for use in a number of embodiments, in addition to those embodiments wherein the peptide epitope is used for cross-linking assembly units of adjacent nanostructures together. Therefore, peptide epitopes provide versatility to assembly units into which they are incorporated.

For example, in certain embodiments, peptide epitopes can serve as joining elements for junctions that can be initiation points for the assembly of new branches of a nanostructure from a pre-existing branch. Such branching may be used to generate one,- two- or three-dimensional structures. It may be used to expand beyond a simple one-dimensional structure or to attach functional units to a one-dimensional structure. Alternatively, such joining elements can serve as the binding sites for the addition of separately-fabricated nanostructure sub-assemblies to nanostructure intermediates. In other embodiments, they can serve as binding sites for antibodies that have linked or bound functional elements.

In certain embodiments, assembly units comprise antibody fragments that comprise peptide epitope joining elements. The inherent flexibility within the Fab fragment may be used advantageously for insertion of a joining element that enables various cross-linked geometries between assembly units of nanostructures in a staged assembly. In one embodiment, to incorporate the additional intermolecular binding site on the Fab fragment needed for staged assembly, the C-terminal distal end, or the β-turn regions, are engineered to contain a peptide epitope. Exemplary peptide epitopes are set forth in Table 6.

TABLE 6
Examples of Peptide Epitopes for Use as Joining Elements

| Antibody/Antigenic-Peptide | Sequence | Reference |
|---|---|---|
| (Antibody 8F5) Complexed | VKAETRLNPDLQPTE | Tormo et al., 1994, Crystal |
| With Peptide From Human | (SEQ ID NO: 70) | structure of a human |
| Rhinovirus (Serotype 2) | | rhinovirus neutralizing |
| Viral Capsid Protein Vp2 | | antibody complexed with a |
| (Residues 156-170) | | peptide derived from viral |
| | | capsid protein VP2, EMBO |
| | | J. 13(10): 2247-56 |
| Fab59.complexed with a | YNKRKRIHIGPGRXFYT | Ghiara et al., 1997, |
| peptide mimic of the HIV-1 | TKNIIGC | Structure-based design of a |
| V3 loop neutralization site. | (SEQ ID NO: 71) | constrained peptide mimic |
| | | of the HIV-1 V3 loop |
| | | neutralization site, J. Mol. |
| | | Biol. 266(1): 31-39 |
| Antibody Campath-1H Fab | GTSSPSAD | James et al., 1999, 1.9 Å |
| /Peptide Antigen | (SEQ ID NO: 72) | structure of the therapeutic |
| | | antibody CAMPATH-1H |
| | | Fab in complex with a |
| | | synthetic peptide antigen. J. |
| | | Mol. Biol. 289(2): 293-301 |
| Anti-Prion Fab 3F4 In | APKTNMKHMA | Kanyo et al., 1999, |
| Complex With Its Peptide | (SEQ ID NO: 73) | Antibody binding defines a |
| Epitope | | structure for an epitope that |
| | | participates in the |
| | | PrPC—>PrPSc |
| | | conformational change. J. |
| | | Mol. Biol. 293(4): 855-63 |
| Fab Fragment Monoclonal | YTTSTRGDLAHVTTT | Ochoa et al. 2000, A |
| Antibody 4C4 w/ | (SEQ ID NO: 74) | multiply substituted G-H |
| Fmdv.peptide | | loop from foot-and-mouth |
| | | disease virus in complex |
| | | with a neutralizing |
| | | antibody: a role for water |
| | | molecules. J. Gen. Virol. |
| | | 81 (Pt 6): 1495-505 |
| Igg2A Fab (C3) Poliovirus | CVTIMTVDNPASTTNKDK | Wien et al., 1995, Structure |
| Type 1 Fragment | (SEQ ID NO: 75) | of the complex between the |
| | | Fab fragment of a |
| | | neutralizing antibody for |
| | | type 1 poliovirus and its |
| | | viral epitope. Nat. Struct. |
| | | Biol. 2(3): 232-43 |
| Antibody Sm3 Complex | TSAPDTRPAPGST | Dokurno et al., 1998, |
| With Its Peptide Epitope | (SEQ ID NO: 77) | Crystal structure at 1.95 A |
| | | resolution of the breast |
| | | tumour-specific antibody |
| | | SM3 complexed with its |
| | | peptide epitope reveals |
| | | novel hypervariable loop |
| | | recognition, J. Mol. Biol. |
| | | 284(3): 713-28 |
| Fab 58.2 Complex With | HIGPGRAFGG G | Stanfield et al., 1999, Dual |
| 12-Residue Cyclic Peptide | (SEQ ID NO: 78) | conformations for the |
| | | HIV-1 gp120 V3 loop in |
| | | complexes with different |
| | | neutralizing Fabs, Structure |
| | | Fold. Des. 7(2): 131-42 |
| Monoclonal Antibody | MSLPGRWKPK | Lescar et al., 1997, |
| F11.2.32; Fab; complexed | (SEQ ID NO: 79) | Three-dimensional structure |
| with Hiv-1 Protease | | of an Fab-peptide complex: |
| Peptide; | | structural basis of HIV-1 |
| | | protease inhibition by a |
| | | monoclonal antibody, J. |
| | | Mol. Biol 267(5): 1207-22 |
| Mn12H2 Igg2A Fab | KDTNNNL | van den Elsen et al., 1997, |
| Fragment; complexed with | (SEQ ID NO: 80) | Bactericidal antibody |
| Fluorescein-Conjugated | | recognition of a PorA |
| Peptide | | epitope of Neisseria |
| | | meningitidis: crystal |
| | | structure of a Fab fragment |
| | | in complex with a |
| | | fluorescein-conjugated |
| | | peptide, Proteins 29(1): |
| | | 113-25 |

In one embodiment, a peptide epitope can replace the defined β-turn motifs contained in the fragment directly. Alternatively, a peptide epitope can be linked to the C-terminal amino acid of the CH1 heavy chain (Wallace et al., 2001, Exogenous antigen targeted to FcgammaRI on myeloid cells is presented in association with MHC class I, J. Immunol. Methods 248(1-2): 183-94) by standard methods of molecular biology. Table 7 sets forth examples of identified peptide regions contained in IgG and IgG derivations that are suitable for insertion of joining elements or functional elements.

TABLE 7
Identified Peptide Regions Contained in IgG and IgG
Derivatives for Insertion of Joining Elements or Functional
Elements

| | | |
|---|---|---|
| | | Residue |
| Domain | Secondary Structure | (Chain)² |

IgG1 (Fc)¹

| | | |
|---|---|---|
| C_{H}2 | β-turn | res 265-269 |
| | | res 295-299 |
| | | res 311-317 |
| | | (B, D) |
| C_{H}3 | β-turn | res 408-414 |
| | | res 449-452 |
| | | res 464-466 |
| | | (B, D) |
| C_{H}3 | C-terminal | res 474 |
| | α C | (B, D) |

Fab Fragment³

| | | |
|---|---|---|
| Fv | β-turn | res 14-18 |
| | | (A) |
| | | res 11-16 |
| | | (B) |
| Fab | Extended | res 107-111 |
| Bend | Loop | (A) |
| Region | | res 115-120 |
| | | (B) |
| C_{H}1 | β-turn | res 149-153 |
| | | res 198-202 |
| | | (A) |
| | | res 159-162 |
| | | res 203-207 |
| | | (B) |
| C_{H}1 | C-terminal | res 214 |
| | αC | (A) |
| | | res 217 |
| | | (B) |

scFv⁴

| | | |
|---|---|---|
| V_{H} | β-turn | res 13-16 |
| | | res 88-90 |
| | | res 40-43 |
| | | (D) |
| V_{L} | β-turn | res 12-16 |
| | | res 45-48 |
| | | (C) |
| V_{H} | C-terminal | res 218 |
| | αC | (D) |

Diabody⁵

| | | |
|---|---|---|
| V_{H} | β-turn | res 13-16 |
| | | res 39-44 |
| | | res 62-66 |
| | | res 73, 77 |
| | | (A, C) |
| V_{L} | C-terminal | res 312 |
| | αC | (A, C) |

Table 7 Notes:
¹Residue regions are defined in the Fc fragment of the intact IgG1 from analysis of the atomic coordinates and numbered according to the residue assignments deposited under entry 1IGY at the Brookhaven National Laboratory protein data bank (BNL-pdb) (Berman et al., 2000, The Protein Data Bank, Nucl. Acids Res. 235-42; 1977, The Protein Data Bank. A computer-based archival file for macromolecular structures, Eur. J. Biochem. 80(2): 319-24).
²Chain assignments are labeled in accord with the corresponding deposited pdb coordinates.
³Residue regions are defined in the Fab fragment from analysis of the atomic coordinates and numbered according to the residue assignments deposited under entry 1CIC at the BNL-pdb.
⁴Residue regions are defined within the scFv fragment from analysis of the atomic coordinates and numbered according to the residue assignments deposited under entry 2AP2 at the BNL-pdb.
⁵Residue regions are defined within the diabody fragment from analysis of the atomic coordinates and numbered according to the residue assignments deposited under entry 1LMK at the BNL-pdb.

In another embodiment, the resulting Fab fragment contains an antigen binding domain, at the N-terminal proximal end of the molecule. The Fab fragment also contains a joining element that is a peptide epitope, inserted at a position in the Fab fragment replacing a defined β-turn motif, or linked directly to the distal C-terminal end of the Fab fragment. Thus the peptide epitope fused to the Fab fragment serves as a highly specific joining element that can serve as an attachment point, through the recognition and binding of a cognate immunoconjugated functional moiety.

### 5.6.6. Joining Elements Comprising Bacterial Pilin Proteins or Binding Derivatives or Binding Fragments Thereof

As discussed above in Section 5.5.2, in certain embodiments of the invention, a structural element comprises a bacterial pilin protein or binding derivative or binding fragment thereof. In other embodiments of the invention, joining elements comprise a bacterial pilin protein or binding derivative or binding fragment thereof. In yet other embodiments, an assembly unit may comprise a pilin protein or binding derivative or binding fragment thereof that serves as a structural element and a joining element. The general structure and properties of pilin proteins are described above in Section 5.5.2. Pilins are highly homologous in the region spanning the C-terminal end of their N-terminal extension and the N-terminal end of the pilin body. This region of homology provides guidance for the design of hybrid pilins made of the N-terminal extension of one pilin and the body of the other. A hybrid pilin comprises the N-terminal extension from one pilin and the body of another pilin. In one aspect of the invention, such hybrid pilins may be used for the construction of an assembly unit, and may serve as a structural element, a joining element, or both a structural and a joining element.

Non-limiting examples of the N-terminal extensions of various pilin proteins, and the N-terminal amino acid sequences of various pilin protein bodies lacking the N-terminal extension, are shown in Table 8, below. Hybrid pilins that comprise the N-terminal extension from one pilin and the body of another pilin, may be expressed and purified by methods commonly known in the art (e.g., Bullitt and Makowski, 1995, Structural polymorphism of bacterial adhesion pili, Nature 373: 164-67; Bullitt et al., 1996, Development of pilus organelle sub-assemblies in vitro depends on chaperone uncapping of a beta zipper, Proc. Natl. Acad. Sci. USA 93: 12890-95).

TABLE 8

Amino Acid Sequence of the N-terminal Extension
and the Adjacent Pilin Body of Pilins
papA, papK, papH, papE, and papF

| | | N-Terminal Amino Acid |
|---|---|---|
| | | Sequence of the Pilin Protein |
| | | Body Lacking the N-terminal |
| Pilin | N-Terminal Extension | Extension |
| papA: | AVPQGQGKVTFSGTVVDA | PCGIDAAQSADQSVDFGQLSKVFLDNDGQ |
| | (SEQ ID NO: 81) | TTPKAFDIKLVNCDITNYKKPATG |
| | | (SEQ ID NO: 82) |
| papK: | MIKSTGALLLFAALSAGQAIASDV | PCHVSGDSLNKHVVFKTRASRDFWYPPGR |
| | AFRGNLLDR | SPTESFVI |
| | (SEQ ID NO: 83) | (SEQ ID NO: 84) |
| papH: | MRLRFSVPLFFFGCVFVHGVFAGP | PACTLAMEDAWQIID |
| | FPPPGMSLPEYWGEEHVWWDGRAA | (SEQ ID NO: 86) |
| | FHGEVVR | |
| | (SEQ ID NO: 85) | |
| papE: | MKKIRGLCLPVMLGAVLMSQHVHA | PACTVTKAEVDWGNVEIQTLSPDGSRVIQ |
| | ADNLTFKGKLII | KDFSVG |
| | (SEQ ID NO: 87) | (SEQ ID NO: 88) |
| papF: | MARLSLFISLLLTSVAVLADVQIN | PPCTINNGQNIVVDFGNINPEHVDNSRGE |
| | IRGNVYL | ITKTISISCT |
| | (SEQ ID NO: 89) | (SEQ ID NO: 90) |

Pilins exhibit a well-folded protein structure formed largely of β-sheets, along with the flexible N-terminal peptide domain that is recognized and bound by certain other pilin proteins, as described above. Pilins provide an illustrative example of assembly units that are not fully rigid prior to assembly. In certain embodiments of the invention, protein domains involved in protein-protein interactions are flexible prior to binding. The N-terminal extension of the pilins represents one such case. A pilin protein recognizes and binds to the flexible extension of another pilin protein, and thus can serve as a joining element suitable for use in the staged assembly of nanostructures according to the present invention. After binding, the N-terminal extension is held rigidly through its binding to an adjacent, cognate pilin protein, providing the rigidity needed in a staged assembly. Generally, a pilin protein fragment is unlikely to maintain its structure adequately to provide for specific and tight interactions with other pilin proteins, unless that fragment comprises substantially all of the pilin protein. However, in certain embodiments of the invention, a few amino acids may be altered, added, or deleted to one or more of the beta turns of the pilin without disrupting its overall structure, structural rigidity or recognition properties, thereby providing one or more sites suitable for the insertion of a functional element.

As shown in Table 8 above, the N-terminal extension of papA comprises the first 20 amino acids. The extension is longer in other pilins. PapH has a particularly long extension because it is required for anchoring to the outer membrane of E. coli. Consequently, this long papH extension is not used in preferred embodiments of the present invention. It is included in Table 8 to illustrate the preservation among pilins of the sequences in the region that comprises the second half of the N-terminal extension, and in the region of the N-terminal portion of the pilin protein body. Similarities in the N-terminal extensions indicate that the extensions are used in the same way and interact with the proximal pilin in similar fashion. Differences in the sequences of the N-terminal extensions are responsible for the differences in their binding specificity.

In another embodiment, an assembly unit is fabricated that comprises fragments of multiple pilin proteins, wherein each pilin unit comprises a joining element that is a peptide epitope.

The following are non-limiting examples of hybrid pilin assembly units that may be engineered for use in the compositions and methods of the invention:

(i) PapH with the amino terminus of papK. Using standard methods, the DNA sequence coding for the amino terminal extension of papH is replaced with the DNA sequence encoding the amino terminal extension of papK within a plasmid designed to overproduce papH.

(ii) PapH-papK hybrid with added epitope: DNA coding for a Ras epitope (see Table 11, below) is inserted in the gene for papH between the two codons coding for amino acids 121 and 126 of papH (at the position corresponding to the surface loop in papA).

(iii) PapE with the amino terminus of papA: Using standard methods of recombinant DNA technology, the DNA sequence coding for the amino terminal extension of papE is replaced with the DNA sequence encoding the amino terminal extension of papA within a plasmid designed to overproduce papE.

(iv) PapK with the amino terminus of papF: Using standard methods of recombinant DNA technology, the DNA sequence coding for the amino terminal extension of papK is replaced with the DNA sequence encoding the amino terminal extension of papF within a plasmid designed to overproduce papK.

(v) PapH with the amino terminus of papE. Using standard methods of recombinant DNA technology, the DNA sequence coding for the amino terminal extension of papH is replaced with the DNA sequence encoding the amino terminal extension of papE within a plasmid designed to overproduce papH.

(vi) PapH-papE hybrid with added epitope: DNA coding for a Ras epitope is inserted in the gene for papH between the two codons coding for amino acids 121 and 126 of papH (at the position corresponding to the surface loop in papA).

Hybrid pilin assembly units may be assembled to form nanostructures by staged assembly using, in one embodiment, the method disclosed in Section 6 (Example 1). This embodiment is also depicted in FIG. 17 and provides a schematic representation of the nanostructure intermediates formed.

### 5.6.7. Joining Elements Comprising Peptide Nucleic Acids (PNAs)

In certain embodiments of the invention, a joining element comprises a peptide nucleic acid (PNA) and may have any of a number of general forms, such as that shown in FIG. 18. PNA is a structural homologue of DNA that was first described by Nielsen et al. (1991, Sequence-selective recognition of DNA by strand displacement with a thymine-substituted polyamide, Science 254: 1497-1500) and has a neutral peptide or peptide-like backbone instead of a negatively-charged sugar-phosphate backbone (FIG. 18). Therefore, a PNA may be viewed as a protein or oligopeptide in which the amino acid side chains have been replaced with the pyrimidine and purine bases of DNA. The same nitrogenous bases (i.e. adenine, guanine, cytosine and thymine) are used in PNAs as are found in DNA and RNA; PNAs bind to DNA and RNA molecules according to Watson-Crick and/or Hoogsteen base pairing rules. PNAs are not generally recognized as substrates by DNA polymerases, nucleic acid binding proteins, or other enzymes, including proteases and nucleases, although some exceptions do exist (see, e.g., Lutz et al., 1997, Recognition of uncharged polyamide-linked nucleic acid analogs by DNA polymerases and reverse transcriptases, J. Am. Chem. Soc. 119: 3177-78). The biology of PNAs has been reviewed extensively (see, e.g., Nielsen et al., 1992, Peptide nucleic acids (PNA). DNA analogues with a polyamide backbone, In Antisense Research and Applications, Crooke and Lebleu, eds., CRC Press, pp. 363-72; Nielsen et al., 1993, Peptide nucleic acids (PNAs): potential antisense and anti-gene agents, Anticancer Drug Des. 8(1): 53-63; Buchardt et al., 1993, Peptide nucleic acids and their potential applications in biotechnology, Trends Biotechnol. 11(9): 384-86; Nielsen et al., 1994, Peptide nucleic acid (PNA). A DNA mimic with a peptide backbone, Bioconjug. Chem. 5(1): 3-7; Nielsen et al., 1996, Peptide nucleic acid (PNA): A lead for gene therapeutic drugs, in Antisense Therapeutics Vol. 4, Trainor, ed., SECOM Science Publishers B. V., Leiden, pp. 76-84; Nielsen, 1995, DNA analogues with nonphosphodiester backbones, Ann. Rev. Biophys. Biomol. Struct. 24: 167-83; Hyrup and Nielsen, 1996, Peptide nucleic acids (PNA): synthesis, properties and potential applications, Bioorg. Med. Chem. 4: 5-23; De Mesmaeker et al., 1995, Backbone modifications in oligonucleotides and peptide nucleic acid systems, Curr. Opin. Struct. Biol. 5: 343-55; Dueholm and Nielsen, 1997, Chemical aspects of peptide nucleic acid, New J. Chem. 21: 19-31; Knudsen and Nielsen, 1997, Application of PNA in cancer therapy, Anti-Cancer Drug 8: 113-18; Nielsen, 1997, Design of Sequence Specific DNA Binding Ligands, Chemistry 3: 505-08; Corey, 1997, Peptide nucleic acids: expanding the scope of nucleic acid recognition. Trends Biotechnol. 15(6):224-29; Nielsen and Orum, 1995, Peptide nucleic acid (PNA) as new biomolecular tools, in Molecular Biology: Current Innovations and Future Trends, Part 2, (Griffin, H., Ed.), Horizon Scientific Press, UK, pp. 73-86; Nielsen and Haaima, 1997, Peptide Nucleic Acid (PNA). A DNA Mimic with a Pseudopeptide Backbone, Chem. Soc. Rev.: 73-78).

In PNA, as shown in FIG. 18, the phosphoribose backbone may be replaced, for example, by repeating units of N-(2-aminoethyl)-glycine linked by amide bonds (Egholm et al., 1992, Peptide nucleic acids (PNA), Oligonucleotide analogues with an achiral peptide backbone, J. Am. Chem. Soc. 114: 1895-97). Other substitutions in PNA of a neutral peptide or peptide-like backbone for a negatively-charged sugar-phosphate backbone are commonly known in the art and will be readily apparent to the skilled artisan. PNAs with modified polyamide backbones have been described, for example, in Hyrup et al. (1994, Structure-Activity studies of the binding modified Peptide Nucleic Acids, Journal of the American Chemical Society 116: 7964-70); Dueholm et al. (1994, Peptide Nucleic Acid (PNA) with a chiral backbone based on alanine, Bioorg. Med. Chem. Lett. 4: 1077-80); Peyman et al. (1996, Phosphonic Esters Nucleic Acids (PHONAs): Oligonucleotide Analogues with an Achiral Phosphonic Acid Ester Backbone, Angew. Chem. Int. Ed. Engl. 35: 2636-38); van der Laan et al. (1996, An approach towards the synthesis of oligomers containing a N-2-hydroxyethyl-aminomethylphosphonate backbone—A novel PNA analogue, Tetrahedron Letters 37: 7857-60); Jordan et al. (1997, Synthesis of new building blocks for peptide nucleic acids containing monomers with variations in the backbone, Bioorg. Med. Chem. Lett. 7: 681-86); Goodnow et al. (1997, Oligomer Synthesis and DNA/RNA Recognition Properties of a Novel Oligonucleotide Backbone Analog: Glucopyranosyl Nucleic Amide (GNA), Tetrahedron Lett. 38: 3199-3202); Zhang et al. (1999, Studies on the synthesis and properties of new PNA analogs consisting of L- and D-lysine backbones, Bioorg. Med. Chem. Lett. 9: 2903-08); Stammers et al. (1999, Synthesis of enantiomerically pure backbone alkyl substituted peptide nucleic acids utilizing the Et-DuPHOS-Rh+ hydrogenation of enamido esters, Tetrahedron Lett., 40, 3325-3328); Puschl et al. (2000, Pyrrolidine PNA: A Novel Conformationally Restricted PNA Analogue, Organic Letters 2: 4161-63); Vilaivan et al. (2000, Synthesis and properties of chiral peptide nucleic acids with a N-aminoethyl-D-proline backbone, Bioorg Med Chem Lett 10(22):2541-45); Yu et al., 2001, Synthesis and characterization of a tetranucleotide analogue containing alternating phosphonate-amide backbone linkages, Bioorg. Med. Chem. 9(1):107-19); Fader et al. (2001, Backbone modifications of aromatic peptide nucleic acid (APNA) monomers and their hybridization properties with DNA and RNA, J. Org. Chem. 66: 3372-79).

The nitrogenous bases of a PNA are attached to the neutral backbone by methylene carbonyl linkages. Because PNA does not have a highly-charged sugar-phosphate backbone, PNA binding to a target nucleic acid is stronger than with conventional nucleic acids, and that binding, once established, is virtually independent of salt concentration. This is reflected, quantitatively, by a high thermal stability of duplexes containing PNA.

Because the peptide backbone is uncharged, base-pairing between two complementary PNA molecules, or between, e.g., DNA and PNA in a DNA/PNA hybrid, is much stronger than in the corresponding DNA/DNA hybrid. Binding of a PNA to its complementary DNA or RNA target will occur more quickly than binding of the equivalent nucleic acid probe. The affinity of the PNA is so high that it can displace the corresponding strand in double stranded DNA (Nielsen et al., 1991, Sequence-selective recognition of DNA by strand displacement with a thymine substituted polyamide, Science 254: 1497-1500).

PNAs generally have a melting temperature that is higher than the corresponding DNA duplex, by approximately −1° C. per base at moderate salt conditions (e.g., 100 mM NaCl) (Nielsen et al., 1991, Sequence-selective recognition of DNA by strand displacement with a thymine-substituted polyamide, Science 254: 1497-1500; Peffer et al., 1993, Strand-invasion of duplex DNA by peptide nucleic acid oligomers, Proc. Natl. Acad. Sci. USA 90: 10648-52; Demidov et al., 1995, Kinetics and mechanism of polyamide (“peptide”) nucleic acid binding to duplex DNA, Proc. Nati. Acad. Sci. USA 92: 2637-41). Thermal stability of a DNA-DNA duplex (as indicated by Tₘ) is approximated using an estimate of 2° C. per AT base pair and 4° C. per GC base pair, whereby a 10 bp DNA duplex with 50% GC content would be estimated to have melting temperature of about 30° C. Accordingly, the corresponding PNA therefore would have a melting temperature of about 40° C. Similarly an 18 residue PNA duplex (50% GC) would be estimated to have a melting temperature of about 72° C. Therefore, in certain embodiments of the present invention a PNA joining element has about 8 residues to about 20 residues, about 10 residues to about 18 residues, or about 12 residues to about 16 residues.

In other embodiments, PNAs having fewer residues can be designed that have higher melting temperatures by taking advantage of the PNA's ability to form triple helices. In a specific embodiment, three PNA strands (two polypyrimidine, one polypurine) form this extremely stable structure. The structure can be further stabilized by using two PNA's such that one has two polypyrimidine PNA stretches separated by a glycine spacer, wherein the glycine spacer generally comprises three to five glycine residues. When mixed with the corresponding polypurine PNA, the two polypyrimidine PNA segments fold around the glycine space to form this triple helix. Having the “two” polypyrimidine strands on the same molecule raises the effective concentration and hence the rate of formation and strength of the triplex helix. For a staged assembly joining pair, one joining element of the joining pair would contain the polypurine strand while the other joining element of the joining pair is a double-length polypyrimidine PNA joining element.

PNAs may be synthesized by methods well known in the art using chemistries similar to those used for synthesis of nucleic acids and peptides. PNA monomers used in such syntheses are hybrids of nucleosides and amino acids. PNA products, services (such as custom-synthesis of PNA molecules), and technical support are commercially available from PerSeptive Biosystems, Inc. (a division of Applied Biosystems, Foster City, Calif.). PNA may be synthesized using commercially available reagents and equipment or can be purchased from contract manufacturers such as PerSeptive Biosystems, Inc. PNA oligomers may also be manually synthesized using either Fmoc or t-Boc protected monomers using standard peptide chemistry protocols. Similarly, standard peptide purification conditions may be used to purify PNA following synthesis.

In certain embodiments, a PNA used in the methods of the invention is a chimeric PNA or a binding derivative or modified version thereof. A chimeric PNA is a molecule that is modified at the base moiety or the peptide backbone, and that may include other appending groups or labels. A chimeric PNA also may be a molecule that comprises a PNA sequence linked by a covalent bond(s) to one or more amino acids or to a sequence of two or more contiguous amino acids.

For example, a chimeric or modified PNA may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), S-methyl-2-thiouracil, 3-(3-amino-3-N-2-carboxypropyl) uracil, (acp3)w, and 2,6-diaminopurine.

In a specific embodiment, a modified or chimeric PNA contains the “universal base” 3-nitropyrrole (Zhang et al., 2001, Peptide nucleic acid-DNA duplexes containing the universal base 3-nitropyrrole, Methods 23: 132-40).

Once a desired PNA is synthesized, it is cleaved from the solid support on which it was synthesized and treated, by methods known in the art, to remove any protecting groups present. The PNA may then be purified by any method known in the art, including extraction and gel purification. The concentration and purity of the PNA may be determined by examining PNA that has been separated on an acrylamide gel, or by measuring the optical density in a spectrophotometer.

In certain embodiments of the invention, a joining pair comprises a complementary pair of PNA joining elements that are capable of binding via standard Watson-Crick and/or Hoogsteen base-pairing. A PNA moiety can serve as a joining element, while an oligopeptide, protein, or protein fragment provides a small structural element and, in specific embodiments, the structural element further comprises a functional element, as depicted schematically in FIGS. 19(A-B). As shown in FIGS. 19(A-B), two PNA/oligopeptide units can dimerize to form a single assembly unit. The PNA portion provides joining elements A and B′, while the oligopeptide portion forms two coiled coil structural elements.

Like DNA, PNA/PNA molecules bind most stably in an antiparallel fashion (Wittung et al., 1994, DNA-like double helix formed by peptide nucleic acid, Nature 368: 561-63). For PNA molecules the amino terminus is equivalent to the 5′ end of a corresponding DNA sequence (FIG. 18). Leucine zipper dimers normally bind in a parallel fashion (amino terminus adjacent to amino terminus) (Harbury et al., 1993, A switch between two-, three-, and four-stranded coiled coils in GCN4 leucine zipper mutants, Science 262: 1401-07). Therefore, all the molecules depicted in the assembly units shown in FIG. 19 are shown in a parallel orientation (the amino terminals are the 5′ ends to the left and the carboxy terminals are the 3′ ends to the right).

In certain embodiments, the assembly unit can have a randomly coiled peptide that comprises a functional element, F, in the internal or center portion of the dimer (FIG. 19A) or at the end of the PNA molecule opposite the end comprising the joining element. The two functional elements may be the same or different. The joining elements are designed to obviate uncontrolled assembly to allow for staged assembly using such an assembly unit. In this illustration, at least two complementary pairs of PNA sequences are used. There must be no self-complementation or cross-complementation between the joining pairs.

FIG. 20 shows the order of elements of the upper synthetic protein monomer forming the staged assembly subunit shown in FIG. 19A. The order of the elements in the corresponding lower unit would be identical except that the PNA element is at the C-terminus. This reflects the parallel arrangement of the leucine zippers aligning the two units. The functionality sequence encodes the region at which a functional element may be added to the assembly subunit. Glycines separate each element to reduce steric interference between elements. Numbers below the line indicate the typical length in residues of each element.

Formation of a PNA/oligopeptide assembly unit structure may be monitored using the same methodologies commonly known in the art that are used for monitoring protein folding. For example, the oligopeptide portion can be modeled with software that predicts the formation of coiled-coils, e.g. Multicoil (Wolf et al., 1997, MultiCoil: A program for predicting two- and three-stranded coiled coils, Protein Science 6: 1179-89), Paircoil (Berger et al., 1995, Predicting coiled coils by use of pairwise residue correlations, Proc. Natl. Acad. Sci. USA, 92: 8259-63), COILS (Lupas et al., 1991, Predicting coiled coils from protein sequences, Science 252: 1162-64; Lupas, 1996, Prediction and analysis of coiled-coil structures, Meth. Enzymology 266: 513-25) and Macstripe (Lupas et al, 1991, Predicting Coiled Coils from Protein Sequences, Science 252: 1162-64). Standard techniques such as measurement of circular dichroism (CD), e.g., a CD spectrum, can also be used to monitor oligopeptide folding. Moreover, modeling of formation of a joining pair comprising PNA joining elements follows the same rules as DNA-DNA complementary pairing. PNA joining pairs are preferably evaluated using any of a variety of commercial software packages, e.g., Amplify (University of Wisconsin, Madison Wis.), Vector NTI (InforMax, Bethesda Md.), and GCG Wisconsin Package (Accelrys Inc., Burlington Mass.).

PNA/oligopeptide assembly units differ from those derived from pilin proteins or from immunoglobulins, as disclosed herein, in several aspects. PNA/oligopeptide assembly units are hybrids of two different classes of biological molecules—PNA and oligopeptide—and are, therefore, chemically synthesized rather than biologically synthesized. Accordingly, a strict level of quality control and testing for each batch of such PNA-containing assembly units is required. These tests include, e.g., sandwich ELISAs and tests for circular dichroism for protein/protein interactions, evaluation of melting temperatures for PNA joining elements, and SDS-PAGE for determining the percent of full-length molecules.

The α-helical oligopeptide portion of an assembly unit is about 1 nm long per heptad repeat in embodiments where, for example, leucine zipper protein domains are used as structural elements in the construction of an assembly unit (Harbury et al., 1994, Crystal structure of an isoleucine-zipper trimer, Nature 371: 80-83). In embodiments in which an assembly unit has four to six heptads (28-42 amino acids), the structural element is about 4-6 nm long. The PNA joining element is structurally similar to DNA and has a length of about 0.34 nm/base. Therefore, in certain embodiments, a joining element of 10-18 residues will be about 3 to 6 nm in length and, therefore, such an assembly unit will be about 7-12 nm long.

PNA/oligopeptide assembly units also differ from other embodiments of the invention disclosed herein in that they are generally less rigid.

In a specific embodiment, a PNA-peptide assembly unit has a structural element comprising a leucine zipper structure. Such a PNA-peptide assembly unit has an alpha helical portion that has some flexibility although, in certain embodiments, the presence of two or three helix bundles is not as flexible as an isolated a-helical coil. The PNA portion is relatively flexible, so that a structure assembled according to the staged assembly method of the invention from these units may be more analogous to a string of soft beads than to a rigid rod. In addition, a flexible domain (e.g., a tri-, tetra- or pentaglycine) which, in certain embodiments, links joining elements to structural elements, will add to the flexibility of the assembly unit and higher order structures. Two- and three-dimensional nanostructures made of these units are somewhat flexible as free units. However, upon attachment at multiple points to a solid support or matrix, the nanostructure can be made rigid by applying tension to the overall structure, in a manner analogous to the stiffening of a rope net or a spider web by application of a tensioning force.

The coiled coil structural elements also allow for flexibility in the design and construction of assembly units and the nanostructures fabricated from those assembly units. Generally, simple leucine zipper type coiled coils, as disclosed above, are not stable enough to hold the assembly units together by themselves but are stabilized by disulfide bridges (see above). Four helical bundles that are found, for example, in the Rop protein, are generally stable enough, at normal room temperature and can be lengthened, as needed, to provide the stability that is required for formation of assembly units. In addition, the distance between functional elements can be adjusted by changing the length of the coiled coils and by adding flexible peptide segments between, e.g., joining and functional elements. This would lead, in certain embodiments, to a flexible nanostructure more akin to a beads-on-a-string type of architecture.

Because the PNA/protein assembly molecule shares a common backbone, it can be synthesized as a single molecule. It is unnecessary to join the two components together after they are synthesized separately. Custom, contract PNA/protein synthesis is available commercially from PerSeptive Biosystems (division of Applied Biosystems, Framingham Mass.).

The sequence of each PNA joining element is critical to correct assembly. While designing complementary pairs is relatively easy to those skilled in the art, it is important to ascertain that there is no complementary base pairing between PNAs that will be part of the same assembly unit. There are a variety of DNA software packages known to skilled in the art, that can be used to analyze nucleotide sequences for complementarity, e.g., Amplify (University of Wisconsin, Madison Wis.), Vector NTI (InforMax, Bethesda Md.), and GCG Wisconsin Package (Accelrys Inc., Burlington Mass.). PNA segments that have internal complementarity can form hairpin loops and are preferably avoided according to the staged-assembly methods disclosed herein.

Table 9 below lists exemplary PNA sequences that can be comprised in joining elements in PNA/protein assembly units, and gives examples of usable and unusable sequences. In preferred embodiments, one member of the PNA joining pair is attached to a single assembly unit. The corresponding member of the joining pair is the direct complementary sequence, and is attached to another assembly unit. The sequences in Table 9 are listed in amino to carboxy (5′ to 3′) orientation.

TABLE 9

PNA Sequences for Use as Joining Elements in PNA/Protein Assembly
Units

| | |
|---|---|
| Complementary binding pair 1 | Complementary binding pair 2 |

| | | | |
|---|---|---|---|
| A | A′ | B | B′ |

| | | | |
|---|---|---|---|
| *gggggggggg | cccccccccc* | *aaaaaaaaaa | tttttttttt |
| (SEQ ID NO: 91) | (SEQ ID NO: 92) | (SEQ ID NO: 93) | (SEQ ID NO: 94) |
| *gggggttttt | cccccaaaaa* | *ttttttggggg | aaaaaccccc* |
| (SEQ ID NO: 95) | (SEQ ID NO: 96) | (SEQ ID NO: 97) | (SEQ ID NO: 98) |
| *acacacacac | tqtqtgtgtg* | *tctctctctc | agagaqagag* |
| (SEQ ID NO: 99) | (SEQ ID NO: 100) | (SEQ ID NO: 101) | (SEQ ID NO: 102) |
| *atagacagat | tatctgtcta* | *cgctgagatg | gcgactctac* |
| (SEQ ID NO: 103) | (SEQ ID NO: 104) | (SEQ ID NO: 105) | (SEQ ID NO: 106) |
| *aacaqctaac | ttgtcgattg* | *tttggatatg | aaacctatac* |
| (SEQ ID NO: 107) | (SEQ ID NO: 108) | (SEQ ID NO: 109) | (SEQ ID NO: 110) |
| *gttctqgtaa | caagaccatt* | *ttttgcgaac | aaaacgctta* |
| (SEQ ID NO: 111) | (SEQ ID NO: 112) | (SEQ ID NO: 113) | (SEQ ID NO: 114) |
| *ctcaatttgc | gagttaaacg* | *tggggatgtt | acccctacaa* |
| (SEQ ID NO: 115) | (SEQ ID NO: 116) | (SEQ ID NO: 117) | (SEQ ID NO: 118) |
| *cacacaggaa | gtgtgtcctt* | *acagctatga | tgtcgatact* |
| (SEQ ID NO: 119) | (SEQ ID NO: 120) | (SEQ ID NO: 121) | (SEQ ID NO: 122) |
| *gagcctccag | ctcggaggtc* | *ttgttgaacc | aacaacttgg* |
| (SEQ ID NO: 123) | (SEQ ID NO: 124) | (SEQ ID NO: 125) | (SEQ ID NO: 126) |
| *gggtgcagqt | cccacgtcca* | *tcatttqctt | agtaaacqaa* |
| (SEQ ID NO: 127) | (SEQ ID NO: 128) | (SEQ ID NO: 129) | (SEQ ID NO: 130) |
| *ccaagttcac | ggttcaagtg* | *gctttatcca | cgaaataggt* |
| (SEQ ID NO: 131) | (SEQ ID NO: 132) | (SEQ ID NO: 133) | (SEQ ID NO: 134) |
| *cgggtacggt | gcccatgcca* | *cagaatgact | gtcttactga* |
| (SEQ ID NO: 135) | (SEQ ID NO: 136) | (SEQ ID NO: 137) | (SEQ ID NO: 138) |
| *ccccaagcat | ggggttcgta* | *cagaatgact | gtcttactga* |
| (SEQ ID NO: 139) | (SEQ ID NO: 140) | (SEQ ID NO: 141) | (SEQ ID NO: 142) |

Compatible binding element pairs (for two assembly units having the general form of A . . . B′ and B . . . A′; *represents the remainder of the assembly unit).

Complementary binding pairs forming triple helices. “OOOO” represents residues with no base, essentially glycines that allow the PNA to fold back on itself to form the triple helix.

| A | A′ |
|---|---|
| *cccccccOOOOccccccc | ggggggg* |
| (SEQ ID NO: 143) | (SEQ ID NO: 144) |
| *cccttttOOOOttttccc | gggaaaa* |
| (SEQ ID NO: 145) | (SEQ ID NO: 146) |
| *tctctctOOOOtctctct | agaqaga* |
| (SEQ ID NO: 147) | (SEQ ID NO: 148) |
| *cttcctcOOOOctccttc | qaaggag* |
| (SEQ ID NO: 149) | (SEQ ID NO: 150) |

Sequences Unsuitable As Binding Elements

Sequences With Cross-complementation (Complementary Sequences Underlined)

| A | B′ |
|---|---|
| *ggactatgtt | gatacaagat* |
| (SEQ ID NO: 151) | (SEQ ID NO: 152) |
| *tctgtattgg | ataacctgac* |
| (SEQ ID NO: 153) | (SEQ ID NO: 154) |

Sequences Forming Hairpin Loops

*gggttttccc
(SEQ ID NO: 155)
*gatcttggtc
(SEQ ID NO: 156)

FIGS. 19(A-B) contains line diagrams of two possible embodiments of synthetic molecules that can be used in the construction of an assembly unit useful for the present staged assembly methods. As shown in FIGS. 19(A-B), two PNA/oligopeptide units can dimerize to form a single assembly unit. Two possible assembly units are shown in FIG. 19A and FIG. 19B. The PNA portion provides joining elements A and B′, while the oligopeptide portion forms two coiled coil structural elements (S) stabilized by disulfide bonds at either end. One or more functional units (F), comprised of, e.g., protein segments, may also be incorporated into the assembly unit. In certain embodiments, the assembly unit can have a randomly coiled peptide that comprises a functional element, F, in the internal or center portion of the dimer (FIG. 19A) or at the end of the PNA molecule opposite the end comprising the joining element (FIG. 19B).

In this example, the order of elements (i.e., joining structural, and/or functional elements) in the corresponding next assembly unit (i.e., one to be added next during staged assembly) would be identical, except that the PNA element would be at the C-terminus. This reflects the parallel arrangement of the leucine zippers. Glycines separate each element to reduce steric interference between elements.

### 5.6.8. Methods for Characterizing Joining Elements

### 5.6.8.1. Methods for Identifying Joining-Element Interactions by Antibody-Phage-Display Technology

In certain embodiments of the invention, joining elements suitable for use in the methods of the invention are screened and their interactions identified using antibody-phage-display technology. Phage-display technology for production of recombinant antibodies, or binding derivatives or binding fragments thereof, can be used to produce proteins capable of binding to a broad range of diverse antigens, both organic and inorganic (e.g. proteins, peptides, nucleic acids, sugars, and semiconducting surfaces, etc.). Methods for phage-display technology are well known in the art (see, e.g., Marks et al., 1991, By-passing immunization: human antibodies from V-gene libraries displayed on phage, J. Mol. Biol. 222: 581-97; Nissim et al., 1994, Antibody fragments from a “single pot” phage display library as immunochemical reagents, EMBO J. 13: 692-98; De Wildt et al., 1996, Characterization of human variable domain antibody fragments against the U1 RNA-associated A protein, selected from a synthetic and patient derived combinatorial V gene library, Eur. J. Immunol. 26: 629-39; De Wildt et al., 1997, A new method for analysis and production of monoclonal antibody fragments originating from single human B-cells, J. Immunol. Methods. 207: 61-67; Willems et al., 1998, Specific detection of myeloma plasma cells using anti-idiotypic single chain antibody fragments selected from a phage display library, Leukemia 12: 1295-1302; van Kuppevelt et al., 1998, Generation and application of type-specific anti-heparin sulfate antibodies using phage display technology, further evidence for heparin sulfate heterogeneity in the kidney, J. Biol. Chem. 273: 12960-66; Hoet et al., 1998, Human monoclonal autoantibody fragments from combinatorial antibody libraries directed to the U1snRNP associated U1C protein, epitope mapping, immunolocalization and V-gene usage, Mol. Immunol. 35: 1045-55).

Whereas recombinant antibody technology permits the isolation of antibodies with known specificity from hybridoma cells, it does not allow for the rapid creation of specific mAbs. Separate immunizations, followed by cell fusions to generate hybridomas are required to generate each mAb of interest. This can be time consuming as well as laborious.

In preferred embodiments, antibody-phage-display technology is used to overcome these limitations, so that mAbs that recognize particular antigens of interest can be generated more effectively (for methods, see Winter et al., 1994, Making antibodies by phage display technology, Ann. Rev. Immunol. 12: 433-55; Hayashi et al., 1995, A single expression system for the display, purification and conjugation of single-chain antibodies, Gene 160(1): 129-30; McGuinness et al., 1996, Phage diabody repertoires for selection of large numbers of bispecific antibody fragments, Nat. Biotechnol. 14(9): 1149-54; Jung et al., 1999, Selection for improved protein stability by phage display, J. Mol. Biol. 294(1): 163-80;Viti et al., 2000, Design and use of phage display libraries for the selection of antibodies and enzymes, Methods Enzymol. 326: 480-505). Generally, in antibody-phage-display technology, the Fv or Fab antigen-binding portions of V_{L} and the V_{H} genes are “rescued” by PCR amplification using the appropriate primers, from cDNA derived from human spleen or human peripheral blood lymphocyte cells. The rescued V_{L} and the V_{H} gene repertoires (DNA sequences) are spliced together and inserted into the minor coat protein of a bacteriophage (e.g., M13 or fd, or a binding derivative thereof) to create a fusion bacteriophage coat protein (Chang et al., 1991, Expression of antibody Fab domains on bacteriophage surfaces. Potential use for antibody selection, J. Immunol. 147(10): 3610-14; Kipriyanov and Little, 1999, Generation of recombinant antibodies, Mol. Biotechnol. 12(2): 173-201). The resulting bacteriophage contain a functional antibody fused to the outer surface of the phage protein coat and a copy of the gene fragment encoding the antibody V_{L} and V_{H} incorporated into the phage genome.

Using these methods, bacteriophage displaying antibodies that have affinity towards a particular antigen of interest can be isolated by, e.g., affinity chromatography, via the binding of a population of recombinant bacteriophage carrying the displayed antibody to a target epitope or antigen, which is immobilized on a solid surface or matrix. Repeated cycles of binding, removal of unbound or weakly-bound phage particles, and phage replication yield an enriched population of bacteriophage carrying the desired V_{L} and V_{H} gene fragments.

Antigens of interest may include peptides, proteins, immunoglobulin constant regions, CDRs (for production of anti-idiotypic antibodies) other macromolecules, haptens, small molecules, inorganic particles and surfaces.

Once purified, the linked V_{L} and V_{H} gene fragments can be rescued from the bacteriophage genome by standard DNA molecular techniques known in the art, cloned and expressed. The number of antibodies created by this method is directly correlated to the size and diversity of the gene repertoire and offers an optimal method by which to create diverse antibody libraries that can be screened for antigenicity towards virtually any target molecule. mAbs that have been created by antibody-phage-display technology often demonstrate specific binding towards antigen in the picomolar to nanomolar range (Sheets et al., 1998, Efficient construction of a large nonimmune phage antibody library: the production of high-affinity human single-chain antibodies to protein antigens, Proc. Natl. Acad. Sci. USA 95(11): 6157-62).

Antibodies, or binding derivatives or binding fragments thereof, that are useful in the methods of the invention may be selected using an antibody or fragment phage display library constructed and characterized as described above. Such an approach has the advantage of providing methods for efficiently screening a library having a high complexity (e.g. 10⁹), so as to dramatically increase identification of antibodies or fragments suitable for use in the methods of the invention.

In certain embodiments, methods for cloning an immunoglobulin repertoire (“repertoire cloning”) are used to produce an antibody for use in the staged-assembly methods of the invention. Repertoire cloning may be used for the production of virtually any kind of antibody without involving an antibody-producing animal. Methods for cloning an immunoglobulin repertoire (“repertoire cloning”) are well known in the art, and can be performed entirely in vitro. In general, to perform repertoire cloning, messenger RNA (mRNA) is extracted from B lymphocytes obtained from peripheral blood. The mRNA serves as a template for CDNA synthesis using reverse transcriptase and standard protocols (see, e.g., Clinical Gene Analysis and Manipulation, Tools, Techniques and Troubleshooting, Sections IA, IC, IIA, IIB, IIC and IIIA, Editors Janusz A. Z. Jankowski, Julia M. Polak, Cambridge University Press 2001; Sambrook et al., 2001, Molecular Cloning, A Laboratory Manual, Third Edition, Chapters 7, 11, 14 and 18, Cold Spring Harbor Laboratory Press, N.Y.; Ausubel et al., 1989, Current Protocols in Molecular Biology, Chapters 3, 4, 11, 15 and 24, Green Publishing Associates and Wiley Interscience, NY). Immunoglobulin cDNAs are specifically amplified by PCR, using the appropriate primers, from this complex mixture of cDNA. In order to construct immunoglobulin fragments with the desired binding properties, PCR products from genes encoding antibody light (L) and heavy (H) chains are obtained. The products are then introduced into a phagemid vector. Cloned genes or gene fragments incorporated into the bacteriophage genome as fusions with a phage coat protein, are expressed in a suitable bacterial host leading to the synthesis of a hybrid scFv immunoglobulin molecule that is carried on the surface of the bacteriophage. Therefore the bacteriophage population represents a mixture of immunoglobulins with all specificities included in the repertoire.

Antigen-specific immunoglobulin is selected from this population by an iterative process of antigen immunoadsorption followed by phage multiplication. A bacteriophage specific only for an antigen of interest will remain following multiple rounds of selection, and may be introduced into a new vector and/or host for further engineering or to express the phage-encoded protein in soluble form and in large amounts.

Antibody phage display libraries can thus be used, as described above, for the isolation, refinement, and improvement of epitope-binding regions of antibodies that can be used as joining elements in the construction of assembly units for use in the staged assembly of nanostructures, as disclosed herein.

### 5.6.8.2. Methods for Characterizing Joining-Element Interactions using X-Ray Crystallography

In many instances, molecular recognition between proteins or between proteins and peptides may be determined experimentally. In one aspect of the invention, the protein-protein interactions that define the joining element interactions, and are critical for formation of a joining pair are characterized and identified by X-ray crystallographic methods commonly known in the art. Such characterization enables the skilled artisan to recognize joining pair interactions that may be useful in the compositions and methods of the present invention.

### 5.6.8.3. Methods for Characterizing Joining-Element Specificity and Affinity

Verification that two complementary joining elements interact with specificity may be established using, for example, ELISA assays, analytical ultracentrifugation, or BIAcore methodologies (Abraham et al., 1996, Determination of binding constants of diabodies directed against prostate-specific antigen using electrochemiluminescence-based immunoassays, J. Mol. Recognit. 9(5-6): 456-61; Atwell et al., 1996, Design and expression of a stable bispecific scFv dimer with affinity for both glycophorin and N9 neuraminidase, Mol. Immunol. 33(17-18): 1301-12; Muller et al. 1998), A dimeric bispecific miniantibody combines two specificities with avidity, FEBS Lett. 432(1-2): 45-49), or other analogous methods well known in the art, that are suitable for demonstrating and/or quantitating the strength of intermolecular binding interactions.

### 5.7. Functional Elements

A “functional element,” as defined herein, is a moiety exhibiting any desirable physical, chemical or biological property that may be placed, through specific interactions at well-defined sites in a nanostructure. In certain embodiments, any part of an assembly, initiator or capping unit may comprise a functional elements, including, but not limited to, part of the structural element or part of a joining element of a complementary joining pair. Functional elements may be incorporated into assembly units and, ultimately into one-, two-, and three-dimensional nanostructures in such a manner as to provide well-defined spatial relationships between and among the functional elements. These well-defined spatial relationships between and among the functional elements permit them to act in concert to provide activities and properties that are not attainable individually or as unstructured mixtures.

In one aspect of the invention, functional elements include, but are not limited to, peptides, proteins, protein domains, small molecules, inorganic nanoparticles, atoms, clusters of atoms, magnetic, photonic or electronic nanoparticles. The specific activity or property associated with a particular functional element, which will generally be independent of the structural attributes of the assembly unit to which it is attached, can be selected from a very large set of possible functions, including but not limited to, a biological property such as those conferred by proteins (e.g., a transcriptional, translational, binding, modifying or catalyzing property). In other embodiments, functional groups may be used that confer chemical, organic, physical electrical, optical, structural, mechanical, computational, magnetic or sensor properties to the assembly unit.

In another aspect of the invention, functional elements include, but are not limited to: metallic or metal oxide nanoparticles (Argonide Corporation, Sanford, Fla.; NanoEnergy Corporation, Longmont, Col.; Nanophase Technologies Corporation, Romeoville, Ill.; Nanotechnologies, Austin, Tex.; TAL Materials, Inc., Ann Arbor, Mich.); gold or gold-coated nanoparticles (Nanoprobes, Inc., Yaphank, N.Y.; Nanospectra LLC, Houston Tex.); immunoconjugates (Nanoprobes, Inc., Yaphank, N.Y.); non-metallic nanoparticles (Nanotechnologies, Austin, Tex.); ceramic nanofibers (Argonide Corporation, Sanford, Fla.); fullerenes or nanotubes (e.g., carbon nanotubes) (Materials and Electrochemical Research Corporation, Tucson, Ariz.; Nanolab, Brighton Mass.; Nanosys, Inc., Cambridge Mass.; Carbon Nanotechnologies Incorporated, Houston, Tex.); nanocrystals (NanoGram Corporation, Fremont, Calif.; Quantum Dot Corporation, Hayward Calif.); silicon or silicate nanocrystals or powders (MTI Corporation, Richmond, Calif.); nanowires (Nanosys, Inc., Cambridge Mass.); or quantum dots (Quantum Dot Corporation, Hayward Calif.; Nanosys, Inc., Cambridge Mass.).

Functional elements may also comprise any art-known detectable marker, including radioactive labels such as ³²P, ³⁵S, ³H, and the like; chromophores; fluorophores; chemiluminescent molecules; or enzymatic markers.

In certain embodiment of this invention, a functional element is a fluorophore. Exemplary fluorophore moieties that can be selected as labels are set forth in Table 10.

TABLE 10
Fluorophore Moieties That Can Be Used as Functional Elements

4-acetamido-4′-isothiocyanatostilbene-2,2′disulfonic acid
acridine and derivatives:
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
(Lucifer Yellow VS)-(4-anilino-1-naphthyl)maleimide
anthranilamide
Brilliant Yellow
coumarin and derivatives:
coumarin
7-amino-4-methylcoumarin (AMC, Coumarin 120)
7-amino-4-trifluoromethylcoumarin (Coumarin 151)
Cy3
Cy5
cyanosine
4′,6-diaminidino-2-phenylindole(DAPI)
5′,5″-dibromopyrogallol-sulfonephthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansyl
chloride)
4-(4′-dimethylaminophenylazo)benzoic acid (DABCYL)
4-dimethylaminophenylazophenyl-4′-isothiocyanate (DABITC)
eosin and derivatives:
eosin
eosin isothiocyanate
erythrosin and derivatives:
erythrosin B
erythrosin isothiocyanate
ethidium
fluorescein and derivatives:
5-carboxyfluorescein (FAM)
5-(4,6-dichlorotriazin-2-yl)aminofluorescein (DTAF)
2′7′-dimethoxy-4′5′-dichloro-6-carboxyfluorescein (JOE)
fluorescein
fluorescein isothiocyanate
QFITC (XRITC)
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:
pyrene
pyrene butyrate
succinimidyl 1-pyrene butyrate
Reactive Red 4 (Cibacron ® Brilliant Red 3B-A)
rhodamine and derivatives:
6-carboxy-X-rhodamine (ROX)
6-carboxyrhodamine (R6G)
lissamine rhodamine B sulfonyl chloride
rhodamine (Rhod)
rhodamine B
rhodamine 110
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
terbium chelate derivatives

In other embodiments, a functional element is a chemiluminescent substrate such as luminol (Amersham Biosciences), BOLD™ APB (Intergen), Lumigen APS (Lumigen), etc.

In another embodiment, the functional element is an enzyme. The enzyme, in certain embodiments, may produce a detectable signal when a particular chemical reaction is conducted, such as the enzymes alkaline phosphatase, horseradish peroxidase, P-galactosidase, etc.

In another embodiment, a functional element is a hapten or an antigen (e.g., ras). In yet another embodiment, a functional element is a molecule such as biotin, to which a labeled avidin molecule or streptavidin may be bound, or digoxygenin, to which a labeled anti-digoxygenin antibody may be bound.

In another embodiment, a functional element is a lectin such as peanut lectin or soybean agglutinin. In yet another embodiment, a functional element is a toxin, such as Pseudomonas exotoxin (Chaudhary et al., 1989, Å recombinant immunotoxin consisting of two antibody variable domains fused to Pseudomonas exotoxin, Nature 339(6223): 394-97).

Peptides, proteins or protein domains may be added to proteinaceous assembly units using the tools of molecular biology commonly known in the art to produce fusion proteins in which the functional elements are introduced at the N-terminus of the proteins, the C-terminus of the protein, or in a loop within the protein in such a way as to not disrupt folding of the protein. Non-peptide functional elements may be added to an assembly unit by the incorporation of a peptide or protein moiety that exhibits specificity for said functional element, into the proteinaceous portion of the assembly unit.

In certain embodiments, a functional unit is attached by splicing a protein domain or peptide into the proteinaceous portion of an assembly unit. In such embodiments, the position for insertion must be chosen such that it does not disrupt the folding of the protein unit, since the binding specificity and affinity of the assembly unit will depend on the ability of the assembly unit to fold correctly. Also preferably, the site at which an insert is added does not cause disruption of the folding of the protein unit. Preferably, the site of insertion is a surface loop having little interaction with the remainder of the protein. When the three-dimensional structure of the protein is known, e.g., in the case of the pilin papK, such sites may be identified by visual examination of the protein structure using a computer graphics program, such as RasMol (Sayle et al., 1995, RasMol: Biomolecular graphics for all, Trends Biochem. Sci. (TIBS) 20(9): 374-76). The coordinates defining the three-dimensional positions of the atoms of papK are included in the PDB file 1PDK, which also provides the three-dimensional structure of the chaperone papD that is complexed with papK in the solved crystal structure. Upon such an analysis, it is apparent that there is a surface loop that includes residues 109-113 (sequence NKGQGE (SEQ ID NO: 157) according to the PDB file), which represents a site with high potential for accepting the insertion of a peptide such as the ras antigen.

In a specific embodiment, one or more functional elements is added to an assembly unit comprising a pilin protein at a position identified as being (i) on the surface of the unit; (ii) unimportant to the interaction of the unit with other pilin-comprising assembly unit; and (iii) unimportant for the stability of the unit itself. It has been shown that large loop insertions are tolerated and many recombinant proteins have been expressed that are able to fold successfully into stable, active protein structures. In some instances, such recombinant proteins have been designed and produced without further genetic manipulation, while other approaches have incorporated a randomization and selection step to identify optimal sequence alterations (Regan, 1999, Protein redesign, Curr. Opin. Struct. Biol. 9: 494-99). For example, one pilin region amenable to re-engineering is a surface loop on papA comprising the sequence gly107-ala108-gly109. This loop satisfies all the above-described criteria as a position at which a heterologous peptide may be inserted.

In another embodiment, an entire antibody variable domain (e.g. a single-chain variable domain) is incorporated into an assembly unit, e.g. into the joining or structural element thereof, in order to act as an affinity target for a functional element. In this embodiment, wherein an entire antibody variable domain is inserted into a surface loop of, e.g., a joining element or a structural element, a flexible segment (e.g., a polyglycine peptide sequence) is preferably added to each side of the variable domain sequence. This polyglycine linker will act as a flexible spacer that facilitates folding of the original protein after synthesis of the recombinant fusion protein. The antibody domain is chosen for its binding specificity for a functional element, which can be, but is not limited to, a protein or peptide, or to an inorganic material.

In another embodiment of the present invention, a functional element may be a quantum dot (semiconductor nanocrystal, e.g., QDOT™, Quantum Dot Corporation, Hayward, Calif.) with desirable optical properties. A quantum dot can be incorporated into a nanostructure through a peptide that has specificity for a particular class of quantum dot. As would be apparent to one of ordinary skill, identification of such a peptide, having a required affinity for a particular type of quantum dot, is carried out using methods well known in the art. For example, such a peptide is selected from a large library of phage-displayed peptides using an affinity purification method. Suitable purification methods include, e.g., biopanning (Whaley et al., 2000, Selection of peptides with semiconductor binding specificity for directed nanocrystal assembly, Nature 405(6787): 665-68) and affinity column chromatography. In each case, target quantum dots are immobilized and the recombinant phage display library is incubated against the immobilized quantum dots. Several rounds of biopanning are carried out and phage exhibiting affinity for the quantum dots are identified by standard methods after which the specificity of the peptides are tested using standard ELISA methodology.

Typically, the affinity purification is an iterative process that uses several affinity purification steps. Affinity purification may been used to identify peptides with affinity for particular metals and semiconductors (Belcher, 2001, Evolving Biomolecular Control of Semiconductor and Magnetic Nanostructure, presentation at Nanoscience: Underlying Physical Concepts and Properties, National Academy of Sciences, Washington, D.C., May 18-20, 2001; Belcher et al., 2001, Abstracts of Papers, 222nd ACS National Meeting, Chicago, Ill., United States, Aug. 26-30, 2001, American Chemical Society, Washington, D.C.).

An alternate method is directed toward the use of libraries of phage-displayed single chain variable domains, and to carry out the same type of selection process. Accordingly, in certain embodiments, a functional element is incorporated into a nanostructure through the use of joining elements (interaction sites) by which non-proteinaceous nanoparticles having desirable properties are attached to the nanostructure. Such joining elements are, in two non-limiting examples, derived from the complementarity determining regions of antibody variable domains or from affinity selected peptides.

Routine tests for electronic and photonic functional elements that are commonly used to compare the electronic properties of nanocrystals (single nanoparticles) and assemblies of nanoparticles (Murray et al., 2000, Synthesis and characterization of monodisperse nanocrystals and close-packed nanocrystal assemblies, Ann. Rev. Material Science 30: 545-610), are used for the analysis of nanostructures fabricated using the compositions and methods disclosed herein.

In certain embodiments, the unique, tunable properties of semiconductor nanocrystals make them preferable for use in nanodevices, including photoconductive nanodevices and light emitting diodes. The electrical properties of an individual nanostructure are difficult to measure, and therefore, photoconductivity is used as a measure of the properties of those nanostructures. Photoconductivity is a well-known phenomena used for analysis of the properties of semiconductors and organic solids. Photoconductivity has long been used to transport electrons between weakly interacting molecules in otherwise insulating organic solids.

Photocurrent spectral responses may also be used to map the absorption spectra of the nanocrystals in nanostructures and compared to the photocurrent spectral responses of individual nanocrystals (see, e.g., Murray et al., 2000, Synthesis and characterization of monodisperse nanocrystals and close-packed nanocrystal assemblies, Ann. Rev. Material Science 30: 545-610). In addition, optical and photoluminescence spectra may also be used to study the optical properties of nanostructures (see, e.g., Murray et al., 2000, Synthesis and characterization of monodisperse nanocrystals and close-packed nanocrystal assemblies, Ann. Rev. Material Science 30: 545-610).

The greater the control exerted over the formation of arrays of nanoparticles, the wider the array of optical, electrical and magnetic phenomena that will be produced. With staged assembly of nanostructures into which nanoparticles are incorporated with three-dimensional precision, it is possible to control the properties of solids formed therefrom in three dimensions, thereby giving rise to a host of anisotropic properties useful in the design of nanodevices. Routine tests and methods for characterizing the properties of these assemblages are well-known in the art (see, e.g., Murray et al., 2000, Synthesis and characterization of monodisperse nanocrystals and close-packed nanocrystal assemblies, Ann. Rev. Material Sci. 30: 545-610).

For example, biosensors are commercially available that are made of a combination of proteins and quantum dots (Alivisatos et al., 1996, Organization of ‘nanocrystal molecules’ using DNA, Nature 382: 609-11; Weiss et al., U.S. Pat. No. 6,207,392 entitled “Semiconductor nanocrystal probes for biological applications and process for making and using such probes,” issued Mar. 27, 2001). The ability to complex a quantum dot with a highly specific biological molecule (e.g., a single stranded DNA or an antibody molecule) provides a spectral fingerprint for the target of the molecule. Using different sized quantum dots (each with very different spectral properties), each complexed to a molecule with different specificity, allows multiple sensing of components simultaneously.

Inorganic structures such as quantum dots and nanocrystals of metals or semiconductors may be used in the staged assembly of nanostructures as termini of branches of the assembled nanostructure. Once such inorganic structures are added, additional groups cannot be attached to them because they have an indeterminate stoichiometry for any set of binding sites engineered into a nanostructure. This influences the sequence in which assembly units are added to form a nanostructure being fabricated by staged assembly. For example, once a particular nanocrystal is added to the nanostructure, it is generally not preferred to add additional assembly units with joining elements that recognize and bind that type of nanocrystal, because it is generally not possible to control the positioning of such assembly units relative to the nanocrystal. Therefore, it may be necessary to add the nanocrystals last, or at least after all the assembly units that will bind that particular type of nanocrystal are added. In a preferred embodiment, nanocrystals are added to nanostructures that are still bound to a matrix and are sufficiently separated so that each nanocrystal can only bind to a single nanostructure, thereby preventing multiple cross-linking of nanostructures.

In one embodiment, a rigid nanostructure, fabricated according to the staged assembly methods of the present invention, comprises a magnetic nanoparticle attached as a functional element to the end of a nanostructure lever arm, which acts as a very sensitive sensor of local magnetic fields. The presence of a magnetic field acts to change the position of the magnetic nanoparticle, bending the nanostructure lever arm relative to the solid substrate to which it is attached. The position of the lever arm may be sensed, in certain embodiments, through a change in position of, for example, optical nanoparticles attached as functional elements to other positions (assembly units) along the nanostructure lever arm. The degree of movement of the lever arm is calibrated to provide a measure of the magnetic field.

In other embodiments, nanostructures that are fabricated according to the staged assembly methods of the invention have desirable properties in the absence of specialized functional elements. In such embodiments, a staged assembly process provides a two-dimensional or a three-dimensional nanostructure with small (nanometer-scale), precisely-sized, and well-defined pores that can be used, for example, for filtering particles in a microfluidic system. In further aspects of this embodiment, nanostructures are assembled that not only comprise such well-defined pores but also comprise functional elements that enhance the separation properties of the nanostructure, allowing separations based not only on size but also with respect to the charge and/or hydrophilicity or hydrophobicity properties of the molecules to be separated. Such nanostructures can be used for HPLC separations, providing extremely uniform packing materials and separations based upon those materials. Examples of such functional elements include, but are not limited to, peptide sequences comprising one or more side chains that are positively or negatively charged at a pH used for the desired chromatographic separation; and peptide sequences comprising one or more amino acids having hydrophobic or lipophilic side chains.

Junctions are architectural structures that can serve as “switch points” in microelectronic circuits such as silicon based electronic chips, etc. In certain embodiments, multivalent antibodies or binding derivatives or binding fragments thereof are used as unction structures and are introduced into nanostructures using the methods of the present invention. One non-limiting example of bioelectronic and biocomputational devices comprising these nanostructure junctions are quantum cellular automata (QCA).

### 5.7.1. Functional Elements Comprising Peptide/PNA Fragments

In another embodiment, functional elements (depicted as “F”) comprising peptide sequences are placed in two possible locations in an assembly unit formed by leucine zipper dimerization. Sequences can be added to the opposite end of the peptide from, e.g., a PNA, or can be inserted between two shorter a-helices, as shown in FIG. 19.

Table 11 sets forth several non-limiting, illustrative examples of functional elements.

TABLE 11

Peptides That Can Be Used as Functional Elements in Peptide/PNA
Units

| | |
|---|---|
| Amino acid sequence | Origin/achvity/reference |

Epitopes

| | |
|---|---|
| SGFNADYEASSSRC | human fos |
| (SEQ ID NO: 158) | |
| PIDMESQERIKAERKRM | v-jun |
| (SEQ ID NO: 159) | |
| EQKLISEEDL | c-myc |
| (SEQ ID NO: 160) | |
| EEYSAMRDQYMRTGE | v-H-ras |
| (SEQ ID NO: 161) | |
| QPELAPEDPED | herpes simplex virus |
| (SEQ ID NO: 162) | |
| MASMTGGQQMG | bacteriophage T7 gene 10 |
| (SEQ ID NO: 163) | |
| YGGFL | β-endorphin |
| (SEQ ID NO: 164) | |

Biotin analogues (bind to streptavidin)

| | |
|---|---|
| ISFENTWLWI-IPQFSS | Devlin et al, 1990, Random peptide libraries: A |
| (SEQ ID NO: 165) | source of specific protein binding molecules, |
| | Science 249: 404-406 |
| TPHPQ | Lam et. al., 1991, A new type of synthetic peptide |
| (SEQ ID NO: 166) | library for identifying ligand-binding activity, |
| | Nature 354: 82-84 |
| MHPMA | Lam et. al., 1991, A new type of synthetic peptide |
| (SEQ ID NO: 167) | library for identifying ligand-binding activity, |
| | Nature 354: 82-84 |

His tags (bind to nickel and nickel conjugates)

H₆₋₁₀

Peptides (bind to specific protein targets)

| | |
|---|---|
| KETAAAKFERQHMDS | binds S-protein conjugate |
| (SEQ ID NO: 168) | Richards and Wyckoff, in “The Enzymes” Vol. IV, |
| | P. D. Boyer ed., Academic Press, New York, pp. |
| | 647-806 |
| RRASV | protein kinase A phosphorylation target |
| (SEQ ID NO: 169) | de Arruda and Burgess, 1996, pET-33B(+): A pET |
| | vector that contains a protein kinase A recognition |
| | sequence, Novagen Innovations 4a: 7-8 |
| Peptides (bind to GaAs) | Whaley et al., 2000, Selection of peptides |
| | with semiconductor binding specificity for |
| | directed nanocrystal assembly, Nature 405: |
| | 665-668 |

VTSPDSTTGAMA (SEQ ID NO: 170)
AASPTQSMSQAP (SEQ ID NO: 171)
AQNPSDNNTHTH (SEQ ID NO: 172)
ASSSRSHFGQTD (SEQ ID NO: 173)
WAHAPQLASSST (SEQ ID NO: 174)
ARYDLSIPSSES (SEQ ID NO: 175)
TPPRPIQYNHTS (SEQ ID NO: 176)
SSLQLPENSFPH (SEQ ID NO: 177)
GTLANQQIFLSS (SEQ ID NO: 178)
HGNPLPMTPFPG (SEQ ID NO: 179)
RLELAIPLQGSG (SEQ ID NO: 180)

In one embodiment, the functional element comprises a PNA segment. Just as PNA can be placed at the end of the monomer during synthesis to serve as a joining element, a segment of PNA, comprising residues capable of base-paring, can be placed into the middle of a synthesized peptide subunit to serve as a functional element. This permits the fabrication of a precisely branched nanostructure, or a nanostructure comprising a PNA-conjugated joining element that is precisely attached to the nanostructure by base-pairing interactions with the structural element-embedded PNA functional element. In preferred embodiments, functional elements, and/or bridging cysteine residues, are generally separated from neighboring structural and/or joining elements by a peptide segment of about two to five glycine residues, so that the protein/peptide domains can form independently.

### 5.8. Design and Engineering of Structural, Joining and Functional Elements

Design of structural, joining and functional elements of the invention, and of the assembly units that comprise them, is facilitated by analysis and determination of those amino acid residues in the desired binding interaction, as revealed in a defined crystal structure, or through homology modeling based on a known crystal structure of a highly homologous protein. The crystal structure of, e.g., a pilin-peptide complex, may be used to predict the structure and geometry of pilin-pilin interactions. Although a complex between two pilin proteins has yet to be crystallized, energy calculations and solid-body modeling can be used to predict the structure of a complex made up of multiple pilins (Sauer et al., 1999, Structural basis of chaperone function and pilus biogenesis; Science 285: 1058-1061; Choudhury et al., 1999, X-ray structure of the FimC-FimH chaperone-adhesin complex from uropathogenic Escherichia coli, Science 285: 1061-1066).

Many crystal structures of antibodies interacting with antigens, antigen fragments or other antibodies are available from the Brookhaven Protein Data Bank (Berman et al., 2000, The Protein Data Bank, Nucl. Acids Res. 235-42; 1977, The Protein Data Bank. A computer-based archival file for macromolecular structures, Eur. J. Biochem. 80(2): 319-24) and may be used by ordinarily skilled artisans as guides for predicting the structures of antibody-antigen or antibody-antibody complexes.

Design of a useful assembly unit comprising one or more functional elements preferably involves a series of decisions and analyses that may include, but are not limited to, some or all of the following steps:

(i) selection of the functional elements to be incorporated based on the desired overall function of the nanostructure;

(ii) selection of the desired geometry based on the target function, in particular, determination of the relative positions of the functional elements;

(iii) selection of joining elements through determination, identification or selection of those peptides or proteins, e.g. from a combinatorial library, that have specificity for the functional nanoparticles to be incorporated into the desired nanostructure;

(iv) based on the needed separations between functional elements comprising, e.g. nanoparticles such as quantum dots, etc., selection of structural elements that will provide a suitably rigid structure with correct dimensions and having positions for incorporation of joining elements with the correct geometry and stoichiometry;

(v) design of fusion proteins incorporating peptide or protein joining elements, from step (iii) and the structural element selected in step (iv) such that the folding of the structural and joining elements of the assembly unit are not disrupted (e.g., through incorporation at β-turns);

(vi) computer modeling of the resultant fusion proteins in the context of the overall design of the nanostructure and refining of the design to optimize the structural dimensions as required by the functional specifications; or

(vii) design of the assembly sequence for staged assembly.

Modification of a structural element protein, for example, usually involves insertion, deletion, or modification of the amino acid sequence of the protein in question. In many instances, modifications involve insertions or substitutions to add joining elements not extant in the native protein. A non-limiting example of a routine test to determine the success of an insertion mutation is a circular dichroism (CD) spectrum. The CD spectrum of the resultant fusion mutant protein can be compared to the CD of the native protein.

If the insert is small (e.g., a short peptide), then the spectra of a properly folded insertion mutant will be very similar to the spectra of the native protein. If the insertion is an entire protein domain (e.g. single chain variable domain), then the CD spectrum of the fusion protein should correspond to the sum of the CD spectra of the individual components (i.e. that of the native protein and fusion protein comprising the native protein and the functional element). This correspondence provides a routine test for the correct folding of the two components of the fusion protein. Preferably, a further test of the successful engineering of a fusion protein is made. For example, an analysis may be made of the ability of the fusion protein to bind to all of its targets, and therefore, to interact successfully with all joining pairs. This may be performed using a number of appropriate ELISA assays; at least one ELISA is performed to test the affinity and specificity of the modified protein for each of the joining pairs required to form the nanostructure.

### 5.9. Uses of the Staged-Assembly Method and of Nanostructures Constructed Thereby

The staged-assembly methods and the assembly units of the invention have use in the construction of myriad nanostructures. The uses of such nanostructures are readily apparent and include applications that require highly regular, well-defined arrays of one-, two-, and three-dimensional structures such as fibers, cages, or solids, which may include specific attachment sites that allow them to associate with other materials.

In certain embodiments, the nanostructures fabricated by the staged assembly methods of the invention are one-dimensional structures. For example, nanostructures fabricated by staged assembly can be used for structural reinforcement of other materials, e.g., aerogels, paper, plastics, cement, etc. In certain embodiments, nanostructures that are fabricated by staged assembly to take the form of long, one-dimensional fibers are incorporated, for example, into paper, cement or plastic during manufacture to provide added wet and dry tensile strength.

In another embodiment, the nanostructure is a patterned or marked fiber that can be used for identification or recognition purposes. In such embodiments, the nanostructure may contain such functional elements as e.g., a fluorescent dye, a quantum dot, or an enzyme.

In a further embodiment, a particular nanostructure is impregnated into paper and fabric as an anti-counterfeiting marker. In this case, a simple color-linked antibody reaction (such as those commercially available in kits) is used to verify the origin of the material. Alternatively, such a nanostructure could bind dyes, inks or other substances, either before or after incorporation, to color the paper or fabrics or to modify their appearance or properties in other ways.

In another embodiment, nanostructures are incorporated, for example, into ink or dyes during manufacture to increase solubility or miscibility.

In another embodiment, a one-dimensional nanostructure e.g., a fiber, bears one or more enzyme or catalyst functional elements in desired positions. The nanostructure serves as a support structure or scaffold for an enzymatic or catalytic reaction to increase its efficiency. In such an embodiment, the nanostructure may be used to “mount” or position enzymes or other catalysts in a desired reaction order to provide a reaction “assembly line.”

In another embodiment, a one-dimensional nanostructure, e.g., a fiber, is used as an assembly jig. Two or more components, e.g., functional units, are bound to the nanostructure, thereby providing spatial orientation. The components are joined or fused, and then the resultant fused product is released from the nanostructure.

In another embodiment, a nanostructure is a one-, two- or three-dimensional structure that is used as a support or framework for mounting nanoparticles (e.g., metallic or other particles with thermal, electronic or magnetic properties) with defined spacing, and is used to construct a nanowire or nanocircuit.

In another embodiment, the staged assembly methods of the invention are used to accomplish electrode-less plating of a one-dimensional nanostructure (fiber) with metal to construct a nanowire with a defined size and/or shape. For example, a nanostructure could be constructed that comprises metallic particles as functional elements.

In another embodiment, a one-dimensional nanostructure (e.g., a fiber) comprising magnetic particles as functional elements is aligned by an external magnetic field to control fluid flow past the nanostructure. In another embodiment, the external magnetic field is used to align or dealign a nanostructure (e.g., fiber) comprising optical moieties as functional elements for use in LCD-type displays.

In another embodiment, a nanostructure is used as a size standard or marker of precise dimensions for electron microscopy.

In other embodiments, the nanostructures fabricated by the staged assembly methods of the invention are two- or three-dimensional structures. For example, in one embodiment, the nanostructure is a mesh with defined pore size and can serve as a two-dimensional sieve or filter.

In another embodiment, the nanostructure is a three-dimensional hexagonal array of assembly units that is employed as a molecular sieve or filter, providing regular vertical pores of precise diameter for selective separation of particles by size. Such filters can be used for sterilization of solutions (i.e., to remove microorganisms or viruses), or as a series of molecular-weight cut-off filters. In this embodiment, the protein components of the pores, such as structural elements or functional elements, may be modified so as to provide specific surface properties (i.e., hydrophilicity or hydrophobicity, ability to bind specific ligands, etc.). Among the advantages of this type of filtration device is the uniformity and linearity of pores and the high pore to matrix ratio.

It will be apparent to one skilled in the art that the methods and assembly units disclosed herein may be used to construct a variety of two- and three-dimensional structures such as polygonal structures (e.g., octagons), as well as open solids such as tetrahedrons, icosahedrons formed from triangles, and boxes or cubes formed from squares and rectangles (e.g., the cube disclosed in Section 11, Example 6). The range of structures is limited only by the types of joining and functional elements that can be engineered on the different axes of the structural elements.

In another embodiment, a two-or three-dimensional nanostructure may be used to construct a surface coating comprising optical, electric, magnetic, catalytic, or enzymatic moieties as functional units. Such a coating could be used, for example, as an optical coating. Such an optical coating could be used to alter the absorptive or reflective properties of the material coated.

A surface coating constructed using nanostructures of the invention could also be used as an electrical coating, e.g., as a static shielding or a self-dusting surfaces for a lens (if the coating were optically clear). It could also be used as a magnetic coating, such as the coating on the surface of a computer hard drive. Such a surface coating could also be used as a catalytic or enzymatic coating, for example, as surface protection. In a specific embodiment, the coating is an antioxidant coating.

In another embodiment, the nanostructure may be used to construct an open framework or scaffold with optical, electric, magnetic, catalytic, enzymatic moieties as functional elements. Such a scaffold may be used as a support for optical, electric, magnetic, catalytic, or enzymatic moieties as described above. In certain embodiments, such a scaffold could comprise functional elements that are arrayed to form thicker or denser coatings of molecules, or to support soluble micron-sized particles with desired optical, electric, magnetic, catalytic, or enzymatic properties.

In another embodiments, a nanostructure serves as a framework or scaffold upon which enzymatic or antibody binding domains could be linked to provide high density multivalent processing sites to link to and solubilize otherwise insoluble enzymes, or to entrap, protect and deliver a variety of molecular species.

In another embodiment, the nanostructure may be used to construct a high density computer memory with addressable locations.

In another embodiment, the nanostructure may be used to construct an artificial zeolite, i.e., a natural mineral (hydrous silicate) that has the capacity to absorb ions from water, wherein the design of the nanostructure promotes high efficiency processing with reactant flow-through an open framework.

In another embodiment, the nanostructure may be used to construct an open framework or scaffold that serves as the basis for a new material, e.g., the framework may possess a unique congruency of properties such as strength, density, determinate particle packing and/or stability in various environments.

Inc certain embodiments, the staged-assembly methods of the invention can also be used for constructing computational architectures, such as quantum cellular automata (QCA) that are composed of spatially organized arrays of quantum dots. In QCA technology, the logic states are encoded by positions of individual electrons, contained in QCA cells composed of spatially positioned quantum dots, rather than by voltage levels. Staged assembly can be implemented in an order that spatially organizes quantum dot particles in accordance with the geometries necessary for the storage of binary information. Examples of logic devices that can be fabricated using staged assembly for the spatially positioning and construction of QCA cells for quantum dot cellular automata include QCA wires, QCA inverters, majority gates and full adders (Amlani et al., 1999, Digital logic gate using quantum-dot cellular automata, Science 284(5412): 289-91; Cowburn and Welland, 2000), Room temperature magnetic quantum cellular automata, Science 287(5457): 1466-68; Orlov et al., 1997, Realization of a Functional Cell for Quantum-Dot Automata, Science 277: 928-32).

### 6. EXAMPLE 1

### Staged Assembly of Hybrid Pilin Assembly Units

In this example, hybrid pilin assembly units are constructed using the following steps of the staged-assembly methods of the invention.

With the immobilized papA and the hybrid proteins engineered as disclosed above, it is possible to assemble a filament comprising five pilin units and having two ras epitopes positioned, one each, on the second and fifth units in the assembly (FIG. 17).

(1) In the first step, PapA units are immobilized on a solid matrix using methods well known in the art. For example, a biotin moiety may be added to the amino terminus of papA; the papA then incubated in the presence of a surface coated with streptavidin. The very strong interaction of biotin with streptavidin will lead to the immobilization of papA on the surface. Many other methods for the immobilization of a protein on a solid surface are available and well known to those of ordinary skill in the art.

(2) In the second step, a solution of papH-papK hybrid protein displaying the ras epitope is incubated with the immobilized papA. In order to solubilize the papH-papK hybrid, it may be necessary to complex it with the chaperone papD. During incubation, papD will exchange with the immobilized papA to deposit the hybrid protein onto papA. After an appropriate incubation period, generally from seconds to minutes, in most cases, and upwards to several hours in unusual cases, any excess protein is washed off. The product of this step will be a pilin dimer comprising the immobilized papA and the hybrid papH-papK with ras epitope.

(3) In the third step, a solution of papE-papA hybrid protein (possibly in complex with papD) is incubated with the immobilized product of Step 2. After incubation any excess protein is washed off. The result of this step will be a pilin trimer comprising the immobilized papA, the hybrid papH-papK with ras epitope and the hybrid papE-papA protein (FIG. 17).

(4) In the fourth step, a solution of papK-papF hybrid protein is incubated, as described above in Step 3, with the immobilized product of Step 3. After incubation, any excess protein is washed off. This step produces a pilin tetramer comprising the immobilized papA, the hybrid papH-papK with ras epitope, the hybrid papE-papA protein and the hybrid papK-papF protein (FIG. 17).

(5) In the fifth step, a solution of papH-papE hybrid protein (possibly in complex with papD) with inserted ras epitope is incubated the immobilized product of Step 4. After incubation, any excess protein is washed off. The result of this step will be a pilin pentamer comprising the immobilized papA, the hybrid papH-papK with ras epitope, the hybrid papE-papA protein, the hybrid papK-papF protein and the papH-papE hybrid with ras epitope (FIG. 17).

It is possible to construct more complex structures through the continued addition of pilin units in a manner analogous to that used in steps 2-5.

### 7. EXAMPLE 2

### Staged-Assembly of a Nanostructure Having a Joining Element Comprising a Peptide Epitope

This example discloses staged assembly using monovalent Fab fragments (“Fab1” and “Fab2,”) each with a different peptide epitope fused at their C-terminus (FIG. 7).

The CDR of Fab1 has specificity for the peptide fused to the C-terminus of Fab2. Likewise, the CDR of Fab2 has specificity for the peptide fused to the C-terminus of Fab1.

The two joining pairs provide specific interactions between these two assembly units. The first Fab can be immobilized to a solid substrate using standard methods. This surface can then be incubated with a solution containing Fab2 which has fused a peptide exhibiting specificity for Fab1. This incubation will result in the formation of a nanostructure intermediate comprised of one copy of Fab1 (immobilized) and one copy of Fab2. The intermediate can then be incubated against a solution containing Fab1, resulting in the formation of an intermediate comprised of a copy of Fab1 attached to a copy of Fab2 that is sequentially attached to a copy of Fab1. This assembly process may then continue iteratively for as long as is necessary to achieve the size of linear structure required.

Assembly unit-1 is a monovalent assembly unit comprising an antibody Fab fragment with CDR (CDR1) that specifically binds to peptide 2 with a linked C-terminal peptide epitope (peptide 1).

Assembly unit-2 is a monovalent assembly unit comprising an antibody Fab fragment with CDR (CDR2) that specifically binds to peptide 1 with a linked C-terminal peptide epitope (peptide 2).

Joining pairs.

Joining pair 1: Joining element peptide 1 interacts with joining element CDR 2.

Joining pair 2: Joining element peptide 2 interacts with joining element CDR 1.

| Staged Assembly Steps | Procedure |
|---|---|
| Step 1 | a) Add assembly unit-1 |
| | b) Wash |
| Step 2 | a) Add assembly unit-2 |
| | b) Wash |
| Step 3 | a) Repeat Step 1 |
| Step 4 | a) Repeat Step 2 |

### 8. EXAMPLE 3

### Staged Assembly Using Multispecific Protein Assembly Units

This example discloses an embodiment of the staged assembly methods of the invention that uses multispecific protein assembly units. Permutations and combinations of multispecific protein assembly units may be used for the construction of complex one-, two-, and three-dimensional macromolecular nanostructures, including, for example, the staged assembly illustrated in FIG. 21, which utilizes bivalent and tetravalent assembly units.

Staged assembly of a nanostructure comprising a four-point junction only requires a minimum of five assembly units and four joining pairs. The five assembly units required include four bispecific and one tetraspecific assembly unit. In this example, the joining pairs employed to join adjacent assembly units are idiotope/anti-idiotope in nature. A minimum of four such idiotope/anti-idiotope joining pairs are needed for staged-assembly in this example.

### 8.1. Assembly Units

In FIG. 21:

Assembly unit-1 is a bivalent protein assembly unit comprising a non-interacting (idiotope/anti-idiotope) joining pair A and B.

Assembly unit-2 is a bivalent assembly unit comprising a non-interacting idiotope/anti-idiotope) joining pair B′ and A′.

Assembly unit-3 is a tetravalent assembly unit comprising non-interacting (idiotope/anti-idiotope) joining pair B′ and A′ and non-interacting (idiotope/anti-idiotope) joining pair C and D.

Assembly unit-4 is a bivalent assembly unit comprising a non-interacting (idiotope/anti-idiotope)joining pair C′ and A.

Assembly unit-5 is a bivalent assembly unit with non-interacting (idiotope/anti-idiotope) joining pair D′ and B′.

### 8.2. Complementary Joining Pairs

A interacts with A′ in complementary joining pair 1.

B interacts with B′ in complementary joining pair 2.

C interacts with C′ in complementary joining pair 3.

D interacts with D′ in complementary joining pair 4.

### 8.3. Protocol for Staged Assembly Using Multispecific Protein Assembly Units

The following steps of staged assembly are illustrated in FIG. 21. The resultant nanostructure is illustrated FIG. 21, Step 11.

| Staged Assembly Steps | Procedure |
|---|---|
| Step 1 | a) Add assembly unit-1 |
| | b) Wash |
| Step 2 | a) Add assembly unit-2 |
| | b) Wash |
| Step 3 | a) Repeat Step 1 |
| Step 4 | a) Add assembly unit-3 |
| | b) Wash |
| Step 5 | a) Repeat Step 1 |
| Step 6 | a) Add assembly unit-4 |
| | b) Wash |
| Step 7 | a) Repeat Step 2 |
| Step 8 | a) Add assembly unit-5 |
| | b) Wash |
| Step 9 | a) Repeat Step 1 |
| Step 10 | a) Repeat Step 2 |
| Step 11 | a) Repeat Step 1 |

### 9. EXAMPLE 4

### Fabrication of a Macromolecular Nanostructure

To build a macromolecular assembly, two assembled nanostructures intermediates can be joined to one another using the staged assembly methods of the invention. This example describes the fabrication of a macromolecular nanostructure from two nanostructure intermediates.

FIG. 22 illustrates the staged assembly of the two nanostructure intermediates fabricated from the staged assembly protocol illustrated in FIG. 21. Nanostructure intermediate-1 is illustrated as Step-11 in FIG. 21. Nanostructure intermediate-2 is illustrated as Step-8 in FIG. 21. The protocol in Section 9.1 below describes the addition of two nanostructure intermediates by the association of a complementary joining pair.

### 9.1. Protocol for the Addition of Two Nanostructure Intermediates by the Association of a Complementary Joining Pair

The following steps of staged assembly are illustrated in FIG. 22. The resultant macromolecular nanostructure is illustrated FIG. 22, Step 5.

| Staged Assembly Steps | Procedure |
|---|---|
| Step 1 | Steps 1-11 of staged assembly protocol |
| | described above in Section 8 (Example 3) |
| Step 2 | a) Add A' capping unit |
| | b) Wash |
| Step 3 | Remove nanostructure intermediate-1 from |
| | the support matrix and isolate |
| Step 4 | Perform Steps 1-8 of staged assembly |
| | protocol described above in Section 8 |
| | (Example 3), leaving nanostructure |
| | intermediate-2 attached to the support |
| | matrix |
| Step 5 | a) Add nanostructure intermediate-1 |
| | b) Wash |

### 10. EXAMPLE 5

### Demonstration of Self-Assembly and Staged Assembly of a Bivalent and Bispecific Diabody Joining Pair

### 10.1. Demonstration of Self-Assembly

As disclosed hereinabove, staged assembly may be carried out using two non-cross-reacting diabody assembly unit constructs that are expressed and purified. Solutions of each diabody unit protein alone should remain clear, since the single diabody assembly units will not self-polymerize (i.e., self-assemble).

If the two solutions are mixed, however, the diabody units are capable of oligomerization as linked units and form long fibers in which the two diabody units alternate (FIG. 16). This self-assembly is readily observable by eye, by simple light scattering or turbidity experiments and can be readily confirmed by electron microscopy of negatively stained polymer rods.

### 10.2 Demonstration of Staged Assembly

Staged assembly is carried out by immobilizing the initiator to a sepharose solid support matrix and then contacting the matrix-bound initiator with diabody assembly unit-1. This is followed by a wash step, in which excess diabody unit-1 is removed from the bound nanostructure (containing the initiator unit and bound diabody unit-1). The nanostructure is then incubated with diabody assembly unit-2, followed by washing and incubating in the presence of additional copies of diabody assembly unit-1, etc., through a number of cycles (FIG. 2). Electron microscopy is used to determine the length and geometry of the polymers assembled through different numbers of binding and wash cycles. These lengths are precisely proportional to the number of cycles.

### 10.3. Analysis of Polymerization by Light Scattering

The extent polymerization of macromolecular monomers, such as the diabodies used in this example, may be analyzed by light scattering. Light scattering measurements from a light scattering photometer, e.g., the DAWN-DSP photometer (Wyatt Technology Corp., Santa Barbara, Calif.), provides information for determination of the weight average molecular weight, determination of particle size, shape and particle-particle pair correlations.

### 10.4. Molecular Weight Determination (Degree of Polymerization) by Sucrose Gradient Sedimentation

Linked diabody units of different lengths sediment at different rates in a sucrose gradient in zonal ultracentrifugation. The quantitative relationship between the degree of polymerization and sedimentation in Svedberg units is then calculated. This method is useful for characterizing the efficiency of self-assembly in general, as well as the process of staged assembly at each step of addition of a new diabody unit.

### 10.5. Morphology and Length of Rods by Electron Microscopy

After sucrose gradient fractionation and SDS-PAGE analysis, the partially purified fractions containing rods are apparent. Samples of the appropriate fractions are placed on EM grids and stained or shadowed to look for large structures using electron microscopy in order to determine their morphology.

### 11. EXAMPLE 6

### Staged Assembly of a Three-Dimensional Cube

This example discloses the fabrication of a three-dimensional cubic structure by staged assembly from assembly units comprising structural elements from engineered triabody and diabody fragments. The joining elements of the assembly units are the multispecific binding domains of triabodies or diabodies.

Triabodies are trivalent and make up the vertices of the cubic-like structure. Diabodies are bivalent and, in this example, two are used to construct the edges of the cubic structure, thereby spanning the space between the triabodies.

In the case of the initiator unit, an added peptide epitope is engineered as a joining element within the triabody structural element for immobilization to a solid support (and defined as the first vertex of the cube in the staged assembly). Therefore the joining elements for the triabody initiator unit comprise four non-complementary joining elements, three of which are comprised of the trispecific binding domains of the triabody and the fourth from a peptide epitope engineered within the triabody structure designed specifically to interact with solid support matrix. The peptide epitope comprised in the initiator unit can be engineered to contain a pre-designed releasing moiety (e.g. a protease site) that can be cleaved from the initiator unit and joined to the nanostructure from the solid support matrix upon complete nanofabrication of the three-dimensional nanocube. Since the three-dimensional structure of a triabody has been well characterized (Pei et al., 1997, The 2.0-Å resolution crystal structure of a trimeric antibody fragment with noncognate V_{H}-V_{L} domain pairs shows a rearrangement of V_{H} CDR3, Proc. Natl. Acad. Sci. USA 94(18): 9637-42), the insertion points within the protein structure can be identified for engineering additional joining elements, as discussed hereinabove, by visual investigation of the available X-ray coordinates.

Another triabody comprised of three trispecific binding domains as the joining elements makes up another assembly unit (the other 7 vertices of the cube). The other assembly units, namely the diabody units comprised of two bispecific binding domains as joining elements, will form the edges of the cube (edges can be defined as the vectorial lattices between defined vertices of the cube). Each edge of the cube will be fabricated from two diabody assembly units). In this example, a total of 32 assembly units are required for the nanofabrication of a three-dimensional nanocube: 8 triabodies (one initiator unit and 7 assembly units making up the 8 vertices) and 24 diabodies (all assembly units making up the 12 edges). A total of 7 non-cross-reacting, complementary joining pairs required for the fabrication of the nanocube.

Triabodies are three dimensional, equilateral triangle prism-shaped proteins that contain one joining element (CDR) at each of the three vertices. Diabodies, on the other hand, are rectangular prism shaped proteins with two opposing joining elements (CDRs). The nanofabrication of a three-dimensional (3-D) cube composed of triabodies and diabodies requires geometric and spatial relationships of the associated assembly units to be within defined design specifications of the three-dimensional cube shown in FIG. 23.

Particular geometries and spatial orientations of associated triabodies and Fab fragments have been physically characterized (Lawrence et al., 1998, Orientation of antigen binding sites in dimeric and trimeric single chain Fv antibody fragments, FEBS Lett. 425(3): 479-84). The three Fab arms, when associated to the vertices of a triabody, are not coplanar but, instead, are angled together in one direction and appear as the legs of a tripod (Lawrence et al., 1998, Orientation of antigen binding sites in dimeric and trimeric single chain Fv antibody fragments, FEBS Lett. 425(3): 479-84). The angles between adjacent Fab arms associated to the triabody was measured to be between 80-136° (i.e this falls within the required geometric and spatial relationships of the associated assembly units for the formation of a vertex associated with three edges of a cube) and that of a diabody and a Fab fragment associations was measured between 60 and 180° (this falls within the required geometric and spatial relationships for the formation of one edge of the cube upon the association (joining) of two adjacent diabody elements). The angle between planar edges of the cube is defined as 90° and that of a cubic edge as 180°. Therefore, utilizing triabodies as the vertices of a cube and diabodies as the edges, taking into consideration the limited structural flexibility inherent within antibody fragments, and the characteristic geometrical and spatial associations of antibody fragments observed, it will be possible to construct a three-dimensional cube as disclosed herein.

The cube is constructed by first identifying 7 non-cross-reacting, complementary joining element pairs. In this embodiment, idiotope/anti-idiotope pairs are constructed using standard methods disclosed above. The 14 joining elements that are elements of these pairs are incorporated into bispecific diabodies and trispecific triabodies as indicated by the architecture disclosed below. FIG. 23 is a diagram of the assembly of a cubic structure with the joining pairs indicated by letters (A being complementary to A′; B complementary to B′, etc.); and the order of assembly indicated by numbers. The first unit is the initiator unit, and it is indicated by the number ‘1’, and comprises joining elements A, B and C. The second unit (‘2’) comprises joining elements A′ and D. When a surface on which a unit 1 is immobilized is incubated with a solution containing element 2, the element will be added to the complementary binding site ‘A’ on unit 1 resulting in a nanostructure intermediate comprising units 1 and 2. After washing off excess copies of assembly unit 2, the intermediate is incubated against assembly unit 3, comprising joining elements D′ and A. This unit will bind with specificity to the complementary joining element on unit 2, resulting in a nanostructure intermediate comprising units 1, 2, and 3. This process is then continued with alternating steps of incubation and washing, until the entire structure is formed. Since 32 assembly units are added one at a time, there will be 31 steps in the assembly process (not counting the immobilization of unit 1 to a solid substrate).

A key element in planning a staged assembly of a nanostructure is the tracking of which joining elements are exposed after each step in the process. In the assembly of this nanocubic structure, the following joining elements are exposed after each step:

| Last added unit | Joining elements exposed | | | | |
|---|---|---|---|---|---|
| 1 | A | B | C | | |
| 2 | D | B | C | | |
| 3 | A | B | C | | |
| 4 | A | D | C | | |
| 5 | A | B | C | | |
| 6 | A | B | D | | |
| 7 | A | B | C | | |
| 8 | E | F | B | C | |
| 9 | E | D | B | C | |
| 10 | E | F | B | C | |
| 11 | E | F | B | A | G |
| 12 | E | F | B | D | G |
| 13 | E | F | B | A | G |
| 14 | C | E | B | G | |
| 15 | C | D | B | G | |
| 16 | C | E | B | G | |
| 17 | C | E | A | F | G |
| 18 | C | E | D | F | G |
| 19 | C | E | A | F | G |
| 20 | C | B | F | G | |
| 21 | C | D | F | G | |
| 22 | C | B | F | G | |
| 23 | D | B | F | G | |
| 24 | C | B | F | G | |
| 25 | A | F | G | | |
| 26 | A | F | D | | |
| 27 | A | F | G | | |
| 28 | A | D | G | | |
| 29 | A | F | G | | |
| 30 | D | F | G | | |
| 31 | A | F | G | | |
| 32 | — | — | — | | |

After unit 32 is added, no joining elements are exposed.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for staged assembly of a nanostructure comprising:
(a) contacting a surface-bound nanostructure intermediate comprising at least one unbound joining element with a solution comprising an assembly unit comprising a plurality of different joining elements, wherein:
(i) none of the joining elements of said plurality of different joining elements can interact with itself or with another joining element of said plurality,
(ii) a single joining element of said plurality can bind non-covalently to a single unbound joining element of the surface-bound nanostructure intermediate, and
(iii) the joining elements do not consist of or comprise T-even or T-even-like bacteriophage tail fiber proteins or binding fragments thereof;
(b) removing unbound assembly units; and
(c) repeating steps (a) and (b) to form a nanostructure.

2. The method of claim 1, wherein the surface-bound nanostructure intermediate consists essentially of an initiator assembly unit.

3. The method of claim 1, comprising the additional step of:
(d) capping the nanostructure with at least one capping unit.

4. The method of claim 1, wherein the assembly unit comprises at least one structural element covalently linked to at least one joining element.

5. The method of claim 1, wherein the assembly unit comprises at least one functional element.

6. The method of claim 4, wherein the structural element is covalently linked to a first joining element and to a second joining element, and wherein the first and second joining elements cannot bind to each other.

7. The method of claim 1, wherein non-covalent binding is specific non-covalent binding.

8. The method of claim 7, wherein specific non-covalent interactions are stabilized post-assembly by conversion to covalent linkages.

9. The method of claim 4, wherein the assembly unit comprises a first structural element that is bound to a second structural element to form a stable complex, and wherein said first structural element is covalently linked to said at least one joining element.

10. The method of claim 1, wherein the assembly unit comprises a plurality of assembly units that bind to each other to form a stable complex.

11. The method of claim 4, wherein the assembly unit comprises at least one peptide segment disposed between the structural element and the joining element.

12. The method of claim 5, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

13. The method of claim 1, wherein the joining element comprises a hapten, antigen, peptide, peptide epitope, PNA, DNA, RNA, aptamer, or polymer, or a binding derivative or binding fragment thereof.

14. The method of claim 13, wherein the peptide epitope is selected from the group consisting of SEQ ID NOS: 70-80.

15. The method of claim 4 wherein the structural element comprises a four-helix bundle.

16. The method of claim 4 wherein the structural element comprises a leucine zipper-type coiled coil domain.

17. The method of claim 16, wherein the leucine zipper-type coiled coil domain is selected from the group consisting of SEQ ID NOS: 1-69.

18. The method of claim 5, wherein a functional clement is inserted between two leucine zipper-type coiled coil domains.

19. The method of claim 17, wherein the joining element is a hapten or a PNA.

20. A nanostructure assembly unit comprising a plurality of different joining elements, wherein:
(a) none of the joining elements of said plurality can interact with itself or with another joining element of said plurality;
(b) a single joining element of said plurality can bind non-covalently to a single unbound joining element of a surface-bound nanostructure intermediate; and
(c) the joining elements do not consist of or comprise T-even or T-even-like bacteriophage tail fiber proteins or binding fragments thereof.

21. The assembly unit of claim 20, wherein the assembly unit comprises at least one structural element covalently linked to at least one joining element.

22. The assembly unit of claim 20, wherein the assembly unit comprises at least one functional element.

23. The assembly unit of claim 21, wherein the structural element is covalently linked to a first joining element and to a second joining element, and wherein the first and second joining elements cannot bind to each other.

24. The assembly unit of claim 21, wherein specific non-covalent interactions are stabilized post-assembly by conversion to covalent linkages.

25. The assembly unit of claim 21, wherein the assembly unit comprises a first structural element that is bound to a second structural element to form a stable complex, and wherein said first structural element is covalently linked to said at least one joining element.

26. The assembly unit of claim 20, wherein the assembly unit comprises a plurality of assembly units that bind to each other to form a stable complex.

27. The assembly unit of claim 26, wherein at least one of the plurality of assembly units is a capping unit.

28. The assembly unit of claim 21, wherein the assembly unit comprises at least one peptide segment disposed between the structural element and the joining element.

29. The assembly unit of claim 22, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

30. The assembly unit of claim 20, wherein the joining element comprises a hapten, antigen, peptide, peptide epitope, PNA, DNA, RNA, aptamer, or polymer, or a binding derivative or binding fragment thereof.

31. The assembly unit of claim 30, wherein the peptide epitope is selected from the group consisting of SEQ ID NOS: 70-80.

32. The assembly unit of claim 21, wherein the structural element comprises a four-helix bundle.

33. The assembly unit of claim 21, wherein the structural element comprises a leucine zipper-type coiled coil domain.

34. The assembly unit of claim 33, wherein the leucine zipper-type coiled coil domain is selected from the group consisting of SEQ ID NOS: 1-69.

35. The assembly unit of claim 22, wherein a functional element is inserted between two leucine zipper-type coiled coil domains.

36. The assembly unit of claim 32, wherein the joining element is a hapten or a PNA.

37. The method of claim 1, wherein at least one joining element comprises a binding domain of an antibody or binding derivative or binding fragment thereof.

38. The method of claim 37, wherein the surface-bound nanostructure intermediate consists essentially of an initiator assembly unit.

39. The method of claim 37, comprising the additional step of:
(d) capping the nanostructure with at least one capping unit.

40. The method of claim 37, wherein the assembly unit comprises at least one structural element covalently linked to at least one joining element.

41. The method of claim 37, wherein the assembly unit comprises at least one functional element.

42. The method of claim 40, wherein the structural element is covalently linked to a first joining element and to a second joining element, and wherein the first and second joining elements cannot bind to each other.

43. The method of claim 37, wherein non-covalent binding is specific non-covalent binding.

44. The method of claim 43, wherein specific non-covalent interactions are stabilized post-assembly by conversion to covalent linkages.

45. The method of claim 40, wherein the assembly unit comprises a first structural element that is bound to a second structural element to form a stable complex, and wherein said first structural element is covalently linked to said at least one joining element.

46. The method of claim 37, wherein the assembly unit comprises a plurality of assembly units that bind to each other to form a stable complex.

47. The method of claim 40, wherein the assembly unit comprises at least one peptide segment disposed between the structural element and the joining element.

48. The method of claim 41, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

49. The method of claim 37, wherein a joining element of said plurality or an unbound joining element of the surface-bound nanostructure intermediate comprises a monoclonal antibody domain or binding derivative or binding fragment thereof.

50. The method of claim 49, wherein the monoclonal antibody domain is a humanized monoclonal antibody domain.

51. The method of claim 37, wherein a joining element of said plurality or an unbound joining element of the surface-bound nanostructure intermediate comprises an IgG binding domain.

52. The method of claim 37, wherein a joining element of said plurality or an unbound joining element of the surface-bound nanostructure intermediate comprises a single-chain antibody domain or binding derivative or binding fragment thereof.

53. The method of claim 37, wherein a joining element of said plurality or an unbound joining element of the surface-bound nanostructure intermediate comprises a multispecific antibody domain or binding derivative or binding fragment thereof.

54. The method of claim 37, wherein a joining element of said plurality or an unbound joining element of the surface-bound nanostructure intermediate comprises a scFv.

55. The method of claim 37, wherein a joining element of said plurality or an unbound joining element of the surface-bound nanostructure intermediate comprises a Fv.

56. The method of claim 37, wherein a joining element of said plurality or an unbound joining element of the surface-bound nanostructure intermediate comprises a Fab.

57. The method of claim 37, wherein a joining element of said plurality or an unbound joining element of the surface-bound nanostructure intermediate comprises a F(ab′)₂.

58. The method of claim 37, wherein a joining element of said plurality or an unbound joining element of the surface-bound nanostructure intermediate comprises a heterologous-F(ab′)₂.

59. The method of claim 37, wherein a joining element of said plurality or an unbound joining element of the surface-bound nanostructure intermediate comprises a Fab-scFv fusion.

60. The method of claim 37, wherein a joining element of said plurality or an unbound joining element of the surface-bound nanostructure intermediate comprises a F(ab′)₂-scFv fusion.

61. The method of claim 37, wherein a joining element of said plurality or an unbound joining element of the surface-bound nanostructure intermediate comprises a CDR of an IgG.

62. The method of claim 37, wherein a joining element of said plurality or an unbound joining element of the surface-bound nanostructure intermediate is formed by a fusion of an scFv and a binding derivative of an IgG.

63. The method of claim 37, wherein a joining element of said plurality or an unbound joining element of the surface-bound nanostructure intermediate is formed by a fusion of a cytokine and a binding derivative of an IgG.

64. The method of claim 37, wherein a joining element of said plurality or an unbound joining element of the surface-bound nanostructure intermediate is formed by a fusion of a scFv and a leucine zipper.

65. The method of claim 37, wherein a joining element of said plurality or an unbound joining element of the surface-bound nanostructure intermediate is formed by a fusion of a scFv and a Rop protein.

66. The method of claim 37, wherein a joining element of said plurality or an unbound joining element of the surface-bound nanostructure intermediate comprises a binding domain derived from a diabody.

67. The method of claim 37, wherein a joining element of said plurality or an unbound joining element of the surface-bound nanostructure intermediate comprises a binding domain derived from a triabody.

68. The method of claim 37, wherein a joining element of said plurality or an unbound joining element of the surface-bound nanostructure intermediate comprises a binding domain derived from a tetrabody.

69. The method of claim 37, wherein a joining element of said plurality or an unbound joining element of the surface-bound nanostructure intermediate comprises a multimeric scFv.

70. The method of claim 41, wherein the functional element is bound to a peptide region comprised in a binding derivative or binding fragment of an IgG.

71. The method of claim 41, wherein the functional element is bound to a peptide region comprised in a diabody or binding derivative or binding fragment thereof.

72. The method of claim 41, wherein the functional element is bound to a peptide region comprised in a triabody or binding derivative or binding fragment thereof.

73. The method of claim 41, wherein the functional element is bound to a peptide region comprised in a tetrabody or binding derivative or binding fragment thereof.

74. The method of claim 37, wherein a joining element of said plurality or an unbound joining element of the surface-bound nanostructure intermediate comprises an idiotope.

75. The method of claim 74, wherein a joining element of said plurality or an unbound joining element of the surface-bound nanostructure intermediate comprises an anti-idiotope directed against the idiotope.

76. The assembly unit of claim 20 wherein at least one joining element comprises a binding domain of an antibody or binding derivative or binding fragment thereof.

77. The assembly unit of claim 76, wherein the assembly unit comprises at least one structural element covalently linked to at least one joining element.

78. The assembly unit of claim 76, wherein the assembly unit comprises at least one functional element.

79. The assembly unit of claim 77, wherein the structural element is covalently linked to a first joining element and to a second joining element, and wherein the first and second joining elements cannot bind to each other.

80. The assembly unit of claim 77, wherein specific non-covalent interactions are stabilized post-assembly by conversion to covalent linkages.

81. The assembly unit of claim 77, wherein the assembly unit comprises a first structural element that is bound to a second structural element to form a stable complex, and wherein said first structural element is covalently linked to said at least one joining element.

82. The assembly unit of claim 76, wherein the assembly unit comprises a plurality of assembly units that bind to each other to form a stable complex.

83. The assembly unit of claim 82, wherein at least one of the plurality of assembly units is a capping unit.

84. The assembly unit of claim 77, wherein the assembly unit comprises at least one peptide segment disposed between the structural element and the joining element.

85. The assembly unit of claim 78, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

86. The assembly unit of claim 76, wherein a joining element of said plurality or an unbound joining element of the surface-bound nanostructure intermediate comprises a monoclonal antibody domain or binding derivative or binding fragment thereof.

87. The assembly unit of claim 86, wherein the monoclonal antibody domain is a humanized monoclonal antibody domain.

88. The assembly unit of claim 76, wherein a joining element of said plurality or an unbound joining element of the surface-bound nanostructure intermediate comprises an IgG binding domain.

89. The assembly unit of claim 76, wherein a joining element of said plurality or an unbound joining element of the surface-bound nanostructure intermediate comprises a single-chain antibody domain or binding derivative or binding fragment thereof.

90. The assembly unit of claim 76, wherein a joining element of said plurality or an unbound joining element of the surface-bound nanostructure intermediate comprises a multispecific antibody domain or binding derivative or binding fragment thereof.

91. The assembly unit of claim 76, wherein a joining element of said plurality or an unbound joining element of the surface-bound nanostructure intermediate comprises a scFv.

92. The assembly unit of claim 76, wherein a joining element of said plurality or an unbound joining element of the surface-bound nanostructure intermediate comprises a Fv.

93. The assembly unit of claim 76, wherein a joining element of said plurality or an unbound joining element of the surface-bound nanostructure intermediate comprises a Fab.

94. The assembly unit of claim 76, wherein a joining element of said plurality or an unbound joining element of the surface-bound nanostructure intermediate comprises a F(ab′)₂.

95. The assembly unit of claim 76, wherein a joining element of said plurality or an unbound joining element of the surface-bound nanostructure intermediate comprises a heterologous-F(ab′)₂.

96. The assembly unit of claim 76, wherein a joining element of said plurality or an unbound joining element of the surface-bound nanostructure intermediate comprises a Fab-scFv fusion.

97. The assembly unit of claim 76, wherein a joining element of said plurality or an unbound joining element of the surface-bound nanostructure intermediate comprises a F(ab′)₂-scFv fusion.

98. The assembly unit of claim 76, wherein a joining element of said plurality or an unbound joining element of the surface-bound nanostructure intermediate comprises a CDR of an IgG.

99. The assembly unit of claim 76, wherein a joining element of said plurality or an unbound joining element of the surface-bound nanostructure intermediate is formed by a fusion of an scFv and a binding derivative of an IgG.

100. The assembly unit of claim 76, wherein a joining element of said plurality or an unbound joining element of the surface-bound nanostructure intermediate is formed by a fusion of a cytokine and a binding derivative of an IgG.

101. The assembly unit of claim 76, wherein a joining element of said plurality or an unbound joining element of the surface-bound nanostructure intermediate is formed by a fusion of a scFv and a leucine zipper.

102. The assembly unit of claim 76, wherein a joining element of said plurality or an unbound joining element of the surface-bound nanostructure intermediate is formed by a fusion of a scFv and a Rop protein.

103. The assembly unit of claim 76, wherein a joining element of said plurality or an unbound joining element of the surface-bound nanostructure intermediate comprises a binding domain derived from a diabody.

104. The assembly unit of claim 76, wherein a joining element of said plurality or an unbound joining element of the surface-bound nanostructure intermediate comprises a binding domain derived from a triabody.

105. The assembly unit of claim 76, wherein a joining element of said plurality or an unbound joining element of the surface-bound nanostructure intermediate comprises a binding domain derived from a tetrabody.

106. The assembly unit of claim 76, wherein a joining element of said plurality or an unbound joining element of the surface-bound nanostructure intermediate comprises a multimeric scFv.

107. The assembly unit of claim 78, wherein the functional element is bound to a peptide region comprised in a binding derivative or binding fragment of an IgG.

108. The assembly unit of claim 78, wherein the functional element is bound to a peptide region comprised in a diabody or binding derivative or binding fragment thereof.

109. The assembly unit of claim 78, wherein the functional element is bound to a peptide region comprised in a triabody or binding derivative or binding fragment thereof.

110. The assembly unit of claim 78, wherein the functional element is bound to a peptide region comprised in a tetrabody or binding derivative or binding fragment thereof.

111. The assembly unit of claim 76, wherein a joining element of said plurality or an unbound joining element of the surface-bound nanostructure intermediate comprises an idiotope.

112. The assembly unit of claim 111, wherein a joining element of said plurality or an unbound joining element of the surface-bound nanostructure intermediate comprises an anti-idiotope directed against the idiotope.

113. The method of claim 1 wherein at least one joining element comprises a pilin protein or binding derivative or binding fragment thereof.

114. The method of claim 113, wherein the surface-bound nanostructure intermediate consists essentially of an initiator assembly unit.

115. The method of claim 113, comprising the additional step of:
(d) capping the nanostructure with at least one capping unit.

116. The method of claim 113, wherein the assembly unit comprises at least one structural element covalently linked to at least one joining element.

117. The method of claim 113, wherein the assembly unit comprises at least one functional element.

118. The method of claim 116, wherein the structural element is covalently linked to a first joining element and to a second joining element, and wherein the first and second joining elements cannot bind to each other.

119. The method of claim 113, wherein non-covalent binding is specific non-covalent binding.

120. The method of claim 119, wherein specific non-covalent interactions are stabilized post-assembly by conversion to covalent linkages.

121. The method of claim 116, wherein the assembly unit comprises a first structural element that is bound to a second structural element to form a stable complex, and wherein said first structural element is covalently linked to said at least one joining element.

122. The method of claim 113, wherein the assembly unit comprises a plurality of assembly units that bind to each other to form a stable complex.

123. The method of claim 116, wherein the assembly unit comprises at least one peptide segment disposed between the structural element and the joining element.

124. The method of claim 117, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

125. The method of claim 113, wherein a joining element of said plurality or an unbound joining element of the surface-bound nanostructure intermediate comprises a pilin protein or binding derivative or binding fragment thereof.

126. The method of claim 125, wherein the pilin protein is selected from the group consisting of SEQ ID NOS: 81-90

127. The method of claim 125, wherein the pilin protein is a hybrid pilin protein.

128. The method of claim 127, wherein the hybrid pilin protein comprises an N-terminal extension sequence selected from the group consisting of SEQ ID NOS: 81, 83, 85, 87 and 89.

129. The method of claim 127, wherein the hybrid pilin protein comprises a pilin protein body sequence selected from the group consisting of SEQ ID NOS: 82, 84, 86, 88 and 90.

130. The method of claim 113, wherein a joining element of said plurality or an unbound joining element of the surface-bound nanostructure intermediate comprises a hybrid pilin protein, wherein the hybrid pilin protein comprises the pilin amino terminal extension of a first pilin protein and the pilin protein body of a second pilin protein and lacks the pilin protein body of the first pilin protein and the pilin amino terminal extension of the second pilin protein, wherein the amino terminal extension of the first pilin protein does not bind to the pilin protein body of the second pilin protein.

131. The method of claim 117, wherein the functional element is inserted at a peptide region comprised in a pilin protein or binding derivative or fragment thereof.

132. The assembly unit of claim 20 wherein at least one joining element comprises a pilin protein or binding derivative or binding fragment thereof.

133. The assembly unit of claim 132, wherein the assembly unit comprises at least one structural element covalently linked to at least one joining element.

134. The assembly unit of claim 132, wherein the assembly unit comprises at least one functional element.

135. The assembly unit of claim 133, wherein the structural element is covalently linked to a first joining element and to a second joining element, and wherein the first and second joining elements cannot bind to each other.

136. The assembly unit of claim 133, wherein specific non-covalent interactions are stabilized post-assembly by conversion to covalent linkages.

137. The assembly unit of claim 133, wherein the assembly unit comprises a first structural element that is bound to a second structural element to form a stable complex, and wherein said first structural element is covalently linked to said at least one joining element.

138. The assembly unit of claim 132, wherein the assembly unit comprises a plurality of assembly units that bind to each other to form a stable complex.

139. The assembly unit of claim 138, wherein at least one of the plurality of assembly units is a capping unit.

140. The assembly unit of claim 133, wherein the assembly unit comprises at least one peptide segment disposed between the structural element and the joining element.

141. The assembly unit of claim 134, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

142. The assembly unit of claim 132, wherein a joining element of said plurality or an unbound joining element of the surface-bound nanostructure intermediate comprises a pilin protein or binding derivative or binding fragment thereof.

143. The assembly unit of claim 142, wherein the pilin protein is selected from the group consisting of SEQ ID NOS: 81-90

144. The assembly unit of claim 142, wherein the pilin protein is a hybrid pilin protein.

145. The assembly unit of claim 142, wherein the hybrid pilin protein comprises an N-terminal extension sequence selected from the group consisting of SEQ ID NOS: 81, 83, 85, 87 and 89.

146. The assembly unit of claim 142, wherein the hybrid pilin protein comprises a pilin protein body sequence selected from the group consisting of SEQ ID NOS: 82, 84, 86, 88 and 90.

147. The assembly unit of claim 132, wherein a joining element of said plurality or an unbound joining element of the surface-bound nanostructure intermediate comprises a hybrid pilin protein, wherein the hybrid pilin protein comprises the pilin amino terminal extension of a first pilin protein and the pilin protein body of a second pilin protein and lacks the pilin protein body of the first pilin protein and the pilin amino terminal extension of the second pilin protein, wherein the amino terminal extension of the first pilin protein does not bind to the pilin protein body of the second pilin protein.

148. The assembly unit of claim 134, wherein the functional element is inserted at a peptide region comprised in a pilin protein or binding derivative or binding fragment thereof.

149. The method of claim 1, wherein at least one joining element comprises a peptide nucleic acid (hereinafter “PNA”) or binding derivative thereof.

150. The method of claim 149, wherein the surface-bound nanostructure intermediate consists essentially of an initiator assembly unit.

151. The method of claim 149, comprising the additional step of:
(d) capping the nanostructure with at least one capping unit.

152. The method of claim 149, wherein the assembly unit comprises at least one structural element covalently linked to at least one joining element.

153. The method of claim 149, wherein the assembly unit comprises at least one functional element.

154. The method of claim 152, wherein the structural element is covalently linked to a first joining element and to a second joining element, and wherein the first and second joining elements cannot bind to each other.

155. The method of claim 149, wherein non-covalent binding is specific non-covalent binding.

156. The method of claim 155, wherein specific non-covalent interactions are stabilized post-assembly by conversion to covalent linkages.

157. The method of claim 152, wherein the assembly unit comprises a first structural element that is bound to a second structural element to form a stable complex, and wherein said first structural element is covalently linked to said at least one joining element.

158. The method of claim 149, wherein the assembly unit comprises a plurality of assembly units that bind to each other to form a stable complex.

159. The method of claim 152, wherein the assembly unit comprises at least one peptide segment disposed between the structural element and the joining element.

160. The method of claim 153, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

161. The method of claim 149, wherein a joining element of said plurality or an unbound joining element of the surface-bound nanostructure intermediate comprises a PNA or binding derivative thereof.

162. The method of claim 153, wherein the functional element comprises a sequence selected from the group consisting of SEQ ID NOS:158-180.

163. The method of claim 149, wherein a joining element of said plurality or an unbound joining element of the surface-bound nanostructure intermediate comprises a PNA or binding derivative thereof that is capable of dimerizing with another PNA or binding derivative via Watson-Crick or Hoogsteen base-pairing.

164. The method of claim 149, wherein a joining element of said plurality or an unbound joining element of the surface-bound nanostructure intermediate comprises a PNA or binding derivative thereof that is capable of dimerizing with another PNA or binding derivative thereof to form a triple-helical structure.

165. The assembly unit of claim 20 wherein at least one joining element comprises a PNA or a binding derivative thereof.

166. The assembly unit of claim 165, wherein the assembly unit comprises at least one structural element covalently linked to at least one joining element.

167. The assembly unit of claim 165, wherein the assembly unit comprises at least one functional element.

168. The assembly unit of claim 166, wherein the structural element is covalently inked to a first joining element and to a second joining element, and wherein the first and second joining elements cannot bind to each other.

169. The assembly unit of claim 166, wherein specific non-covalent interactions are stabilized post-assembly by conversion to covalent linkages.

170. The assembly unit of claim 166, wherein the assembly unit comprises a first structural element that is bound to a second structural element to form a stable complex, and wherein said first structural element is covalently linked to said at least one joining element.

171. The assembly unit of claim 165, wherein the assembly unit comprises a plurality of assembly units that bind to each other to form a stable complex.

172. The assembly unit of claim 171, wherein at least one of the plurality of assembly units is a capping unit.

173. The assembly unit of claim 166, wherein the assembly unit comprises at least one peptide segment disposed between the structural element and the joining element.

174. The assembly unit of claim 167, wherein the functional element comprises a photoactive molecule, photonic nanoparticle, inorganic ion, inorganic nanoparticle, magnetic ion, magnetic nanoparticle, electronic nanoparticle, metallic nanoparticle, metal oxide nanoparticle, gold nanoparticle, gold-coated nanoparticle, carbon nanotube, nanocrystal, nanowire, quantum dot, peptide, protein, protein domain, enzyme, hapten, antigen, biotin, digoxygenin, lectin, toxin, radioactive label, fluorophore, chromophore, or chemiluminescent molecule.

175. The assembly unit of claim 165, wherein a joining element of said plurality or an unbound joining element of the surface-bound nanostructure intermediate comprises a PNA or binding derivative thereof.

176. The assembly unit of claim 167, wherein the functional element comprises a sequence selected from the group consisting of SEQ ID NOS: 158-180.

177. The assembly unit of claim 165, wherein a joining element of said plurality or an unbound joining element of the surface-bound nanostructure intermediate comprises a PNA or binding derivative thereof that is capable of dimerizing with another PNA or binding derivative via Watson-Crick or Hoogsteen base-pairing.

178. The assembly unit of claim 165, wherein a joining element of said plurality or an unbound joining element of the surface-bound nanostructure intermediate comprises a PNA or binding derivative thereof that is capable of dimerizing with another PNA or binding derivative thereof to form a triple-helical structure.
